(19) United States
(12) Patent Application Publication
(10) Pub. No.: US 2009/0143413 A1
(43) Pub. Date: Jun. 4, 2009

(54) Thiazole Derivatives as CXCR3 Receptor Modulators

(76) Inventors: Alan D. Adams, Cranford, NJ (US); Ahren I. Green, Penlyn, PA (US); Jason W. Szewczyk, New York, NY (US)

Correspondence Address: MERCK AND CO., INC, P O BOX 2000, RAHWAY, NJ, 07065-0907, US

(21) Appl. No.: 12/085,650

(22) PCT Filed: Nov. 22, 2006
(86) PCT No.: PCT/US2006/045254

§ 371 Date: May 28, 2008

Related U.S. Application Data
(60) Provisional application No. 60/740,300, filed on Nov. 29, 2005.

(51) Int. Cl. A61K 31/52; C07D 417/14; C07D 473/30; A61P 17/06; A61K 31/506

(52) U.S. Cl. 514/263.22; 544/333; 514/256; 544/276

(57) ABSTRACT

The invention encompasses compounds of Formula I (I) or pharmaceutically acceptable salts thereof, which are antagonist of the CXCR3 chemokine receptor useful for the treatment or prevention of pathogenic inflammatory processes, autoimmune diseases or graft rejection processes. Methods of use and pharmaceutical compositions are also encompassed.

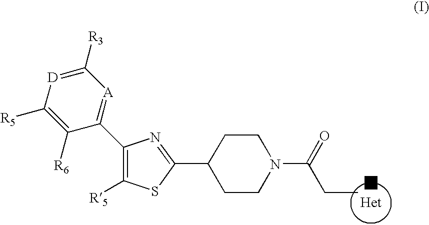

### BACKGROUND OF THE INVENTION

The chemokines are a family of small (70-120 amino acids), pro-inflammatory cytokines, with potent chemotactic activities. As their name implies, one function of chemokines, which are released by a wide variety of cells at sites of inflammation, is to attract leukocytes, including monocytes, macrophages, T lymphocytes, eosinophils, basophils and neutrophils and to promote their migration through endothelial layers. (reviewed in Schall, Cytokine, 3, 165-183 (1991) and Murphy, Rev. Immun., 12, 593-633 (1994)). In addition to their well characterized role in leukocyte trafficking, it is now also appreciated that that chemokines play a role in a number of other biological processes including cellular proliferation, hematopoiesis, angiogenesis, tumor metastasis and host defense.

These polypeptides were originally defined as having four conserved aminoterminal cysteines, and divided into two major and two minor subfamilies based on the spacing arrangement of the first cysteine pair. The two major subfamilies consist of the CXC (or α) and CC (or β) chemokines. In the CXC-chemokine family, which includes CXCL1 (MGSA or GROα), CXCL7 (NAP-2), CXCL8 (interleukin-8 or IL-8), CXCL9 (MIG), CXCL10 (IP-10) and CXCL11 (1-TAC), these two cysteines are separated by a single amino acid, while in the CC-chemokine family, which includes CCL5 (RANTES), CCL2 (monocyte chemotactic protein-1 or MCP-1), CCL8 (MCP-2), CCL7 (MCP-3), CCL3 (MIP-1a), CCL4 (MIP-11B) and CCL11 (eotaxin), these two residues are adjacent.

Some CXC-chemokines, such as CXCL1, CXCL7 and CXC1-8 are chemotactic primarily for neutrophils while another subset of CXC chemokines, including CXCL9, CXCL10 and CXCL11, are chemotactic primarily for T-lymphocytes. In comparison, the CC_chemokines, such as CCL5, CCL3, CCL4, CCL2, CCL8, CCL7 and CCL11, are more broad in their action and are chemotactic for macrophages, monocytes, T-lymphocytes, eosinophils and basophils (Deng, et al., Nature, 381, 661-666 (1996), Murphy et al. Pharmacol Revw. 52(1) 145-176, (2000).).

The chemokines bind to specific G-protein coupled receptors (GPCRs) present on leukocytes and other cells. (reviewed in Horuk, Trends Pharm. Sci., 15, 159-165 (1994), Murphy et al. Pharmacol Revw. 52(1) 145-176, (2000).) Upon interaction with their cognate ligands, chemokine receptors transduce an intracellular signal though their associated heterotrimeric G proteins, resulting in a rapid cellular responses, including an increase in intracellular calcium concentration. These chemokine receptors form a sub-family of GPCRs, which, at present, consists of a number of well characterized members with known ligands as well as a number of orphans. Unlike receptors for promiscuous classical chemoattractants such as C5a, fMLP, PAF, and LTB4, chemokine receptors are more selectively expressed on subsets of leukocytes. Thus, generation of specific chemokines provides a mechanism for recruitment of particular leukocyte subsets. The restricted expression and defined function of the chemokine receptors has focused attention on intervention in the chemokine signaling pathways as a method for highly selective intervention in pathological immunological and inflammatory processes.

Chemokine receptors, such as CCR1, CCR2A, CCR2B, CCR3, CCR4, CCR5, CXCR3, CXCR4, have been implicated as important mediators of inflammatory diseases and immunoregulatory disorders, including asthma, allergic rhinitis and atherosclerosis. They are also purported to play a role in the pathogenesis of autoimmune disorders such as rheumatoid arthritis, psoriasis, multiple sclerosis. An extensive review of the role of chemokines in disease is provided by in Seminars in Immunology., 15(1), 1-55 (2003).

A subset of chemokines are potent chemoattractants for lymphocytes. For example CXCR3 (CD183) is expressed in activated T lymphocytes, some B lymphocytes and NK cells. Expression and receptor responsiveness are both increased by activation of the T lymphocytes. The potent inflammatory cytokines CXCL10 and CXCL11 are chemoattractant for T lymphocytes and tumor infiltrating lymphocytes. The relatively restricted expression of the CXCR3 expression on these pro-inflammatory cell types mark CXCR3 as a very promising target for selective intervention in the inflammatory process. A connection with disease processes, particularly Th-1 mediated processes, is indicated by the presence of the CXCR3 on most activated T lymphocytes within inflamed joint synovium in rheumatoid arthritis as well as within inflamed tissue present in other inflammatory disorders including ulcerative colitis, Graves' disease, MS and rejecting graft tissues. (Qin, J. Clin. Invest., 101(4), 746-754 (1998), Garcia-Lopez, Lab. Investig. 81(3), 409-418 (2001), Balashov, PNAS, 96, 6873-6878 (1999), DeVries, Seminars in Immunology, 15(1), 33-48 (2003)) A similar but somewhat less pronounced association is shown with the CCR5 receptor and its ligand CCL5

Accordingly, agents which inhibit or modulate the function of chemokine receptors such as the CXCR3 receptor would be useful in treating or preventing such disorders and diseases. Data from animal models of inflammation further supports the hypothesis regarding the effectiveness of chemokine blockade, specifically CXCR3 inhibition, in diseases with clear T-lymphocyte mediated tissue damage such as transplant rejection, graft versus host disease, multiple sclerosis, optic neuritis and rheumatoid or psoriatic arthritis Many other diseases are characterized by T lymphocyte infiltrates, and by inference are therefore also good candidates for interventions which prevent the migration of T lymphocytes. These diseases include psoriasis and other chronic inflammatory diseases of the skin such as atopic dermatitis, lichen planus and bullous pemphigoid, inflammatory bowel diseases such as ulcerative colitis and Crohn's disease and autoimmune diseases such as systemic and cutaneous lupus erythematosus, Behcet's disease, type I diabetes or Graves' disease.

Many inflammatory lung diseases such as chronic obstructive pulmonary disease, hypersensitivity pneumonitis, chronic eosinophilic pneumonia, pulmonary sarcoidosis, bronchiolitis obliterans syndrome, asthma, kidney diseases such as glomerulonephritis, pathogenesis of chronic HCV infection and atherosclerosis show a dependence on T lymphocytes and are promising targets for agents which modulate the function of chemokine receptors such as the CXCR3 receptor.

The expression of CXCR3 in some B cell tumors indicates that intervention in CXCR3 function could have beneficial effects in these cancers, particularly in suppressing metastasis.

Several methods are under investigation for modulation of chemokine receptor function. These include antibodies binding to and neutralizing the chemokine ligands, antibodies binding to and modulating the function of the chemokine receptors and small molecules which bind to and inhibit function of the chemokine receptor. The ideal method for intervention in CXCR3 mediated chemotaxis is the binding of orally bioavailable small molecules which prevent the function of the receptor. Molecules with affinity for the CXCR3 chemokine receptor and ability to modulate the function of the receptor are described here.

### SUMMARY OF THE INVENTION

The invention encompasses compounds of Formula I

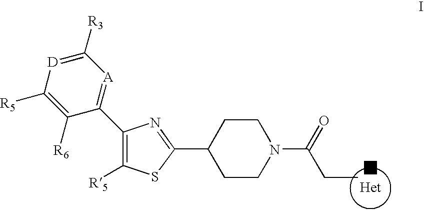

or pharmaceutically acceptable salts thereof, which are modulators of the CXCR3 chemokine receptor function useful for the treatment or prevention of pathogenic inflammatory processes, auto immune diseases or graft rejection processes. Methods of use and pharmaceutical compositions are also encompassed.

### DETAILED DESCRIPTION OF THE INVENTION

The invention encompasses a genus of compounds of Formula I

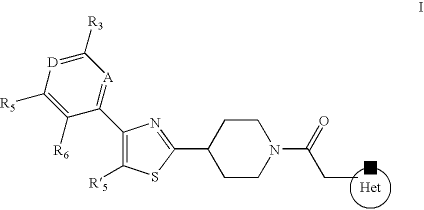

or a pharmaceutically acceptable salt thereof, wherein:

### A is CH or N;

### D is CR₄ or N;

R₃ is selected from the group consisting of: C₁₋₄alkyl, C₃₋₆cycloalkyl, —CF₃, —OCF₃ and —S(O)ₙCF₃, wherein n is 0 or 2;
R₄ is selected from the group consisting of: —H, —OH, —OCH₃, —OCH₂CF₃ and —CF₃;
or R₃ and R₄ may be joined together with the carbon atoms to which they are attached to form a five- or six-membered monocyclic ring, said rings tetra-substituted with methyl groups as follows:

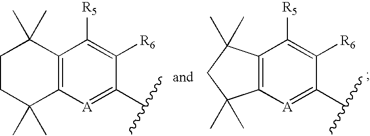

R₅ is selected from the group consisting of: —H, C₁₋₄alkyl, C₃₋₆cycloalkyl, CF₃, —Br, —CF₂CH₃, —OCF₃ and —SCF₃;
R₆ is selected from the group consisting of —H and —OCH₃,
or R₅ and R₆ may be joined together with the carbon atoms to which they are attached to form a monocyclic 5-membered ring, said ring di-substituted with methyl as follows:

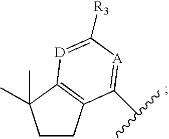

R′₅ is selected from the group consisting of: H, Cl, Br and CH₃; and

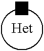

is selected from the group consisting of:

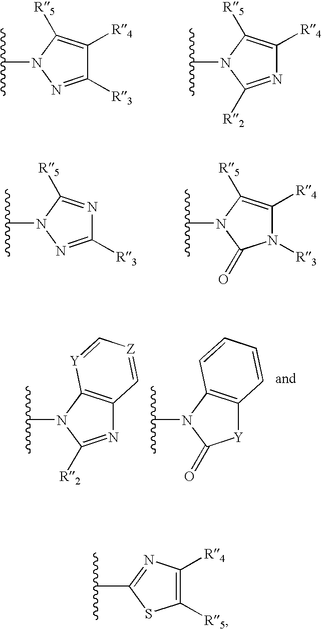

wherein Y and Z are independently C or N, and
R″₂, R″₃, R″₄ and R″₅ are selected from the group consisting of: —H, carboxy, —CF₃, halo and C₁₋₃alkyl optionally substituted with carboxy.

Within the genus, the invention encompasses a sub-genus of compounds of Formula I wherein A and D are both N.

Also within the genus, the invention encompasses a sub-genus of compounds of Formula I wherein A is CH and D is CR₄.

Within this sub-genus, the invention encompasses a class of compounds of Formula I wherein

### R₅ and R₆ are —H; and

R₃ and R₄ are joined together with the carbon atoms to which they are attached to form a six-membered monocyclic ring, said ring tetra-substituted with methyl groups as follows:

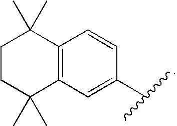

Also within this sub-genus, the invention encompasses a class of compounds of Formula I wherein:

R₃ is selected from the group consisting of: C₁₋₄alkyl, C₃₋₆cycloalkyl, —CF₃, —OCF₃ and —S(O)ₙCF₃, wherein n is 0 or 2;
R₄ is selected from the group consisting of: —H, —OH, —OCH₃, —OCH₂CF₃ and —CF₃;
R₅ is selected from the group consisting of: C₁₋₄alkyl, C₃₋₆cycloalkyl, CF₃, —Br, —CF₂CH₃, —OCF₃ and —SCF₃; and

### R₆ is —H.

Within this class, the invention encompasses a sub-class of compounds of Formula I wherein

R₃ is selected from the group consisting of: tert-butyl, —CF₃, —OCF₃ and —S(O)ₙCF₃, wherein n is 0 or 2; and
R₅ is selected from the group consisting of: tert-butyl, cyclopropyl, 1-methylcyclopropyl, CF₃, —Br, —CF₂CH₃, —OCF₃ and —SCF₃.

In another embodiment, the invention encompasses a sub-genus of compounds of Formula I within the genus wherein R₆ is —H.

In another embodiment, the invention encompasses a sub-genus of compounds of Formula I within the genus wherein:

### A is CH;

### D is CR₄;

R₃ is selected from the group consisting of: tert-butyl, —CF₃, —OCF₃ and —S(O)ₙCF₃, wherein n is 0 or 2;
R₄ is selected from the group consisting of: —H, —OH, —OCH₃, —OCH₂CF₃ and —CF₃;
or R₃ and R₄ may be joined together with the carbon atoms to which they are attached to form a six-membered monocyclic ring, said ring tetra-substituted with methyl groups as follows:

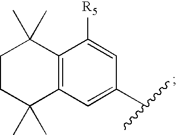

R₅ is —H when R₃ and R₄ are joined together to form a six-membered monocyclic ring, otherwise R₅ is selected from the group consisting of: tert-butyl, cyclopropyl, 1-methylcyclopropyl, CF₃, —Br, —CF₂CH₃, —OCF₃ and —SCF₃; and

### R₆ is —H.

Within this sub-genus, the invention encompasses class of compounds of Formula I wherein: R₃ and R₄ are joined together with the carbon atoms to which they are attached to form a six-membered monocyclic ring, said ring tetra-substituted with methyl groups as follows:

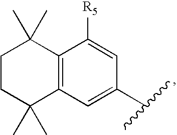

wherein R5 is —H.

Also within this sub-genus, the invention encompasses a class of compounds of Formula I wherein:

(1) R₃ is tert-butyl, R₄ is —H and R₅ is tert-butyl;
(2) R₃ is tert-butyl, R₄ is —OCH₃ and R₅ is tert-butyl;
(3) R₃ is —SCF₃, R₄ is —H and R₅ is tert-butyl;
(4) R₃ is —SO₂CF₃, R₄ is —H and R₅ is tert-butyl; or

### (5) R₃ is —OCF₃, R₄ is —CF₃ and R₅ is Br.

Also within this sub-genus, the invention encompasses a class of compounds of Formula I wherein R′₅ is —H.

Also within this sub-genus, the invention encompasses a class of compounds of Formula I wherein R′₅ is CH₃.

Also within this sub-genus, the invention encompasses a class of compounds of Formula I wherein R′₅ is Cl or Br.

Also within this sub-genus, the invention encompasses a class of compounds of Formula I wherein

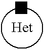

is selected from the group consisting of:

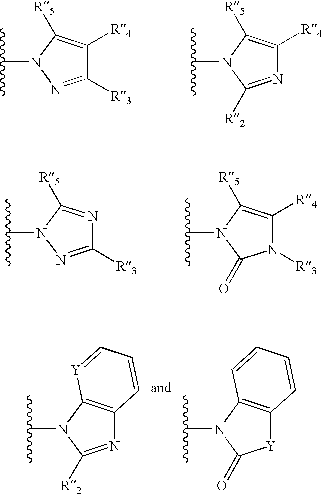

Within this class, the invention encompasses a sub-class of compounds of Formula I wherein:

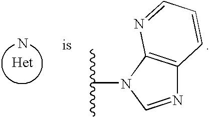

In another embodiment, the invention encompasses a compound selected from the following group:
(1) 3-{2-[4-(4-{3-tert-butyl-5-[(trifluoromethyl)thio]phenyl}-5-chloro-1,3-thiazol-2-yl)piperidin-1-yl]-2-oxoethyl}-3H-imidazo[4,5-b]pyridine;
(2) 3-{2-[4-(5-bromo-4-{3-tert-butyl-5-[(trifluoromethyl)thio]phenyl}-1,3-thiazol-2-yl)piperidin-1-yl]-2-oxoethyl}-3H-imidazo[4,5-b]pyridine;
(3) 3-{2-[4-(4-{3-tert-butyl-5-[(trifluoromethyl)sulfonyl]phenyl}-5-chloro-1,3-thiazol-2-yl)piperidin-1-yl]-2-oxoethyl}-3H-imidazo[4,5-b]pyridine;
(4) 3-{2-[4-(5-bromo-4-{3-tert-butyl-5-[(trifluoromethyl)sulfonyl]phenyl}-1,3-thiazol-2-yl)piperidin-1-yl]-2-oxoethyl}-3H-imidazo[4,5-b]pyridine;
(5) 3-(2-{4-[5-bromo-4-(3,5-di-tert-butyl-4-methoxyphenyl)-1,3-thiazol-2-yl]piperidin-1-yl}-2-oxoethyl)-3H-imidazo[4,5-b]pyridine;
(6) 3-(2-{4-[5-chloro-4-(3,5-di-tert-butyl-4-methoxyphenyl)-1,3-thiazol-2-yl]piperidin-1-yl}-2-oxoethyl)-3H-imidazo[4,5-b]pyridine;
(7) 3-[2-(4-{4-[3,5-bis(trifluoromethyl)phenyl]-5-bromo-1,3-thiazol-2-yl}piperidin-1-yl)-2-oxoethyl]-3H-imidazo[4,5-b]pyridine;
(8) 3-[2-(4-{4-[3,5-bis(trifluoromethyl)phenyl]-5-chloro-1,3-thiazol-2-yl}piperidin-1-yl)-2-oxoethyl]-3H-imidazo[4,5-b]pyridine;
(9) 3-[2-(4-{4-[3,5-bis(trifluoromethyl)phenyl]-5-fluoro-1,3-thiazol-2-yl}piperidin-1-yl)-2-oxoethyl]-3H-imidazo[4,5-b]pyridine;
(10) 3-(2-{4-[4-(3-bromo-5-tert-butylphenyl)-5-chloro-1,3-thiazol-2-yl]piperidin-1-yl}-2-oxoethyl)-3H-imidazo[4,5-b]pyridine;
(11) 3-(2-{4-[4-(3-tert-butyl-5-cyclopropylphenyl)-5-chloro-1,3-thiazol-2-yl]piperidin-1-yl}-2-oxoethyl)-3H-imidazo[4,5-b]pyridine;
(12) 3-(2-{4-[5-bromo-4-(3-tert-butyl-5-cyclopropylphenyl)-1,3-thiazol-2-yl]piperidin-1-yl}-2-oxoethyl)-3H-imidazo[4,5-b]pyridine;
(13) 4-{5-bromo-2-[1-(3H-imidazo[4,5-b]pyridin-3-ylacetyl)piperidin-4-yl]-1,3-thiazol-4-yl}-2,6-di-tert-butylphenol;
(14) 3-[2-(4-{4-[3-bromo-5-(trifluoromethoxy)-4-(trifluoromethyl)phenyl]-5-chloro-1,3-thiazol-2-yl}piperidin-1-yl)-2-oxoethyl]-3H-imidazo[4,5-b]pyridine;
(15) 3-[2-(4-{5-chloro-4-[3-cyclopropyl-5-(trifluoromethoxy)-4-(trifluoromethyl)phenyl]-1,3-thiazol-2-yl}piperidin-1-yl)-2-oxoethyl]-3H-imidazo[4,5-b]pyridine;
(16) 3-[2-(4-{5-chloro-4-[3-cyclopropyl-5-(trifluoromethoxy)phenyl]-1,3-thiazol-2-yl}piperidin-1-yl)-2-oxoethyl]-3H-imidazo[4,5-b]pyridine;
(17) 3-[2-(4-{5-chloro-4-[3-(1,1-difluoroethyl)-5-(trifluoromethoxy)phenyl]-1,3-thiazol-2-yl}piperidin-1-yl)-2-oxoethyl]-3H-imidazo[4,5-b]pyridine;
(18) 3-[2-(4-{4-[3-tert-butyl-5-(trifluoromethoxy)phenyl]-5-chloro-1,3-thiazol-2-yl}piperidin-1-yl)-2-oxoethyl]-3H-imidazo[4,5-b]pyridine;
(19) 3-(2-{4-[5-bromo-4-(3,5-di-tert-butylphenyl)-1,3-thiazol-2-yl]piperidin-1-yl}-2-oxoethyl)-3H-imidazo[4,5-b]pyridine;
(20) 3-{2-[4-(4-{3-bromo-5-[(trifluoromethyl)sulfonyl]phenyl}-5-chloro-1,3-thiazol-yl)piperidin-1-yl]-2-oxoethyl}-3H-imidazo[4,5-b]pyridine;
(21) 3-[2-(4-{4-[3-bromo-4-methoxy-5-(trifluoromethyl)phenyl]-5-chloro-1,3-thiazol-2-yl}piperidin-1-yl)-2-oxoethyl]-3H-imidazo[4,5-b]pyridine;
(22) 3-(2-{4-[4-(6-tert-butyl-1,1-dimethyl-2,3-dihydro-1H-inden-4-yl)-5-chloro-1,3-thiazol-2-yl]piperidin-1-yl}-2-oxoethyl)-3H-imidazo[4,5-b]pyridine;
(23) 3-[2-(4-{4-[3-bromo-4-methoxy-5-(1-methylcyclopropyl)phenyl]-5-chloro-1,3-thiazol-2-yl}piperidin-1-yl)-2-oxoethyl]-3H-imidazo[4,5-b]pyridine;
(24) 3-(2-{4-[5-chloro-4-(2,6-di-tert-butylpyrimidin-4-yl)-1,3-thiazol-2-yl]piperidin-1-yl}-2-oxoethyl)-3H-imidazo[4,5-b]pyridine;
(25) 3-[2-(4-{4-[2-tert-butyl-6-(trifluoromethyl)pyrimidin-4-yl]-5-chloro-1,3-thiazol-2-yl}piperidin-1-yl)-2-oxoethyl]-3H-imidazo[4,5-b]pyridine;
(26) 3-[2-(4-{4-[3-bromo-4-methoxy-5-(trifluoromethoxy)phenyl]-5-chloro-1,3-thiazol-2-yl}piperidin-1-yl)-2-oxoethyl]-3H-imidazo[4,5-b]pyridine;
(27) 3-[2-(4-{5-chloro-4-[3-cyclopropyl-4-methoxy-5-(trifluoromethoxy)phenyl]-1,3-thiazol-2-yl}piperidin-1-yl)-2-oxoethyl]-3H-imidazo[4,5-b]pyridine;
(28) 3-{2-[4-(4-{3-bromo-4-methoxy-5-[(trifluoromethyl)thio]phenyl}-5-chloro-1,3-thiazol-2-yl)piperidin-1-yl]-2-oxoethyl}-3H-imidazo[4,5-b]pyridine;
(29) 3-[2-(4-{5-bromo-4-[6-tert-butyl-2-(1-methylcyclopropyl)pyrimidin-4-yl]-1,3-thiazol-2-yl}piperidin-1-yl)-2-oxoethyl]-3H-imidazo[4,5-b]pyridine;
(30) 3-(2-{4-[5-chloro-4-(5,5,8,8-tetramethyl-5,6,7,8-tetrahydronaphthalen-2-yl)-1,3-thiazol-2-yl]piperidin-1-yl}-2-oxoethyl)-3H-imidazo[4,5-b]pyridine;
(31) 3-(2-{4-[5-bromo-4-(5,5,8,8-tetramethyl-5,6,7,8-tetrahydronaphthalen-2-yl)-1,3-thiazol-2-yl]piperidin-1-yl}-2-oxoethyl)-3H-imidazo[4,5-b]pyridine;
(32) [1-(2-{4-[5-chloro-4-(3,5-di-tert-butyl-4-methoxyphenyl)-1,3-thiazol-2-yl]piperidin-1-yl}-2-oxoethyl)-5-methyl-1H-1,2,4-triazol-3-yl]acetic acid;
(33) [1-(2-{4-[5-chloro-4-(3,5-di-tert-butyl-4-methoxyphenyl)-1,3-thiazol-2-yl]piperidin-1-yl}-2-oxoethyl)-5-methyl-1H-pyrazol-3-yl]acetic acid;
(34) {1-[2-(4-{4-[3,5-bis(trifluoromethyl)phenyl]-5-chloro-1,3-thiazol-2-yl}piperidin-1-yl)-2-oxoethyl]-5-methyl-1H-pyrazol-3-yl}acetic acid;
(35) [1-(2-{4-[5-bromo-4-(3,5-di-tert-butyl-4-methoxyphenyl)-1,3-thiazol-2-yl]piperidin-1-yl}-2-oxoethyl)-5-methyl-1H-pyrazol-3-yl]acetic acid;
(36) [1-(2-{4-[5-bromo-4-(3,5-di-tert-butyl-4-hydroxyphenyl)-1,3-thiazol-2-yl]piperidin-1-yl}-2-oxoethyl)-5-methyl-1H-pyrazol-3-yl]acetic acid;
(37) [4-bromo-1-(2-{4-[5-chloro-4-(3,5-di-tert-butyl-4-methoxyphenyl)-1,3-thiazol-2-yl]piperidin-1-yl}-2-oxoethyl)-5-methyl-1H-pyrazol-3-yl]acetic acid;
(38) 3-[1-(2-{4-[5-chloro-4-(3,5-di-tert-butyl-4-methoxyphenyl)-1,3-thiazol-2-yl]piperidin-1-yl}-2-oxoethyl)-5-methyl-1-pyrazol-3-yl]propanoic acid;
(39) 3-{2-[4-(5-chloro-4-{3-(trifluoromethoxy)-5-[(trifluoromethyl)thio]phenyl}-1,3-thiazol-2-yl)piperidin-1-yl]-2-oxoethyl}-3H-imidazo[4,5-b]pyridine;
(41) [2-(2-{4-[5-chloro-4-(3,5-di-tert-butyl-4-methoxyphenyl)-1,3-thiazol-2-yl]piperidin-1-yl}-2-oxoethyl)-1,3-thiazol-4-yl]acetic acid;
(42) [2-(2-{4-[5-chloro-4-(3,5-di-tert-butyl-4-methoxyphenyl)-1,3-thiazol-2-yl]piperidin-1-yl}-2-oxoethyl)-5-methyl-1,3-thiazol-4-yl]acetic acid;
(43) [1-(2-{4-[5-chloro-4-(3,5-di-tert-butyl-4-methoxyphenyl)-1,3-thiazol-2-yl]piperidin-1-yl}-2-oxoethyl)-4,5-dimethyl-1H-pyrazol-3-yl]acetic acid;
(44) [2-(2-{4-[5-bromo-4-(3,5-di-tert-butyl-4-methoxyphenyl)-1,3-thiazol-2-yl]piperidin-1-yl}-2-oxoethyl)-5-methyl-1,3-thiazol-4-yl]acetic acid;
(45) 7-(2-{4-[5-chloro-4-(3,5-di-tert-butyl-4-methoxyphenyl)-1,3-thiazol-2-yl]piperidin-1-yl}-2-oxoethyl)-7H-purin-6-ol;
(46) 1-(2-{4-[5-chloro-4-(3,5-di-tert-butyl-4-methoxyphenyl)-1,3-thiazol-2-yl]piperidin-1-yl}-2-oxoethyl)-1,3-dihydro-2H-indol-2-one;
(47) [1-(2-{4-[5-chloro-4-(3,5-di-tert-butylphenyl)-1,3-thiazol-2-yl]piperidin-1-yl}-2-oxoethyl)-5-methyl-1H-pyrazol-3-yl]acetic acid;
(48) [1-(2-{4-[5-chloro-4-(3,5-di-tert-butyl-4-hydroxyphenyl)-1,3-thiazol-2-yl]piperidin-1-yl}-2-oxoethyl)-5-methyl-1H-pyrazol-3-yl]acetic acid;
(49) (1-{2-[4-(4-{3-tert-butyl-5-[(trifluoromethyl)thio]phenyl}-5-chloro-1,3-thiazol-2-yl)piperidin-1-yl]-2-oxoethyl}-5-methyl-1H-pyrazol-3-yl)acetic acid;
(50) 1-(2-{4-[5-chloro-4-(3,5-di-tert-butyl-4-methoxyphenyl)-1,3-thiazol-2-yl]piperidin-1-yl}-2-oxoethyl)-1,3-dihydro-2H-benzimidazol-2-one;
(51) (2-{2-[4-(4-{3-tert-butyl-5-[(trifluoromethyl)thio]phenyl}-5-chloro-1,3-thiazol-2-yl)piperidin-1-yl]-2-oxoethyl}-1,3-thiazol-4-yl)acetic acid;
(52) (2-{2-[4-(4-{3-tert-butyl-5-[(trifluoromethyl)thio]phenyl}-5-chloro-1,3-thiazol-2-yl)piperidin-1-yl]-2-oxoethyl}-4-methyl-1,3-thiazol-4-yl)acetic acid;
(53) [2-(2-{4-[5-chloro-4-(3,5-di-tert-butyl-4-methoxyphenyl)-1,3-thiazol-2-yl]piperidin-1-yl}-2-oxoethyl)-5-methyl-1,3-thiazol-4-yl](difluoro)acetic acid;
(54) {2-[2-(4-{4-[3-bromo-5-(trifluoromethoxy)-4-(trifluoromethyl)phenyl]-5-chloro-1,3-thiazol-2-yl}piperidin-1-yl)-2-oxoethyl]-5-methyl-1,3-thiazol-4-yl}acetic acid;
(55) {1-[2-(4-{4-[3-bromo-5-(trifluoromethoxy)-4-(trifluoromethyl)phenyl]-5-chloro-1,3-thiazol-2-yl}piperidin-1-yl)-2-oxoethyl]-5-methyl-1H-pyrazol-3-yl}acetic acid;
(56) 1-{2-[4-(4-{3-tert-butyl-5-[(trifluoromethyl)thio]phenyl}-5-chloro-1,3-thiazol-2-yl)piperidin-1-yl]-2-oxoethyl}-1,3-dihydro-2H-benzimidazol-2-one;
(57) 1-{2-[4-(5-bromo-4-{3-tert-butyl-5-[(trifluoromethyl)thio]phenyl}-1,3-thiazol-2-yl)piperidin-1-yl]-2-oxoethyl}-1,3-dihydro-2H-benzimidazol-2-one; and
(58) (1-{2-[4-(5-bromo-4-{3-tert-butyl-5-[(trifluoromethyl)thio]phenyl}-1,3-thiazol-2-yl)piperidin-1-yl]-2-oxoethyl}-5-methyl-1H-pyrazol-3-yl)acetic acid;
(59) [1-(2-{4-[5-chloro-4-(3,5-di-tert-butyl-4-methoxyphenyl)-1,3-thiazol-2-yl]piperidin 1-yl}-2-oxoethyl)-4-iodo-5-methyl-1H-pyrazol-3-yl]acetic acid;
(60) 3-(2-{4-[5-chloro-4-(2,6-di-tert-butylpyrimidin-4-yl)-1,3-thiazol-2-yl]piperidin-1-yl}-2-oxoethyl)-3H-imidazo[4,5-b]pyridine;
(61) 1-(2-{4-[5-chloro-4-(2,6-di-tert-butylpyrimidin-4-yl)-1,3-thiazol-2-yl]piperidin-1-yl}-2-oxoethyl)-1,3-dihydro-2H-benzimidazol-2-one;
(62) [1-(2-{4-[5-chloro-4-(2,6-di-tert-butylpyrimidin-4-yl)-1,3-thiazol-2-yl]piperidin-1-yl}-2-oxoethyl)-5-methyl-1H-pyrazol-3-yl]acetic acid;
(63) 1-(2-{4-[5-bromo-4-(2,6-di-tert-butylpyrimidin-4-yl)-1,3-thiazol-2-yl]piperidin-1-yl}-2-oxoethyl)-1,3-dihydro-2H-benzimidazol-2-one;
(64) 3-[5-methyl-1-(oxo-2-{4-[4-(5,5,8,8-tetramethyl-5,6,7,8-tetrahydronaphthalen-2-yl)-1,3-thiazol-2-yl]piperidin-1-yl}ethyl)-1H-pyrazol-3-yl]propanoic acid;
(65) [5-methyl-1-(2-oxo-2-{4-[4-(5,5,8,8-tetramethyl-5,6,7,8-tetrahydronaphthalen-2-yl)-1,3-thiazol-2-yl]piperidin-1-yl}ethyl)-1H-pyrazol-3-yl]acetic acid;
(66) 3-(2-{4-[4-(2,6-di-tert-butylpyrimidin-4-yl)-1,3-thiazol-2-yl]piperidin-1-yl}-2-oxoethyl)-3H-imidazo[4,5-b]pyridine;
(67) 3-[2-(4-{4-[2-tert-butyl-6-(trifluoromethyl)pyrimidin-4-yl]-1,3-thiazol-2-yl}piperidin-1-yl)-2-oxoethyl]-3H-imidazo[4,5-b]pyridine;
(68) 3-[2-(4-{4-[3,5-bis(trifluoromethyl)phenyl]-1,3-thiazol-2-yl}piperidin-1-yl)-2-oxoethyl]-3H-imidazo[4,5-b]pyridine;
(69) 3-[2-(4-{4-[3-bromo-4-methoxy-5-(trifluoromethoxy)phenyl]-1,3-thiazol-2-yl}piperidin-1-yl)-2-oxoethyl]-3H-imidazo[4,5-b]pyridine;
(70) [2-(2-{4-[4-(3,5-di-tert-butyl-4-methoxyphenyl)-1,3-thiazol-2-yl]piperidin-1-yl}-2-oxoethyl)-5-methyl-1,3-thiazol-4-yl]acetic acid;
(71) 3-[2-(4-{4-[2-tert-butyl-6-(1-methylcyclopropyl)pyrimidin-4-yl]-1,3-thiazol-2-yl}piperidin-1-yl)-2-oxoethyl]-3H-imidazo[4,5-b]pyridine;
(72) [1-(2-{4-[4-(2,6-di-tert-butylpyrimidin-4-yl)-1,3-thiazol-2-yl]piperidin-1-yl}-2-oxoethyl)-5-methyl-1H-pyrazol-3-yl]acetic acid;
(73) 3-(2-{4-[4-(3,5-di-tert-butyl-4-methoxyphenyl)-1,3-thiazol-2-yl]piperidin-1-yl}-2-oxoethyl)-3H-imidazo[4,5-b]pyridine;
(74) 3-[2-(4-{4-[6-tert-butyl-2-(1-methylcyclopropyl)pyrimidin-4-yl]-1,3-thiazol-2-yl}piperidin-1-yl)-2-oxoethyl]-3H-imidazo[4,5-b]pyridine;
(75) 3-(2-{4-[4-(3,5-di-tert-butylphenyl)-1,3-thiazol-2-yl]piperidin-1-yl}-2-oxoethyl)-3H-imidazo[4,5-b]pyridine;
(76) 3-(2-oxo-2-{4-[4-(5,5,8,8-tetramethyl-5,6,7,8-tetrahydronaphthalen-2-yl)-1,3-thiazol-2-yl]piperidin-1-yl}ethyl)-3H-imidazo[4,5-b]pyridine.
   or a pharmaceutically acceptable salt of any of the above.

The invention also encompasses a pharmaceutical composition comprising a compound of Formula I in combination with a pharmaceutically acceptable carrier.

In another embodiment, the invention encompasses a method for treating a disease or condition mediated by the CXCR3 chemokine receptor comprising administering to a patient in need of such treatment a therapeutically effective amount of a compound of Formula I. Within this embodiment, the invention encompasses the above mentioned method wherein the disease or condition is selected from the group consisting of: acute and chronic transplant rejection, psoriasis, rheumatoid arthritis and multiple sclerosis.

For purposes of this specification, the below terms have the indicated meaning as follows:

The term “halogen” or “halo” includes F, Cl, Br, and I.

The term “alkyl” means linear or branched structures and combinations thereof, having the indicated number of carbon atoms. Thus, for example, C₁₋₆alkyl includes methyl, ethyl, propyl, 2-propyl, s- and t-butyl, butyl, pentyl, hexyl and 1,1-dimethylethyl.

The term “cycloalkyl” means mono-, bi- or tri-cyclic structures, optionally combined with linear or branched structures, having the indicated number of carbon atoms. Examples of cycloalkyl groups include cyclopropyl, cyclopentyl, cycloheptyl, adamantyl, cyclododecylmethyl, 2-ethyl-1-bicyclo[4.4.0]decyl, cyclobutylmethyl, cyclopropylmethyl 1-methylcyclopropyl and the like.

### Optical Isomers—Diastereomers—Geometric Isomers—Tautomers

Compounds of Formula I may contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Some of the compounds described herein may exists as mixtures of tautomers. The term “tautomers” embraces the standard meaning of the term, i.e. a type of isomerism in which two or more isomers are rapidly interconverted so that they ordinarily exist together in equilibrium. Tautomers include, e.g., compounds that undergo facile proton shifts from one atom of the compound to another atom of the compound. Some of the compounds described herein may exist as tautomers with different points of attachment of hydrogen. Such an example might be a ketone and its enol form known as keto-enol tautomers or an amide and its hydroxy imine tautomer. The individual tautomers of the compounds of Formula I, as well as mixtures thereof, are included in the scope of this invention. By way of illustration, tautomers included in this definition include, but are not limited to:

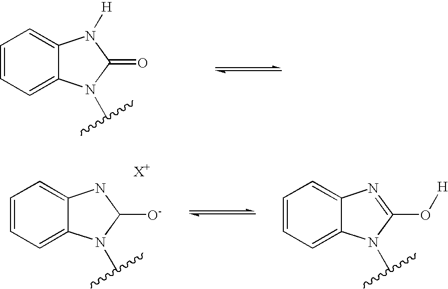

The term “rac” means racemic mixture, which is defined as a mixture comprised of equal amounts of enantiomers. If desired, racemic mixtures of compounds of Formula I may be separated by the coupling of a racemic mixture of the compounds of Formula I to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage and removal of the added chiral residue. The racemic mixture of the compounds of Formula I can also be separated directly by chromatographic methods utilizing chiral stationary phases, which methods are well known in the art.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Utilities

The compounds of the present invention are modulators of CXCR3 chemokine receptor function and are of use in antagonizing chemokine mediated cell signalling and in particular are of use in the prophylaxis and/or treatment of diseases or disorders involving inappropriate T-cell trafficking. The invention extends to such a use and to the use of the compounds of Formula I for the manufacture of a medicament for treating such diseases and disorders. Particular diseases include inflammatory, autoimmune and immunoregulatory disorders.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases or conditions of humans or other species which can be treated with compounds of Formula I, include, but are not limited to: autoimmune mediated inflammatory or allergic diseases and conditions, including respiratory diseases such as asthma, particularly bronchial asthma, systemic lupus erythematosus, ankylosing spondylitis, systemic sclerosis, autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; acute and chronic graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis); vasculitis (e.g., necrotizing, cutaneous, and hypersensitivity vasculitis); cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, and polymyositis.

The compounds of the present invention are accordingly useful in treating, preventing, ameliorating, controlling or reducing the risk of a wide variety of inflammatory and immunoregulatory disorders and diseases as well as autoimmune pathologies. In a specific embodiment, the present invention is directed to the use of the subject compounds for treating, preventing, ameliorating, controlling or reducing the risk of autoimmune diseases, such as rheumatoid arthritis, multiple sclerosis, psoriasis or psoriatic arthritis.

In another aspect, the instant invention may be used to evaluate putative specific agonists or antagonists of chemokine receptors, including CXCR3. Accordingly, the present invention is directed to the use of these compounds in the preparation and execution of screening assays for compounds which modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CXCR3. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for treating CXCR3 mediated diseases in humans and animals comprising combining a compound of the present invention with a pharmaceutical carrier or diluent.

In a preferred aspect of the present invention, a subject compound may be used in a method of inhibiting the binding of a chemokine to a chemokine receptor, such as CXCR3, of a target cell, which comprises contacting the target cell with an amount of the compound which is effective at inhibiting the binding of the chemokine to the chemokine receptor.

The subject treated in the methods above is a mammal, preferably a human being, male or female, in whom modulation of chemokine receptor activity is desired. “Modulation” as used herein is intended to encompass antagonism, agonisin, partial antagonism, inverse agonism and/or partial agonism. In a preferred aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

### Dose Ranges

The magnitude of prophylactic or therapeutic dose of a compound of Formula I will, of course, vary with the nature and severity of the condition to be treated, and with the particular compound of Formula I used and its route of administration. The dose will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.01 mg to about 25 mg (preferably from 0-1 mg to about 10 mg) of a compound of Formula I per kg of body weight per day.

In the case where an oral composition is employed, a suitable dosage range is, e.g. from about 0.01 mg to about 100 mg of a compound of Formula I per kg of body weight per day, preferably from about 0.1 mg to about 10 mg per kg.

For use where a composition for sublingual administration is employed, a suitable dosage range is from 0.01 mg to about 25 mg (preferably from 0.1 mg to about 5 mg) of a compound of Formula I per kg of body weight per day.

### Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, sublingual, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like.

In practical use, the compounds of Formula I can be combined as the active ingredient in intimate admixture with a pharmaceutical carrier according to conventional pharmaceutical compounding techniques. The carrier may take a wide variety of forms depending-on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or non-aqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

### Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206, as well as natalizumab; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) immunomodulatory antibody therapies including anti-TNF therapies such as Etanercept (Enbrel®), Infliximab (Remicade®), Adalimumab (Humira®) or other TNF peptide or receptor sequestrants; Efalizumab (Raptiva®), Daclizumab (Zenapax®), Basiliximab (Simulect®), Rituximab (Rituxan®), visilizumab (Nuvion®), Abatacept (Orencia®) or other interleukin peptide or receptor binding antibodies; (e) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (f) non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (g) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac and zidometacin), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine, olsalazine, mesalamine and balsalazide) and the pyrazolones (apazone, bezpiperylon, feprazone, mofebutazone, oxyphenbutazone, phenylbutazone); (h) cyclooxygenase-2 (COX-2) inhibitors such as celecoxib, rofecoxib, and parecoxib; (i) inhibitors of phosphodiesterase type IV (PDE-IV); (j) antagonists of the other chemokine receptors, especially CCR1, CCR2, CCR5 and CCR3; (k) cholesterol lowering agents such as HMG-CoA reductase inhibitors (lovastatin, simvastatin, pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (1) anti-diabetic agents such as insulin, sulfonylureas, biguanides (metformin), α-glucosidase inhibitors (acarbose), glitazars (muraglitazar) and glitazones (troglitazone, pioglitazone, englitazone, MCC-555, BRL49653 and the like); (m) preparations of interferon beta (Avonex®, Rebif®, interferon beta-1a, Betaseron®, interferon beta-1b); (n) anticholinergic agents such as muscarinic antagonists (ipratropium and tiatropium); (o) current treatments for multiple sclerosis, including prednisolone, glatiramer, deoxyadenosine, mitoxantrone, methotrexate, and cyclophosphamide; (p) p38 kinase inhibitors; (q) DMARDs, such as methotrexate, leflunamide or plaquenil; (r) other compounds such as 5-aminosalicylic acid and prodrugs thereof, antimetabolites such as azathioprine, mycophenolate and 6-mercaptopurine, cytotoxic cancer chemotherapeutic agents and cytokine sequestrants.

The weight ratio of the compound of the Formula I to the second active ingredient may be varied and will depend upon the effective dose of each ingredient. Generally, an effective dose of each will be used. Thus, for example, when a compound of the Formula I is combined with an NSAID the weight ratio of the compound of the Formula I to the NSAID will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Methods of Synthesis

The following abbreviations are used in the synthetic schemes and examples:

Ac is acetyl [CH₃C(O)—]; Ac₂O is acetic anhydride; 9-BBN is 9-borabicyclo[3.3.1]nonane; Bn is benzyl; DIAD is diisopropylazodicarboxylate; DIBAL is diisobutylaluminum hydride; DMF is N,N-dimethylformamide; DMSO is dimethyl sulfoxide; EDAC (or EDC) is 1-ethyl-3-[3-(dimethylamino)propyl]-carbodiimide HCl; Et₃N is triethylamine; Et is ethyl; EtOAc is ethyl acetate; EtOH is ethanol; HCl is hydrochloric acid; HOBt is 1-hydroxybenzotriazole; HPLC is high performance liquid chromatography; iPrOAc is isopropyl acetate; LiHMDS is Lithium hexamethyldisilazane; LG is leaving group; M is molar; mmol is millimole; Me is methyl; MeOH is methanol; MsCl methanesulfonyl chloride; N is normal; NaHMDS is sodium hexamethyldisiliazide; NaOAc is sodium acetate; NaOtBu is sodium tert-butoxide; NMO is N-methylmorpholine N oxide; PG is protecting group; Pd(dba)₂ is tris(dibenzylideneacetone)dipalladium; PdCl₂(Ph₃P)₂ is dichloro bis-(triphenylphosphine) palladium; Ph is phenyl; PhMe is toluene; PPh₃ is triphenylphosphine; PMB is para-methoxybenzyl; RT is room temperature; TBAF is tetrabutyl ammonium fluoride; TBS is tert-butyldimethylsilyl; tBu is tert-butyl; Tf is triflate; TFA is trifluoroacetic acid; THF is tetrahydrofuran; TLC is thin layer chromatography; TMS is trimethylsilyl; TPAP is tetrapropylammonium perruthenate;

### General Schemes

The substituted thiazole compounds of this invention can be prepared by any of several known methods. The specific examples detailed below employ some of the following general procedures.

Trisubstituted aryl and heteroaryl intermediates 1 may be commercially available or may be prepared from readily accessible anilines, phenols or other simpler congeners via a host of routes which will be obvious to a practicing synthetic chemist. Some complex substitution patterns in heteroaryl intermediates are most readily accessible by de novo ring synthesis as outlined in Scheme 3.

The elaborated substituted thiazoles 7 are accessible from these intermediates as shown in Scheme 1 and 2 or alternate synthetic pathways as reported in the literature. The classic Hantzsch thiazole synthesis [Hantzsch, Annalen 250, 265, (1889), Schwarz, Org Syn Coll Vol 3 p 332.] is exceptionally versatile and is used for synthesis of many of the analogs reported here. The thioamide 4, readily available from the commercially available amide and Lawesson's reagent, is condensed with an alpha halo ketone at RT to generate the desired thiazole nucleus 5. Elaboration of the substituent at R₁₅ is followed by deprotection and coupling with a heteroarylacetate to yield the completed analogs 8. Some analogs will contain functionality which will require a final deprotection step. In many cases this constitutes an ester hydrolysis under typical conditions.

Transition metal catalyzed coupling reactions of an intermediate substituted thiazole 10 are also well suited to the synthesis of intermediate 5 as in Scheme 2. [Bull. Soc. Chim. Fr. 1962, 1735-1740 and Tet Lett 41(11) 1707-1710, 2000.]

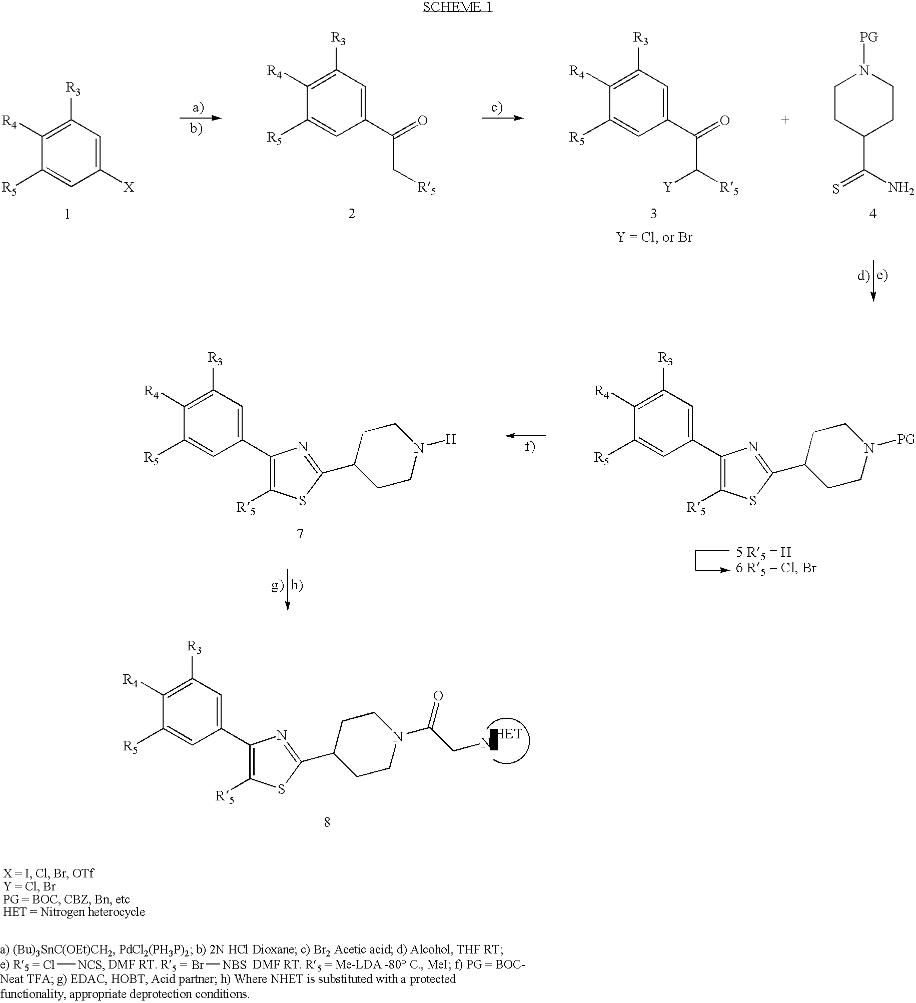

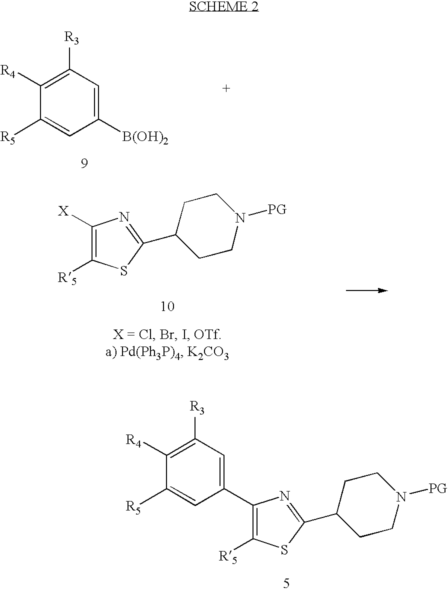

Examples reported here include thiazoles with R′₅ equal to hydrogen, alkyl and halogen substituents. Halogens are introduced by direct halogenation of the thiazole intermediates. Introduction of a halo substituent can occur at several stages in the synthesis. Any of the reagents Cl₂, Br₂ or typical electrophilic halogenation reagents such as NBS or NCS give good results. Alkyl residues are introduced either via synthesis from the corresponding substituted ketone or by metallation of the thiazole ring and introduction of an alkyl residue by alkylation with an alkyl halide or other alkylating agent.

In many of the current examples, polysubstituted heterocyclic fragments will be required Several examples derive from substituted pyrazoles, thiazoles, pyrimidines and imidazopyridines. The methods of synthesis will be well known to a practicing synthetic chemist and are summarized in; Katritzky, Alan R. (1984) Comprehensive heterocyclic chemistry: the structure, reactions, synthesis and uses of heterocyclic compounds, Oxford; Pergamon, New York. In addition to the various published routes to these intermediates, the general procedures of Scheme 3 have been found to give access to the desired intermediate heteroaryls. In Scheme 3, R and R″ are alkyl or aryl substituents as desired, and the ester residue E can also be alkyl or benzyl as needed to allow selective deprotection.

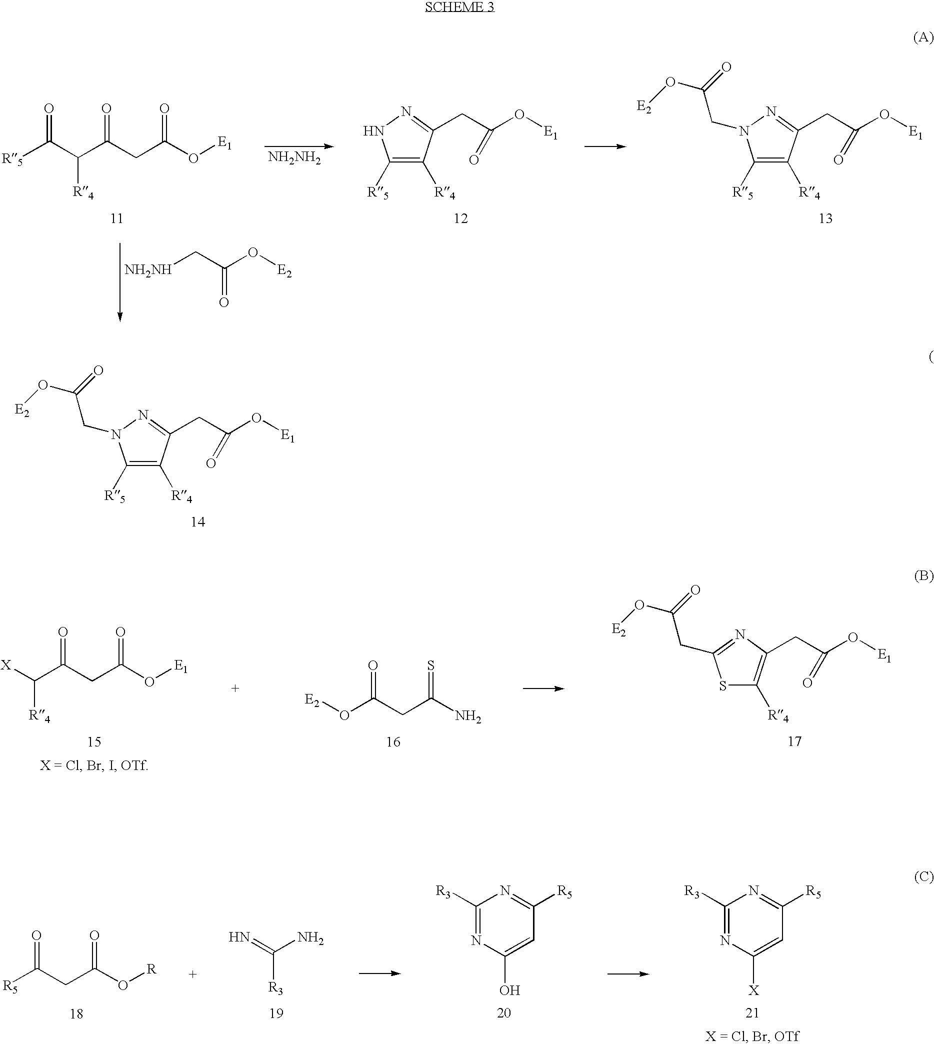

### EXAMPLES

The following compounds exemplify the invention.

### Example 1

### Preparation of 3-{2-[4-(4-{3-tert-butyl-5-[(trifluoromethyl)thio]phenyl}-5-chloro-1,3-thiazol-2-yl)piperidin-1-yl]-2-oxoethyl}-3H-imidazo[4,5-b]pyridine

### Step 1

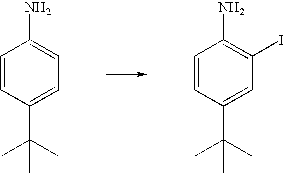

The aniline (75 g, 0.50 mol) was dissolved in 1:1 MeOH:H₂O (280 mL) and K₂CO₃ (72.93 grams; 0.53 mol) was added. ICl (1M in CH₂Cl₂, 528 mL) was added dropwise and the solution stirred for 2 hours at RT. The mixture was reduced in vac, the residue dissolved iPrOAc (2 L) and washed with water until the aqueous phase became clear. The organic fraction was dried over MgSO₄, filtered and volatiles removed in vac. The material was dissolved in CH₂Cl₂, combined with silica, volatiles removed in vac, and the solid placed in a fritted glass filter. The pack was eluted with methyl t-butyl ether:hexanes (1:3) to afford the titled compound.

### Step 2

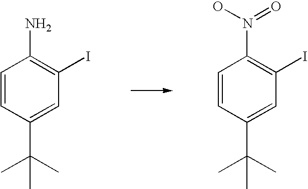

The iodo-aniline (See Example 1 Step 1, 21.1 g, 0.077 mol) was dissolved in CH₂Cl₂ (700 mL). NaHCO₃ (6.45 g; 0.077 mol) was added. Meta-chloro peroxybenzoic acid (52.3 g, 0.231 mol) was added in portions and the mixture stirred at RT overnight. The slurry was filtered and the volatiles removed in. vac. The mixture was dissolved in iPrOAc, washed with saturated NaHCO₃ (5 times), dried over MgSO₄, filtered and volatiles removed in. vac. The material was dissolved in CH₂Cl₂, combined with silica, the volatiles removed in. vac., and the solid placed in a fritted glass filter. The pack was eluted with methyl t-butyl ether:hexanes (1:6) to afford the titled compound.

### Step 3

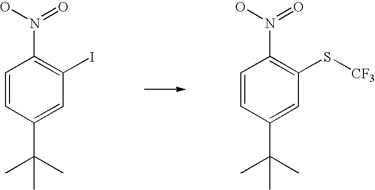

The nitro compound (See Example 1 Step 2, 76-9 g, 0.252 mol) and copper trifluoromethanethiol (31.1 g, 0.189 mol) were combined in N-methylpyrrolidinone (830 mL) and the mixture was heated at 160° C. for 1 hour. The mixture was cooled to RT and diluted with excess iPrOAc, filtered through a celite pad, and washed with pH 7 buffer (3×). The organic phase was dried over MgSO₄, filtered and the volatiles removed in. vac. The material was dissolved in CH₂Cl₂, combined with silica, volatiles removed in. vac., and the solid placed in a fritted glass filter. The pack was eluted with methyl t-butyl ether:hexanes (1:10) to afford the titled compound.

### Step 4

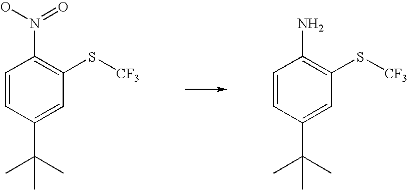

The nitro compound (See Example 1 Step 3, 57.3 g, 0.205 mol) was dissolved in MeOH and solid zinc (134-1 grams; 2.05 mol) was added. To the stirred solution AcOH in MeOH (25% vol/vol, 144 mL) was added over 25 minutes at RT from a dropping funnel through a condenser. The mixture was filtered through a celite pad and volatiles removed in. vac. The resulting tar was slurried in CH₂Cl₂ and filtered through a celite pad. The material was dissolved in CH₂Cl₂, combined with silica (300 g), volatiles removed in. vac. and the solid placed in a fritted glass filter. The pack was eluted with methyl t-butyl ether:hexanes (1:10). Silica gel chromatography (methyl t-butyl ether:hexanes (1:20)) affords the titled compound.

### Step 5

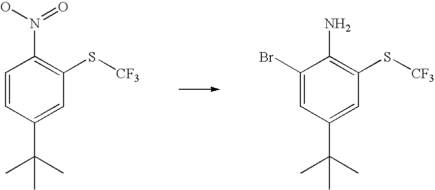

The aniline (See Example 1 Step 4, 16 g, 0.064 mol) was dissolved in CH₂Cl₂ (215 mL) with stirring. NBS (10.3 g, 0.058 mol) was added as a solid in portions at RT. A second addition of NBS (1.14 g, 6.4 mmol) was made. The mixture was stirred at RT for 40 min and the volatiles removed in. vac. The crude material was slurried in methylcyclohexane (320 mL), and the volatiles removed in. vac. to afford the titled compound.

### Step 6

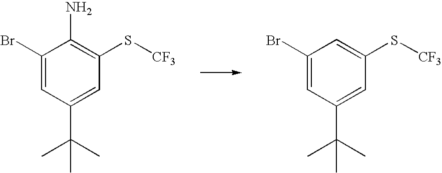

A 500 mL three necked flask with a mechanical stirrer was charged with isoamyl nitrite (17.2 mL, 0.168 mol), DMF (10 mL) added and the solution heated to 70° C. The aniline (See Example 1 Step 5, 21.2 g, 0.065 mol) was dissolved in DMF (106 mL) and added dropwise over 1 hour with constant heating. The mixture was stirred for an additional 1.5 hours, cooled to RT, diluted with iPrOAc and washed with pH 7 buffer (400 mL, 4 times). The organic fraction was dried over MgSO₄, filtered and the volatiles removed in. vac. The crude oil (neat) was placed onto a column of silica gel and eluted with hexanes.

### Step 7

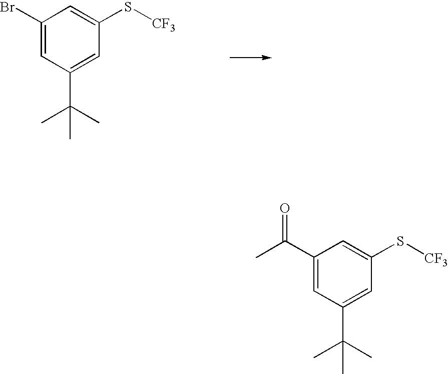

The bromide (See Example 1 Step 6, 3.0 g, 9.6 mmol), tri-nbutyl-1-ethoxyvinyl tin (4.15 g, 11.5 mmol), and PdCl₂(Ph₃P)₂ (672 mg, 0.96 mmol) were dissolved in toluene (19 mL) and the solution heated at 95° C. for 3 hours. The solvent was removed in vac. The residue was dissolved in 1,4-dioxane (70 mL), aqueous HCl (2 N, 14 mL) added and the solution stirred rapidly for 1 hour. The mixture was diluted with water (400 mL) and extracted with EtOAc (4 times, 70 mL). The combined organic fraction was washed with brine, dried over Na₂SO, filtered, and the volatiles removed in vac. Silica gel chromatography (diethyl ether:hexanes (1:25)) affords the titled compound.

### Step 8

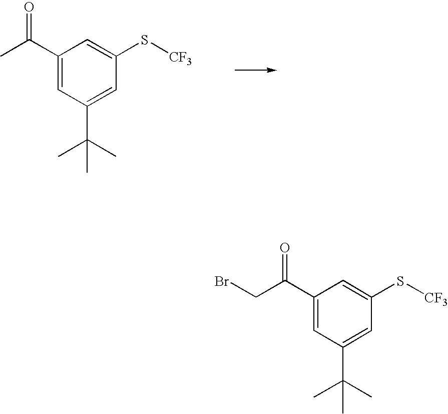

The ketone (See Example 1 Step 7, 1.95 g, 8.5 mmol) was dissolved in AcOH (35 mL) and the flask cooled in an RT water bath. Bromine (1 M in acetic acid, 7.76 mL) was added to the stirred solution drop-wise and the resulting mixture was stirred overnight. The mixture was poured into a solution of concentrated HCl:ice water (10:300 mL), partitioned with EtOAc (3 times, 70 L). The organics were combined, washed with brine, dried over MgSO₄, filtered and the volatiles removed in vac to afford the titled compound.

### Step 9

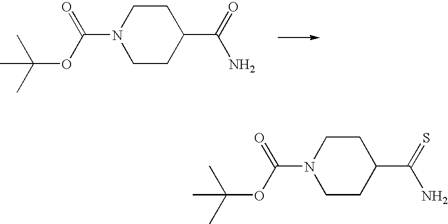

The amide (20 g, 76.3 mmol) was dissolved in 1,4-dioxane with heating and the clear solution placed in a 60° C. oil bath. Lawesson's Reagent was added (15.43 g, 38.15 mmol) and the solution stirred for 2 hours. The solution was cooled to RT, poured into 8:1 water:saturated NaHCO₃, followed by aqueous/EtOAc work-up and silica gel chromatography (EtOAc:hexanes (1:4)) to give the titled compound.

### Step 10

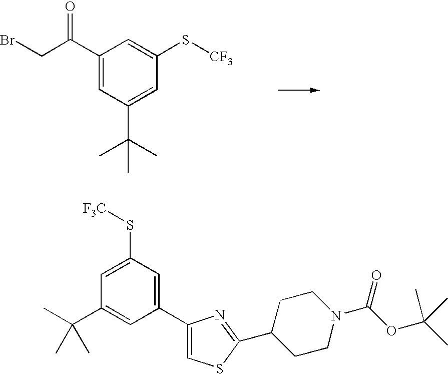

The bromoketone (See Example 1 Step 8, (2.41 g, 5.4 mmol) was dissolved in 1:1 EtOH/THF (34 mL), the thioamide (See Example 1 Step 9 (1.66 g, 6.8 mmol) added, and the mixture stirred overnight at room temperature. The volatiles were removed in vac and the residue dissolved DMF (13.5 mL). Et₃N (6.87 g, 68 mmol) and di-tert-butyl dicarbonate (888 mg, 4.07 mmol) were added and the solution stirred for 1 hour. The mixture was poured into 4:1 water/saturated NaHCO₃ followed by aqueous/EtOAc work-up and silica gel chromatography (diethyl ether:hexanes (1:4)) to afford the titled compound.

### Step 11

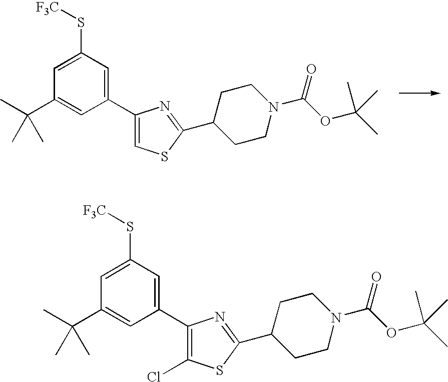

A suspension of the thiazole (See Example 1 Step 10, (1.62 g, 5.1 mmol) in DMF (32 μL) was heated to give a clear solution. The solution was cooled to RT in a water bath and NCS (0.47 g, 3.55 mmol) was added. The clear solution was immediately transferred to a 45° C. oil bath and the solution stirred for 1 hour. Additional NCS (47 mg, 0.35 mmol) was added and the solution stirred for 1 hour. The solution was cooled to RT and the material from this batch was combined with a second similar batch run. The combined material was diluted with 8:1 water/saturated NaHCO₃ (800 mL) followed by aqueous/EtOAc work-up and silica gel chromatography (diethyl ether:hexanes (1:3)) to afford the titled compound.

### Step 12

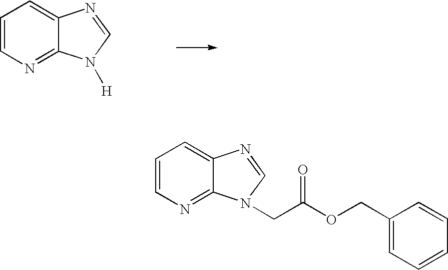

The 4-azabenzimidazole (10 g, 84 mmol) was dissolved in DMF (168 mL) and Cs₂CO₃ (54.7 g, 168 mmol) added. Benzyl 2-bromoacetate was added dropwise and the solution stirred for four hours at RT. The solution was poured into 8:1 water/saturated NaHCO₃ (1700 mL) followed by aqueous/EtOAc work-up and silica gel chromatography (EtOAc:hexanes (2:3)) to give the titled compound.

### Step 13

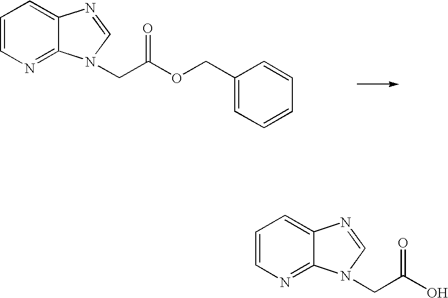

The ester (See Example 1 Step 12, 8.5 g, 32 mmol) was dissolved in MeOH (160 mL) and Pd on carbon (10%, 1.7 g) added. The solution was degassed (3 times) and placed under an atmosphere of hydrogen and stirred or 1 hour at RT. The solution was filtered, Pd on carbon (10%, 1.7 g) added, and the solution stirred for 4 hours. Methanol was added (200 mL), the solution stirred for 10 minutes and filtered. The solvent was removed in vac to give the titled compound.

### Step 14

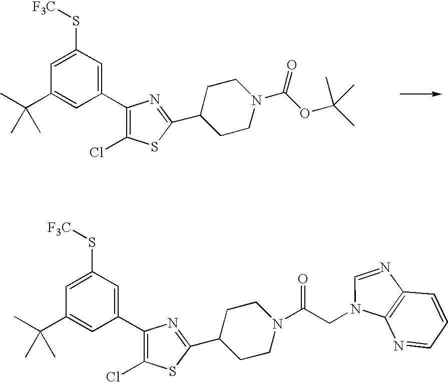

The chloride (See Example 1 Step 10 (2.02 g, 3.8 mmol) was dissolved in TFA (30 mL), stirred at RT for 10 minutes, and the volatiles removed in vac. The residue was dissolved in EtOAc, washed with 4:1 water/saturated NaHCO₃ (400 mL) and extracted (3 times) with EtOAc. The combined organic fraction was washed with brine, dried over Na₂SO, filtered and the volatiles removed in vac. The recovered material, 1-HOBT (0.77 g, 5.66 mmol) and the acid (See Example 1 Step 13, 0.80 g, 4.52 mmol) were combined and the mixture dissolved in DMF (18 mL). Diisopropyl ethyl amine (2.4 g, 18.9 mmol) and EDAC (1.084 g, 5.7 mmol) were added and the solution stirred at RT overnight. The mixture was diluted with 8:1 water/saturated NaHCO₃ followed by aqueous/EtOAc work-up and silica gel chromatography (acetone:hexanes (1:1) plus 6% Et₃N) to give the titled compound. The material was dissolved in a small portion of EtOAc, diluted with 4:1 water/saturated NaHCO₃ (300 mL) and extracted with EtOAc (4 times, 100 mL). The combined organic fraction was washed with brine, dried over Na₂SO, filtered and the volatiles removed in vac.

¹H NMR (500 MHz, CD3OD): δ 8.41 (dd, J=1.3, 4.8 Hz, 1H); 8.38 (s, 1H); 8.21 (t, J=1.6 Hz, 1H); 8.14 (dd, J=1.2, 8.0 Hz, 1H); 8.09 (s, 1H); 7.77 (s, 1H); 7.39 (dd, J=4.8, 8.1 Hz, 1H); 5.43 (ABq, Δδ=25.0, J=16.9 Hz, 2H); 4.55 (d, J=13.4 Hz, 1H); 4.24 (d, J=13.8 Hz, 1H); 3.49 (t, J=13.2 Hz, 1H); 3.42 (tt, J=3.9, 11.4 Hz, 1H); 3.00 (t, J=13.3 Hz, 1H); 2.34 (d, J=11.9 Hz, 1H); 2.21 (d, J=14.2 Hz, 1H); 2.09 (dq, J=3.7, 12.7 Hz, 1H); 1.84 (dq, J=3.9, 12.5 Hz, 1H); 1.42 (s, 9H).

MS: m/z=594.29 (M+H).

### Example 2

### Preparation of 3-{2-[4-(5-bromo-4-{3-tert-butyl-5-[(trifluoromethyl)thio]phenyl}-1,3-thiazol-2-yl)piperidin-1-yl]-2-oxoethyl}-3H-imidazo[4,5-b]pyridine

### Step 1

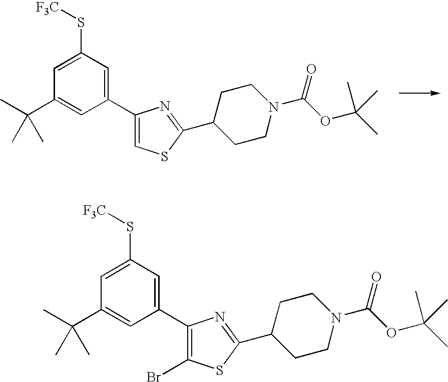

A suspension of the thiazole (See Example 1 Step 10, (1.5 g, 3.0 mmol) in DMF (15 mL) was heated to give a clear solution. The solution was cooled to RT in a water bath and N BS (0.59 g, 3.3 mmol) was added to the well stirred solution in one portion. The solution was transferred to a 45° C. oil bath and stirring was continued for 5 min. The solution was then stirred at RT for 4 hours. Additional NBS (53 mg, 0.30 mmol) was added and the solution stirred for 1 hour. The solution was diluted with 4:1 water/saturated NaHCO₃ (800 mL) followed by aqueous/EtOAc work-up and silica gel chromatography (diethyl ether:hexanes (1:3)) to afford the titled compound.

### Step 2

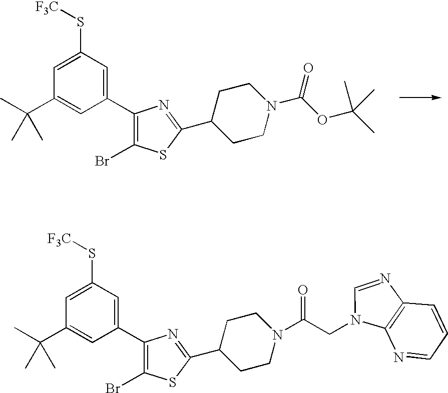

The bromide (See Example 2 Step 1, (2.13 g, 3.7 mmol) was dissolved in TFA (30 mL), stirred at RT for 10 minutes, and the volatiles removed in vac. The residue was dissolved in EtOAc, washed with 4:1 water/saturated NaHCO₃ (400 mL) and extracted (5 times) with EtOAc. The combined organic fraction was washed with brine, dried over Na₂SO, filtered and reduced in vac. The recovered material, 1-HOBT (0.75, 5.5 mmol) and the acid (See Example 1 Step 13, 0.78 g, 4.4 mmol) combined and the mixture dissolved in DMF (18 mL). Diisopropyl ethyl amine (2.4 g, 18.9 mmol) and EDAC (1.084 g, 5.7 mmol) were added and the solution stirred at RT overnight. The mixture was diluted with 8:1 water/saturated NaHCO₃ followed by aqueous/EtOAc work-up and silica gel chromatography (acetone:hexanes (1:1) plus 6% Et₃N) to give the titled compound. The material was dissolved in a small portion of EtOAc, diluted with 4:1 water/saturated NaHCO₃ (300 mL) and extracted with EtOAc (4 times, 100 mL). The combined organic extracts were washed with brine, dried over Na₂SO, filtered and reduced in vac.

¹H NMR (500 MHz, CD3OD): δ 8.37 (s, 1H); 8.36 (s, 1H); 8.17 (s, 1H); 8.10 (d, J=8.0 Hz, 1H); 8.04 (s, 1H); 7.74 (s, 1H); 7.34 (dd, J=4.8, 8.0 Hz, 1H); 5.38 (ABq, Δδ=24.5, J=17.2 Hz, 2H); 4.51 (d, J=13.5 Hz, 1H); 4.19 (d, J=14.0 Hz, 1H); 3.47-3.37 (m, 2H); 2.95 (t, J=12.1 Hz, 1H); 2.29 (d, J=13.5 Hz, 1H); 2.17 (d, J=12.0 Hz, 1H); 2.05 (dq, J=3.9, 12.3 Hz, 1H); 1.79 (dq, J=3.9, 12.5 Hz, 1H); 1.39 (s, 9H).

MS: m/z=638.05 (M+H).

### Example 3

### Preparation of 3-{2-[4-(4-{3-tert-butyl-5-[(trifluoromethyl)sulfonyl]phenyl}-5-chloro-1,3-thiazol-2-yl)piperidin-1-yl]-2-oxoethyl}-3H-imidazo[4,5-b]pyridine

### Step 1

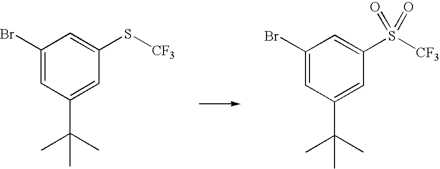

The bromide (See Example 1 Step 6, 1 g, 3.2 mmol) was dissolved in AcOH, chromium (VI) oxide (2.6 g, 26 mmol) added, and the dark slurry heated to 105° C. for 1 hour. The temperature was reduced to 100° C. and the solution stirred overnight. The solution was cooled to RT, diluted with water (250 mL), followed by aqueous/EtOAc work-up and silica gel chromatography (EtOAc:hexanes (1:19)) to give the titled compound.

### Step 2

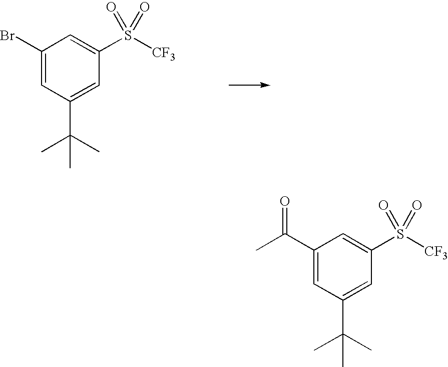

The bromide (See Example 3 Step 1, 0.3 g, 0.89 mmol), tri-nbutyl-1-ethoxyvinyl tin (0.38 g, 1.04 mmol), and PdCl₂(Ph₃P)₂ (61 mg, 0.09 mmol) were dissolved in toluene and the solution heated at 95° C. for 2 hours. The solvent was removed in vac. The residue was dissolved in 1,4-dioxane (4.3 mL), aqueous HCl (2 N, 1.31 mL) added and the mixture stirred rapidly for 1 hr. The mixture was diluted with water (40 mL) and extracted with EtOAc (4 times, 40 mL). The combined organic fraction was washed with brine, dried over Na₂SO₄, filtered, and the volatiles removed in vac Silica gel chromatography (diethyl ether:hexanes (1:9)) afforded the titled compound.

### Step 3

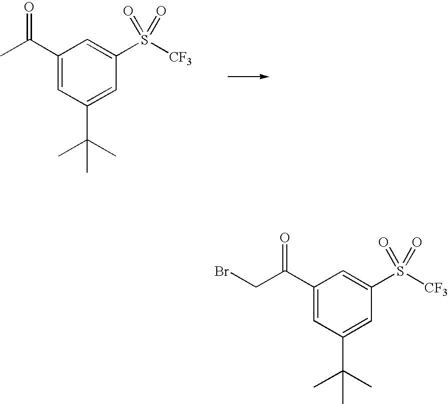

The ketone (See Example 3 Step 2, 0.21 g, 0.69 mmol) was dissolved in AcOH (3.4 mL) and bromine (1 M in AcOH, 0.76 mL, 0.76 mmol) added dropwise. The solution was stirred at 45° C. for 30 minutes. Additional bromine (1 M in AcOH, 0.11 mL, 0.1 mmol) was added and the solution stirred at 45° C. for 30 minutes. The mixture was diluted with a solution of concentrated HCl/ice water (1:15, 32 mL), followed by aqueous/EtOAc work-up to give the titled compound.

### Step 4

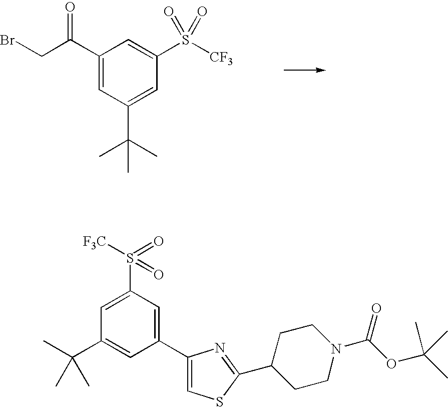

The bromoketone (See Example 3 Step 3, (0.28 g, 0.71 mmol) was dissolved in 1:1 ethyl alcohol/THF (1.7 mL), the thioamide (See Example 1 Step 9 (0.17 g, 0.71 mmol) added, and the mixture stirred at room temperature overnight. The volatiles were removed in vac and the residue dissolved DMF (3.5 mL). Et₃N (0.72 g, 7.1 mmol) and di-tert-butyl dicarbonate (78 mg, 0.36 mmol) were added and the solution stirred at RT for 1 hour. The mixture was poured into 4:1 water/saturated NaHCO₃ followed by aqueous/EtOAc work-up and silica gel chromatography (EtOAc:hexanes (1:4)) to afford the titled compound.

### Step 5

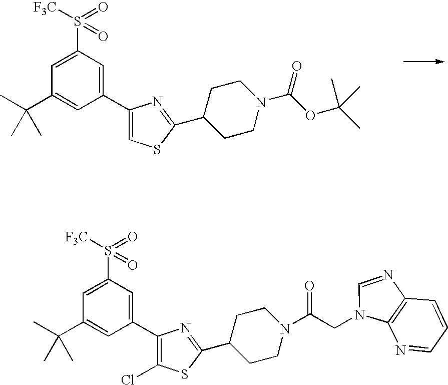

A suspension of the thiazole (See Example 3 Step 4, (27 mg, 0.051 mmol) in DMF (0.5 mL) was heated at 45° C. NCS (7.4 mg, 0.056 mmol) was added to the well stirred solution in one portion and stirred continued for 1 hour. The solution was cooled to RT, diluted with 8:1 water/saturated NaHCO₃ (40 mL), followed by aqueous/EtOAc work-up to afford the titled compound. The material was dissolved in TFA (2 mL), stirred for 10 minutes, and the volatiles removed in vac. The residue was dissolved in EtOAc, washed with 4:1 water/saturated NaHCO₃ (40 mL) and extracted (3 times) with EtOAc. The combined organic fraction was washed with brine, dried over Na₂SO, filtered and the volatiles removed in vac. The recovered material, 1-HOBT (11 mg, 0.08 mmol) and the acid (See Example 1 Step 13, 11 mg, 0.06 mmol) were combined and the mixture dissolved in DMF (0.5 mL). N,N-Diisopropyl ethyl amine (33 mg, 0.26 mmol) and EDAC (15 mg, 0.08 mmol) were added and the solution stirred at RT overnight. The reaction mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.29 (s, 1H); 8.65 (d, J=4.5 Hz, 1H); 8.60 (s, 1H); 8.45 (s, 1H); 8.30 (d, J=8.1 Hz, 1H); 8.06 (s, 1H); 7.63 (dd, J=4.8, 8.2 Hz, 1H); 5.62 (ABq, Δδ=25.4, J=16.7 Hz, 2H); 4.54 (d, J=13.6 Hz, 1H); 4.21 (d, J=14.2 Hz, 1H); 3.51-3.40 (m, 2H); 3.00 (t, J=13.6 Hz 1H); 2.34 (d, J=11.9 Hz, 1H); 2.21 (d, J=12.2 Hz, 1H); 2.08 (dq, J=3.9, 12.8 Hz 1H); 1.82 (dq, J=3.9, 12.5 Hz, 1H); 1.44 (s, 9H).

MS: m/z=626.14 (M+H).

### Example 5

### Preparation of 3-{2-[4-(5-bromo-4-{3-tert-butyl-5-[(trifluoromethyl)sulfonyl]phenyl}-1,3-thiazol-2-yl)piperidin-1-yl]-2-oxoethyl}-3H-imidazo[4,5-b]pyridine

### Step 1

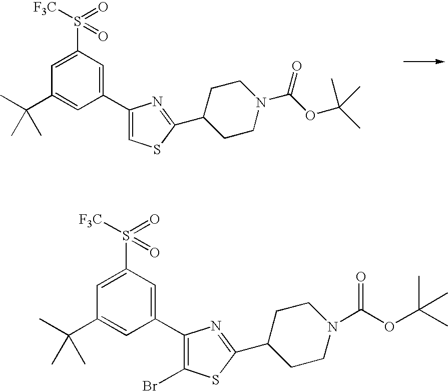

A suspension of the thiazole (See Example 4 Step 4, (80 mg, 0.15 mmol) in DMF (0.75 mL) was heated to 45° C. to give a clear solution. NBS (29 mg, 0.17 mmol) was added to the well stirred solution in one portion and the solution stirred at 45° C. for 2 hours. Additional NBS (8 mg, 0.04 mmol) was added and the solution stirred for 2 hours. The solution was poured into 4:1 water/saturated NaHCO₃ (40 mL), followed by aqueous/EtOAc work-up, and silica gel chromatography (diethyl ether:hexanes (3:7)) to afford the titled compound.

### Step 2.

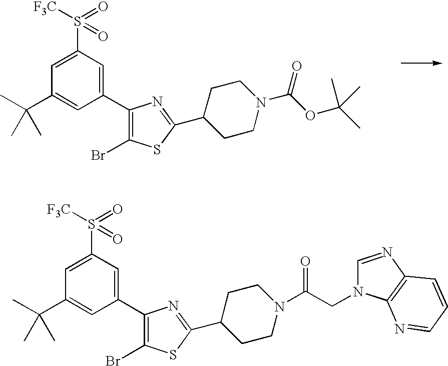

The bromide (28 mg, 0.05 mmol) was dissolved in TFA (2 mL), stirred at RT for 10 minutes and the volatiles removed in vac. The residue was dissolved in EtOAc, washed with 4:1 water/saturated NaHCO₃ (40 mL) and the aqueous wash re-extracted (3 times) with EtOAc. The combined organic fraction was washed with brine, dried over Na₂SO, filtered and the volatiles removed in vac. The recovered material, 1-HOBT (9.4 mg, 0.07 mmol) and the acid (See Example 1 Step 13, 9.8 mg, 0.06 mmol) were combined and the mixture dissolved in DMF (0.5 mL). Diisopropyl ethyl amine (30 mg, 0.23 mmol) and EDAC (13 mg, 0.07 mmol) were added and the solution stirred at RT overnight. The reaction mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.18 (s, 1H); 8.62-8.60 (m, 2H); 8.44 (s, 1H); 8.27 (d, J=7.5 Hz, 1H); 8.06 (s, 1H); 7.59 (dd, J=4.8, 8.2 Hz, 1H); 5.59 (ABq, Δδ=25.4, J=16.7 Hz, 2H); 4.58 (t, J=28.5 Hz, 1H); 4.21 (d, J=14.0 Hz, 1H); 3.52-3.42 (m, 2H); 3.00 (tt, J=1.6, 13.5, 1H); 2.34 (d, J=13.4 Hz, 1H); 2.21 (d, J=11.7 Hz, 1H); 2.09 (dq, J=3.9, 12.8, 1H); 1.86-1.78 (dq, J=4.1, 12.8, 1H); 1.44 (s, 9H).

MS: m/z=670.05 (M+H).

### Example 6

### Preparation of 3-(2-{4-[5-bromo-4-(3,5-di-tert-butyl-4-methoxyphenyl)-1,3-thiazol-2-yl]piperidin-1-yl}-2-oxoethyl)-3H-imidazo[4,5-b]pyridine

### Step 1

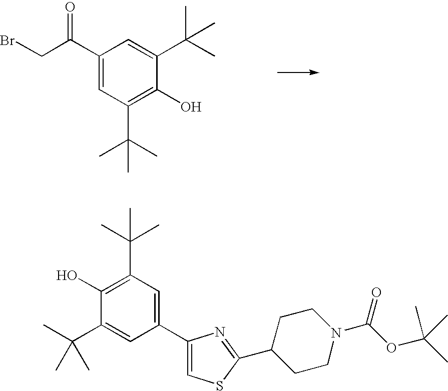

The bromoketone (1.3 g, 4.0 mmol) was dissolved in 1:1 ethyl alcohol/THF (20 mL), the thioamide (See Example 1 Step 9 (0.97 g, 4.0 mmol) added, and the mixture stirred at room temperature overnight. The volatiles were removed in. vac. and the residue dissolved in DMF (6 mL). Et₃N (0.48 g, 4.8 mmol) and di-tert-butyl dicarbonate (0.26 g, 1.2 mmol) were added and the solution stirred at RT for 1 hour. The mixture was diluted with water, followed by aqueous/EtOAc work-up and silica gel chromatography (EtOAc:hexanes (1:9)) to afford the titled compound.

### Step 2

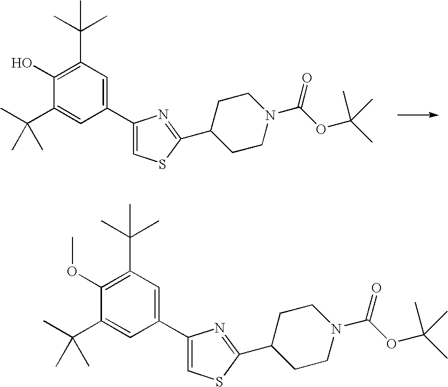

The thiazole (See Example 6 Step 1, (1.6 g, 3.3 mmol) was dissolved in DMF (8.3 mL), Cs₂CO₃ (1.3 g, 4.0) and iodomethane (0.56, 4.0 mmol) added, and the solution stirred at RT overnight. Additional iodomethane (140 mg, 1 mmol) and Cs₂CO₃ (0.32 g, 1 mmol) were added and the solution stirred for 1 hour. The solution was diluted with water (350 mL), followed by aqueous/EtOAc work-up and silica gel chromatography (EtOAc:hexanes (1:9)) to give the titled compound.

### Step 3

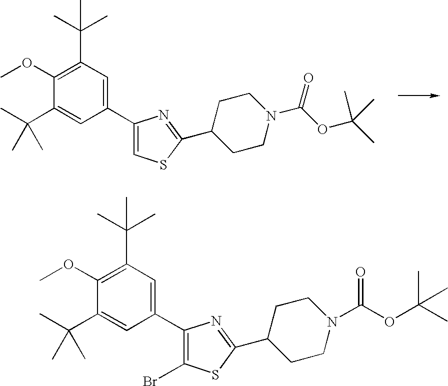

The thiazole (See Example 6 Step 2, (1.2 g, 2.4 mmol) was dissolved in DMF (12 mL), NBS (0.47 g, 2.64 mmol) added, and the solution stirred at RT overnight. Additional NBS (47 mg, 0.26 mmol) was added and the solution stirred for 2 hours. The mixture was poured into 4:1 water/saturated NaHCO₃ (800 mL) followed by aqueous/EtOAc work-up and silica gel chromatography (diethyl ether:hexanes (1:4)) to afford the titled compound.

### Step 4

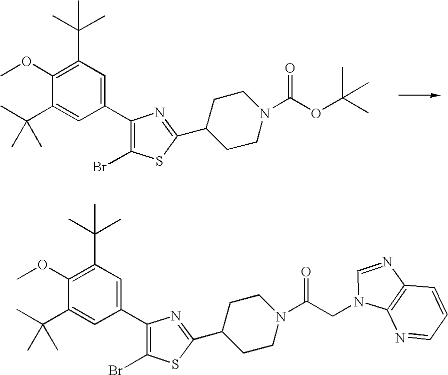

The bromide (See Example 6 Step 3, (2.02 g, 3.8 mmol) was dissolved in TFA (18 mL), stirred at RT for 10 minutes, and the volatiles removed in vac. The residue was dissolved in EtOAc, washed with 4:1 water/saturated NaHCO₃ (400 mL) and the aqueous wash re-extracted (3 times) with EtOAc. The combined organic fraction was washed with brine, dried over Na₂SO, filtered and the volatiles removed in vac. The recovered material, 1-HOBT (0.46 g, 3.4 mmol) and the acid (See Example 1 Step 13, 0.47 g, 2.7 mmol) were combined and the mixture dissolved in DMF (11 mL). Diisopropyl ethyl amine (1.4 g, 11 mmol) and EDAC (0.64 g, 3.4 mmol) were added and the solution allowed to stir at RT overnight. The mixture was poured into 8:1 water/saturated NaHCO₃ followed by aqueous/EtOAc work-up and silica gel chromatography (acetone:hexanes (1:1) plus 6% Et₃N). The material was dissolved in a small portion of EtOAc, diluted with 4:1 water/saturated NaHCO₃ (300 mL) and extracted with EtOAc (4 times, 100 mL). The combined organic fraction was washed with brine, dried over Na₂SO, filtered and reduced in vac.

¹H NMR (500 MHz, CDCl₃): δ 8.36 (d, J=4.8 Hz, 1H); 8.28 (s, 1H); 8.08 (d, J=6.6 Hz, 1H); 7.75 (s, 2H); 7.31 (dd, J=4.9, 7.9 Hz, 2H), 5.31 (ABq, Δδ=26.9, J=17.0 Hz, 2H); 4.53 (d, J=13.3 Hz, 1H); 4.14 (d, J=14.1 Hz, 1H); 3.69 (s, 3H); 3.42 (t, J=11.6 1H); 3.32 (m, 1H); 2.93 (t, J=12.2 Hz, 1H); 2.30 (d, J=12.1 Hz, 1H); 2.18 (d, J=11.6 Hz, 1H); 2.00 (dq, J=3.5, 13.3, 1H); 1.80 (dq, J=3.6, 12.8, 1H); 1.44 (s, 18H).

MS: m/z 624.19 (M+H).

### Example 7

### Preparation of 3-(2-{4-[5-chloro-4-(3,5-di-tert-butyl-4-methoxyphenyl)-1,3-thiazol-2-yl]piperidin-1-yl}-2-oxoethyl)-3H-imidazo[4,5-b]pyridine

### Step 1

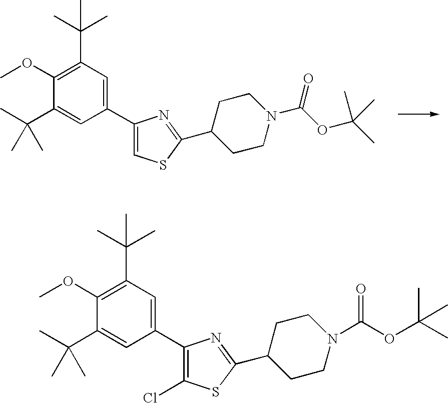

The thiazole (See Example 6 Step 2, (1.6 g, 3.29 mmol) was dissolved in DMF (20 mL) and CH₂Cl₂ (5 mL). NCS (0.484 g, 3.62 mmol) was added and the solution stirred at 50° C. for 1.5 hours. The mixture was cooled to room temperature, diluted with water (400 mL), followed by aqueous/EtOAc work-up and silica gel chromatography (diethyl ether:hexanes (1:4)) to afford the titled compound.

### Step 2

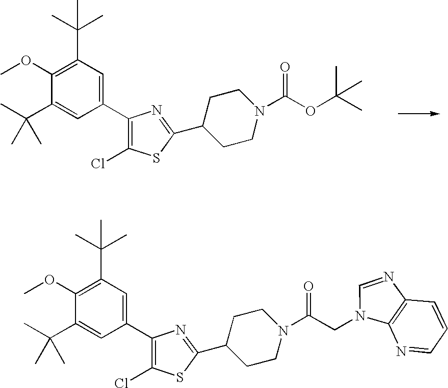

The chloride (See Example 7 Step 1, (36 mg, 0.058 mmol) was dissolved in TFA (2 mL), stirred at RT for 10 minutes, and the volatiles removed in vac. The residue was dissolved in EtOAc, washed with 4:1 water/saturated NaHCO₃ (400 mL) and the aqueous wash re-extracted (3 times) with EtOAc. The combined organic fraction was washed with brine, dried over Na₂SO, filtered and reduced in vac. The recovered material, 1-HOBT (6 mg, 0.044 mmol) and the acid (See Example 1 Step 13, 6 mg, 0.035 mmol) were combined and the mixture dissolved in DMF (0.3 mL). Diisopropyl ethyl amine (19 mg, 0.15 mmol) and EDAC (8.3 mg, 0.044 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.28 (s, 1H); 8.64 (dd, J=1.3, 4.8 Hz, 1H); 8.29 (dd, J=1.2, 8.2 Hz, 1H); 7.81 (s, 2H); 7.62 (dd, J=4.8, 8.2 Hz, 1H); 5.61 (ABq, Δδ=23.0, J=16.7 Hz, 2H); 4.52 (d, J=13.6 Hz, 1H); 4.20 (d, J=14.2 Hz, 1H); 3.72 (s, 3H); 3.48 (t, J=13.0 Hz, 1H); 3.38 (tt, J=3.9, 11.5 Hz, 1H); 2.99 (dt, J=1.6, 13.2 Hz, 3H); 2.32 (d, J=11.8 Hz, 1H); 2.19 (d, J=13.2 Hz, 1H); 2.03 (dq, J=4.2, 13.1 Hz, 1H); 1.79 (dq, J=3.9, 12.8 Hz, 1H); 1.46 (s, 18H).

MS: m/z=580.04 (M+H).

### Example 8

### Preparation of 3-[2-(4-{4-[3,5-bis(trifluoromethyl)phenyl]-5-bromo-1,3-thiazol-2-yl}piperidin-1-yl)-2-oxoethyl]-3H-imidazo[4,5-b]pyridine

### Step 1

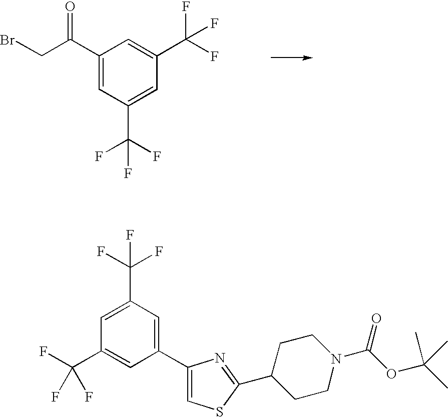

The bromoketone (0.25 g, 0.75 mmol) was dissolved in 1:4 ethyl alcohol/THF (20 mL), the thioamide (See Example 1 Step 9 (0.20 g, 0.82 mmol) added, and the mixture stirred at room temperature overnight. The mixture was diluted with 4:1 water/saturated NaHCO₃ followed by aqueous/EtOAc work-up and silica gel chromatography (EtOAc:hexanes (1:4)) to afford the titled compound.

### Step 2

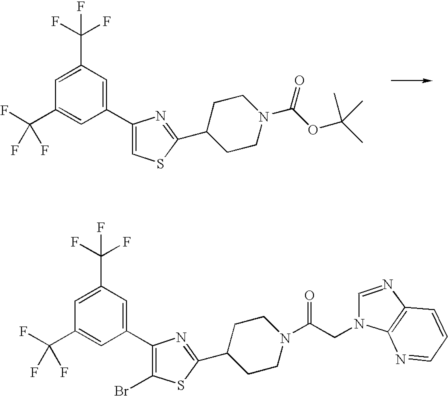

The thiazole (See Example 8 Step 1, (26 mg, 0.054 mmol) was dissolved in TFA (2 mL), stirred at RT for 30 minutes, and the volatiles removed in vac. The material was dissolved in AcOH (0.5 mL), bromine (0.8 M in AcOH, 0.08 mL, 0.06 mmol) added dropwise and the solution stirred at RT for 90 minutes. A second dropwise addition of bromine (0.8 M in AcOH, 0.04 mL, 0.03 mmol) was made and the solution stirred at 59° C. for 0.5 hours. A third addition of bromine (0.8 M in AcOH, 0.04 mL, 0.03 mmol) was made and the heating continued at 59° C. for 2 hours. The mixture was diluted with 4:1 water/saturated NaHCO₃ (30 mL) followed by aqueous/EtOAc work-up. The recovered material, 1-HOBT (11 mg, 0.081 mmol) and the acid (See Example 1 Step 13, 12 mg, 0.065 mmol) were combined and the mixture dissolved in DMF (0.5 mL). Diisopropyl ethyl amine (35 mg, 0.27 mmol) and EDAC (16 mg, 0.081 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.16 (s, 1H); 8.60 (d, J=4.7 Hz, 1H); 8.56 (s, 2H); 8.29 (s, 1H); 8.03 (s, 1H); 7.58 (dd, J=5.1, 7.7 Hz, 1H); 5.58 (ABq, Δδ=21.0, J=16.9 Hz, 2H); 4.53 (d, J=12.3 Hz, 1H); 4.21 (d, J=13.7 Hz, 1H); 3.51-3.43 (m, 2H); 3.00 (t, J=12.1 Hz, 1H); 2.34 (d, J=14.4 Hz, 1H); 2.21 (d, J=13.8 Hz, 1H); 2.09 (dq, J=3.6, 13.0 Hz, 1H), 1.83 (dq, J=3.6, 12.3 Hz, 1H).

MS: m/z 618.02 (M+H).

### Example 9

### Preparation of 3-[2-(4-{4-[3,5-bis(trifluoromethyl)phenyl]-5-chloro-1,3-thiazol-2-yl}piperidin-1-yl)-2-oxoethyl]-3H-imidazo[4,5-b]pyridine

### Step 1

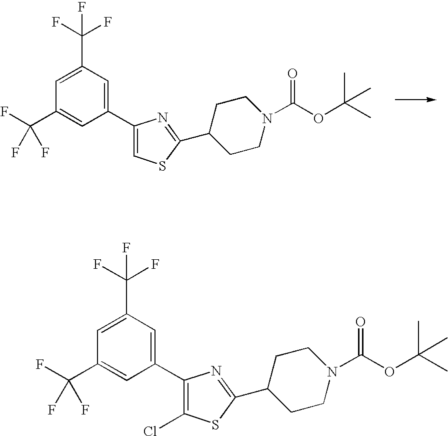

The thiazole (See Example 8 Step 1, (34 mg, 0.071 mmol) was dissolved in CH₂Cl₂ (0.35 mL), NCS (10 mg, 0.078 mmol) added, and the solution stirred overnight at 50° C. The solution was partially concentrated in vac and the remaining solution loaded directly onto a silica gel column and chromatographed (diethyl ether:hexanes (1:4)) to afford the titled compound.

### Step 2

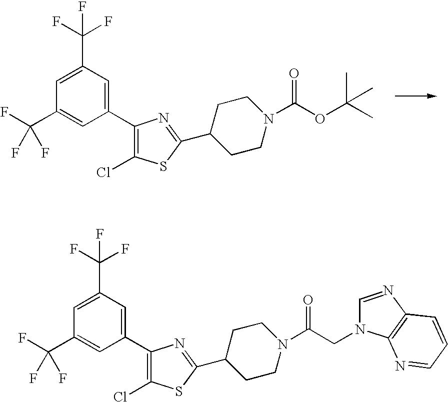

The chloride (See Example 9 Step 1, (22 mg, 0.43 mmol) was dissolved in TFA (2 mL), stirred at RT for 10 minutes and the volatiles removed in vac. The residue was dissolved in EtOAc, washed with 4:1 water/saturated NaHCO₃ (40 mL) and extracted (3 times) with EtOAc. The combined organic fraction was washed with brine, dried over Na₂SO, filtered and the volatiles removed in vac. The recovered material, 1-HOBT (9 mg, 0.07 mmol) and the acid (See Example 1 Step 13, 9 mg, 0.05 mmol) were combined and the mixture dissolved in DMF (0.4 mL). Diisopropyl ethyl amine (28 mg, 0.22 mmol) and EDAC (12 mg, 0.07 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.08 (s, 1H); 8.58 (dd, J=1.2, 4.7 Hz, 1H); 8.55 (s, 2H); 8.26 (d, J=8.1 Hz, 1H); 8.02 (s, 1H); 7.57 (dd, J=4.8, 8.2 Hz, 1H); 5.57 (ABq, Δδ=22.4, J=17.0 Hz, 2H); 4.53 (d, J=13.6 Hz, 1H); 4.21 (d, J=13.8 Hz, 1H); 3.50-3.40 (m, 2H); 3.00 (t, J=3.6, 13.0 Hz, 1H); 2.34 (d, J=12.2 Hz, 1H); 2.21 (d, J=11.6 Hz, 1H); 2.08 (dq, J=3.9, 13.0 Hz, 1H); 1.82 (dq, J=4.1, 12.8 Hz, 1H).

MS: m/z=574.13 (M+H).

### Example 10

### Preparation of 3-[2-(4-{4-[3,5-bis(trifluoromethyl)phenyl]-5-fluoro-1,3-thiazol-2-yl}piperidin 1-yl)-2-oxoethyl]-3H-imidazo[4,5-b]pyridine

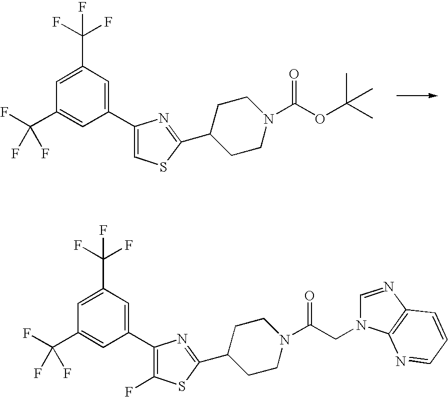

The thiazole (See Example 8 Step 1, (50 mg, 0.10 mmol) was dissolved in 1:1 CH₃CN:dichloroethane (11.0 mL), Selectofluor™ (37 mg, 0.010 mmol) added and the solution stirred for 1 hour at RT. The solution was then stirred overnight at 80° C. A second addition of Selectofluor™ (37 mg, 0.010 mmol) was made and the solution stirred overnight at 80° C. The volatiles were removed in vac and the residue dissolved in 2:1 CH₃CN:water (6 mL) and purified by RP-18 HPLC (CH₃CN:H₂O 15 minute gradient 10 to 100%:0.1% TFA). The recovered material, 1-HOBT (4 mg, 0.03 mmol) and the acid (See Example 1 Step 13, 4 mg, 0.02 mmol) were combined and the mixture dissolved in DMF (0.2 mL). Diisopropyl ethyl amine (23 mg, 0.18 mmol) and EDAC (5 mg, 0.03 mmol) were added and the solution stirred at RT overnight. The mixture was diluted in 2:1 CH₃CN:water (6 mL) and purified by RP-18 HPLC (CH₃CN:H₂O 15 minute gradient 10 to 100%:0.1% TFA) to give the titled compound. 1H NMR (500 MHz, CD3OD): δ 9.09 (s, 1H); 8.57 (d, J=4.7 Hz, 1H); 8.42 (s, 2H); 8.28 (s, 1H); 7.95 (s, 1H); 7.56 (dd, J=4.8, 7.5 Hz, 1H); 5.56 (ABq, Δδ=22.7, J=16.6 Hz, 2H); 4.53 (d, J=13.5 Hz, 1H); 4.21 (d, J=14.1 Hz, 1H); 3.48 (t, J=13.0 Hz, 1H); 3.37 (t, J=10.7 Hz, 1H); 2.99 (t, J=8.8 Hz, 2H); 2.33 (d, J=11.5 Hz, 1H); 2.20 (d, J=12.6 Hz, 1H); 2.08 (dq, J=3.9, 13.0 Hz, 1H); 1.81 (dq, J=3.9, 12.8 Hz, 1H).

MS: m/z=558.17 (M+H).

### Example 11

### Preparation of 3-(2-{4-[4-(3-bromo-5-tert-butylphenyl)-5-chloro-1,3-thiazol-2-yl]piperidin-1-yl}-2-oxoethyl)-3H-imidazo[4,5-b]pyridine

### Step 1

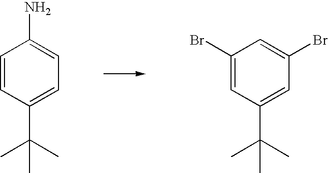

The aniline (75 g, 67 mol) was dissolved in DMF (37 mL), NBS added (25 grams, 142 mmol) and the solution stirred at RT overnight. The solution was diluted with aqueous NaOH (1 N, 370 mL) followed by aqueous/EtOAc work-up to afford the titled compound. The crude oil was dissolved in DMF (24 mL), added dropwise to a 70° C. solution of isoamyl nitrite (11.8 g, 101 mmol) in DMF (24 mL) and stirred for 30 minutes. The solution was cooled to RT, diluted with water (700 mL), acidified with AcOH, followed by aqueous/EtOAc work-up and silica gel chromatography (hexanes, neat) to afford the titled compound.

### Step 2

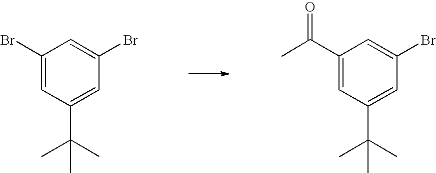

The bromide (See Example 11 Step 1, 0.67 g, 2.3 mmol), tri-nbutyl-1-ethoxyvinyl tin (0.3 g, 1.15 mmol), and PdCl₂(Ph₃P)₂ (81 mg, 0.12 mmol) were dissolved in toluene and the solution heated at 95° C. for 3 hours. The solution was cooled to RT, aqueous HCl (2 N, 2.56 mL) added and the mixture stirred rapidly overnight. The mixture was diluted with water (75 mL) and extracted with EtOAc (4×, 70 mL). The combined organic fraction was washed with brine, dried over Na₂SO, filtered, and the volatiles removed in vac. Silica gel chromatography (diethyl ether:hexanes (1:19)) gave the titled compound.

### Step 3

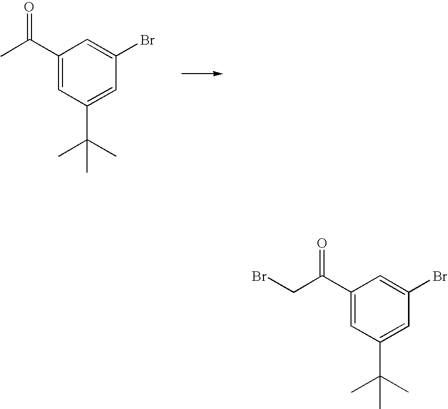

The ketone (See Example 11 Step 2, 0.30 g, 0.1.2 mmol) was dissolved in diethyl ether (7 mL) and aluminum chloride (4.6 mg, 0.05 mmol) was added. Bromine (0.13 g, 0.83 mmol) was added dropwise and the mixture stirred at RT for 10 minutes. The mixture was diluted with a solution of concentrated HCl/ice water (1:10, 40 mL), followed by aqueous/EtOAc workup and silica gel chromatography (diethyl ether:hexanes (1:4)) to afford the titled compound.

### Step 4

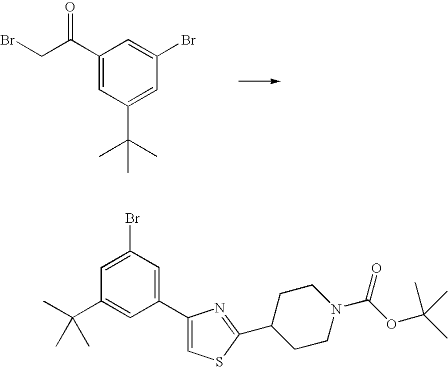

The bromoketone (See Example 11 Step 3, (0.30 g, 0.90 mmol) was dissolved in 1:1 ethyl alcohol/THF (4.5 mL), the thioamide (See Example 1 Step 9 (0.22 g, 0.9 mmol) added, and the mixture stirred at room temperature overnight. The volatiles were removed in. vac. and the residue dissolved in DMF (4.5 mL). Et₃N (0.91 g, 9.0 mmol) and di-tert-butyl dicarbonate (0.20 g, 0.90 mmol) were added and the solution stirred for 1 hour. The mixture was diluted with 4:1 water/saturated NaHCO₃ (300 mL) followed by aqueous/EtOAc work-up and silica gel chromatography (EtOAc:hexanes (1:9)) to afford the titled compound.

### Step 5

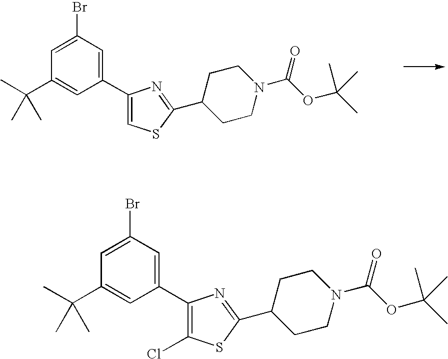

The thiazole (See Example 11 Step 4, (0.20 g, 0.42 mmol) was dissolved in DMF (4.2 mL), NCS (0.061 0.46 mmol) added, and the solution heated at 45° C. for 2 hours. The solution was cooled to RT, diluted with 8:1 water/saturated NaHCO₃ (80 mL) followed by aqueous/EtOAc work-up and silica gel chromatography (EtOAc:hexanes (1:9)) to afford the titled compound.

### Step 6

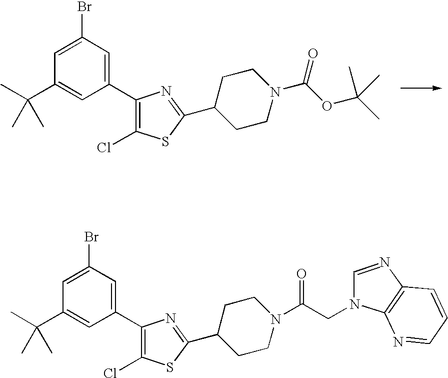

The chloride (See Example 11 Step 5 (0.19 g, 0.38 mmol) was dissolved in TEA (10 mL), stirred at RT for 10 minutes, and the volatiles removed in vac. The residue was dissolved in EtOAc, washed with 4:1 water/saturated NaHCO₃ (80 mL) and extracted (3 times) with EtOAc. The combined organic fraction was washed with brine, dried over Na₂SO, filtered and reduced in vac. Half of the recovered material, 1-HOBT (39 mg, 0.28 mmol) and the acid (See Example 1 Step 13, 40 mg, 0.23 mmol) were combined and the mixture dissolved in DMF (1.9 mL). Diisopropyl ethyl amine (122 mg, 0-95 mmol) and EDAC (54 mg, 0.28 mmol) were added and the solution stirred at RT overnight. The mixture was diluted with 2:1 CH₃CN:water (20 mL) and purified by RP-18 HPLC (CH₃CN:H₂O 15 minute gradient 10 to 100%:0.1% TFA) to give the titled compound.

¹H NMR (600 MHz, CD3OD): δ 9.15 (s, 1H); 8.59 (d, J=4.7 Hz, 1H); 8.28 (s, 1H); 7.96 (t, J=1.6 Hz, 1H); 7.87 (t, J=1.6 Hz, 1H); 7.58-7.56 (m, 2H); 5.57 (ABq, Δδ=25.4, J=16.9 Hz, 2H); 4.52 (d, J=13.4 Hz, 1H); 4.19 (d, J=14.1 Hz, 1H); 3.47 (t, J=13.1 Hz, 1H); 3.38 (tt, J=3.8, 11.4 Hz, 1H); 2.99 (t, J=12.2 Hz, 1H); 2.31 (d, J=13.0 Hz, 1H); 2.18 (d, J=12.1 Hz, 1H); 2.05 (dq, J=3.7, 12.9 Hz, 1H); 1.79 (dq, J=3.7, 12.4 Hz, 1H); 1.35 (s, 9H).

MS: m/z=572.07 (M+H).

### Example 12

### Preparation of 3-(2-{4-[4-(3-tert-butyl-5-cyclopropylphenyl)-5-chloro-1,3-thiazol-2-yl]piperidin-1-yl}-2-oxoethyl)-3H-imidazo[4,5-b]pyridine

### Step 1

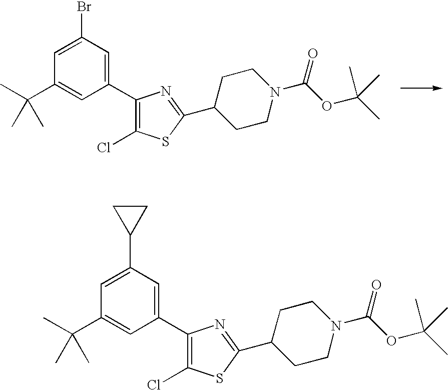

The bromide (See Example 11 Step 5, 0.044 g, 0.086 mmol) was dissolved in 20:1 toluene/water (0.86 mL), and potassium phosphate (76 mg, 0.28), cyclopropylboronic acid (8.13 mg, 0.095 mmol) and tetrakis(triphenylphosphine)palladium(0) (0.050 g, 0.043 mmol) were added. The flask was purged with nitrogen (3 times) and the slurry stirred at 99° C. for 7 hours. A second addition of potassium phosphate (76 mg, 0.28) and cyclopropylboronic acid (8.13 mg, 0.095 mmol) was made and the solution stirred at 99 for 3 hours. The solution was cooled to RT, diluted with water (40 mL), followed by aqueous/EtOAc work-up and silica gel chromatography (diethyl ether:hexanes (3:7)) to give the titled compound.

### Step 2

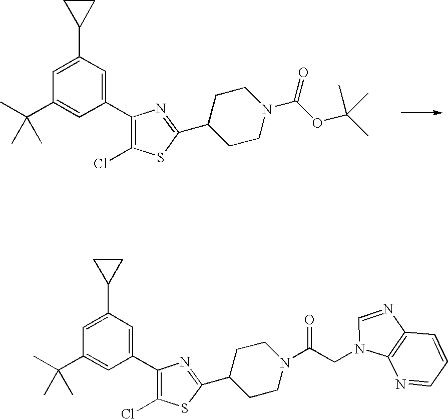

The chloride (See Example 12 Step 1, 20 mg, 0.039 mmol) was dissolved in TFA (2 mL), stirred at RT for 10 minutes, and the volatiles removed in vac. The residue was dissolved in EtOAc, washed with 4:1 water/saturated NaHCO₃ (80 mL) and extracted (3 times) with EtOAc. The combined organic fraction was washed with brine, dried over Na₂SO, filtered and reduced in vac. The recovered material, 1-HOBT (8 mg, 0.058 mmol) and the acid (See Example 1 Step 13, 8.3 mg, 0.047 mmol) were combined and the mixture dissolved in DMF (0.4 mL). Diisopropyl ethyl amine (25 mg, 0.20 mmol) and EDAC (11 mg, 0.055 mmol) were added and the solution stirred at RT overnight. The mixture was diluted with CH₃CN:water (6 mL) and purified by RP-18 HPLC (CH₃CN:H₂O 15 minute gradient 10 to 100%:0.1% TFA) to give the titled compound.

¹H NMR (600 MHz, CD3OD): δ 9.01 (s, 1H); 8.57 (d, J=4.1 Hz, 1H); 8.24 (d, J=8.1 Hz, 1H); 7.69 (t, J=1.7 Hz, 1H); 7.54 (dd, J=4.7, 8.1 Hz, 1H); 7.34 (t, J=1.5 Hz, 1H); 7.20 (t, J=1.7 Hz, 1H); 5.55 (ABq, Δδ=26.2, J=16.9 Hz, 2H); 4.53 (d, J=13.5 Hz, 1H); 4.20 (d, J=14.0 Hz, 1H); 3.47 (t, J=13.0 Hz, 1H); 3.38 (tt, J=3.9, 11.5 Hz, 1H); 2.98 (t, J=14.0 Hz, 1H); 2.31 (d, J=13.0 Hz, 1H); 2.18 (d, J=12.1 Hz, 1H); 2.04 (dq, J=3.8, 12.9 Hz, 1H); 1.99-1.95 (m, 1H); 1.79 (dq, J=4.2, 12.8 Hz, 1H); 1.35 (s, 9H); 1.00-0.98 (m, 2H); 0.72-0.70 (m, 2H).

MS: m/z=534.15 (M+H).

### Example 13

### Preparation of 3-(2-{4-[5-bromo-4-(3-tert-butyl-5-cyclopropylphenyl)-1,3-thiazol-2-yl]piperidin-1-yl}-2-oxoethyl)-3H-imidazo[4,5-b]pyridine

### Step 1

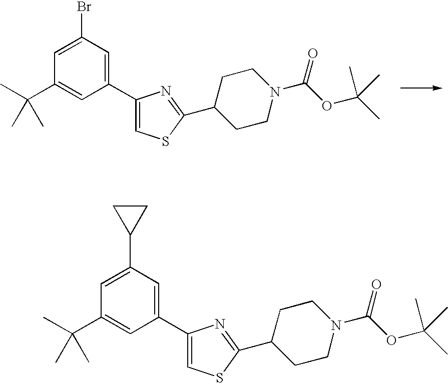

The bromide (See Example 11 Step 4, 0.20 g, 0.42 mmol) was dissolved in 20:1 toluene:water (2.0 mL), and potassium phosphate (0.39 g, 1.48 mmol), cyclopropylboronic acid (47 mg, 0.55 mmol) and tetrakis(triphenylphosphine)palladium(0) (0.20 g, 0.17 mmol), were added. The vessel was purged with nitrogen (3 times) and the slurry stirred at 95° C. for 2 hours. The solution was allowed to cool to RT, diluted with aqueous NaOH (1N, 75 mL), followed by aqueous/EtOAc work-up and silica gel chromatography (diethyl ether:hexanes (3:7)). A second RP-18 HPLC (CH₃CN:H₂O 15 minute gradient 10 to 100%:0.1% TFA) chromatography gave the titled compound.

### Step 2

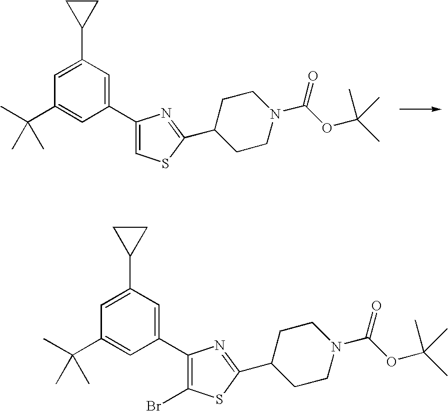

The thiazole (See Example 13 Step 1, 0.049 g, 0.11 mmol) was dissolved in DMF (1.1 mL), NBS (0.022 g, 0.12 mmol) added and the solution stirred at RT overnight. The solution was diluted with 4:1 water/saturated NaHCO₃ (80 mL), followed by aqueous/EtOAc work-up and silica gel chromatography (diethyl ether:hexanes (3:7)) to afford the titled compound.

### Step 3

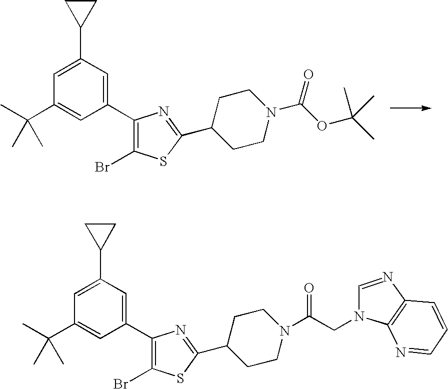

The bromide (See Example 13 Step 2 (23 mg, 0.044 mmol) was dissolved in TFA (2 mL), stirred at RT for 10 minutes, and the volatiles removed in vac. The residue was dissolved in EtOAc, washed with 4:1 water/saturated NaHCO₃ (80 mL) and the aqueous wash re-extracted (3 times) with EtOAc. The combined organic fraction was washed with brine, dried over Na₂SO, filtered and reduced in vac. The recovered material, 1-HOBT (9 mg, 0.066 mmol) and the acid (See Example 1 Step 13, 9.3 mg, 0.053 mmol) were combined and the mixture dissolved in DMF (0.44 mL). Diisopropyl ethyl amine (28 mg, 0.22 mmol) and EDAC (13 mg, 0.066 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.16 (s, 1H); 8.61 (dd, J=1.1, 4.8 Hz, 1H); 8.27 (d, J=8.2 Hz, 1H); 7.67 (t, J=1.6 Hz, 1H); 7.59 (dd, J=4.8, 8.2 Hz, 1H); 7.31 (s, 1H); 7.21 (t, J=1.6 Hz, 1H); 5.58 (ABq, Δδ=20.7, J=17 Hz, 2H); 4.53 (d, J=13.5 Hz, 1H); 4.20 (d, J=14.0 Hz, 1H); 3.47 (t, J=13.2 Hz, 1H); 3.40 (tt, J=3.9, 11.4 Hz, 1H); 2.98 (t, J=13.8 Hz, 1H); 2.31 (d, J=12.8 Hz, 1H); 2.19 (d, J=12.6 Hz, 1H); 2.05 (dq, J=3.9, 12.8 Hz, 1H); 1.99-1.93 (m, 1H); 1.79 (dq, J=3.9, 12.5 Hz, 1H); 1.35 (s, 9H); 1.01-0.97 (m, 2H); 0.73-0.69 (m, 2H).

MS: m/z=578.12 (M+H).

### Example 14

### Preparation of 4-[5-bromo-2-[1-(3H-imidazo[4,5-b]pyridin-3-ylacetyl)piperidin-4-yl]-1,3-thiazol-4-yl]-2,6-di-tert-butylphenol

### Step 1

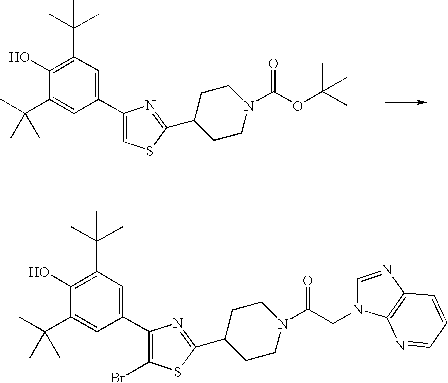

The thiazole (See Example 6 Step 1, 70 mg, 0.15 mmol) was dissolved in TFA (2 mL), stirred at RT for 10 minutes, and the volatiles removed in vac. The residue was dissolved in acetic acid (1.5 mL) and bromine (0.8 M in acetic acid, 0.21 mL, 0.16 mmol) was added dropwise. The solution was stirred for 1 hour at RT. The volatiles were removed in. vac., the residue dissolved in EtOAc, partitioned with aqueous NaOH (1 N, 40 mL) followed by aqueous/EtOAc work-up. The recovered material, 1-HOBT (10 mg, 0.075 mmol) and the acid (See Example 1 Step 13, 11 mg, 0.060 mmol) were combined and the mixture dissolved in DMF (0.5 mL). Diisopropyl ethyl amine (32 mg, 0.25 mmol) and EDAC (14 mg, 0.075 mmol) were added and the solution allowed to stir at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (600 MHz, CDCl₃): δ 9.60 (s, 1H); 8.68 (d, J=4.7 Hz, 1H); 8.40 (d, J=7.7 Hz, 1H); 7.65 (s, 2H); 7.62 (dd, J=4.9, 8.2 Hz, 1H); 5.50 (ABq, Δδ=22.7, J=16.4 Hz, 2H); 4.59 (d, J=13.5 Hz, 1H); 4.06 (d, J=13.4 Hz, 1H); 3.99 (s, 1H); 3.51-3.45 (m, 2H); 2.99 (t, J=11.7 Hz, 1H); 2.39 (d, J=13.3 Hz, 1H); 2.23 (t, J=12.5 Hz, 1H); 1.98 (q, J=10.9 Hz, 1H); 1.82 (q, J=10.8 Hz, 1H); 1.49 (s, 18H).

MS: i/z 610.16 (M+H).

### Example 15

### Preparation of 3-[2-(4-{4-[3-bromo-5-(trifluoromethoxy)-4-(trifluoromethyl)phenyl]-5-chloro-1,3-thiazol-2-yl}piperidin-1-yl-2-oxoethyl]-3H-imidazo[4,5-b]pyridine

### Step 1

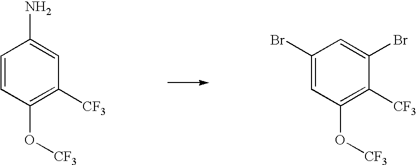

The aniline (2 g, 8.2 mmol) was dissolved in DMF (16 mL), NBS (3.6 g, 20 mmol) added and the solution stirred at RT overnight. The mixture was diluted with aqueous NaOH (1N, 250 mL) followed by aqueous/EtOAc work-up to afford the titled compound. The material was then dissolved in DMF (3.6 mL), added to a 70° C. solution of isoamyl nitrite (1.4 g, 12 mmol) in DMF (2 mL), and stirred with constant heating overnight. The solution was cooled to RT, diluted with water (150 mL), followed by aqueous/EtOAc work-up and silica gel chromatography (hexanes, neat) to afford the titled compound.

### Step 2

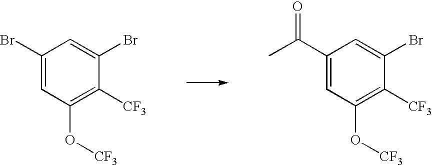

The bromide (See Example 15 Step 1, 0.52 g, 1.3 mmol), tri-nbutyl-1-ethoxyvinyl tin (0.51 g, 1.41 mmol), and PdCl₂(Ph₃P)₂ (94 mg, 0.13 mmol) were dissolved in toluene and the solution stirred at 95° C. for 4 hours. The volatiles were removed in vac. The residue was dissolved in 1,4-dioxane (20 mL), aqueous HCl (2 N, 2 mL) added, and the solution stirred rapidly at RT for 3 hours. The mixture was diluted with water (150 mL), followed by aqueous/EtOAc work-up, and silica gel chromatography (EtOAc:hexanes (1:19)) to afford the titled compound.

### Step 3

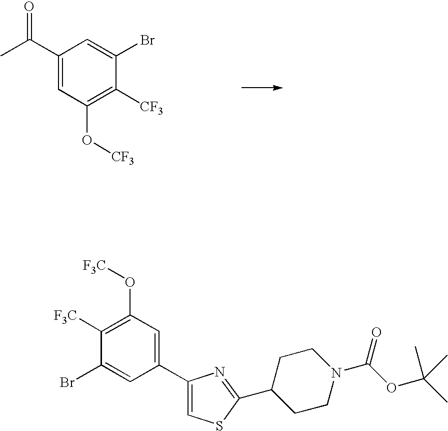

The ketone (See Example 15 Step 2, 0.16 g, 0.46 mmol) was dissolved at RT in diethyl ether (2.3 mL), and aluminum chloride (3.0 mg, 0.02 mmol) added. Bromine (80 mg, 0.50 mmol) was added dropwise to the solution and the mixture stirred at RT for 10 minutes. The mixture was diluted with a solution of concentrated HCl/ice water (1:10, 40 mL), followed by aqueous/EtOAc work-up. The resulting material was dissolved in 1:1 ethyl alcohol/THF (2.3 mL), the thioamide (See Example 1 Step 9 (0.11 g, 0.46 mmol) added, and the mixture stirred at 45° C. for 3 hours. The volatiles were removed in. vac. and the residue dissolved DMF (3.0 mL). Et₃N (0.46 g, 4.6 mmol) and di-tert-butyl dicarbonate (100 mg, 0.46 mmol) were added and the solution stirred at RT for 1 hour. The mixture was diluted with 4:1 water/saturated NaHCO₃, followed by aqueous/EtOAc work-up and silica gel chromatography (diethyl ether:hexanes (3:7)) to afford the titled compound.

### Step 4

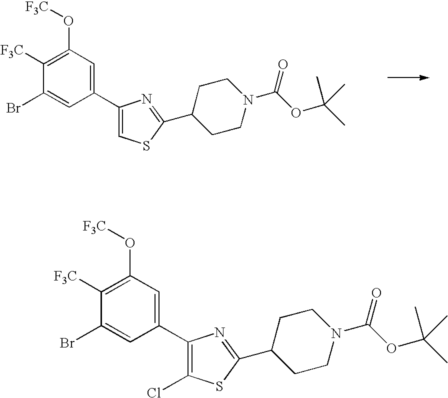

The thiazole (See Example 15 Step 3, (0.19 g, 0.33 mmol) was dissolved in DMF (3.0 mL), NCS (0.47 g, 3.55 mmol) added and the solution stirred at RT for 2.5 hours. A second addition of NCS (0.16 g, 1.2 mmol) was made and the solution stirred at 55° C. for 2.5 hours. The solution was cooled to RT, diluted with aqueous NaOH (1N, 60 mL), followed by aqueous/EtOAc work-up and silica gel chromatography (diethyl ether:hexanes (3:7)) to afford the titled compound.

### Step 5

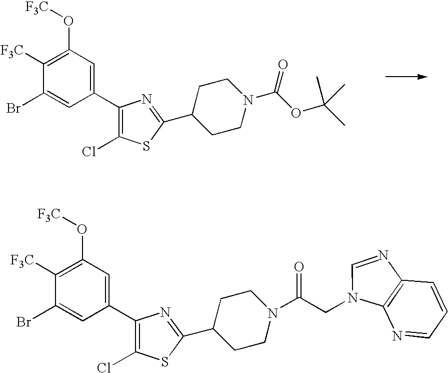

The chloride (See Example 13 Step 4 30 mg, 0.049 mmol) was dissolved in TFA (2 mL), stirred for 10 minutes and the volatiles removed in vac. The residue was dissolved in EtOAc, diluted with aqueous NaOH (1N, 40 mL), followed by aqueous/EtOAc work-up. The recovered material, 1-HOBT (10 mg, 0.074 mmol) and the acid (See Example 1 Step 13, 0.10 g, 0.059 mmol) were combined and the mixture dissolved in DMF (0.49 mL). Diisopropyl ethyl amine (32 mg, 0.25 mmol) and EDAC (14 mg, 0.074 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.00 (s, 1H); 8.56 (dd, J=1.2, 4.8 Hz, 1H); 8.49 (s, 1H); 8.24 (dd, J=1.2, 8.2 Hz, 1H); 8.14 (s, 1H); 7.54 (dd, J=4.8, 8.2 Hz, 1H); 5.55 (ABq, Δδ=46.7, J=12.8 Hz, 2H); 4.53 (d, J=13.8 Hz, 1H); 4.21 (d, J=14.2 Hz, 1H); 3.48 (t, J=13.0 Hz, 1H); 3.42 (tt, J=3.9, 11.4 Hz, 1H); 3.00 (t, J=13.6 Hz, 1H); 2.32 (d, J=13.4 Hz, 1H); 2.20 (d, J=11.8 Hz, 1H); 2.06 (dq, J=3.9, 13.0 Hz, 1H); 1.81 (dq, J=3.8, 12.8 Hz, 1H).

MS: m/z=668.13 (M+H).

### Example 16

### Preparation of 3-[2-(4-{5-chloro-4-[3-cyclopropyl-5-(trifluoromethoxy)-4-(trifluoromethyl)phenyl]-1,3-thiazol-2-yl}piperidin-1-yl)-2-oxoethyl]-3H-imidazo[4,5-b]pyridine

### Step 1

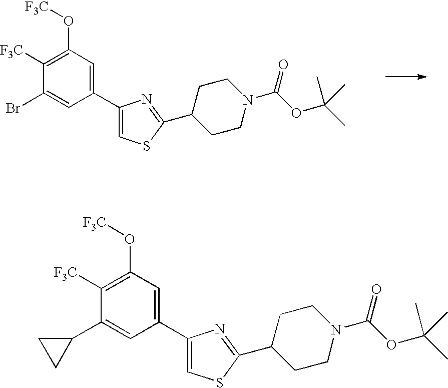

The bromide (See Example 15 Step 3, 0.057 g, 0.10 mmol) was dissolved in 20:1 toluene:water (1.0 mL), and potassium phosphate (0.16 g, 0.59 mmol), cyclopropylboronic acid (17 mg, 0.2 mmol) and tetrakis(triphenylphosphine)palladium(0) (0.070 g, 0.059 mmol) added. The flask was purged with nitrogen (3 times) and the slurry stirred at 95° C. for 3 hours. The solution was cooled to RT, diluted with aqueous NaOH (1N, 75 mL), followed by aqueous/EtOAc work-up and silica gel chromatography (diethyl ether:hexanes (1:4)) to give the titled compound.

### Step 2

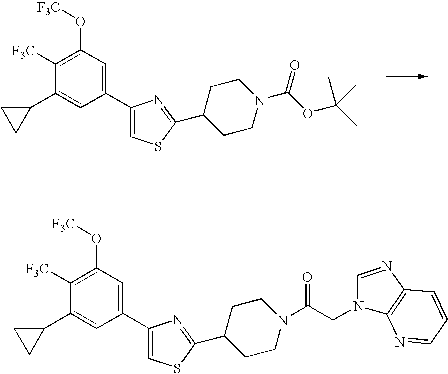

The chloride (See Example 16 Step 1, 787 (21 mg, 0.039 mmol) was dissolved in TFA (2 mL), stirred for 10 minutes, and the volatiles removed in vac. The residue was dissolved in EtOAc, washed with aqueous NaOH (1N, 40 mL) and the aqueous phase re-extracted with EtOAc (3 times). The combined organic fraction was washed with brine, dried over MgSO₄, filtered and reduced in vac. The recovered material, 1-HOBT (8 mg, 0.059 mmol) and the acid (See Example 1 Step 13, 8.3 mg, 0.047 mmol) were combined and the mixture dissolved in DMF (0.40 mL). Diisopropyl ethyl amine (25 mg, 0.20 mmol) and EDAC (11 mg, 0.059 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

### Step 3

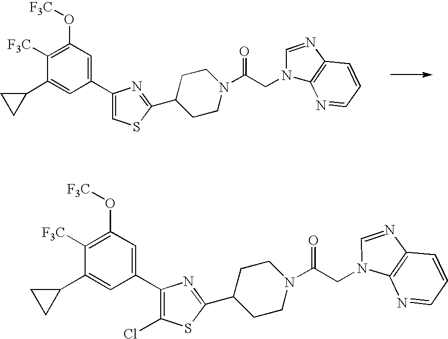

The amide (See Example 16 Step 2, 8 mg, 0.011 mmol) was dissolved in DMF (0.11 mL), NCS (1.7 mg, 3.55 mmol) added and the solution stirred at 45° C. for 2.5 hours. The mixture was cooled to RT, diluted with 2:1 CH₃CN:water (6 mL), and purified by RP-18 HPLC (CH₃CN:H₂O 15 minute gradient 10 to 100%:0.1% TFA) to give the titled compound.

¹H NMR (500 MHz, CD3OD): δ 9.02 (s, 1H); 8.57 (d, J=4.7 Hz, 1H); 8.25 (d, J=82 Hz, 1H); 7.90 (s, 1H); 7.87 (s, 1H); 7.55 (dd, J=4.8, 8.1 Hz, 1H); 5.55 (ABq, Δδ=24.7, J=17.0 Hz, 2H); 4.53 (d, J=13.4 Hz, 1H); 4.21 (s, J=13.9 Hz, 1H); 3.48 (t, J=12.9 Hz, 1H); 3.42 (tt, J=3.6, 11.5 Hz, 1H); 3.00 (t, J=11.4 Hz, 1H); 2.32 (m, 2H); 2.20 (d, J=12.4 Hz, 1H); 2.05 (dq, J=4.2, 13.1 Hz, 1H); 1.80 (dq, J=4.1, 12.8 Hz, 1H); 1.13 (m, 2H); 0.85 (m, 2H).

MS: m/z=630.25 (M+H).

### Example 17

### Preparation of 3-[2-(4-{5-chloro-4-[3-cyclopropyl-5-(trifluoromethoxy)phenyl]-1,3-thiazol-2-yl}piperidin-1-yl)-2-oxoethyl]-3H-imidazo[4,5-b]pyridine

### Step 1

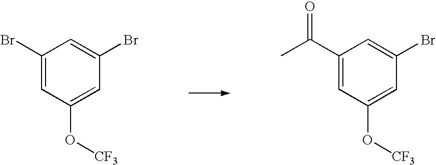

The bromide (3.0 g, 9.38 mmol), tri-nbutyl-1-ethoxyvinyl tin (3.56 g, 9.85 mmol), and PdCl₂(Ph₃P)₂ (0.66 g, 0.94 mmol) were dissolved in toluene and the solution stirred at 95° C. for 3 hours. The volatiles were removed in vac., the residue dissolved in 1,4-dioxane (20 mL), aqueous HCl (2 N, 14 mL) added, and the solution stirred rapidly at RT for 1 hour. The mixture was diluted with water (400 mL), followed by aqueous/EtOAc work-up and silica gel chromatography (EtOAc:hexanes (1:19)) to give the titled compound.

### Step 2

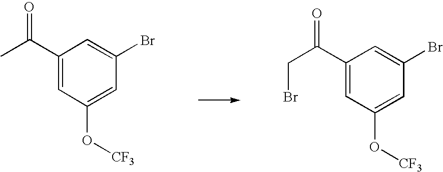

The ketone (See Example 17 Step 1, 0.40 g, 1.4 mmol) was dissolved in diethyl ether (7 mL), and aluminum chloride (2.0 mg, 0.014 mmol) added. Bromine (0.24 g, 1.5 mmol) was added dropwise and the resulting mixture stirred for 10 minutes at RT. The mixture was diluted with a solution of concentrated HCl/ice water (1:10, 80 mL) followed by aqueous/EtOAc work-up.

### Step 3

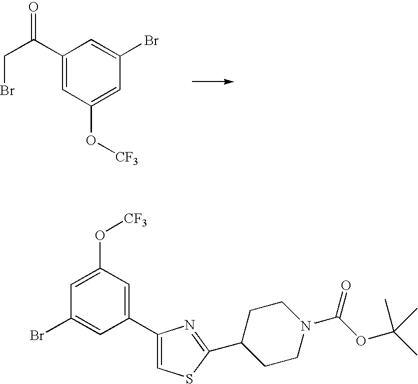

The bromoketone (See Example 17 Step 2, (0.50 g, 0.1.38 mmol) was dissolved in 1:1 ethyl alcohol/THF (7.0 mL), the thioamide (See Example 1 Step 9, 0.34 g, 1.4 mmol) added, and the mixture stirred at room temperature for 1 hour. The solution was diluted with aqueous NaOH (1N, 150 mL), followed by aqueous/EtOAc work-up and silica gel chromatography (diethyl ether:hexanes (3:7)) to afford the titled compound.

### Step 4

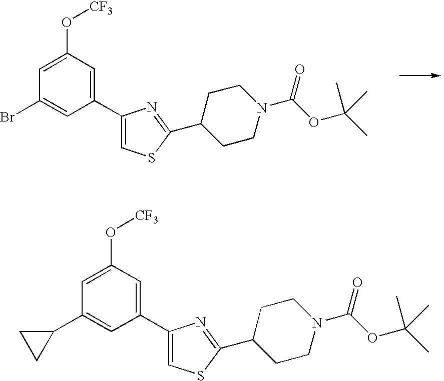

The bromide (See Example 17 Step 3, 0.10 g, 0.20 mmol) was dissolved in 20:1 toluene:water (1.0 mL), and potassium phosphate (0.18 g, 0.69 mmol), cyclopropylboronic acid (22 mg, 0.26 mmol), and tetrakis(triphenylphosphine)palladium(0) (0.091 g, 0.079 mmol) added. The flask was purged with nitrogen (3 times) and the slurry stirred at 95° C. for 2 hours. The solution was cooled to RT, diluted with aqueous NaOH (1N, 75 mL), followed by aqueous/EtOAc work-up and silica gel chromatography (diethyl ether:hexanes (3:7)) to give the titled compound.

### Step 5

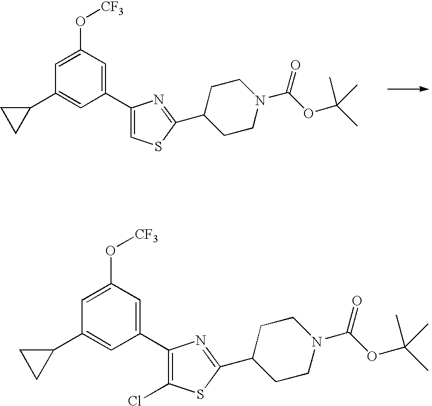

The thiazole (See Example 17 Step 4, (0.079 g, 0.17 mmol) was dissolved in DMF (1.7 mL), NCS (0.025 0.19 mmol) added, and the solution stirred at 45° C. for 3 hours. The solution was cooled to RT, diluted with aqueous NaOH (1N, 80 mL), followed by aqueous/EtOAc work-up and silica gel chromatography (EtOAc:hexanes (1:9)) to afford the titled compound.

### Step 6

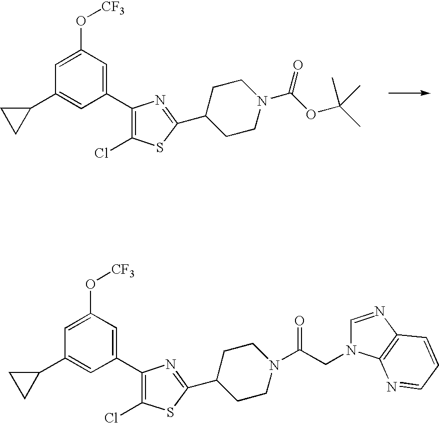

The chloride (See Example 17 Step 5, 0.29 g, 0.058 mmol) was dissolved in TFA (2 mL), stirred for 10 minutes, and the volatiles removed in vac. The residue was dissolved in EtOAc, washed with 4:1 water/saturated NaHCO₃ (80 mL) and the aqueous wash re-extracted (3 times) with EtOAc. The combined organic fraction was washed with brine, dried over Na₂SO, filtered and reduced in vac. The recovered material, 1-HOBT (12 mg, 0.087 mmol) and the acid (See Example 1 Step 13, 12 mg, 0.070 mmol) were combined and the mixture dissolved in DMF (0.58 mL). Diisopropyl ethyl amine (38 mg, 0.29 mmol) and EDAC (17 mg, 0.087 mmol) were added and the solution stirred at RT overnight. The mixture was diluted with 2:1 CH₃CN:water (6 mL) and purified by RP-18 HPLC (CH₃CN:H₂O 15 minute gradient 10 to 00%:0.1% TFA) to give the titled compound.

¹H NMR (500 MHz, CD3OD): δ 9.16 (s, 1H); 8.61 (dd, J=1.1, 4.7 Hz, 1H); 8.27 (d, J=7.4 Hz, 1H); 7.65 (s, 1H); 7.61-7.57 (m, 2H); 7.01 (s, 1H); 5.58 (ABq, Δδ=21.3, J=16.9 Hz, 2H); 4.52 (d, J=13.5 Hz, 1H); 4.19 (d, J=14.1 Hz, 1H); 3.48 (t, J=13.1 Hz, 1H); 3.39 (tt, J=3.9, 11.4 Hz, 1H); 2.99 (t, J=13.6 Hz, 1H); 2.31 (d, J=11.9 Hz, 1H); 2.18 (d, J=11.7 Hz, 1H); 2.08-1.99 (m, 2H); 1.83-1.74 (dq, J=3.9, 12.9 Hz, 1H); 1.09-1.05 (m, 2H); 0.78-0.74 (m, 2H).

MS: m/z=562.33 (M+H).

### Example 18

### Preparation of 3-[2-(4-{5-chloro-4-[3-(1,1-difluoroethyl)-5-(trifluoromethoxy)phenyl]-1,3-thiazol-2-yl}piperidin-1-yl)-2-oxoethyl]-3H-imidazo[4,5-b]pyridine

### Step 1

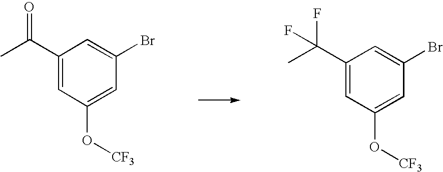

The bromide (See Example 17 Step 1, 0.30 g, 1.1 mmol) was dissolved in Deoxyfluor (0.28 g, 1.3 mmol) and the solution stirred at 85° C. for 5 hours. The mixture was diluted with saturated NaHCO₃ (40 mL) followed by aqueous/EtOAc work-up and silica gel chromatography (diethyl ether:hexanes (1:9)) to give the titled compound.

### Step 2

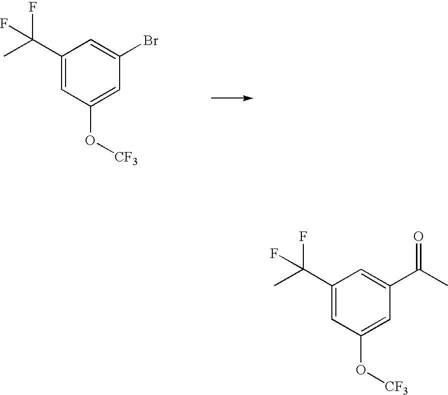

The bromide (See Example 18 Step 2, 160 mg, 0.52 mmol), tri-nbutyl-1-ethoxyvinyl tin (0.23 g, 0.63 mmol), and PdCl₂(Ph₃P)₂ (37 mg, 0.052 mmol) were dissolved in toluene 1.1 mL) and the solution stirred at 95° C. for 3 hours. The volatiles were removed in vac. and the residue dissolved in 1,4-dioxane (3 mL). Aqueous HCl (2 N, 0.79 mL) was added and the resulting mixture stirred rapidly for 1 hour. The mixture was diluted with water (40 mL), followed by aqueous/EtOAc work-up and silica gel chromatography (diethyl ether:hexanes (1:9)) to give the titled compound.

### Step 3

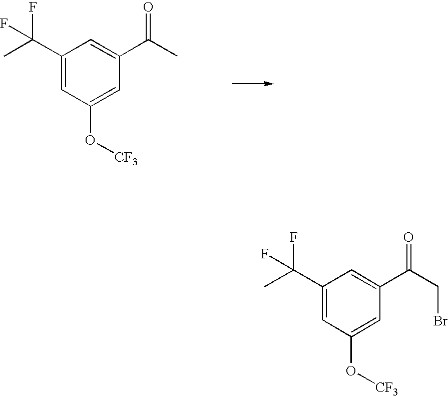

The ketone (See Example 18 Step 3, 0.11 g, 0.40 mmol) was dissolved in diethyl ether (2 mL), and aluminum chloride (1.0 mg, 0-014 mmol) added. Bromine (0.068 g, 0.42 mmol) was added dropwise and the solution stirred at RT for 10 minutes. The mixture was diluted with a solution of concentrated HCl:ice water (1:10, 40 mL) followed by aqueous/EtOAc work-up to afford the titled compound.

### Step 4

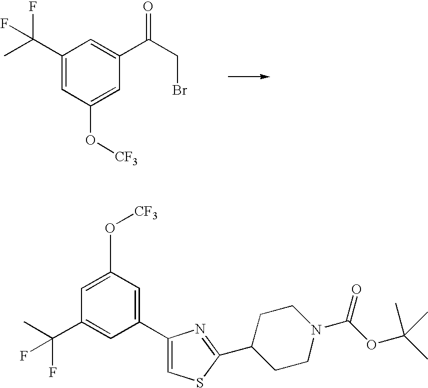

The bromoketone (See Example 18 Step 3, 0.13 g, 0.39 mmol) was dissolved in 1:1 ethyl alcohol/THF (4.0 mL), the thioamide (See Example 1 Step 13, 0.094 g, 0.39 mmol) added, and the solution stirred at room temperature overnight. The mixture was diluted with aqueous NaOH (1N, 40 mL), followed by aqueous/EtOAc work-up and silica gel chromatography (diethyl ether:hexanes (3:17)) to afford the titled compound.

### Step 5

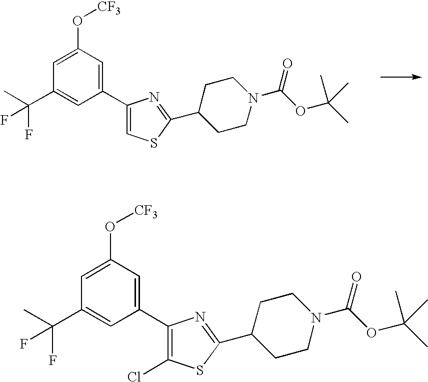

The thiazole (See Example 18 Step 4, 0.057 g, 0.12 mmol) was dissolved in DMF (1.1 mL), NCS (0.017 0.13 mmol) added, and the solution stirred at 45° C. for 1 hour. The solution was cooled to RT, diluted with aqueous NaOH (1N, 40 mL), followed by aqueous/EtOAc work-up and silica gel chromatography (EtOAc:hexanes (1:4)) to afford the titled compound.

### Step 6

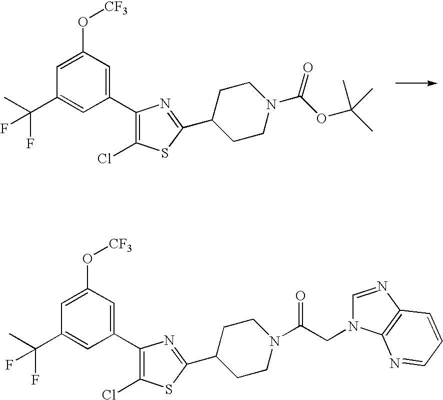

The chloride (See Example 18 Step 5, 787 (0.023 g, 0.044 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash re-extracted with EtOAc (3 times). The combined organic fraction was washed with brine, dried over Na₂SO, filtered and the volatiles removed in vac. The recovered material, 1-HOBT (9.0 mg, 0.066 mmol) and the acid (See Example 1 Step 13, 9-0 mg, 0.053 mmol) were combined and the mixture dissolved in DMF (0.44 mL). Diisopropyl ethyl amine (28 mg, 0.22 mmol) and EDAC (13 mg, 0.066 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.12 (s, 1H); 8.59 (d, J=4.8 Hz, 1H); 8.26 (d, J=8.2 Hz, 1H); 8.15 (s, 1H); 7.98 (s, 1H); 7.58 (dd, J=5.2, 8.2 Hz, 1H); 7.47 (s, 1H); 5.57 (ABq, Δδ=21.9, J=17.0 Hz, 2H); 4.53 (d, J=13.0 Hz, 1H); 4.20 (d, J=13.5 Hz, 1H); 3.48 (t, J=13.2 Hz, 1H); 3.41 (tt, J=3.9, 11.4 Hz, 1H); 3.00 (t, J=13.6 Hz, 1H); 2.32 (d, J=11.9 Hz, 1H); 2.19 (d, J=13.0 Hz, 1H); 2.07 (dq, J=3.9, 12.8 Hz, 1H); 1.98 (t, J=18.5 Hz, 3H); 1.81 (dq, J=3.9, 12.6 Hz, 1H).

MS: m/z=586.24 (M+H).

### Example 19

### Preparation of 3-[2-(4-{4-[3-tert-butyl-5-(trifluoromethoxy)phenyl]-5-chloro-1,3-thiazol-2-yl}piperidin-1-yl)-2-oxoethyl]-3H-imidazo[4,5-b]pyridine

### Step 1

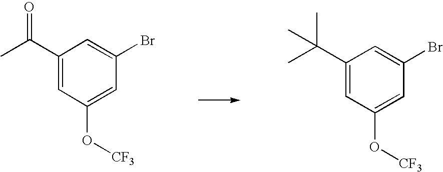

An oven dried flask was charged with CH₂Cl₂ (1.4 mL) and titanium tetrachloride (134 mg, 0.71 mmol). The solution was cooled to −30° C., followed by dimethyl zinc (10% wt in hexanes, 0.67 g, 0.71 mmol) added dropwise, and stirred at constant temperature for 30 minutes. A solution of the ketone (See Example 17 Step 1, 0.25 M in CH₂Cl₂, 1.41 mL) was added dropwise and the solution then allowed to warm to 0° C. over 40 minutes. The solution was stirred for 2 hours at RT, 45 minutes at 45° C., and overnight at 40° C. The slurry was diluted with saturated NaHCO₃ (40 mL) and extracted with EtOAc (3 times). The combined organic fraction was washed with equal volume of aqueous HCl (2N), brine, dried over MgSO₄, filtered, reduced in vac, followed by silica gel chromatography (diethyl ether:hexanes (1:19)) to give the titled compound.

### Step 2

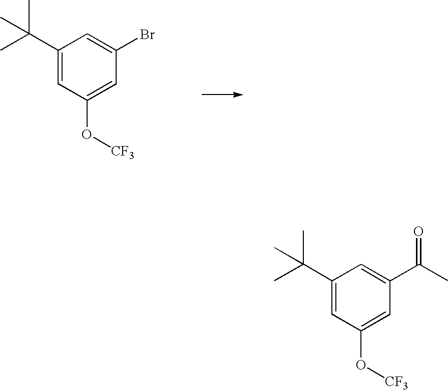

The bromide (See Example 19 Step 1, 0.17 g, 0.58 mmol), tri-nbutyl-1-ethoxyvinyl tin (0.27 g, 0.76 mmol), and dichloro bis(triphenylphosphine)palladium (11) (41 mg, 0.058 mmol) were dissolved in toluene (1.2 mL) and the solution stirred at 95° C. for 3 hours. The volatiles were removed in vac., the residue dissolved in 1,4-dioxane (2 mL), aqueous HCl (2 N, 0.88 mL) added, and the solution stirred rapidly at RT for 1 hour. The mixture was diluted with water (40 mL) followed by aqueous/EtOAc work-up and silica gel chromatography (EtOAc:hexanes (1:19)) to give the titled compound.

### Step 3

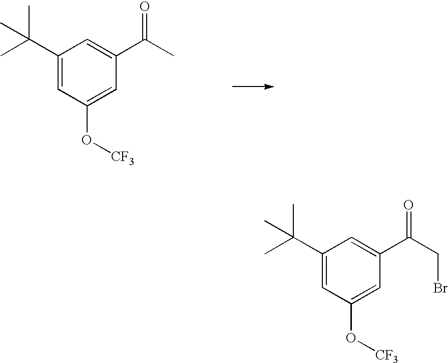

The ketone (See Example 19 Step 2, 0.060 g, 0.23 mmol) was dissolved in diethyl ether (1.2 mL) and aluminum chloride (2.0 mg, 0.014 mmol) added at RT. Bromine (0.039 g, 0.24 mmol) was added dropwise and the resulting mixture was stirred for 10 minutes. The mixture was diluted with solution of concentrated HCl/ice water (1:10, 40 mL) followed by aqueous/EtOAc work-up to give the titled compound.

### Step 4

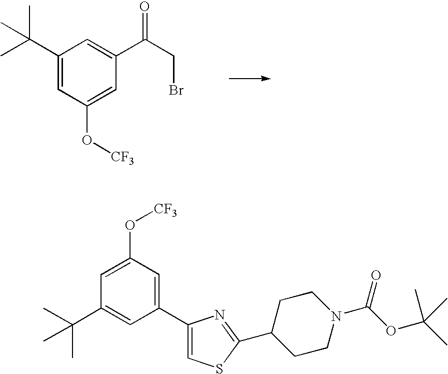

The bromoketone (See Example 19 Step 3, (0.078 g, 0.23 mmol) was dissolved in 1:1 ethyl alcohol/THF (1.1 mL), the thioamide (See Example 1 Step 9, 0.056 g, 0.23 mmol) added, and the mixture stirred at room temperature overnight. The volatiles were removed in. vac. and the residue dissolved in DMF (1.0 mL). Et₃N (0.23 g, 2.3 mmol) and di-tert-butyl dicarbonate (50 mg, 0.23 mmol) were added and the solution stirred at RT for 1 hour. The solution was diluted with aqueous NaOH (1N, 40 mL), followed by aqueous/EtOAc work-up and silica gel chromatography (diethyl ether:hexanes (3:17)₆) to afford the titled compound.

### Step 5

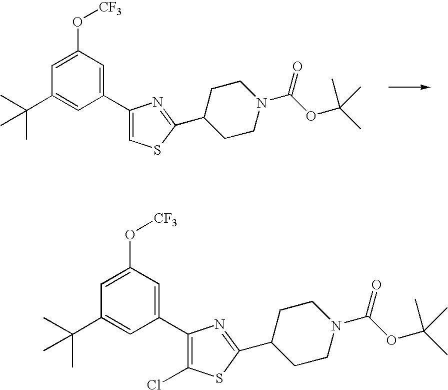

The thiazole (See Example 19 Step 4, (0.069 g, 0.14 mmol) was dissolved in DMF (1.4 mL), NCS (0.021 0.16 mmol) added, and the solution stirred at 45° C. for 1 hour. The solution was cooled to RT, diluted with aqueous NaOH (1N, 40 μL), followed by aqueous/EtOAc work-up and silica gel chromatography (diethyl ether:hexanes (1:4)) to afford the titled compound.

### Step 6

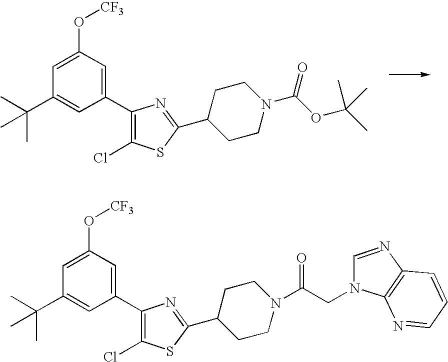

The chloride (See Example 19 Step 5, 0.034 g, 0.070 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash re-extracted (3 times) with EtOAc. The combined organic fraction was washed with brine, dried over Na₂SO, filtered and reduced in vac. The recovered material, 1-HOBT (14 mg, 0.11 mmol) and the acid (See Example 1 Step 13, 15 mg, 0.084 mmol) were combined and the mixture dissolved in DMF (0.70 mL). Diisopropyl ethyl amine (45 mg, 0.35 mmol) and EDAC (20 mg, 0.11 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.17 (s, 1H); 8.60 (d, J=4.7 Hz, 1H); 8.28 (d, J=7.5 Hz, 1H); 8.01 (s, 1H); 7.64 (s, 1H); 7.58 (dd, J=4.8, 8.0 Hz, 1H); 7.32 (s, 1H); 5.58 (ABq, Δδ=21.8, J=16.9 Hz, 2H); 4.52 (d, J=13.4 Hz, 1H); 4.20 (d, J=13.9 Hz, 1H); 3.48 (t, J=13.1 Hz, 1H); 3.40 (tt, J=3.9, 11.4 Hz, 1H); 3.00 (t, J=13.3 Hz, 1H); 2.32 (d, J=12.1 Hz, 1H); 2.19 (d, J=12.6 Hz, 1H); 2.05 (dq, J=3.9, 12.8 Hz, 1H); 1.80 (dq, J=3.8, 12.5 Hz, 1H); 1.38 (s, 9H).

MS: m/z=578.13 (M+H).

### Example 20

### Preparation of 3-(2-{4-[5-bromo-4-(3,5-di-tert-butylphenyl)-1,3-thiazol-2-yl]piperidin-1-yl}-2-oxoethyl)-3H-imidazo[4,5-b]pyridine

### Step 1

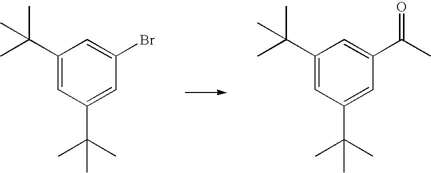

The bromide (2.0 g, 7.4 mmol), tri-nbutyl-1-ethoxyvinyl tin (3.2 g, 8.9 mmol), and PdCl₂(Ph₃P)₂ (522 mg, 0.74 mmol) were dissolved in toluene 3.7 mL and the solution stirred at 95° C. for 3 hours. The volatiles were removed in vac and the residue dissolved in 1,4-dioxane (33 mL). Aqueous HCl (2 N, 11 mL) was added and the solution stirred rapidly at RT for 1 hour. The mixture was diluted with water (350 mL), followed by aqueous/EtOAc work-up and silica gel chromatography (diethyl ether:hexanes (1:9)) to give the titled compound.

### Step 2

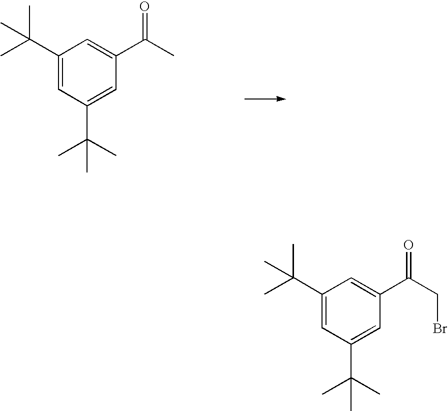

The ketone (See Example 20 Step 1, 1.0 g, 4.3 mmol) was dissolved in diethyl ether (22 mL), and aluminum chloride (29 mg, 0.22 mmol) added at RT. Bromine (0.72 g, 4.5 mmol) was added dropwise and the resulting mixture stirred for 10 minutes. The mixture was diluted with a solution of concentrated HCl:ice water (1:10, 40 mL) followed by aqueous/EtOAc work-up to give the titled compound.

### Step 3

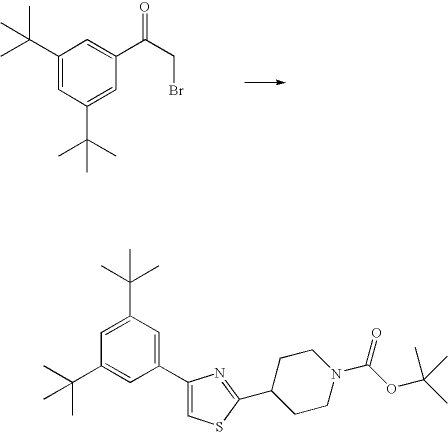

The bromoketone (See Example 20 Step 2, 0.67 g, 2.2 mmol) was dissolved in 1:1 ethyl alcohol/THF (22 mL), the thioamide (See Example 1 Step 9, 0.52 g, 2.2 mmol)) added, and the mixture stirred at room temperature overnight. The solution was diluted with aqueous NaOH (1N, 300 mL), followed by aqueous/EtOAc work-up and silica gel chromatography (EtOAc:hexanes (1:9)) to afford the titled compound.

### Step 4

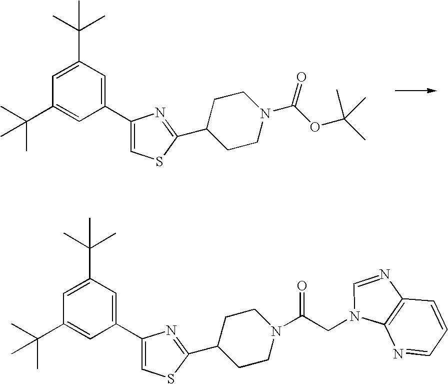

The thiazole (See Example 20 Step 3, 18 mg, 0.039 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash re-extracted (3 times) with EtOAc. The combined organic fraction was washed with brine, dried over Na₂SO, filtered and reduced in vac. The recovered material, 1-HOBT (8.0 mg, 0.059 mmol) and the acid (See Example 1 Step 13, 8.3 mg, 0.047 mmol) were combined and the mixture dissolved in DMF (0.40 mL). Diisopropyl ethyl amine (25 mg, 0.20 mmol) and EDAC (11 mg, 0.059 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR δ (ppm) (CD3OD): 9.33 (s, 1H), 8.66 (dd, J=1.2, 4.8 Hz, 1H), 8.30 (d, J=8.2 Hz, 1H), 7.75 (d, J=1.8 Hz, 2H), 7.66 (s, 1H), 7.64 (dd, J=4.8, 8.2 Hz, 1H), 7.45 (t, J=1.8 Hz, 1H), 5.63 (ABq, Δδ=23.6, J=16.9 Hz, 2H), 4.56 (d, J=13.4 Hz, 1H), 4.22 (d, J=13.6 Hz, 1H), 3.53-3.45 (m, 2H), 3.01 (t, J=13.0 Hz, 1H), 2.36 (d, J=12.0 Hz, 1H), 2.23 (d, J=13.5 Hz, 1H), 2.08 (dq, J=3.9, 12.8 Hz, 1H), 1.84 (dq, J=4.1, 13.0 Hz, 1H), 1.37 (s, 18H).

MS: m/z=516.27 (M+H).

### Step 5

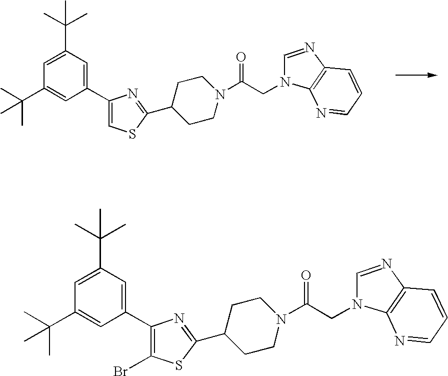

The amide (See Example 20 Step 4, 5.5 mg, 0.0111 mmol) was dissolved in DMF (0.12 mL), NBS (2.1 mg, 0.012 mmol) added and the solution stirred at RT for 1 hour. The mixture was diluted with 2:1 CH₃CN:water (6 mL) and purified by RP-18 HPLC (CH₃CN:—H₂O 15 minute gradient 10 to 100%:0.11% TFA) to give the titled compound.

¹H NMR (600 MHz, CD3OD): δ 9.15 (s, 1H); 8.60 (d, J=4.4 Hz, 1H); 8.27 (d, J=7.9 Hz, 1H); 7.70 (d, J=1.8 Hz, 2H); 7.58 (dd, J=4.7, 8.1 Hz, 1H); 7.51 (t, J=1.8 Hz, 1H); 5.58 (ABq, Δδ=26.3, J=16.9 Hz, 2H); 4.53 (d, J=13.5 Hz, 1H); 4.20 (d, J=14.0 Hz, 1H); 3.47 (t, J=13.1 Hz, 1H); 3.42 (tt, J=3.8, 11.5 Hz, 1H); 2.98 (t, J=13.9 Hz, 1H); 2.32 (d, J=13.3 Hz, 1H); 2.19 (d, J=11.4 Hz, 1H); 2.05 (dq, J=4.0, 13.1 Hz, 1H); 1.80 (dq, J=3.9, 12.7 Hz, 1H); 1.37 (s, 18H).

MS: m/z=593.97 (M+H).

### Example 21

### Preparation of 3-{2-[4-(4-{3-bromo-5-[(trifluoromethyl)sulfonyl]phenyl}-5-chloro-1,3-thiazol-2-yl)piperidin-1-yl]-2-oxoethyl}-3H-imidazo[4,5-b]pyridine

### Step 1

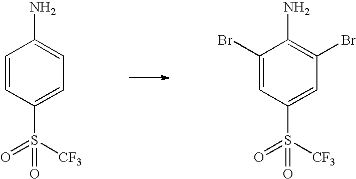

The aniline (5 g, 22 mmol) was dissolved in DMF (44 mL), NBS (9.1 g, 51 mmol) added and the solution stirred at RT overnight. The mixture was diluted with aqueous NaOH (1N, 500 mL) followed by aqueous/EtOAc work-up to afford the titled compound.

### Step 2

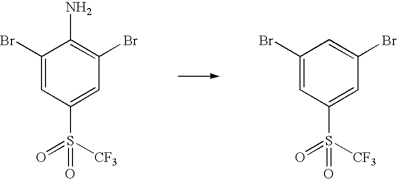

The aniline (See Example 21 Step 1, 2 g, 5.2) was dissolved in DMF (6.5 mL) and added to a 70° C. solution of isoamyl nitrite (0.91 g, 7.8 mmol) in DMF (3.5 mL) and the mixture stirred for 1 hour. The solution was cooled to RT, diluted with aqueous NaOH (200 mL), followed by aqueous/EtOAc work-up and silica gel chromatography (hexanes, neat) to afford the titled compound.

### Step 3

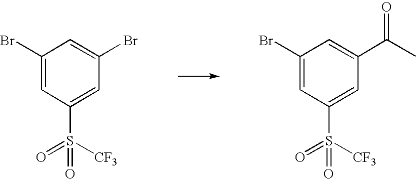

The bromide (See Example 21 Step 2, 0.6 g, 1.6 mmol), tri-nbutyl-1-ethoxyvinyl tin (0.59 g, 1.6 mmol), and PdCl₂(Ph₃P)₂ (114 mg, 0.16 mmol) were dissolved in toluene 3.2 mL) and stirred at 95° C. for 3 hours. The volatiles were removed in vac., the residue dissolved in 1,4-dioxane (10 mL), aqueous HCl (2 N, 2.4 mL) added, and the solution stirred rapidly at RT for 1 hour. The mixture was diluted with water (150 mL) followed by aqueous/EtOAc work-up and silica gel chromatography (diethyl ether:hexanes (3:7)). A second silica gel chromatography was run (EtOAc:hexanes (3:7)) to give the titled compound.

### Step 4

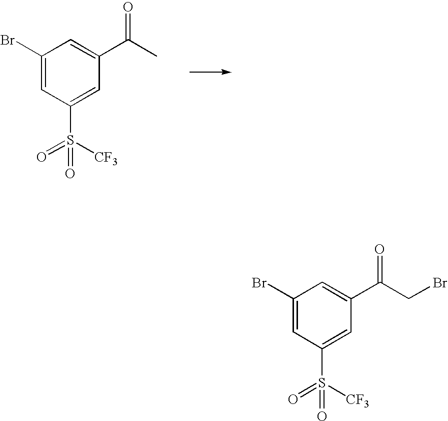

The ketone (See Example 21 Step 3, 60 mg, 0.18 mmol) was dissolved in acetic acid (0.91 mL) and the flask cooled in an RT water bath. Bromine (0.20 mL, 1 M in acetic acid) was added drop-wise and the resulting mixture was stirred for 2 hours. The mixture was diluted with a solution of concentrated HCl:ice water (1:10, 40 mL), followed by aqueous/EtOAc work-up to afford the titled compound.

### Step 5

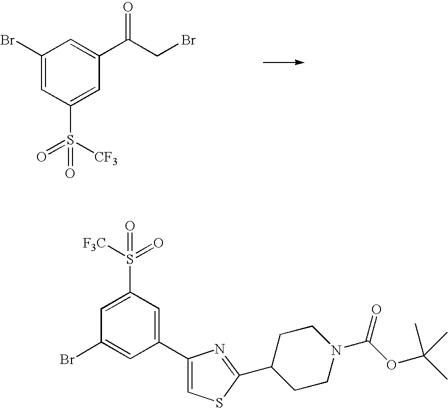

The bromoketone (See Example 21 Step 4, 0.074 g, 0.18 mmol) was dissolved in 1:1 ethyl alcohol/THF (1.0 mL), the thioamide (See Example 1 Step 9, 0.044 g, 0.18 mmol)) added and the mixture stirred at room temperature overnight. The solution was diluted with aqueous NaOH (1N, 40 mL) followed by aqueous/EtOAc work-up and silica gel chromatography (diethyl ether:hexanes (3:7)) to afford the titled compound.

### Step 6

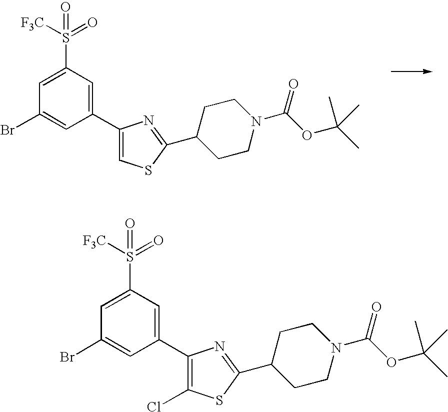

The thiazole (See Example 21 Step 5, 55 mg, 0.10 mmol) was dissolved in DMF (1.0 mL), NCS (14 mg, 0.10 mmol) added, and the solution stirred at 45° C. for 1 hour. A second addition of NCS (2.5 mg, 0.019 mmol) was made and the solution stirred for 1 hour with constant heating. The solution was cooled to RT, diluted with aqueous NaOH (1N, 40 mL) followed by aqueous/EtOAc work-up and silica gel chromatography (diethyl ether:hexanes (3:7)) to afford the titled compound.

### Step 7

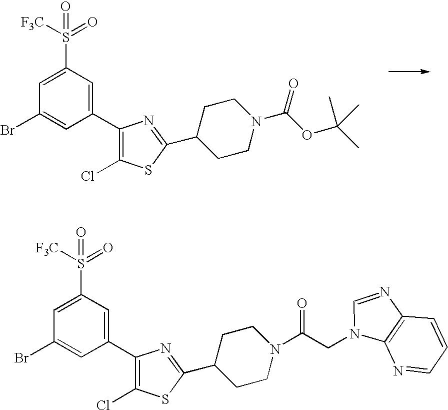

The thiazole (See Example 21 Step 6, 26 mg, 0.044 mmol) was dissolved in TFA (2 mL), stirred for 10 minutes, and the volatiles removed in vac. The residue was dissolved in EtOAc, washed with aqueous NaOH (1N, 40 mL) and the aqueous wash extracted (3 times) with EtOAc. The combined organic fraction was washed with brine, dried over MgSO₄, filtered and reduced in vac. The recovered material, 1-HOBT (9.0 mg, 0.066 mmol) and the acid (See Example 1 Step 13, 9.0 mg, 0.053 mmol) were combined and the mixture dissolved in DMF (0.44 mL). Diisopropyl ethyl amine (29 mg, 0.22 mmol) and EDAC (13 mg, 0.066 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.06 (s, 1H); 8.72 (t, J=1.6 Hz, 1H); 8.64 (s, 1H); 8.58 (dd, J=1.1, 4.7 Hz, 1H); 8.25 (m, 2H); 7.56 (dd, J=4.8, 8.2 Hz, 1H); 5.56 (ABq, Δδ=24.6, J=16.7 Hz, 2H); 4.53 (d, J=13.3 Hz, 1H); 4.21 (d, J=13.5 Hz, 1H); 3.51-3.40 (m, 2H); 3.00 (t, J=11.4 Hz, 1H); 2.33 (d, J=12.7 Hz, 1H); 2.20 (d, J=11.9 Hz, 1H); 2.08 (dq, J=3.9, 12.8 Hz, 1H); 1.82 (dq, J=3.9, 12.8 Hz, 1H).

MS: m/z=648.18 (M+H).

### Example 22

### Preparation of 3-[2-(4-{4-[3-bromo-4-methoxy-5-(trifluoromethyl)phenyl]-5-chloro-1,3-thiazol-2-yl}piperidin-1-yl)-2-oxoethyl]-3H-imidazo[4,5-b]pyridine

### Step 1

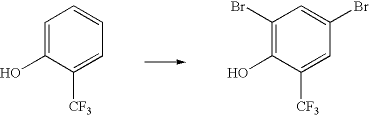

The phenol (2 g, 12 mmol) was dissolved in CH₃CN (50 mL), NBS (4.4 g, 25 mmol) added, and the solution stirred at RT overnight. The mixture was diluted with water (1N, 500 mL) followed by aqueous/EtOAc work-up to afford the titled compound.

### Step 2

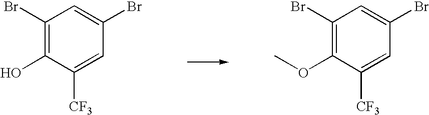

The bromide (See Example 22 Step 1, 3.9 g, 12 mmol) was dissolved in DMF (25 mL). Iodomethane (7.0 g, 49 mmol) and Cs₂CO₃ (12 g, 37 mmol) were added and the solution stirred at RT overnight. The solution was diluted with water (500 mL) followed by aqueous/EtOAc work-up to afford the titled compound.

### Step 3

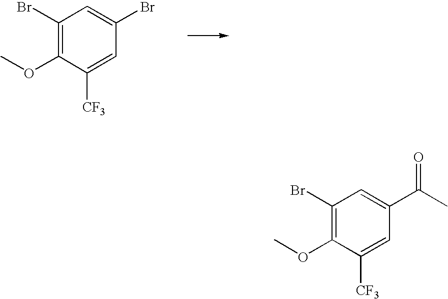

The bromide (See Example 22 Step 2, 0.50 g, 1.6 mmol), tri-nbutyl-1-ethoxyvinyl tin (0.62 g, 1.7 mmol) and PdCl₂(Ph₃P)₂ (0.11 mg, 0.16 mmol) were dissolved in toluene 3.1 mL) and the solution stirred at 90° C. for 5 hours. The solution was cooled to RT, aqueous HCl (2 N, 2.6 mL) added and the mixture stirred rapidly overnight. The mixture was poured into water (75 mL) followed by aqueous/EtOAc work-up and silica gel chromatography (EtOAc:hexanes (1:9)) to give the titled compound.

### Step 4

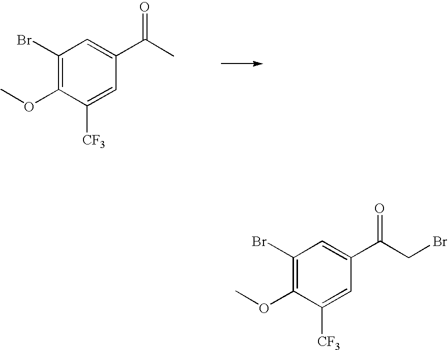

The ketone (See Example 22 Step 3, 0.15 g, 0.51 mmol) was dissolved in diethyl ether (2.5 mL), and aluminum chloride (3.4 mg, 0.025 mmol) added at RT. Bromine (97 mg, 0.61 mmol) was added dropwise and the resulting mixture stirred for 1 hour. The mixture was diluted with a solution of concentrated HCl:ice water (1:10, 40 mL) followed by aqueous/EtOAc work-up and silica gel chromatography (diethyl ether:hexanes (1:9)) to afford the titled compound.

### Step 5

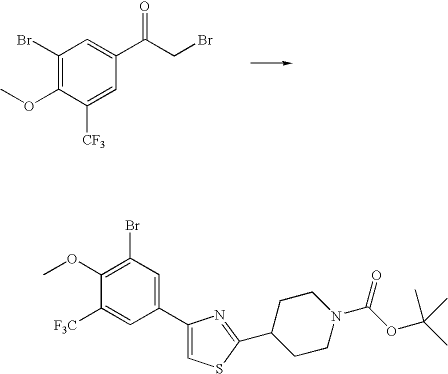

The bromoketone (See Example 22 Step 4, 0.19 g, 51 mmol) was dissolved in 1:1 ethyl alcohol/THF (1.3 mL), the thioamide (See Example 1 Step 9, 0.12 g, 0.51 mmol)) added and the solution stirred overnight. The volatiles were removed in. vac. and the residue dissolved DMF (2-5 mL). Et₃N (0.15 g, 1.5 mmol) and di-tert-butyl dicarbonate (110 mg, 0.51 mmol) were added and the solution stirred at RT for 1 hour. The mixture was diluted with 4:1 water/saturated NaHCO₃ followed by aqueous/EtOAc work-up and silica gel chromatography (EtOAc:hexanes (3:7)) to afford the titled compound.

### Step 6

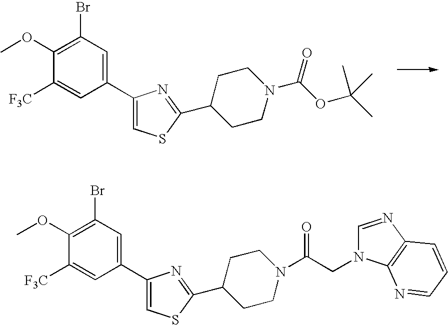

The thiazole (See Example 22 Step 5, 29 mg, 0.056 mmol) was dissolved in TFA (2 mL), stirred for 10 minutes, and the volatiles removed in vac. The residue was dissolved in EtOAc, washed with 4:1 water/saturated NaHCO₃ (40 mL) and extracted (3 times) with EtOAc. The combined organic fraction was washed with brine, dried over MgSO₄, filtered and reduced in vac. The recovered material, 1-HOBT (11 mg, 0-084 mmol) and the acid (See Example 1 Step 13, 12 mg, 0.067 mmol) were combined and the mixture dissolved in DMF (0.50 mL). Diisopropyl ethyl amine (36 mg, 0.28 mmol) and EDAC (16 mg, 0.084 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

### Step 7

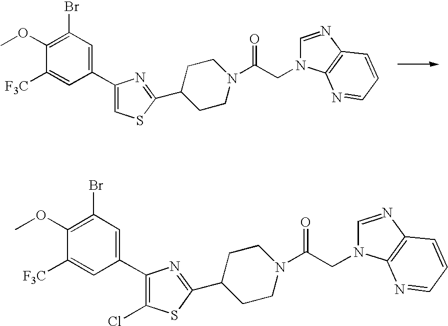

The amide (See Example 22 Step 6, 10 mg, 0.017 mmol) was dissolved in DMF (0.17 mL), NCS (2.5 mg, 0.019 mmol) added and the mixture stirred at 50° C. for 1 hour. The solution was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.32 (s, 1H); 8.65 (d, J=4.6 Hz, 1H); 8.46 (d, J=2.1 Hz, 1H); 8.31 (d, J=8.2 Hz, 1H); 8.23 (d, J=2.0 Hz, 1H); 7.63 (dd, J=4.7, 8.1 Hz, 1H); 5.62 (ABq, Δδ=21.2, J=16.7 Hz, 2H); 4.52 (d, J=13.8 Hz, 1H); 4.20 (d, J=13.5 Hz, 1H); 3.98 (s, 3H); 3.49 (t, J=11.5 Hz, 1H); 3.40 (tt, J=3.9, 11.5 Hz, 1H); 3.00 (t, J=13.8 Hz, 1H); 2.32 (d, J=13.0 Hz, 1H); 2.19 (d, J=12.3 Hz, 1H); 2.06 (dq, J=3.9, 13.1 Hz, 1H); 1.82 (dq, J=3.9, 12.8 Hz, 1H).

MS: m/z=613.92 (M+H).

### Example 23

### Preparation of 3-(2-{4-[4-(6-tert-butyl-1,1-dimethyl-2,3-dihydro-1H-inden-4-yl)-5-chloro-3-thiazol-2-yl]piperidin-1-yl}-2-oxoethyl)-3H-imidazo[4,5-b]pyridine

### Step 1

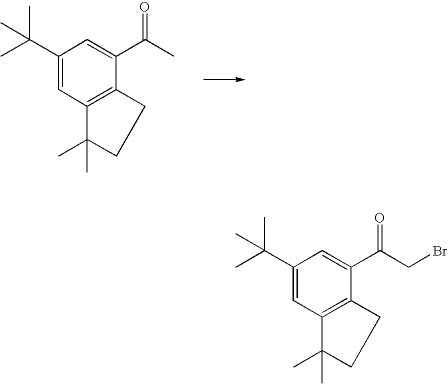

The ketone (0.10 g, 0.41 mmol) was dissolved in diethyl ether (1.0 mL), cooled to 0° C., and aluminum chloride (5.5 mg, 0.041 mmol) added. Bromine (72 mg, 0.45 mmol) was added dropwise and the resulting mixture stirred at 0° C. for 5 minutes, and RT for 15 minutes. The mixture was diluted with a solution of concentrated HCl:ice water (1:10, 40 mL), followed by aqueous/EtOAc work-up and silica gel chromatography (diethyl ether:hexanes (1:19)) to afford the titled compound.

### Step 2

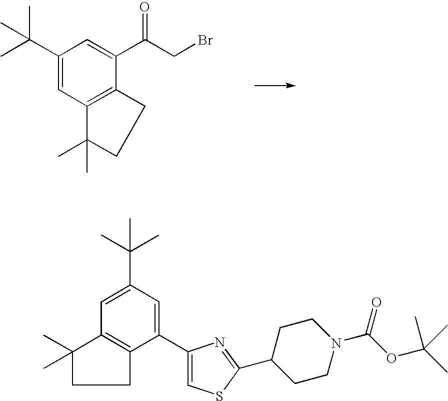

The bromoketone (See Example 23 Step 1, (0.043 g, 0.13 mmol) was dissolved in 1:1 ethyl alcohol/THF (1.5 mL), the thioamide (See Example 1 Step 9, 0.036 g, 0.15 mmol) and NaHCO₃ (12 mg, 0.15 mmol) added and the solution stirred at RT overnight. The mixture was poured into 4:1 water/saturated NaHCO₃ (40 mL) followed by aqueous/EtOAc work-up and silica gel chromatography (EtOAc:hexanes (1:9)) to afford the titled compound.

### Step 3

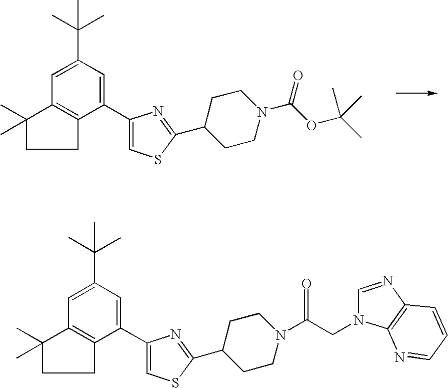

The thiazole (See Example 23 Step 2, 14 mg, 0.030 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and the volatiles removed in vac. The recovered material, 1-HOBT (6.0 mg, 0.045 mmol) and the acid (See Example 1 Step 13, 6.4 mg, 0.036 mmol) were combined and the mixture dissolved in DMF (0.30 mL). Diisopropyl ethyl amine (19 mg, 0.15 mmol) and EDAC (9.0 mg, 0.045 mmol) were added and the solution stir at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

### Step 4

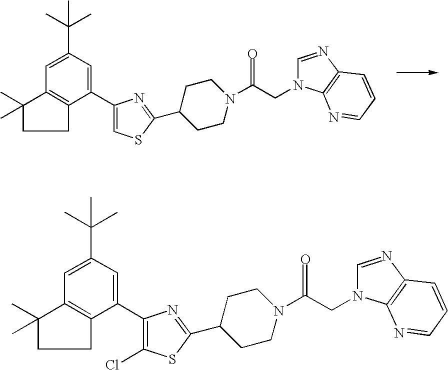

The amide (See Example 23 Step 3, (8.0 mg, 0.015 mmol) was dissolved in DMF (0.15 mL), NCS (2.0 mg, 0.015 mmol) added and the solution stirred for at 50° C. 1 hour. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.21 (s, 1H); 8.61 (d, J=4.7 Hz, 1H); 8.28 (d, J=7.7 Hz, 1H); 7.60 (dd, J=4.8, 8.1 Hz, 1H); 7.35 (d, J=1.8 Hz, 1H); 7.27 (t, J=2.7 Hz, 1H); 5.62-5.54 (ABq, Δδ=17.0, J=16.9 Hz, 2H); 4.51 (d, J=13.4 Hz, 1H); 4.19 (d, J=13.9 Hz, 1H); 3.47 (t, J=13.2 Hz, 1H); 3.37 (tt, 3.9, 11.4 Hz, 2H); 2.99 (t, J=13.7 Hz, 1H); 2.85 (t, J=7.2 Hz, 2H); 2.30 (d, J=12.2 Hz, 1H); 2.18 (d, J=10.9 Hz, 1H); 2.03 (dq, J=3.9, 13.0 Hz, 1H); 1.92 (t, J=7.2 Hz, 2H); 1.79 (dq, J=3.9, 13.0 Hz, 1H); 1.35 (s, 9H); 1.29 (s, 6H).

MS: m/z=562.13 (M+H).

### Example 24

### Preparation of 3-[2-(4-{4-[3-bromo-4-methoxy-5-(1-methylcyclopropyl)phenyl]-5-chloro-1,3-thiazol-2-yl}piperidin-1-yl)-2-oxoethyl]-3H-imidazo[4,5-b]pyridine

### Step 1

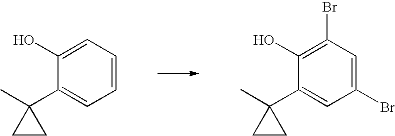

The phenol (0.20 g, 1.4 mmol) was dissolved in DMF (2.7 mL), NBS (0.53 g, 3.0 mmol) added and the solution stirred at RT overnight. The mixture was diluted with water (100 mL) followed by aqueous/EtOAc work-up to afford the titled compound.

### Step 2

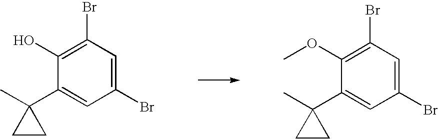

The bromide (See Example 24 Step 1, 0.413 g, 1.35 mmol) was dissolved in DMF (7.0 mL). Iodomethane (0.58 g, 4.1 mmol) and Cs₂CO₃ (0.88 g, 2.7 mmol) were added and the solution stirred at RT overnight. The solution was diluted with water (200 mL) followed by aqueous/EtOAc work-up and silica gel chromatography (diethyl ether:hexanes (1:4)) to give the titled compound.

### Step 3

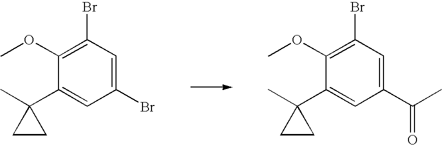

The bromide (See Example 24 Step 2, 0.14 g, 0.44 mmol), tri-nbutyl-1-ethoxyvinyl tin (0.17 g, 0.46 mmol), and PdCl₂(Ph₃P)₂ (31 mg, 0.044 mmol) were dissolved in toluene (1.0 mL) and the solution stirred at 95° C. for 3 hours. The solution was cooled to RT, aqueous HCl (2 N, 0.65 mL) added and the mixture stirred rapidly overnight.

The mixture was diluted with water (40 mL) followed by aqueous/EtOAc work-up and silica gel chromatography (diethyl ether:hexanes (1:9)) to give the titled compound.

### Step 4

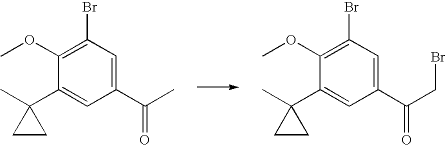

The ketone (See Example 24 Step 3, 0.070 g, 0.20 mmol) was dissolved in diethyl ether (1.0 mL), and aluminum chloride (1.4 mg, 0.010 mmol) added at RT. Bromine (34 mg, 0.21 mmol) was added dropwise and the mixture was stirred for 10 minutes. The mixture was diluted with a solution of concentrated HCl:ice water (1:10, 40 mL) followed by aqueous/EtOAc work-up and silica gel chromatography (diethyl ether:hexanes (1:19)) to afford the titled compound.

### Step 5

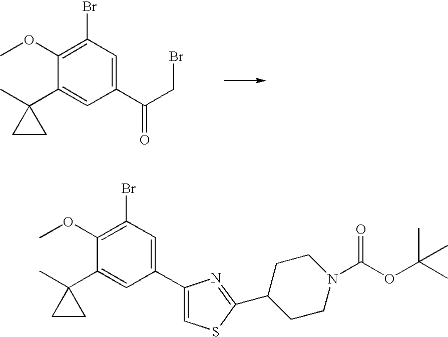

The bromoketone (See Example 24 Step 4, (0.073 g, 0.20 mmol) was dissolved in 1:1 ethyl alcohol/THF (1.0 mL), the thioamide (See Example 1 Step 9, 0.050 g, 0.20 mmol) added and the solution stirred at RT overnight. Et₃N (0.20 g, 2.0 mmol) and di-tert-butyl dicarbonate (0.044 g, 0.20 mmol) were added and the solution stirred for 1 hour. The mixture was diluted with 4:1 water/saturated NaHCO₃ followed by aqueous/EtOAc work-up and silica gel chromatography (diethyl ether:hexanes (3:7)) to afford the titled compound.

### Step 6

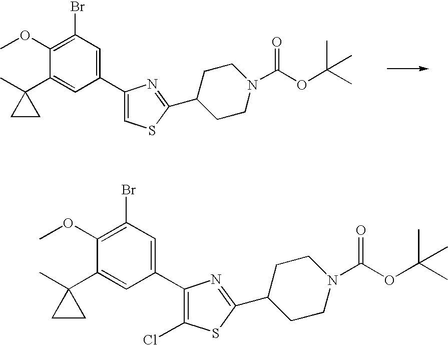

The thiazole (See Example 24 Step 5, 75 mg, 0.15 mmol) was dissolved in DMF (1.5 mL), NCS (22 mg, 0.16 mmol) added, and the solution stirred at 45° C. for 1 hour. The solution was cooled to RT, poured into aqueous NaOH (1N, 40 mL) followed by aqueous/EtOAc work-up and silica gel chromatography (diethyl ether:hexanes (3:7)) to afford the titled compound.

### Step 7

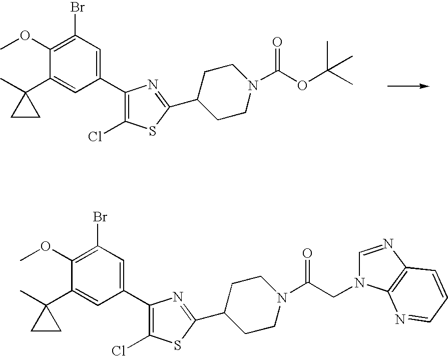

The thiazole (See Example 24 Step 6, 30 mg, 0.055 mmol) was dissolved in TFA (2 mL), stirred for 10 minutes, and the volatiles removed in vac. The residue was dissolved in EtOAc, washed with 4:1 water/saturated NaHCO₃ (40 mL) and aqueous wash extracted (3 times) with EtOAc. The combined organic fraction was washed with brine, dried over MgSO₄, filtered and reduced in vac. The recovered material, 1-HOBT (11 mg, 0.083 mmol) and the acid (See Example 1 Step 13, 12 mg, 0.066 mmol) were combined and the mixture dissolved in DMF (0.55 mL). Diisopropyl ethyl amine (36 mg, 0.28 mmol) and EDAC (16 mg, 0.083 mmol) were added and the solution stirred at RT overnight. The mixture was diluted in 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.26 (s, 1H); 8.64 (dd, J=1.3, 4.8 Hz, 1H); 8.29 (dd, J=1.3, 8.2 Hz, 1H); 7.99 (d, J=2.2 Hz, 1H); 7.86 (d, J=2.2 Hz, 1H); 7.62 (dd, J=4.8, 8.2 Hz, 1H); 5.60 (ABq, Δδ=21.1, J=16.9 Hz, 2H); 4.52 (d, J=13.4 Hz, 1H); 4.19 (d, J=14.0 Hz, 1H); 3.97 (s, 3H); 3.48 (t, J=11.6 Hz, 1H); 3.34 (m, 1H); 2.99 (t, J=12.7 Hz, 1H); 2.31 (d, J=13.1 Hz, 1H); 2.18 (d, J=13.1 Hz, 1H); 2.04 (dq, J=3.9, 13.1 Hz, 1H); 1.79 (dq, J=4.1, 13.1 Hz, 1H); 1.41 (s, 3H); 0.88 (t, J=5.1 Hz, 2H); 0.78-0.76 (t, J=4.8 Hz, 2H).

MS: m/z=600.22 (M+H.

### Example 25

### Preparation of 3-(2-{4-[5-chloro-4-(2,6-di-tert-butylpyrimidin-4-yl)-1,3-thiazol-2-yl]piperidin-1-yl}-2-oxoethyl)-3H-imidazo[4,5-b]pyridine

### Step 1

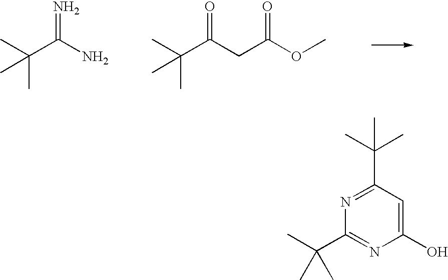

2,2-Dimethylpropanimidamide hydrochloride (1.0 g, 7.3 mmol) was dissolved in ethyl alcohol (18 mL), sodium methoxide (0.5 M in methanol, 29 mL) added and the solution stirred at RT for 30 minutes. Methyl 4,4-dimethyl-3-oxovalerate (1.2 g, 7.3 mmol) was added and the solution stirred at 78° C. for 3 hours. The mixture was cooled to RT, poured into water (500 mL), acidified with acetic acid, followed by aqueous/EtOAc work-up to afford the titled compound.

### Step 2

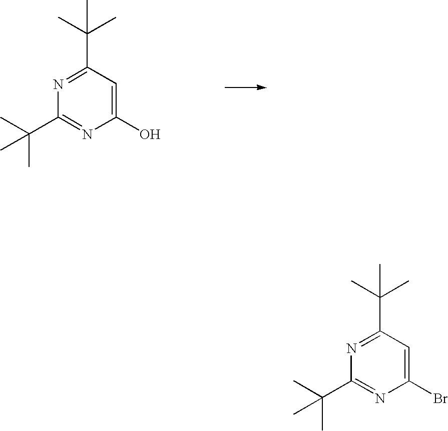

The pyrimidine (See Example 25 Step 1, 0.25 g, 1.2 mmol) and phosphorus oxybromide (0.36 g, 1.3 mmol) were placed in a sealed screw-capped vial and heated at 90° C. for 1 hour The mixture was cooled to RT, the residue dissolved in EtOAc, washed with 4:1 water/saturated NaHCO₃ (400 mL), and the aqueous wash extracted (3 times) with EtOAc. The combined organic fraction was washed with brine, dried over MgSO₄, filtered and reduced in vac. Silica gel chromatography (EtOAc:hexanes (1:50)) affords the titled compound.

### Step 3

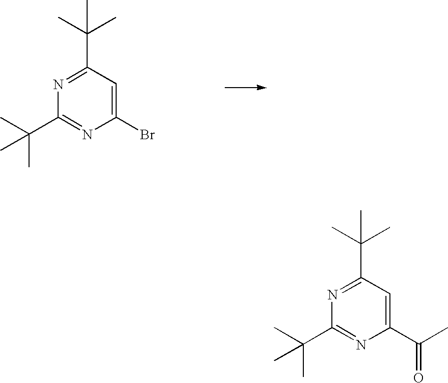

The bromide (See Example 25 Step 2, 0.25 g, 0.92 mmol), tri-nbutyl-1-ethoxyvinyl tin (0.43 g, 0.1.2 mmol), and PdCl₂(Ph₃P)₂ (65 mg, 0.092 mmol) were dissolved in toluene (1.8 mL) and the solution stirred at 95° C. for 3 hours. The mixture was cooled to RT and the volatiles removed in vac. The residue was dissolved in 1,4-dioxane (8.0 mL), aqueous HCl (2 N, 1.38 mL) added and the mixture stirred rapidly at RT for 1 hour. The mixture was poured into water (200 mL) followed by aqueous/EtOAc work-up and silica gel chromatography (diethyl ether:hexanes (1:19)) to give the titled compound.

### Step 4

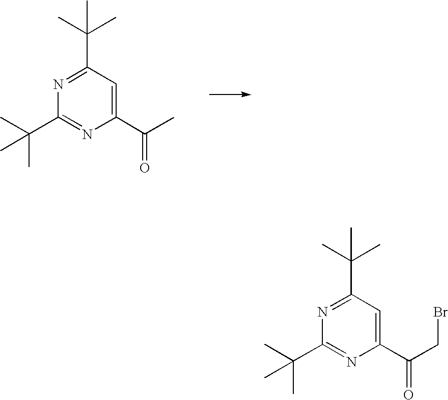

The ketone (See Example 25 Step 3, 0.15 g, 0.64 mmol) was dissolved in acetic acid (3.2 mL), bromine (0.7 mL, 1 M in acetic acid) added drop-wise, and the solution stirred at 90° C. for 1 hour. The mixture was cooled to RT, diluted with a solution of concentrated HCl:ice water (1:10, 40 mL), followed by aqueous/EtOAc work-up and to afford the titled compound.

### Step 5

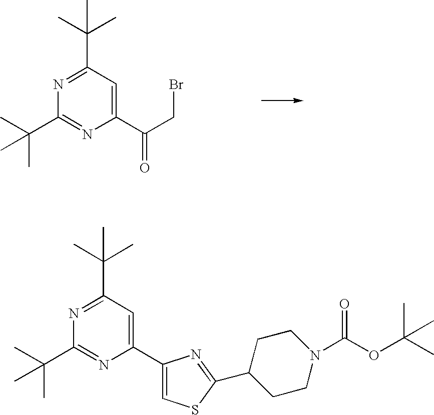

The bromoketone (See Example 25 Step 4, 0.025 g, 0.080 mmol) was dissolved in 1:1 ethyl alcohol/THF (1.0 mL), the thioamide (See Example 1 Step 9, 0.020 g, 0.080 mmol) added and the solution stirred at RT overnight. The solvent was removed in vac. and the residue dissolved in DMF (1.0 mL). Et₃N (0.024 g, 0.24 mmol) and di-tert-butyl dicarbonate (0.018 g, 0.080 mmol) were added and the solution stirred at RT for 1 hour. The mixture was poured into saturated NaHCO₃ (50 mL) followed by aqueous/EtOAc work-up and silica gel chromatography (diethyl ether:hexanes (3:7)) to afford the titled compound.

### Step 6

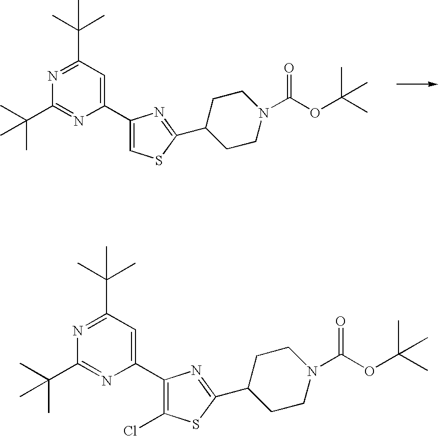

The thiazole (See Example 25 Step 5, 63 mg, 0.14 mmol) was dissolved in DMF (1.4 mL), NCS (20 mg, 0.15 mmol) added and the mixture stirred at 45° C. for 2 hours. A second addition of NCS (4.0 mg, 0.030 mmol) was made and the solution stirred at 45° C. for 2 hours. The solution was cooled to RT, poured into water:saturated NaHCO₃ (4:1, 40 mL), followed by aqueous/EtOAc work-up and silica gel chromatography (EtOAc:hexanes (1:9)) to afford the titled compound.

### Step 7

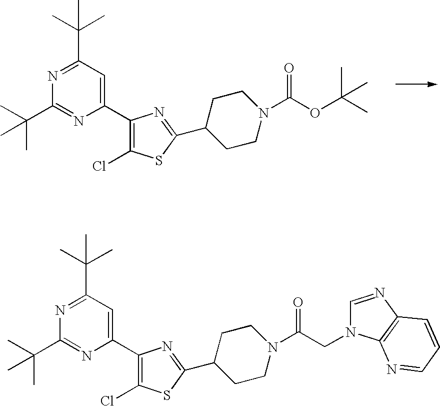

The thiazole (See Example 25 Step 6, 31 mg, 0.063 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (13 mg, 0.094 mmol) and the acid (See Example 1 Step 13, 13 mg, 0.076 mmol) were combined and the mixture dissolved in DMF (0.63 mL). Diisopropyl ethyl amine (41 mg, 0.32 mmol) and EDAC (18 mg, 0.094 mmol) were added and the solution stirred at RT overnight. The mixture was diluted in 2:1 CH₃CN:water (6 mL) and purified by RP-18 HPLC (CH₃CN:H₂O 15 minute gradient 10 to 100%:0.1% TFA) to give the titled compound.

¹H NMR (600 MHz, CD3OD): δ 9.37 (s, 1H); 8.67 (d, J=3.7 Hz, 1H); 8.31 (d, J=8.1 Hz, 1H); 7.82 (s, 1H); 7.65 (dd, J=4.7, 8.2 Hz, 1H); 5.64 (ABq, Δδ=28.6, J=16.9 Hz, 2H); 4.54 (d, J=13.7 Hz, 1H); 4.21 (d, J=14.0 Hz, 1H); 3.49 (t, J=13.0 Hz, 1H); 3.42 (tt, J=3.9, 11.5 Hz, 1H); 3.01 (t, J=13.6 Hz, 1H); 2.34 (d, J=11.9 Hz, 1H); 2.21 (d, J=13.0 Hz, 1H); 2.06 (dq, J=3.9, 13.0 Hz, 1H); 1.85-1.79 (dq, J=4.0, 12.8 Hz, 1H); 1.44 (s, 9H); 1.38 (s, 9H).

MS: m/z=552.21 (M+H).

### Example 26

### Preparation of 3-[2-(4-{4-[2-tert-butyl-6-(trifluoromethylpyrimidin-4-yl]-5-chloro-1,3-thiazol-2-yl}piperidin-1-yl)-2-oxoethyl]-3H-imidazo[4,5-b]pyridine

### Step 1

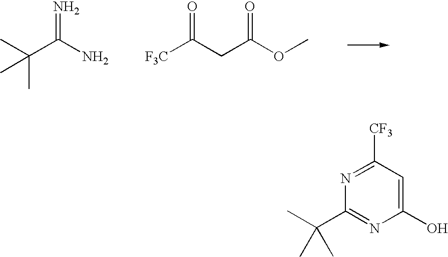

2,2-Dimethylpropanimidamide hydrochloride (1.0 g, 7.3 mmol) was dissolved in ethyl alcohol (18 mL), sodium methoxide (0.5 M in methanol, 29 mL) added and the solution stirred at RT for 30 minutes. Ethyl 4,4,4-trifluoroacetoacetate (1.3 g, 7.3 mmol) was added and the solution stirred at 78° C. for 3 hours. The mixture was cooled to RT, poured into water (500 mL), acidified with acetic acid, followed by aqueous/EtOAc work-up to afford the titled compound.

### Step 2

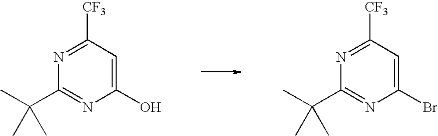

The pyrimidine (See Example 26 Step 1, 0.40 g, 1.82 mmol) and phosphorus oxybromide (0.55 g, 1.9 mmol) were placed in a screw capped vial and stirred at 90° C. for 1 hour The vessel was cooled to RT, the residue dissolved in EtOAc, washed with 4:1 water/saturated NaHCO₃ (400 mL), and the aqueous wash extracted (3 times) with EtOAc. The combined organic fraction was washed with brine, dried over MgSO₄, filtered and reduced in vac to afford the titled compound.

### Step 3

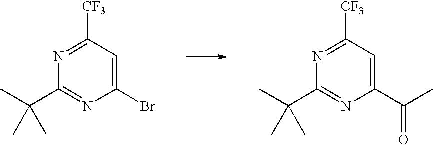

The bromide (See Example 26 Step 2, 0.20 g, 0.71 mmol), tri-nbutyl-1-ethoxyvinyl tin (0.31 g, 0.85 mmol), and PdCl₂(Ph₃P)₂ (50 mg, 0.071 mmol) were dissolved in toluene (1.5 mL) and stirred at 90° C. for 3 hours. The mixture was cooled to RT and the volatiles removed in vac. The residue was dissolved in 1,4-dioxane (2 mL), aqueous HCl (2 N, 2 mL) added and the mixture stirred rapidly at 70° C. for 1 hour. The mixture was diluted with water (40 mL) followed by aqueous/EtOAc work-up and silica gel chromatography (diethyl ether:hexanes (1:19)) to give the titled compound.

### Step 4

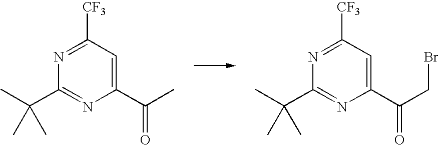

The ketone (See Example 26 Step 3, 0.25 g, 1.0 mmol) was dissolved in acetic acid (5.0 mL) and bromine (1 M in acetic acid, 1.2 mL) was added to the stirred solution drop-wise. The mixture was heated to 45° C. for 1 hour. The mixture was poured into a solution of concentrated HCl:ice water (4:40 mL), partitioned with EtOAc (3×, 70 mL). The organics were combined, washed with brine, dried over MgSO₄, filtered and stripped to afford the titled compound.

### Step 5

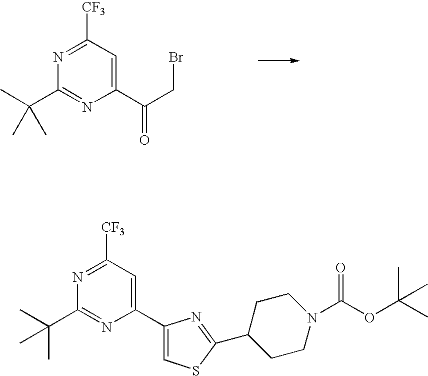

The bromoketone (See Example 26 Step 4, 0.24 g, 0.74 mmol) was dissolved in 1:1 ethyl alcohol/THF (3.7 mL), the thioamide (See Example 1 Step 9, 0.18 g, 0.74 mmol) added and the solution stirred at RT overnight. The solvent was removed in vac. and the residue dissolved in DMF (3.7 mL). Et₃N (0.75 g, 7.4 mmol) and di-tert-butyl dicarbonate (0.16 g, 0.74 mmol) were added and the solution stirred for 1 hour. The mixture was diluted with saturated NaHCO₃ (100 mL) followed by aqueous/EtOAc work-up and silica gel chromatography (diethyl ether:hexanes (1:7)) to afford the titled compound.

### Step 6

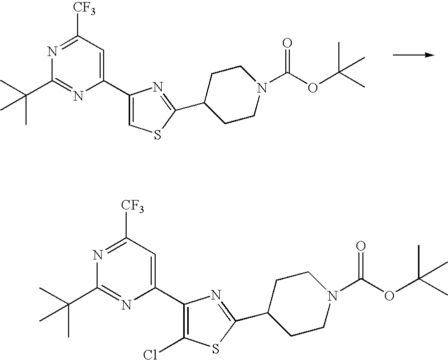

The thiazole (See Example 26 Step 5, 49 mg, 0.10 mmol) was dissolved in DMF (1.0 mL), NCS (17 mg, 0.13 mmol) added, and the solution stirred at 45° C. overnight. A second addition of NCS (17 mg, 0.13 mmol) was made and the solution stirred at 45° C. for 1 hours. The solution was cooled to RT, diluted with water:saturated NaHCO₃ (4:1, 40 mL), followed by aqueous/EtOAc work-up and silica gel chromatography (diethyl ether:hexanes (3:7)) to afford the titled compound.

### Step 7

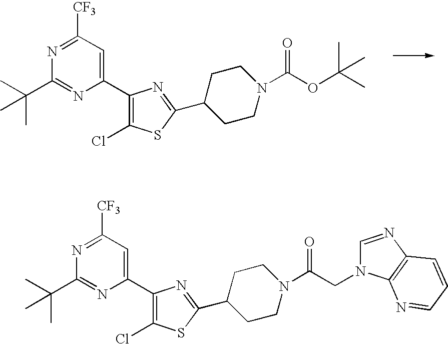

The thiazole (See Example 26 Step 6, 26 ing, 0.051 mmol) was dissolved in TFA (2 mL), stirred for 10 minutes and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (11 mg, 0.077 mmol) and the acid (See Example 1 Step 13, 11 mg, 0.061 mmol) were combined and the mixture dissolved in DMF (0.50 mL). Diisopropyl ethyl amine (33 mg, 0.26 mmol) and EDAC (15 mg, 0.077 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹HNMR (500 MHz, CD3OD): δ 9.26 (s, 1H); 8.63 (d, J=4.6 Hz, 1H); 8.29 (d, J=8.0 Hz, 1H); 8.17 (s, 1H); 7.61 (dd, J=4.8, 8.1 Hz, 1H); 5.61 (ABq, Δδ=22.3, J=16.7 Hz, 2H); 4.53 (d, J=13.5 Hz, 1H); 4.21 (d, J=13.5 Hz, 1H); 3.53-3.41 (m, 2H); 3.01 (t, J=13.4 Hz, 1H); 2.34 (d, J=13.1 Hz, 1H); 2.22 (d, J=13.0 Hz, 1H); 2.09 (dq, J=4.0, 12.9 Hz, 1H); 1.83 (dq, J=3.7, 12.6 Hz, 1H); 1.48 (s, 9H).

MS: m/z=564.17 (M+H).

### Example 27

### Preparation of 3-[2-(4-{4-[3-bromo-4-methoxy-5-(trifluoromethoxy)phenyl]-5-chloro-1,3-thiazol-2-yl}piperidin-1-yl)-2-oxoethyl]-3H-imidazo[4,5-b]pyridine

### Step 1

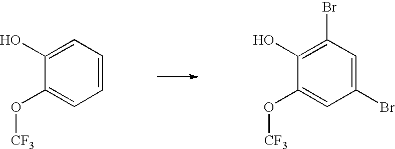

The phenol (1.0 g, 5.6 mmol) was dissolved in DMF (28 mL), NBS (2.2 g, 12 mmol) added and the solution stirred at RT overnight. The mixture was diluted with water (100 mL) followed by aqueous/EtOAc work-up to afford the titled compound.

### Step 2

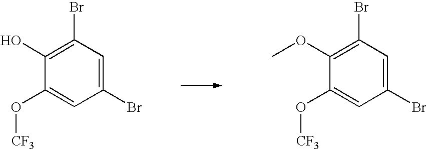

The bromide (See Example 27 Step 1, 1.9 g, 5.6 mmol) was dissolved in DMF (11 mL). Cs₂CO₃ (5.5 g, 17 mmol) and iodomethane (3.2 g, 22 mmol) were added and the solution stirred 4 hours at RT. The solution was diluted with aqueous NaOH (1N, 2001 mL) followed by aqueous/EtOAc work-up to afford the titled compound.

### Step 3

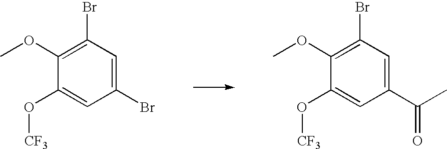

The bromide (See Example 27 Step 2, 0.59 g, 1.7 mmol), tri-nbutyl-1-ethoxyvinyl tin (0.60 g, 1.7 mmol), and PdCl₂(Ph₃P)₂ (120 mg, 0.17 mmol) were dissolved in toluene (3.3 mL) and the solution stirred at 95° C. for 3 hours. The solution was cooled to RT, aqueous HCl (2 N, 0.2.5 mL) added and the mixture stirred rapidly at RT overnight. The mixture was diluted with water (40 mL) followed by aqueous/EtOAc work-up and silica gel chromatography (diethyl ether:hexanes (1:9)) to give the titled compound.

### Step 4

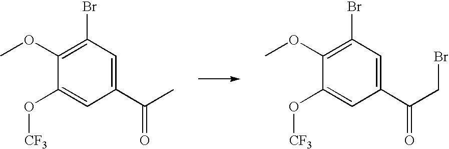

The ketone (See Example 27 Step 3, 0.24 g, 0.68 mmol) was dissolved in diethyl ether (3.4 mL) and aluminum chloride (5.0 mg, 0.034 mmol) added at RT. Bromine (0.12 g, 0.75 mmol) was added dropwise and the resulting mixture stirred for 10 minutes. The mixture was diluted with a solution of concentrated HCl:ice water (4:40 mL) followed by aqueous/EtOAc work-up and silica gel chromatography (diethyl ether:hexanes (1:9)) to afford the titled compound.

### Step 5

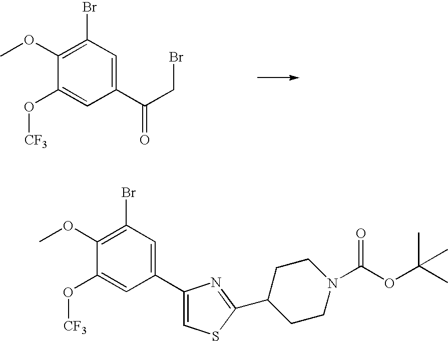

The bromoketone (See Example 27 Step 4, 0.20 g, 0.51 mmol) was dissolved in 1:1 ethyl alcohol/THF (2.6 mL), the thioamide (See Example 1 Step 9, 0.13 g, 0.51 mmol)) added and the solution stirred at RT overnight. The solvent was removed in vac. and the residue dissolved in DMF (2.6 mL). Et₃N (0.52 g, 5.1 mmol) and di-ter-butyl dicarbonate (0.11 g, 0.51 mmol) were added and the solution stirred at RT for 1 hour. The mixture was diluted with 4:1 water/saturated NaHCO₃ (100 mL) followed by aqueous/EtOAc work-up and silica gel chromatography (diethyl ether:hexanes (1:4)) to afford the titled compound.

### Step 6

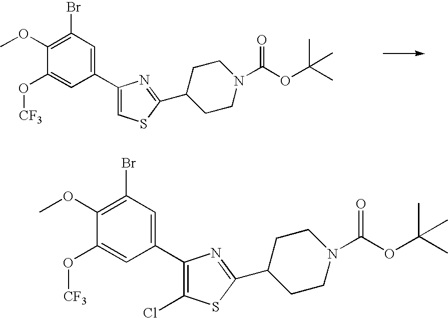

The thiazole (See Example 27 Step 5, 80 mg, 0.15 mmol) was dissolved in DMF (1.5 mL), NCS (22 mg, 0.16 mmol) added, and the solution stirred at 45° C. for 2 hours. The solution was cooled to RT, diluted with aqueous NaOH (1N, 40 mL), followed by aqueous/EtOAc work-up and silica gel chromatography (diethyl ether:hexanes (3:7)) to afford the titled compound.

### Step 7

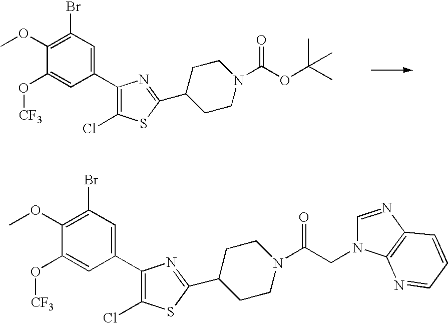

The thiazole (See Example 27 Step 6, 37 mg, 0.065 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (13 mg, 0.10 mmol) and the acid (See Example 1 Step 13, 14 mg, 0.080 mmol) were combined and the mixture dissolved in DMF (0.65 mL). Diisopropyl ethyl amine (42 mg, 0.33 mmol) and EDAC (19 mg, 0.10 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.17 (s, 1H); 8.61 (d, J=4.3 Hz, 1H); 8.27 (d, J=8.2 Hz, 1H); 8.20 (d, J=2.0 Hz, 1H); 7.94 (s, 1H); 7.59 (dd, J=4.8, 8.2 Hz, 1H); 5.58 (ABq, Δδ=22.1, J=16.9 Hz, 2H); 4.51 (d, J=13.6 Hz, 1H); 4.19 (d, J=14.2 Hz, 1H); 3.94 (s, 3H); 3.48 (t, J=13.3 Hz, 1H); 3.39 (tt, J=3.9, 11.4 Hz, 1H); 2.99 (t, J=13.5 Hz, 1H); 2.31 (d, J=12.0 Hz, 1H); 2.17 (d, J=12.1 Hz, 1H); 2.08-2.00 (dq, J=3.6, 12.8 Hz, 1H); 1.83-1.75 (dq, J=3.9, 13.1 Hz, 1H).

MS: m/z=630.10 (M+H).

### Example 28

### Preparation of 3-[2-(4-{5-chloro-4-[3-cyclopropyl-4-methoxy-5-(trifluoromethoxy)phenyl]-1,3-thiazol-2-yl}piperidin-1-yl)-2-oxoethyl]-3H-imidazo[4,5-b]pyridine

### Step 1

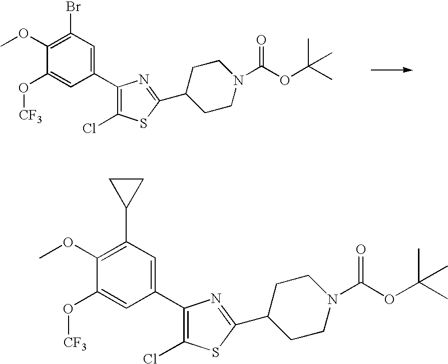

The bromide (See Example 27 Step 6, 0.035 g, 0.061 mmol) was dissolved in 20:1 toluene:water (0.61 mL). Potassium phosphate (0.097 g, 0.37 mmol), cyclopropylboronic acid (11 mg, 0.12 mmol) and tetrakis(triphenylphosphine)palladium(0) (0.035 g, 0.031 mmol) were added, the vessel purged with nitrogen (3×) and the slurry stirred at 95° C. for 2 hours. The solution was cooled to RT, diluted with water (40 mL) followed by aqueous/EtOAc work-up and silica gel chromatography (diethyl ether:hexanes (3:7)) to give the titled compound.

### Step 2

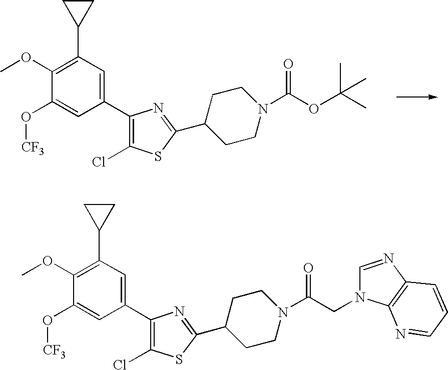

The thiazole (See Example 28 Step 1, 24 mg, 0.024 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (5.0 mg, 0.037 mmol) and the acid (See Example 1 Step 13, 5.1 mg, 0.029 mmol) were combined and the mixture dissolved in DMF (0.24 mL). Diisopropyl ethyl amine (16 mg, 0.12 mmol) and EDAC (7.1 mg, 0.037 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.05 (s, 1H); 8.58 (dd, J=1.2, 4.7 Hz, 1H); 8.25 (dd, J=1.2, 8.2 Hz, 1H); 7.67 (t, J=1.6 Hz, 1H); 7.56 (dd, J=4.8, 8.2 Hz, 1H); 7.42 (d, J=2.0 Hz, 1H); 5.55 (ABq, Δδ=22.8, J=16.9 Hz, 2H); 4.52 (d, J=13.4 Hz, 1H); 4.20 (d, J=13.9 Hz, 1H); 3.93 (s, 3H); 3.47 (t, J=12.7 Hz, 1H); 3.38 (tt, J=3.9, 11.4 Hz, 1H); 2.98 (t, J=13.3 Hz, 1H); 2.31-2.25 (m, 2H); 2.17 (d, J=12.0 Hz, 1H); 2.03 (dq, J=3.7, 12.8 Hz, 1H); 1.78 (dq, J=4.1, 13.0 Hz, 1H); 1.10-1.06 (m, 2H); 0.76-0.73 (m, 2H).

MS: m/z=592.31 (M+H).

### Example 29

### Preparation of 3-{2-[4-(4-{3-bromo-4-methoxy-5-[(trifluoromethyl)thio]phenyl}-5-chloro-1,3-thiazol-2-yl)piperidin-1-yl]-2-oxoethyl}-3H-imidazo[4,5-b]pyridine

### Step 1

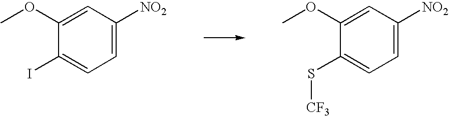

The nitro compound (0.33 g, 0.1.2 mmol) and CuSCF₃ (0.38 g, 0.2.3 mmol) were combined in N-methylpyrrolidinone (1.2 mL) and the mixture was stirred at 150° C. for 1 hour. The mixture was cooled to RT, diluted with 2:1 water:saturated NaHCO₃ (40 mL), followed by aqueous/EtOAc work-up and silica gel chromatography (diethyl ether:hexanes (1:19)) to afford the titled compound.

### Step 2

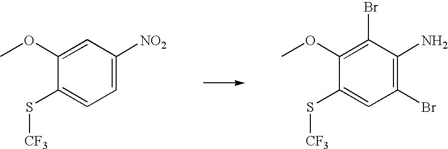

The nitro compound (See Example 29 Step 1, 0.25 g, 1.0 mol) was dissolved in methanol (6 mL), Pd(OH)₂ (20%, 50 mg) added and the slurry stirred under a hydrogen atmosphere for 1.5 hours at RT. The mixture was filtered and the volatiles removed in. vac. to afford the titled compound.

### Step 3

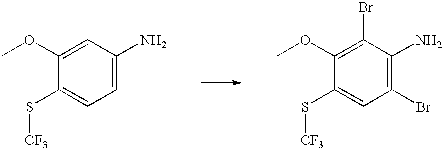

The aniline (See Example 29 Step 2, 0.23 g, 1.1 mmol) was dissolved in DMF (3.0 mL), NBS (0.19 g, 1.1 mmol) added, and the solution stirred at RT for 1 hour. A second addition of NBS (0.12 g, 0.55 mmol) was made and the solution stirred at RT for 1 hour. The mixture was diluted with water (100 mL), followed by aqueous/EtOAc work-up and silica gel chromatography (EtOAc:hexanes (1:4)) to afford the titled compound.

### Step 4

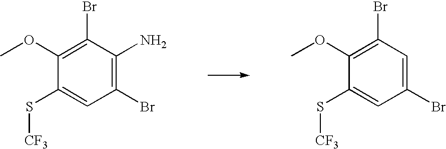

The aniline (See Example 29 Step 3, 0.23 g, 0.62 mmol) was dissolved in DMF (0.3 mL) and the resulting solution added to stirred, 70° C. solution of isoamyl nitrite (0.16 g, 0.92 mmol) in DMF (1.2 mL). The mixture was stirred at 70° C. for 1 hour. The solution was cooled to RT, poured into aqueous NaOH (50 mL), followed by aqueous/EtOAc work-up and silica gel chromatography (EtOAc:hexanes (1:19)) to afford the titled compound.

### Step 5

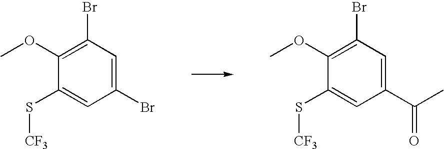

The bromide (See Example 29 Step 4, 0.16 g, 0.44 mmol), tri-nbutyl-1-ethoxyvinyl tin (0.16 g, 0.44 mmol), and PdCl₂(Ph₃P)₂ (31 mg, 0.044 mmol) were dissolved in toluene (0.90 mL) and stirred at 90° C. for 3 hours. The solution was cooled to RT, aqueous HCl (2 N, 0.66 mL) added and the mixture stirred rapidly at RT overnight. The mixture was diluted with water (40 mL), followed by aqueous/EtOAc work-up and silica gel chromatography (EtOAc:hexanes (1:9)) to give the titled compound.

### Step 6

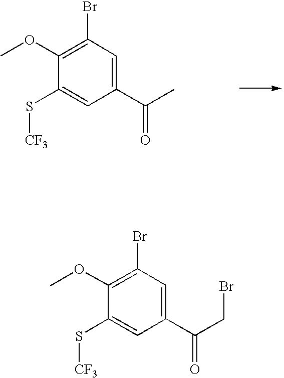

The ketone (See Example 29 Step 5, 0.095 g, 0.29 mmol) was dissolved in diethyl ether (1.5 mL), and aluminum chloride (2.0 mg, 0.014 mmol) added at RT. Bromine (0.048 g, 0.30 mmol) was added dropwise and the resulting mixture was stirred for 10 minutes. The mixture was diluted with a solution of concentrated HCl:ice water (1:10, 40 mL) followed by aqueous/EtOAc work-up and silica gel chromatography (diethyl ether:hexanes (1:9)) to afford the titled compound.

### Step 7

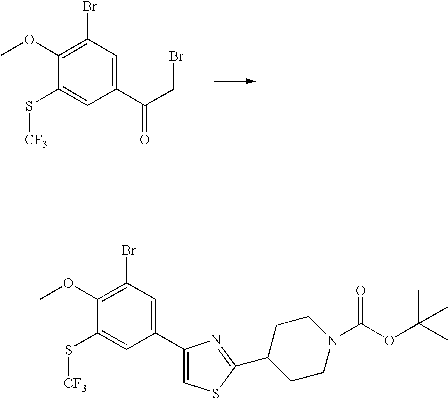

The bromoketone (See Example 29 Step 6, 0.050 g, 0.15 mmol) was dissolved in 1:1 ethyl alcohol/THF (0.75 mL), the thioamide (See Example 1 Step 9, 0.037 g, 0.15 mmol)) added and the solution stirred at RT overnight. The solvent was removed in vac. and the residue dissolved in DMF (0.75 mL). Et₃N (0.15 g, 1.5 mmol) and di-tert-butyl dicarbonate (0.033 g, 0.15 mmol) were added and the solution stirred at RT for 1 hour. The mixture was diluted with 4:1 water/saturated NaHCO₃, followed by aqueous/EtOAc work-up and silica gel chromatography (EtOAc:hexanes (1:9)) to afford the titled compound.

### Step 8

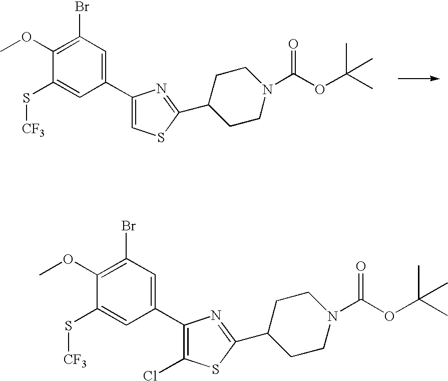

The thiazole (See Example 29 Step 7, (36 mg, 0.065 mmol) was dissolved in DMF (0.65 mL), NCS (10 mg, 0.072 mmol) added, and the solution stirred at 45° C. for 1 hour. The solution was cooled to RT, diluted with aqueous NaOH (1N, 40 mL), followed by aqueous/EtOAc work-up and silica gel chromatography (diethyl ether:hexanes (3:7)) to afford the titled compound.

### Step 9

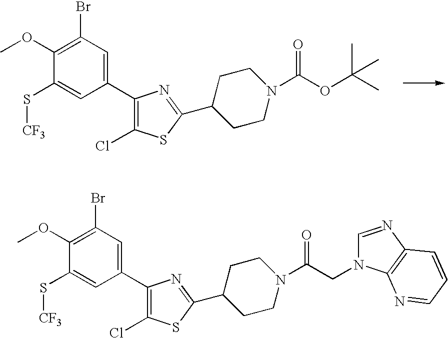

The thiazole (See Example 29 Step 8, 23 mg, 0.039 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (8.0 mg, 0.059 mmol) and the acid (See Example 1 Step 13, 8.3 mg, 0.047 mmol) were combined and the mixture dissolved in DMF (0.40 mL). Diisopropyl ethyl amine (25 mg, 0.20 mmol) and EDAC (11 mg, 0.059 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.24 (s, 1H); 8.63 (dd, J=1.3, 4.8 Hz, 1H); 8.36 (d, J=2.1 Hz, 1H); 8.29 (m, 2H); 7.61 (dd, J=4.8, 8.2 Hz, 1H); 5.60 (ABq, Δδ=22.1, J=16.7 Hz, 2H); 4.52 (d, J=13.5 Hz, 1H); 4.20 (d, J=14.0 Hz, 1H); 3.96 (s, 3H); 3.48 (t, J=12.9 Hz, 1H); 3.40 (tt, J=3.9, 11.5 Hz, 1H); 3.00 (t, J=13.7 Hz, 1H); 2.32 (d, J=13.3 Hz, 1H); 2.19 (d, J=11.9 Hz, 1H); 2.06 (dq, J=3.9, 13.1 Hz, 1H); 1.80 (dq, J=4.1, 13.0 Hz, 1H).

MS: m/z=646.11 (M+H).

### Example 30

### Preparation of 3-[2-(4-{5-bromo-4-[6-tert-butyl-2-(1-methylcyclopropyl)pyrimidin-4-yl]-1,3-thiazol-2-yl}piperidin-1-yl)-2-oxoethyl]-3H-imidazo[4,5-b]pyridine

### Step 1

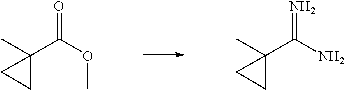

Ammonium chloride (2.1 g, 39 mmol) was suspended in toluene (10 mL) and the slurry cooled to 0° C. Trimethyl aluminum (2.0 M in toluene, 19.5 mL) was added at 0° C., the mixture allowed to warm to RT, and stirring continued until gas evolution had ceased. The ethyl 1-methylcyclopropane-1-carboxylate (1 g, 7.8 mmol) was then added and the mixture stirred at 80° C. overnight. The mixture was then cooled to 0° C., methanol added, and the slurry stirred at RT for 1 hour. The solution was filtered, the solids washed with methanol, and the volatiles removed in vac. to afford the titled compound.

### Step 2

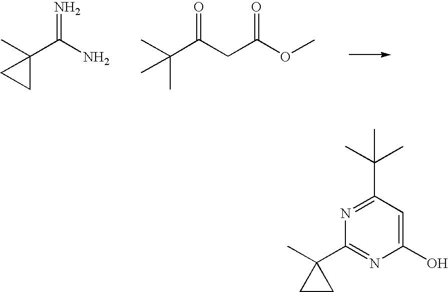

The amidine (See Example 30 Step 1, 1.0 g, 7.4 mmol) was dissolved in EtOH (19 mL), sodium methoxide (0.5 M in MeOH, 30 mL) added and the solution stirred at RT for 30 minutes. Methyl 4,4-dimethyl-3-oxovalerate (0.78 g, 5.0 mmol) was added and the solution stirred at 75° C. overnight. The mixture was cooled to RT, diluted with water (500 mL), acidified with acetic acid, followed by aqueous/CH₂Cl₂ work-up to afford the titled compound.

### Step 3

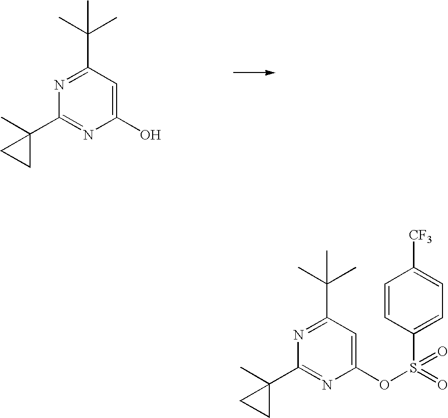

The pyrimidine (See Example 30 Step 2, 0.71 g, 3.5 mmol) was dissolved in pyridine (14 mL), trifluorosulfonic anhydride (1.5 g, 3.5 mmol) added, and the solution stirred at RT for 1 hour. The solution was diluted with saturated NaHCO₃ (250 mL), followed by aqueous/CH₂Cl₂ work-up and silica gel chromatography (diethyl ether:hexanes (1:33)) to give the titled compound.

### Step 4

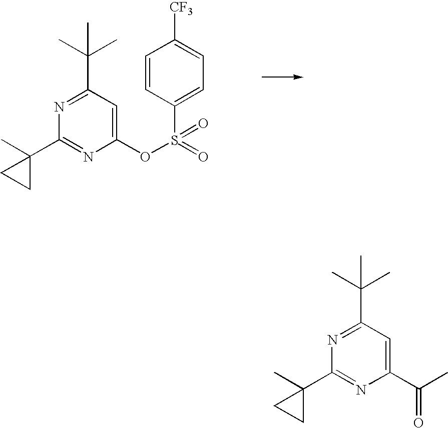

The pyrimidine (See Example 30 Step 3, 0.43 g, 2.1 mmol), tri-nbutyl-1-ethoxyvinyl tin (0.98 g, 2.7 mmol), and PdCl₂(Ph₃P)₂ (0.15 mg, 2.7 mmol) were dissolved in toluene (4.0 mL) and the solution stirred at 95° C. for 1.5 hours. The mixture was cooled to RT and the volatiles removed in vac. The residue was dissolved in 1,4-dioxane (12 mL), aqueous HCl (2 N, 3.0 mL) added and the mixture stirred rapidly at RT overnight. The mixture was diluted with water (250 mL) followed by aqueous/EtOAc work-up and silica gel chromatography (EtOAc:hexanes (1:33)) to give the titled compound.

### Step 5

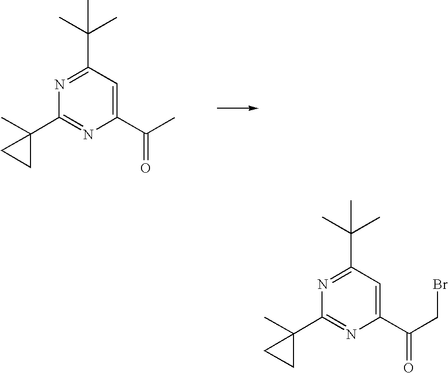

The ketone (See Example 30 Step 4, 0.25 g, 1.1 mmol) was dissolved in THF (2.2 mL), phenyl trimethyl ammonium tribromide (0.45 g, 1.2 mmol) added, and the solution stirred at 40° C. for 2 hours. A second addition of phenyl trimethyl ammonium tribromide (0.45 g, 1.2 mmol) made, and the solution stirred at 40° C. overnight. The mixture was diluted with water (40 mL), acidified with HCl (conc., 2 mL), followed by aqueous/EtOAc work-up to give the titled compound.

### Step 6

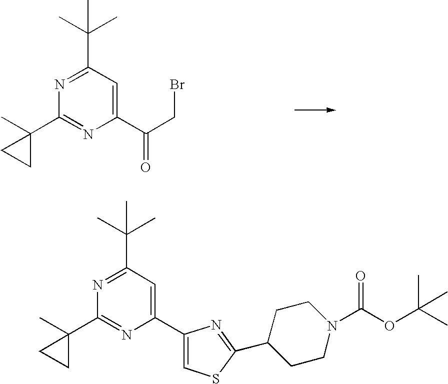

The bromoketone (See Example 30 Step 5, 0.28 g, 0.91 mmol) was dissolved in 1:1 ethyl alcohol/THF (5.0 mL), the thioamide (See Example 1 Step 9, 0.22 g, 0.91 mmol)) added and the solution stirred at RT overnight. The solvent was removed in vac. and the residue dissolved in DMF (4.5 mL). Et₃N (0.91 g, 9.1 mmol) and di-tert-butyl dicarbonate (0.20 g, 0.91 mmol) were added and the solution stirred at RT for 1 hour. The mixture was diluted with saturated NaHCO₃ (100 mL), followed by aqueous/EtOAc work-up and silica gel chromatography (diethyl ether:hexanes (1:4)) to afford the titled compound.

### Step 7

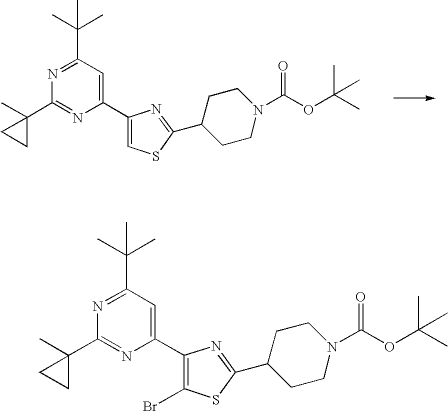

The thiazole (See Example 30 Step 6, 50 mg, 0.11 mmol) was dissolved in DMF (0.5 mL), NBS (21 mg, 0.12 mmol) added and the solution stirred at RT overnight. A second addition of NBS (21 mg, 0.12 mmol) was made and the solution stirred at 45 overnight. The solution was cooled to RT, Et₃N (0.91 g, 9.1 mmol) and di-tert-butyl dicarbonate (0.20 g, 0.91 mmol) added, and the resulting mixture stirred at RT for 1 hour The mixture was poured into 4:1 water:saturated NaHCO₃ (100 mL), followed by aqueous/EtOAc work-up and silica gel chromatography (diethyl ether:hexanes (1:4)) to give the titled compound.

### Step 8

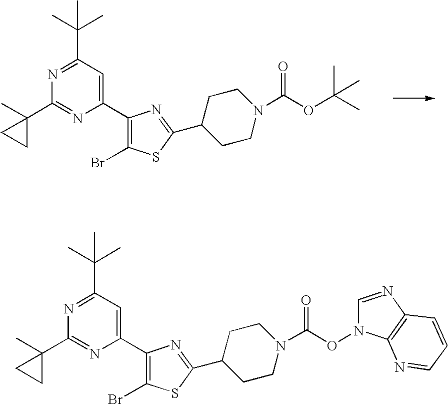

The thiazole (See Example 30 Step 7, 25 mg, 0.055 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (11 mg, 0.083 mmol) and the acid (See Example 1 Step 13, 12 mg, 0.066 mmol) were combined and the mixture dissolved in DMF (0.55 mL). Diisopropyl ethyl amine (36 mg, 0.28 mmol) and EDAC (16 mg, 0.083 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.38 (s, 1H); 8.67 (d, J=4.8 Hz, 1H); 8.32 (d, J=8.1 Hz, 1H); 7.79 (s, 1H); 7.66 (dd, J=4.8, 8.2 Hz, 1H); 5.64 (ABq, Δδ=23.0, J=16.9 Hz, 2H); 4.54 (d, J=13.5 Hz, 1H); 4.20 (d, J=14.0 Hz, 1H); 3.51-3.40 (m, 2H); 3.04-2.98 (t, J=13.5 Hz, 1H); 2.34 (d, J=11.7 Hz, 1H); 2.19 (d, J=12.1 Hz, 1H); 2.06 (dq, J=3.9, 13.0 Hz, 1H); 1.81 (dq, J=4.1, 12.8 Hz, 1H); 1.61 (s, 3H); 1.46 (m, 2H); 1.36 (s, 9H); 0.88 (m, 2H).

MS: m/z 594.15 (M+H).

### Example 31

### Preparation of 3-(2-{4-[5-chloro-4-(5,5,8,8-tetramethyl-5,6,7,8-tetrahydronaphthalen-2-yl)-1,3-thiazol-2-yl]piperidin-1-yl}-2-oxoethyl)-3H-imidazo[4,5-b]pyridine

### Step 1

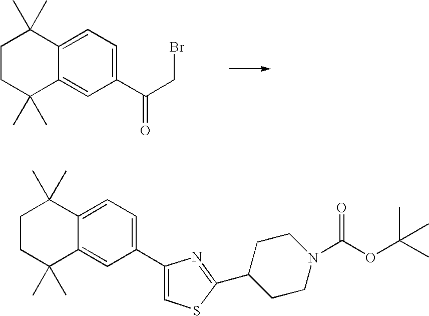

A vessel was charged with 2-bromo-1-(5,5,8,8-tetramethyl-5,6,7,8-tetrahydronaphthalen-2-yl)ethan-1-one (0.70 g, 2.3 mmol), thioamide (See Example 1 Step 9, 0.83 g, 3.4 mmol), and NaHCO₃ (0.21 g, 2.5 mmol), the materials dissolved in 1:1 ethyl alcohol/THF (15 mL). The resulting solution was stirred at RT for 2.5 hours, diluted with water (50 mL), followed by aqueous/EtOAc work-up and silica gel chromatography (diethyl ether:hexanes (1:4)) to afford the titled compound.

### Step 2

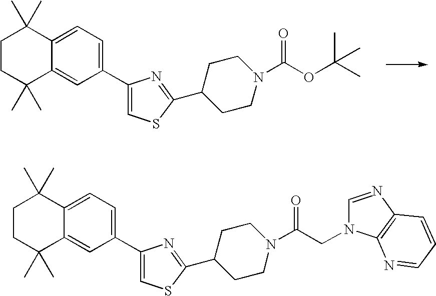

The thiazole (See Example 31 Step 1, 120 mg, 0.264 mmol) was dissolved in TFA (5 mL), stirred for 10 minutes and the volatiles removed in vac. The residue was dissolved in EtOAc, washed with aqueous NaOH (1N, 60 mL), and the aqueous wash extracted (3 times) with EtOAc. The combined organic fraction was washed with brine, dried over Na₂SO, filtered and reduced in vac. The recovered material, 1-HOBT (54 mg, 0.40 mmol) and the acid (See Example 1 Step 13, 61 mg, 0.34 mmol) were combined and the mixture dissolved in DMF (5.0 mL). Diisopropyl ethyl amine (0.17 g, 1.3 mmol) and EDAC (76 mg, 0.40 mmol) were added and the solution stirred at RT overnight. The mixture was diluted with 2:1 CH₃CN:water (20 mL) and purified by RP-18 HPLC (CH₃CN:H₂O 15 minute gradient 10 to 100%:0.1% TFA) to give the titled compound.

¹H NMR (500 MHz, CDCl₃): δ 8.34 (dd, J=1.3, 4.8 Hz, 1H); 8.22 (s, 1H); 8.06 (dd, J=1.3, 8.0 Hz, 1H); 7.77 (d, J=1.9 Hz, 1H); 7.56 (dd, J=1.9, 8.2 Hz, 1H); 7.30 (d, J=8.2 Hz, 1H); 7.21 (m, 2H); 5.16 (s, 2H); 4.54 (d, J=13.7 Hz, 1H); 4.07 (d, J=13.5 Hz, 1H); 3.38-3.27 (m, 2H); 2.93 (t, J=12.6 Hz, 1H); 2.28 (d, J=13.0 Hz, 1H); 2.17 (d, J=11.7 Hz, 1H); 1.94-1.78 (m, 2H); 1.66 (s, 4H); 1.30 (s, 6H); 1.25 (s, 6H).

MS: m/z=514.1 (M+H).

### Step 3

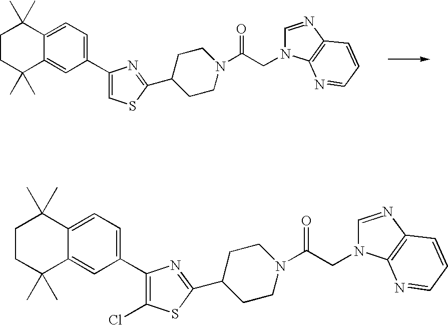

The amide (See Example 31 Step 2, 9.0 mg, 0.018 mmol) was dissolved in DMF (0.20 mL), NCS (2.6 mg, 0.019 mmol) added and the solution stirred at RT overnight. The reaction mixture was diluted with 2:1 CH₃CN:water (1 mL) and purified by RP-18 HPLC (CH₃CN:H₂O 15 minute gradient 10 to 100%:0.1% TFA) to give the titled compound.

¹H NMR (600 MHz, CDCl₃): δ 9.38 (s, 1H); 8.63 (d, J=4.4 Hz, 1H); 8.37 (d, J=8.2 Hz, 1H); 7.85 (d, J=1.9 Hz, 1H); 7.64 (dd, J=1.9, 8.2 Hz, 1H); 7.56 (dd, J=4.8, 8.2 Hz, 1H); 7.38 (d, J=8.3 Hz, 1H); 5.45 (ABq, Δδ=17.6, J=17.1 Hz, 2H); 4.53 (d, J=13.8 Hz, 1H); 4.05 (d, J=13.4 Hz, 1H); 3.46 (t, J=11.7 Hz, 1H); 3.32 (t, J=10.9 Hz, 1H); 3.02 (t, J=11.3 Hz, 1H); 2.35 (d, J=11.7 Hz, 1H); 2.21 (d, J=14.1 Hz, 1H); 2.01 (q, J=11.3 Hz, 1H); 1.84 (q, J=10.8 Hz, 1H); 1.72 (s, 4H); 1.33 (s, 6H); 1.31 (s, 6H).

MS: m/z=548.25 (M+H).

### Example 32

### Preparation of 3-(2-{4-[5-bromo-4-(5,5,8,8-tetramethyl-5,6,7,8-tetrahydronaphthalen-2-yl)-1,3-thiazol-2-yl]piperidin-1-yl}-2-oxoethyl)-3H-imidazo[4,5-b]pyridine

### Step 1

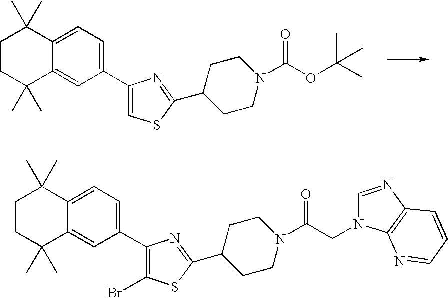

The thiazole (See Example 31 Step 1, 19 mg, 0.042 mmol) was dissolved in TFA (2 mL), stirred for 10 minutes, and the volatiles removed in vac. The residue was dissolved in EtOAc, washed with aqueous NaOH (1N, 40 mL), and the aqueous wash extracted (3 times) with EtOAc. The combined organic fraction was washed with brine, dried over Na₂SO, filtered and reduced in vac. The residue was then dissolved in acetic acid (0.77 mL), bromine (0.11 mL, 1 M in propionic acid) added, and the solution stirred at RT for 1.5 hours. Aqueous NaOH (20 mL) was added, followed by aqueous/EtOAc work-up to afford an oil. The recovered material, 1-HOBT (8.7 mg, 0.064 mmol) and the acid (See Example 1 Step 13, 9.1 mg, 0.051 mmol) were combined and the mixture dissolved in DMF (0.5 mL). Diisopropyl ethyl amine (0.027 g, 0.21 mmol) and EDAC (12 mg, 0.063 mmol) were added and the solution stirred at RT overnight. The mixture was diluted with 2:1 CH₃CN:water (2.5 mL) and purified by RP-18 HPLC (CH₃CN:H₂O 15 minute gradient 10 to 100%:0.1% TFA) to give the titled compound.

¹HNMR (600 MHz, CDCl₃): δ 9.47 (s, 1H); 8.64 (dd, J=1.3, 4.8 Hz, 1H); 8.40 (dd, J=1.2, 8.2 Hz, 1H); 7.84 (d, J=1.9 Hz, 1H); 7.61 (dd, J=1.9, 8.2 Hz, 1H); 7.58 (dd, J=4.8, 8.3 Hz, 1H); 7.38 (d, J=8.3 Hz, 1H); 5.50 (ABq, Δδ=19.8, J=16.3 Hz, 2H); 4.54 (d, J=13.1 Hz, 1H); 4.05 (d, J=13.3 Hz, 2H); 3.47 (t, J=11.5 Hz, 1H); 3.38-3.36 (tt, J=3.7, 11.1 Hz, 1H); 3.03 (t, J=11.0 Hz, 1H); 2.36 (d, J=10.5 Hz, 1H); 2.21 (d, J=12.8 Hz, 1H); 2.03 (q, J=10.1 Hz, 1H); 1.88-1.82 (q, J=12.6 Hz, 1H); 1.72 (s, 4H); 1.33 (s, 6H); 1.31 (s, 6H).

MS: m/z=592.18 (M+H).

### Example 33

### Preparation of [1-(2-{4-[5-chloro-4-(3,5-di-tert-butyl-4-methoxyphenyl)-1,3-thiazol-2-yl]piperidin-1-yl}-2-oxoethyl)-5-methyl-1H-1,2,4-triazol-3-yl]acetic acid

### Step 1

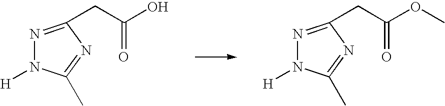

The acid (0.16 g, 1.2 mmol) was slurried in 7:2 benzene:methanol (12 mL), (trimethylsilyl)diazomethane (0.70 mL, 2.0 M in hexanes) added, and the solution stirred at RT for 30 minutes. A second addition of (trimethylsilyl)diazomethane (0.070 mL, 2.0 M in hexanes) was made and the solution stirred at RT for 4 hours. The solvent was removed in vac. to afford the titled compound.

### Step 2

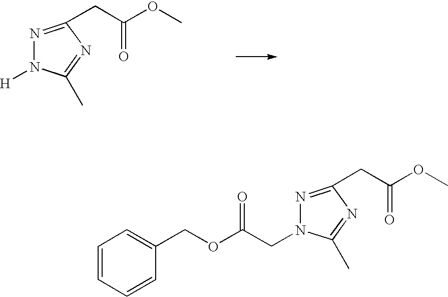

The ester (See Example 33 Step 1, 0.19 g, 1.2 mmol) was dissolved in DMF (12.0 mL), benzyl 2-bromoacetate (0.30 g, 1.31 mmol) and Cs₂CO₃ (0.47 g, 1.4 mmol) added and the solution stirred at RT overnight. The mixture was diluted with water (200 mL), followed by aqueous/EtOAc work-up, and silica gel chromatography (diethyl ether:hexanes (1:4)) to afford the titled compound.

### Step 3

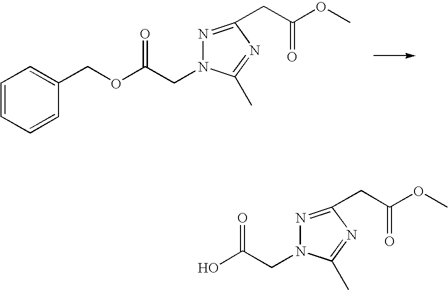

The ester (See Example 33 Step 2, 0.82 g, 0.27 mmol) was dissolved in methanol (10 mL), Pd(OH)₂ (20%, 74 mg) added, and the solution stirred under a hydrogen atmosphere for 0.5 hours at RT. The mixture was filtered and the volatiles removed in. vac. to afford the titled compound.

### Step 4

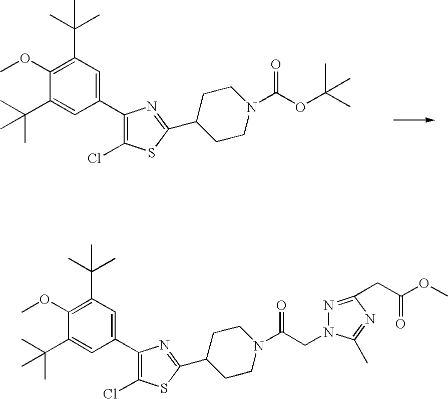

The thiazole (See Example 7 Step 1, 24 mg, 0.045 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (9.2 mg, 0.068 mmol) and the acid (See Example 33 Step 3, 11 mg, 0.052 mmol) were combined and the mixture dissolved in DMF (0.50 mL). Diisopropyl ethyl amine (29 mg, 0.22 mmol) and EDAC (13 mg, 0-068 mmol) were added and the solution stirred at RT overnight. The mixture was diluted with 2:1 CH₃CN:water (2.5 mL) and purified by RP-18 HPLC (CH₃CN:H₂O 15 minute gradient 10 to 100%:0.1% TFA) to give the titled compound.

### Step 5

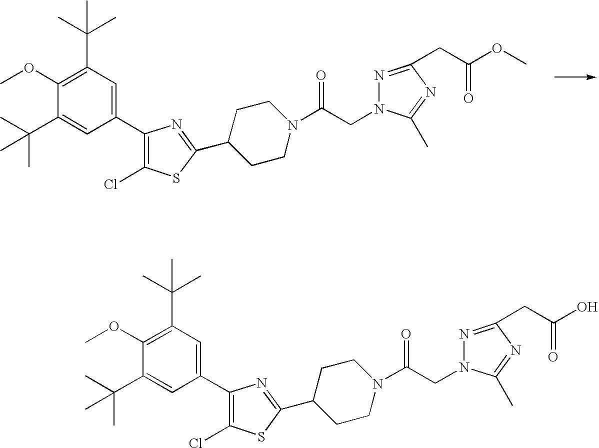

The thiazole (See Example 33 Step 4, 19.2 mg, 0.031 mmol) was dissolved in ethanol (0.5 mL), aqueous NaOH (1N, 0.065 mL) added, and the solution stirred at 45° C. for 2 hours. The mixture was diluted with 2:1 CH₃CN:water (2.5 mL) and purified by RP-18 HPLC (CH₃CN:H₂O 15 minute gradient 10 to 100%:0.1% TFA) to give the titled compound.

¹H NMR (500 MHz, CD3OD): δ 7.79 (s, 2H); 5.31 (ABq, Δδ=36.0, J=16.4 Hz, 2H); 4.52 (d, J=12.9 Hz, 1H); 4.05 (d, J=14.3 Hz, 1H); 3.78 (s, 2H); 3.72 (s, 3H); 3.40-3.34 (m, 2H); 2.96 (t, J=11.2 Hz, 1H); 2.50 (s, 3H); 2.24 (d, J=13.3 Hz, 1H); 2.18 (d, J=12.7 Hz, 1H); 1.93 (q, J=9.9 Hz, 1H); 1.76 (q, J=118 Hz, 1H); 1.46 (s, 18H).

MS: m/z=602.31 (M+H).

### Example 34

### Preparation of [1-(2-{4-[5-chloro-4-(3,5-di-tert-butyl-4-methoxyphenyl)-1,3-thiazol-2-yl]piperidin-1-yl}-2-oxoethyl)-5-methyl-1H-pyrazol-3-yl]acetic acid

### Step 1

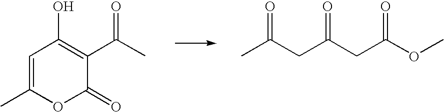

Magnesium methoxide solution (6% wt. % in methanol, 350 mL, 184 mmol) was added dropwise to stirred solution of dehydroacetic acid (20.60 g, 123 mmol) in methanol (400 mL), and the mixture then refluxed for 5 hours. The volatiles were removed in vac., the residue was diluted with aqueous HCl (1N, 1000 mL), followed by aqueous/EtOAc work-up to give the titled compound.

### Step 2

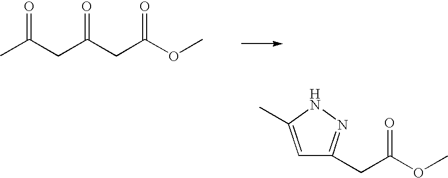

The di-ketone (See Example 34 Step 1, 6.0 g, 38 mmol) was dissolved in ethanol (40 mL), hydrazine hydrate (2.21 mL, 46 mmol) added dropwise, and the solution stirred at reflux for 3 hours. The solvent was removed in vac, and the residue purified by silica gel chromatography (acetone:CH₂Cl₂:acetic acid (1:3:0.1)) to give the titled compound.

### Step 3

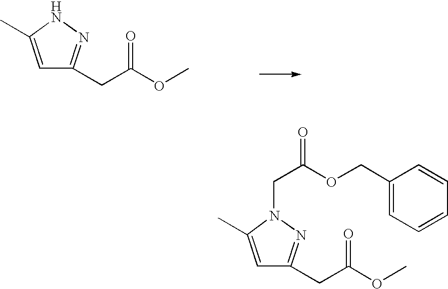

The pyrazole (See Example 34 Step 2, 462 mg, 3.0 mmol) was dissolved in DMF (8 mL), potassium carbonate (414.6 mg, 3.0 mmol) added, and the mixture warmed to 50° C. Benzyl 2-bromoacetate (687 mg, 3.0 mmol) was added dropwise the mixture stirred at 50° C. for 4 hours. The mixture was cooled to room temperature and the stirring continued overnight. The mixture was diluted with water, followed by aqueous/EtOAc work-up and silica gel chromatography (EtOAc:hexanes (1:2)). The material was further purified by chiral HPLC chromatography (OJ column, ethanol:heptane 70 min, 60% isocratic) was performed to give the titled compound.

### Step 4

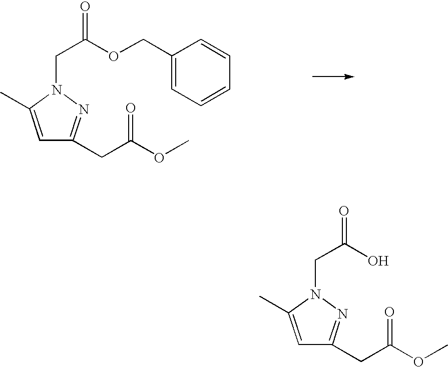

The benzyl ester (See Example 34 Step 3, 194 mg, 0.64 mmol) was dissolved in methanol (50 mL), palladium on carbon (10 wt. %, support activated carbon, 60 mg) added, and the mixture stirred under hydrogen atmosphere at room temperature for 2 hours. The mixture was filtered and the volatiles removed in vac. to give the titled compound.

### Step 5

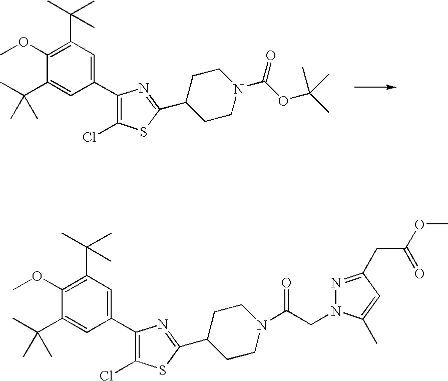

The chloro-thiazole (See Example 7 Step 1, 139 mg, 0.27 mmol) was dissolved in TFA (2 mL), stirred for 10 minutes and the solvent removed in vac. The residue was dissolved in EtOAc, washed with aqueous NaOH (1N, 40 mL) and the aqueous wash extracted (3 times) with EtOAc. The combined organic fraction was washed with brine, dried over Na₂SO, filtered and the volatiles removed in vac. The recovered material, 1-HOBT (55 mg, 0.40 mmol) and the acid (See Example 34 Step 4, 68 mg, 0.32 mmol) were combined and the mixture dissolved in DMF (2 mL). Diisopropyl ethyl amine (173 mg, 1.34 mmol) and EDAC (77 mg, 0.40 mmol) were added and the solution stirred at 45° C. for 1 hour. The mixture was diluted with 2:1 CH₃CN:water (10 mL) and purified by RP-18 HPLC (CH₃CN:H₂O 15 minute gradient 10 to 100%:0.1% TFA) to give the titled compound.

### Step 6

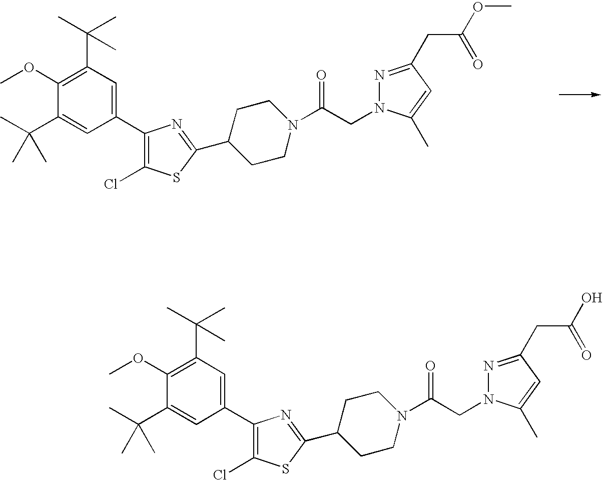

The methyl ester (See Example 34 Step 5, 117 mg, 0.19 mmol) was dissolved in methanol (2 mL), aqueous NaOH (1N, 0.80 mL), and the solution stirred at 45° C. for 40 minutes. The mixture was diluted with 2:1 CH₃CN:water (10 mL) and purified by RP-18 HPLC (CH₃CN:H₂O 15 minute gradient 10 to 100%:0.1% TFA) to give the titled compound.

¹H NMR (CD₃OD) δ 7.81 (s, 2H), 6.18 (s, 1H), 5.12 (ABq, Δδ=36.5, J=17.1 Hz, 2H), 4.55 (d, J=13.2 Hz, 1H), 4.11 (d, J=13.8 Hz, 1H), 3.73 (s, 3H), 3.60 (s, 2H), 3.36 (m, 2H), 2.96 (t, J=12.1 Hz, 1H), 2.28 (s, 3H), 2.20 (m, 2H), 1.90 (m, 1H), 1.75 (m, 1H), 1.49 (s, 18H)

MS: m/z=601.35 (M+H).

### Example 35

### Preparation of {f-[2-(4-{4-[3,5-bis(trifluoromethyl)phenyl]-5-chloro-1,3-thiazol-2-yl}piperidin-1-yl)-2-oxoethyl]-5-methyl-1H-pyrazol-3-yl}acetic acid

### Step 1

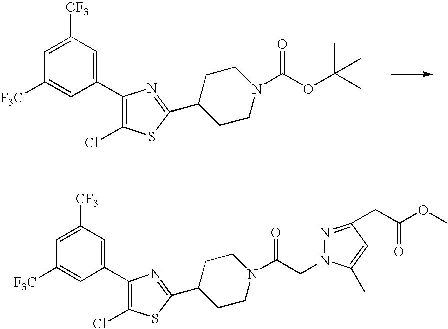

The thiazole (See Example 9 Step 1, 30 mg, 0.058 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and the volatiles removed in vac. The recovered material, 1-HOBT (14 mg, 0.10 mmol) and the acid (See Example 34 Step 4, 16 mg, 0.075 mmol) were combined and the mixture dissolved in DMF (0.70 mL). Diisopropyl ethyl amine (45 mg, 0.35 mmol) and EDAC (19 mg, 0.10 mmol) were added and the solution stirred at RT overnight. The mixture was diluted with 2:1 CH₃CN:water (2.5 mL) and purified by RP-18 HPLC (CH₃CN:H₂O 15 minute gradient 10 to 100%:0.1% TFA) to give the titled compound.

### Step 2

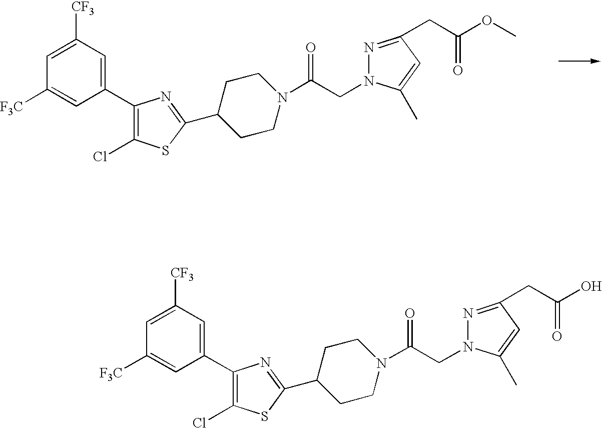

The thiazole (See Example 35 Step 1, 32 mg, 0.053 mmol) was dissolved in ethanol (0.60 mL) and aqueous NaOH (1N, 0.11 mL) added. The solution was stirred at 40° C. for 1 hour. The mixture was diluted with 2:1 CH₃CN:water (3.4 mL) and purified by RP-18 HPLC (CH₃CN:H₂O 15 minute gradient 10 to 100%:0.1% TFA) to give the titled compound.

¹H NMR (500 MHz, CD3OD): δ 8.54 (s, 2H); 8.00 (s, 1H); 6.08 (s, 1H); 5.07 (ABq, Δδ=38.4, J=17.2 Hz, 2H); 4.53 (d, J=13.1 Hz, 1H); 4.10 (d, J=14.4 Hz, 1H); 3.55 (s, 2H); 3.41-3.35 (m, 2H); 2.94 (t, J=11.5 Hz, 1H); 2.23 (s, 3H); 2.20 (m, 2H); 1.91 (dq, J=3.7, 12.6 Hz, 1H); 1.76 (dq, J=3.9, 12.6 Hz, 1H).

MS: m/z=595.00 (M+H).

### Example 36

### Preparation of [1-(2-{4-[5-bromo-4-(3,5-di-tert-butyl-4-methoxyphenyl)-1,3-thiazol-2-yl]piperidin-1-yl}-2-oxoethyl)-5-methyl-1H-pyrazol-3-yl]acetic acid

### Step 1

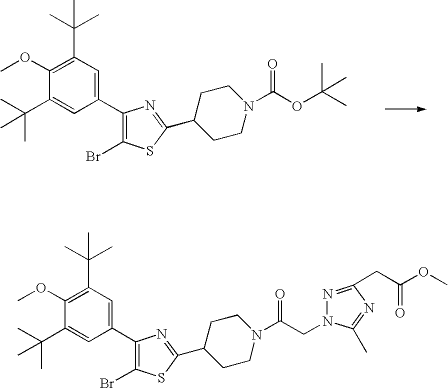

The thiazole (See Example 6 Step 3, 19 mg, 0.034 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and the volatiles removed in vac. The recovered material, 1-HOBT (6.9 mg, 0.051 mmol) and the acid (See Example 34 Step 4, 8.0 mg, 0.038 mmol) were combined and the mixture dissolved in DMF (0.50 mL). Diisopropyl ethyl amine (22 mg, 0.17 mmol) and EDAC (10 mg, 0.052 mmol) were added and the solution stirred at RT overnight. The mixture was diluted with 2:1 CH₃CN:water (2.5 mL) and purified by RP-18 HPLC (CH₃CN:H₂O 15 minute gradient 10 to 100%:0.1% TFA) to give the titled compound.

### Step 2

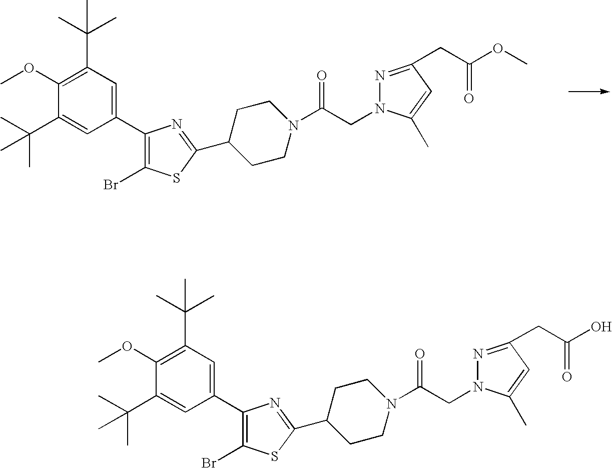

The thiazole (See Example 36 Step 1, 15 mg, 0.023 mmol) was dissolved in ethanol (0.45 mL) and aqueous NaOH (1N, 0.050 mL) added. The solution was stirred at 40° C. for 1 hour. The mixture was diluted with 2:1 CH₃CN:water (2.6 mL) and purified by RP-18 HPLC (CH₃CN:H₂O 15 minute gradient 10 to 100%:0.1% TFA) to give the titled compound.

¹H NMR (500 MHz, CD3OD): δ 7.78 (s, 2H); 6.13 (s, 1H); 5.10 (ABq, Δδ=35.7, J=17.2 Hz, 2H); 4.53 (d, J=13.3 Hz, 1H); 4.08 (d, J=14.1 Hz, 1H); 3.72 (s, 3H); 3.58 (s, 2H); 3.37-3.33 (m, 2H); 2.93 (t, J=11.5 Hz, 1H); 2.25 (s, 3H); 2.23-2.15 (m, 2H); 1.87 (dq, J=3.8, 12.8 Hz, 1H); 1.74 (dq, J=3.9, 12.5 Hz, 1H); 1.46 (s, 18H).

MS: m/z=645.11 (M+H).

### Example 37

### Preparation of [1-(2-{4-[5-bromo-4-(3,5-di-tert-butyl-4-hydroxyphenyl)-1,3-thiazol-2-yl]piperidin-1-yl}-2-oxoethyl)-5-methyl-1H-pyrazol-3-yl]acetic acid

### Step 1

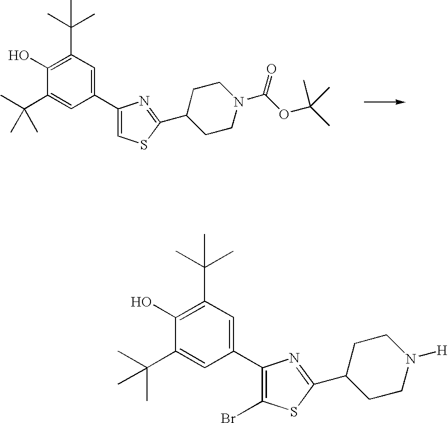

The thiazole (See Example 6 Step 1, 70 mg, 0.15 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and the volatiles removed in vac. The residue was dissolved in acetic acid (1.5 mL), bromine (0.21 mL, 0.78 M in propionic acid) added and the mixture stirred at RT for 1.0 hours. The solution was diluted with aqueous NaOH (1N, 50 mL), followed by aqueous/EtOAc work-up to afford an oil.

### Step 2

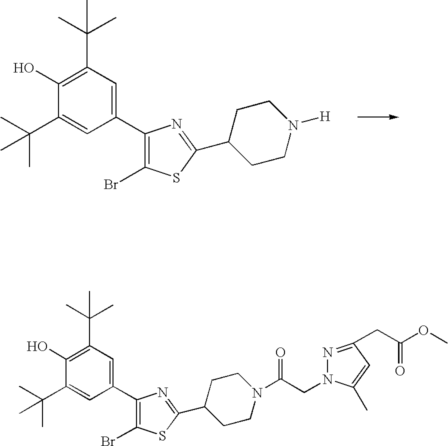

The amine (See Example 37 Step 1, 10 mg, 0.033 mmol), 1-HOBT (6.6 mg, 0.049 mmol) and the acid (See Example 34 Step 4, 10 mg, 0.033 mmol) were combined and the mixture dissolved in DMF (0.50 mL). Diisopropyl ethyl amine (21 mg, 0.16 mmol) and EDAC (9.4 mg, 0.049 mmol) were added and the solution stirred at RT overnight. The mixture was diluted with 2:1 CH₃CN:water (2.5 mL) and purified by RP-18 HPLC (CH₃CN:H₂O 15 minute gradient 10 to 100%:0.1% TFA) to give the titled compound.

### Step 3

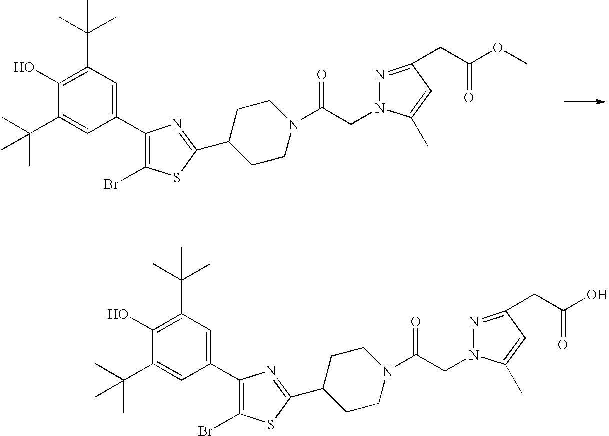

The thiazole (See Example 37 Step 2, 26 mg, 0.038 mmol) was dissolved in ethanol (0.70 mL), aqueous NaOH (1N, 0.085 mL) added, and the solution stirred at 40° C. for 2 hours. The mixture was diluted with 2:1 CH₃CN:water (2.3 mL) and purified by RP-18 HPLC (CH₃CN:H₂O 15 minute gradient 10 to 100%:0.1% TFA) to give the titled compound.

¹H NMR (600 MHz, CDCl₃): δ 7.63 (s, 2H); 6.24 (s, 1H); 5.41 (s, 1H); 5.29-5.13 (ABq, Δδ=84.2, J=16.9 Hz, 2H); 4.57 (d, J=13.7 Hz, 1H); 3.94 (d, J=13.9 Hz, 1H); 3.76 (s, 2H); 3.46-3.42 (tt, J=3.6, 11.4 Hz, 1H); 3.33 (t, J=12.0 Hz, 1H); 2.91 (t, J=11.7 Hz, 1H); 2.30 (s, 3H); 2.26 (d, J=13.5 Hz, 1H); 2.19 (m, 2H); 1.89 (q, J=10.8 Hz, 1H); 1.77 (q, J=10.8 Hz, 1H); 1.47 (s, 18H).

MS: m/z=631.18 (M+H).

### Example 38

### Preparation of [4-bromo-1-(2-{4-[5-chloro-4-(3,5-di-tert-butyl-4-methoxyphenyl)-1,3-thiazol-2-yl]piperidin 1-yl}-2-oxoethyl)-5-methyl-1H-pyrazol-3-yl]acetic acid

### Step 1

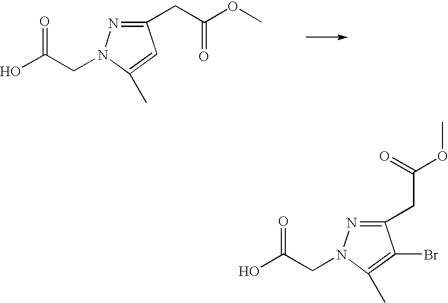

The acid (See Example 34 Step 4, 71 mg, 0.33 mmol) was dissolved in DMF (3.4 mL), NBS (95 mg, 0.53 mmol) added, and the solution stirred at RT for 1 hour. The mixture was diluted with 2:1 CH₃CN:water (9.6 mL) and purified by RP-18 HPLC (CH₃CN:H₂O 15 minute gradient 10 to 100%:0.1% TFA) to give the titled compound.

### Step 2

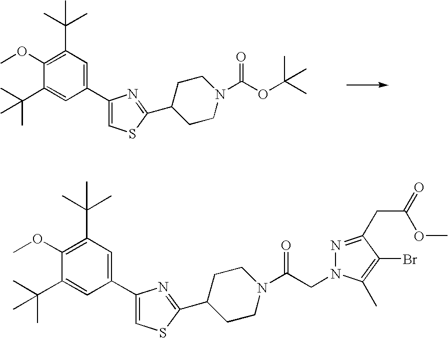

The thiazole (See Example 6 Step 2, 48 mg, 0.045 mmol) was dissolved in TFA (2 mL), stirred for 10 minutes, and the volatiles removed in vac. The residue was dissolved in EtOAc, washed with aqueous NaOH (5 mL) and the aqueous wash extracted (3 times) with EtOAc. The combined organic fraction was washed with brine, dried over MgSO₄, filtered and reduced in vac. The recovered material, 1-HOBT (19 mg, 0.14 mmol) and the acid (See Example 38 Step 1, 30 mg, 0.103 mmol) were combined and the mixture dissolved in DMF (0.90 mL). Diisopropyl ethyl amine (60 mg, 0.47 mmol) and EDAC (27 mg, 0.14 mmol) were added and the solution stirred at RT overnight. The mixture was diluted with water (10 mL) followed by aqueous/EtOAc work-up and to afford the titled compound.

### Step 3

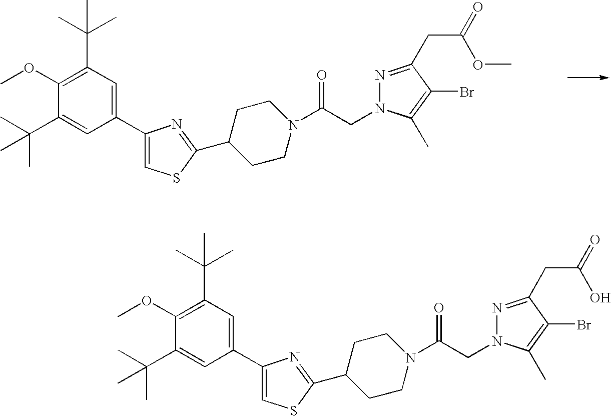

The thiazole (See Example 38 Step 2, 47 mg, 0.071 mmol) was dissolved in ethanol (0.8 mL), aqueous NaOH (1N, 0.15 mL) added and the solution warmed to 40° C. THF (0.5 mL) was added and the solution stirred at 40° C. for 1 hour.

The volatiles were removed in vac., the residue dissolved in 2:1 CH₃CN:water (4.0 mL) and purified by RP-18 HPLC (CH₃CN:H₂O 15 minute gradient 10 to 100%:0.1% TFA) to give the titled compound.

### Step 4

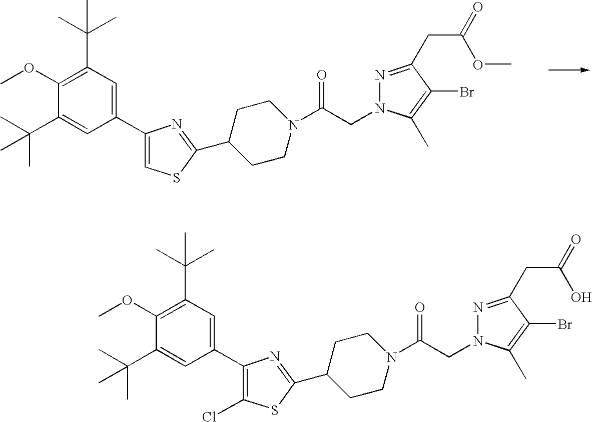

The acid (See Example 38 Step 3, 12 mg, 0.019 mmol) was dissolved in DMF (0.20 mL), NCS (2.8 mg, 0.021 mmol) added and the solution stirred at RT overnight. The mixture was diluted with 2:1 CH₃CN:water (1.8 mL) and purified by RP-18 HPLC (CH₃CN:H₂O 15 minute gradient 10 to 100%:0.1% TFA) to give the titled compound.

¹H NMR (500 MHz, CD3OD): δ 7.79 (s, 2H); 5.19-5.07 (ABq, Δδ=35.1, J=16.8 Hz, 2H); 4.51 (d, J=12.8 Hz, 1H); 4.08 (d, J=14.0 Hz, 1H); 3.71 (s, 3H); 3.58 (s, 2H); 3.37-3.33 (m, 2H); 2.93 (t, J=11.5 Hz, 1H); 2.22 (s, 3H); 2.21 (m, 1H); 2.16 (d, J=9.9 Hz, 1H); 1.88 (q, J=13.2 Hz, 1H); 1.73 (dq, J=12.2 Hz, 1H); 1.46 (s, 18H).

MS: m/z=679.01 (M+H).

### Example 39

### Preparation of 3-[1-(2-{4-[5-chloro-4-(3,5-di-tert-butyl-4-methoxyphenyl)-1,3-thiazol-2-yl]piperidin-1-yl}-2-oxoethyl)-5-methyl-1H-pyrazol-3-yl]propanoic acid

### Step 1

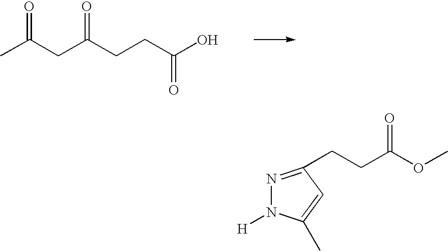

The acid (1.3 g, 8.4 mmol) was slurried in 7:2 benzene:methanol (78 mL), (trimethylsilyl)diazomethane (5.4 mL, 2.0 M in hexanes) added, and the solution stirred at RT for 30 minutes. The volatiles were removed in vac and the residue was dissolved in ethanol (78 mL). Hydrazine hydrate (0.52 g, 10.3 mmol) was added and the solution stirred at RT overnight. The volatiles were removed in. vac. to give the titled compound.

### Step 2

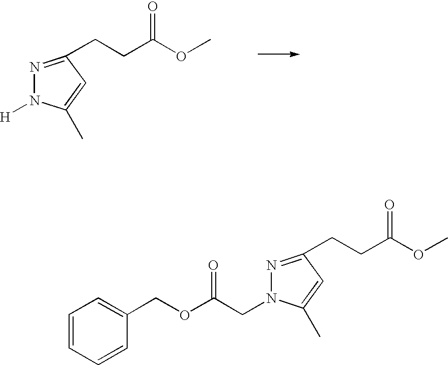

The ester (See Example 39 Step 1, 1.5 g, 8.8 mmol) was dissolved in DMF (80 mL), the solution cooled to 0° C., lithium hexamethyldisilazide (9.68 mL, 1.0 M in THF) added, and the mixture stirred at 0° C. for 10 minutes. Benzyl 2-bromoacetate (2.5 g, 10.9 mmol) was added, the solution allowed to warm to RT, and stirred for 5 hours. The mixture was diluted with water (500 mL), followed by aqueous/EtOAc work-up, silica gel chromatography (acetone:hexanes (1:4)). A second chromatography on chiral HPLC (OD column, ethanol:heptane) gave the titled compound.

### Step 3

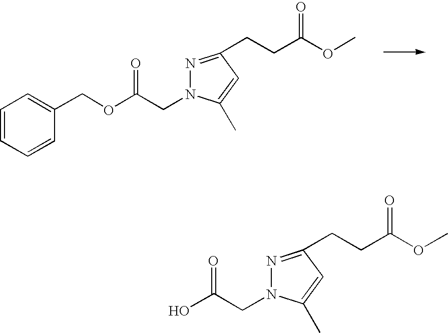

The ester (See Example 39 Step 2, 0.62 g, 2.0 mmol) was dissolved in methanol (20 mL), Pd(OH)₂ (20%, 149 mg) added, and the slurry stirred under a hydrogen atmosphere for 1.0 hour at RT. The mixture was filtered and volatiles removed in. vac. to afford the titled compound.

### Step 4

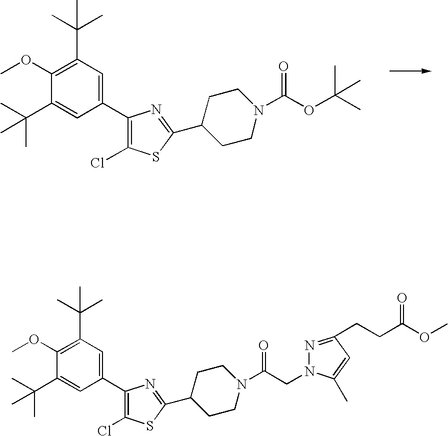

The thiazole (See Example 7 Step 1, 22 mg, 0.042 mmol) was dissolved in TFA (2 mL), stirred for 10 minutes, and the volatiles removed in vac. The residue was dissolved in EtOAc, washed with 4:1 water/saturated NaHCO₃ (40 mL) and the aqueous wash extracted (3 times) with EtOAc. The combined organic fraction was washed with brine, dried over MgSO₄, filtered and reduced in vac. The recovered material, 1-HOBT (8.6 mg, 0.063 mmol) and the acid (See Example 39 Step 3, 11 mg, 0.046 mmol) were combined and the mixture dissolved in DMF (0.50 mL). Diisopropyl ethyl amine (28 mg, 0.22 mmol) and EDAC (12 mg, 0.063 mmol) were added and the solution stirred at RT overnight. The mixture was diluted with DMF (2.5 mL) and purified by RP-18 HPLC (CH₃CN:H₂O 15 minute gradient 10 to 100%:0.1% TFA) to give the titled compound.

### Step 5

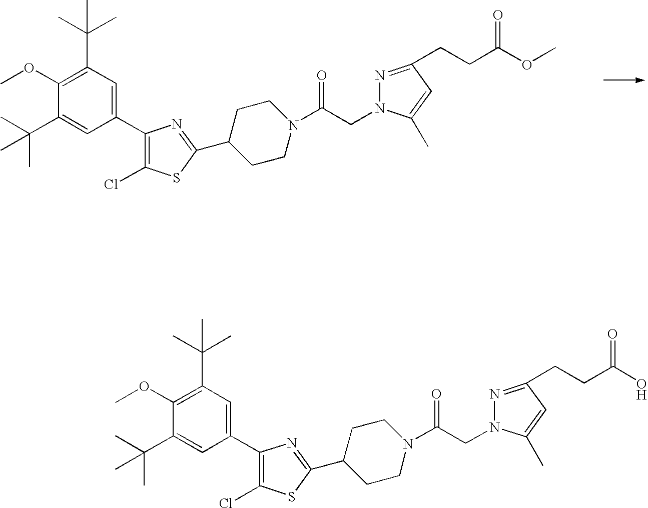

The thiazole (See Example 39 Step 4, 29 mg, 0.045 mmol) was dissolved in ethanol (0.5 mL), aqueous NaOH (1N, 0.095 mL) added, and the solution stirred at 40° C. for 4.5 hours. The mixture was diluted with 2:1 CH₃CN:water (3.5 mL) and purified by RP-18 HPLC (CH₃CN:H₂O 15 minute gradient 10 to 100%:0.1% TFA) to give the titled compound.

¹H NMR (500 MHz, CD3OD): δ 7.79 (s, 2H); 6.10 (s, 1H); 5.13 (ABq, Δδ=32.6, J=17.1 Hz, 2H); 4.53 (d, J=14.1 Hz, 1H); 4.07 (d, J=12.8 Hz, 1H); 3.72 (s, 3H); 3.39-3.31 (m, 2H); 2.94 (t, J=13.2 Hz, 1H); 2.88 (t, J=7.5 Hz, 2H); 2.64 (t, J=7.5 Hz, 2H); 2.25 (s, 3H); 2.19 (m, 2H); 1.88 (dq, J=4.1, 12.8 Hz, 1H); 1.74 (q, J=3.8, 12.6 Hz, 1H); 1.46 (s, 18H).

MS: m/z=615.16 (M+H).

### Example 40

### Preparation 3-{2-[4-(5-chloro-4-{3-(trifluoromethoxy)-5-[(trifluoromethyl)thio]phenyl}-1,3-thiazol-2-yl)piperidin-1-yl]-2-oxoethyl}-3H-imidazo[4,5-b]pyridine

### Step 1

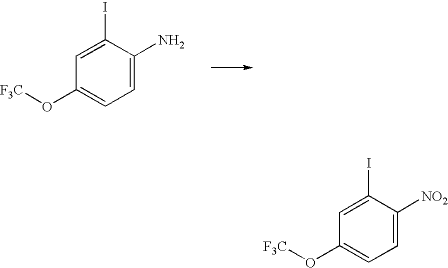

A solution of 4-(trifluoromethoxy)-2-iodoaniline (2.0 g, 6.6 mol) in CH₂Cl₂ (5 mL) was added dropwise at RT to a slurry of meta-chloroperoxybenzoic acid (4.6 g, 26 mmol) in CH₂Cl₂ (28 mL). The solution was stirred at RT overnight. The mixture was diluted with 4:1 water/saturated NaHCO₃, followed by aqueous/EtOAc work-up and silica gel chromatography (diethyl ether:hexanes (1:9)) to afford the titled compound.

### Step 2

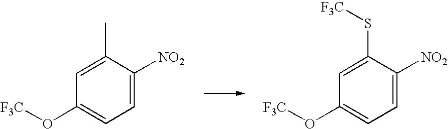

The nitro compound (See Example 40 Step 1, 0.89 g, 2.7 mmol) and copper trifluoromethanethiol (0.57 g, 3.47 mmol) were combined in N-methylpyrrolidinone (2.67 mL) and the mixture stirred at 150° C. for 1 hour. The mixture was cooled to RT, diluted with 4:1 water/saturated NaHCO₃, followed by aqueous/EtOAc work-up and silica gel chromatography (diethyl ether:hexanes (1:19)) to afford the titled compound.

### Step 3

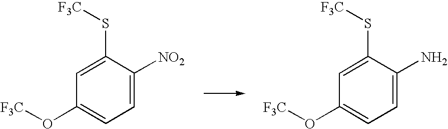

The nitro compound (See Example 40 Step 2, 0.35 g, 1.1 mmol) was dissolved in methanol (11 mL) and solid zinc (0.37 grams; 5.7 mmol) added. Acetic acid (0.30 mL) was added dropwise and the slurry stirred at RT for 45 minutes. The mixture was filtered and the volatiles removed in. vac. The residue was dissolved in EtOAc, washed with aqueous NaOH (1 N), and the aqueous wash extracted with EtOAc (3 times). The combined organic fraction was washed with brine, dried over MgSO₄, filtered, and the volatiles removed in vac. to afford the titled compound.

### Step 4

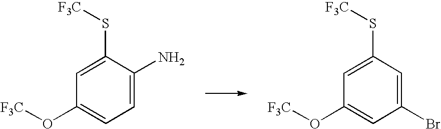

The aniline (See Example 40 Step 3, 0.28 g, 1.02 mmol) was dissolved in DMF (10 mL), NBS (0.20 g, 1.1 mmol) added and the solution stirred at RT overnight. The mixture was diluted with aqueous NaOH (1N, 200 mL) followed by aqueous/EtOAc work-up to afford the titled compound. The aniline was then dissolved in DMF (1.2 mL), added dropwise to a 70° C. solution of isoamyl nitrite (0.17 g, 1.4 mmol) in DMF (0.65 mL) and stirred for 1 hour. The solution was cooled to RT, diluted with water (50 mL) followed by aqueous/EtOAc work-up and silica gel chromatography (hexanes, neat) to afford the titled compound.

### Step 5

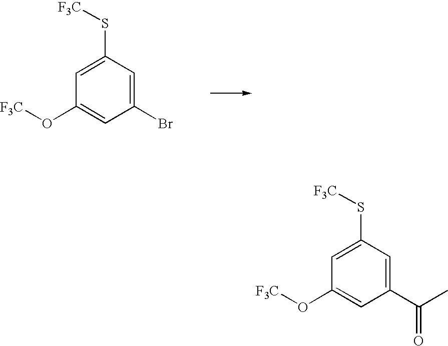

The bromide (See Example 40 Step 4, 0.093 g, 0.27 mmol), tri-nbutyl-1-ethoxyvinyl tin (0.12 g, 0.33 mmol), and PdCl₂(Ph₃P)₂ (19 mg, 0.027 mmol) were dissolved in toluene (0.54 mL) and the mixture stirred at 95° C. for 1 hour. The solution was cooled to RT, aqueous HCl (2 N, 0.66 mL) added and the mixture stirred rapidly for 1 hour. The mixture was diluted with water (40 mL) followed by aqueous/EtOAc work-up and silica gel chromatography (diethyl ether:hexanes (1:9)) to give the titled compound.

### Step 6

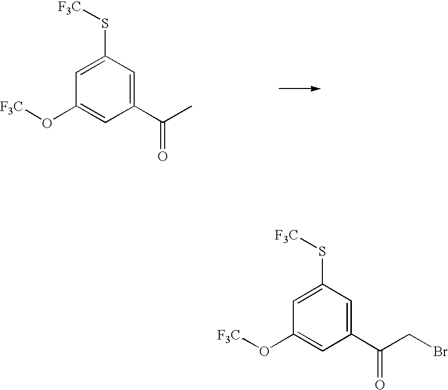

The ketone (See Example 40 Step 5, 58 mg, 0.19 mmol) was dissolved in diethyl ether (1.0 mL), and aluminum chloride (1.1 mg, 0.009 mmol) added at RT. Bromine (30 mg, 0.19 mmol) was added dropwise and the resulting mixture stirred for 15 minutes. The mixture was diluted with a solution of concentrated HCl:ice water (4:40 mL), followed by aqueous/EtOAc work-up.

### Step 7

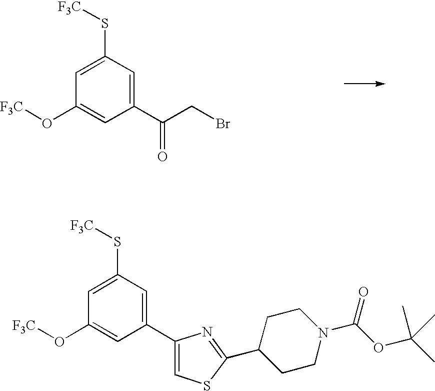

The bromoketone (See Example 40 Step 6, 0.064 g, 0.17 mmol) was dissolved in 1:1 ethyl alcohol/THF (1.7 mL), the thioamide (See Example 1 Step 9, 0.041 g, 0.17 mmol) added, and the mixture stirred at RT overnight. The volatiles were removed in. vac. and the residue dissolved DMF (1.0 mL). Et₃N (0.17 g, 1.7 mmol) and di-tert-butyl dicarbonate (36 mg, 0.17 mmol) were added and the solution stirred at RT for 1 hour. The mixture was diluted with 4:1 water/saturated NaHCO₃ followed by aqueous/EtOAc work-up and silica gel chromatography (diethyl ether:hexanes (1:7)) to afford the titled compound.

### Step 8

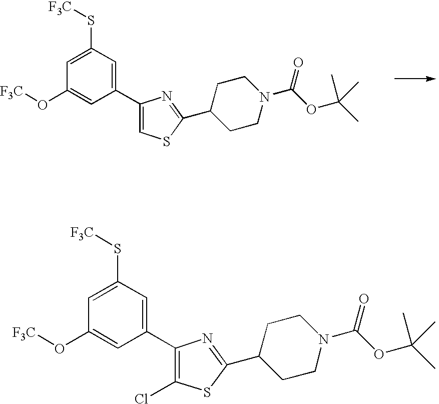

The thiazole (See Example 40 Step 7, 0.067 g, 0.13 mmol) was dissolved in DMF (1.3 mL), NCS (0.018 g, 0.13 mmol) added and the solution stirred at 45° C. for 1 hour. The mixture was cooled to RT, diluted with aqueous NaOH (50 mL), followed by aqueous/EtOAc work-up and silica gel chromatography (EtOAc:hexanes (1:7)) to afford the titled compound.

### Step 9

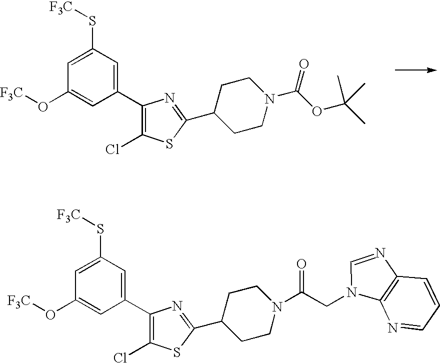

The chloride (See Example 40 Step 8, 0.028 g, 0.050 mmol) was dissolved in TFA (2 mL), stirred for 10 minutes, and the volatiles removed in vac. The residue was dissolved in EtOAc, washed with 4:1 water/saturated NaHCO₃ (400 mL) and the aqueous wash extracted (3 times) with EtOAc. The combined organic fraction was washed with brine, dried over Na₂SO, filtered and reduced in vac. The recovered material, 1-HOBT (0.010 g, 0.075 mmol) and the acid (See Example 1 Step 13, 0.011 g, 0.060 mmol) were combined and the mixture dissolved in DMF (0.50 mL). Diisopropyl ethyl amine (32 mg, 0.25 mmol) and EDAC (14 mg, 0.075 mmol) were added and the solution stirred at RT overnight. The mixture was diluted with 2:1 CH₃CN:water (6 mL) and purified by RP-18 HPLC (CH₃CN:H₂O 15 minute gradient 10 to 100%:0.1% TFA) to give the titled compound.

¹H NMR (500 MHz, CD3OD): δ 9.05 (s, 1H); 8.57 (dd, J=1.2, 4.8 Hz, 1H); 8.32 (s, 1H); 8.25 (dd, J=1.1, 8.2 Hz, 1H); 8.09 (s, 1H); 7.66 (s, 1H); 7.55 (dd, J=4.8, 8.2 Hz, 1H); 5.56 (ABq, Δδ=22.4, J=16.9 Hz, 2H); 4.52 (d, J=13.5 Hz, 1H); 4.20 (d, J=14.0 Hz, 1H); 3.48 (t, J=13.0 Hz, 2H); 3.41 (tt, J=3.9, 11.5 Hz, 2H); 3.00 (t, J=11.9 Hz, 1H); 2.33 (d, J=13.3 Hz, 1H); 2.19 (d, J=11.7 Hz, 1H); 2.06 (dq, J=3.9, 12.7 Hz, 1H); 1.81 (dq, J=3.8, 12.7 Hz, 1H).

MS: m/z=622.17 (M+H).

### Example 42

### Preparation of [2-(2-{4-[5-chloro-4-(3,5-di-tert-butyl-4-methoxyphenyl)-1,3-thiazol-2-yl]piperidin-1-yl}-2-oxoethyl)-1,3-thiazol-4-yl]acetic acid

### Step 1

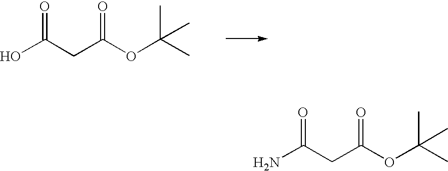

1-HOBT (30 g, 222 mmol) was dissolved in methanol (350 mL), ammonia (2.0 M in methanol, 70 mL, 140 mmol) added at RT and the volatiles removed in vac. A portion of the material (2.0 g, 13.2 mmol) was combined with EDAC (1.89 g, 9.87 mmol) and mono-tert-butyl malonate (1.05 g, 6.6 mmol). The solids dissolved in DMF (10 mL) and the mixture stirred at RT overnight. The mixture was poured into saturated NaHCO₃:water (1:1, 400 mL), followed by aqueous/EtOAc work-up to afford the titled compound.

### Step 2

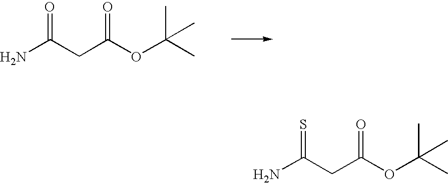

The amide (See Step 1, 400 mg, 2.52 mmol) was dissolved in 1,4-dioxane (3 mL). The mixture was heated at 60° C. until a clear solution results. Lawesson's reagent (508 mg, 1.26 mmol) was added and the mixture stirred at 60° C. for 2 hours. The solution was allowed to cool to room temperature and the solvent was removed in vac. The residue was poured saturated NaHCO₃:water (1:1, 200 mL), followed by aqueous/EtOAc work-up and silica gel chromatography (EtOAc:hexanes (2:3)) to give the titled compound.

### Step 3

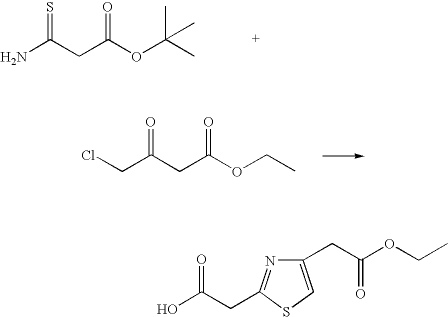

Ethyl 4-chloroacetate (329 mg, 2.0 mmol) was added dropwise to a solution of the thioamide (See Step 2, 350 mg, 2.0 mmol) dissolved in 1:1 THF:n-butanol (10 mL). The solution was heated at reflux for 5 hours, allowed to cool to room temperature and stirred overnight. The solvent was removed in vac and the residue poured into water (200 mL), followed by aqueous/EtOAc work-up and silica gel chromatography (EtOAc:hexanes (2:3)). A portion of the recovered material (29 mg, 0.10 mmol) was dissolved in TFA (1 mL), stirred for 10 minutes, and the volatiles removed in vac to afford the titled compound.

### Step 4

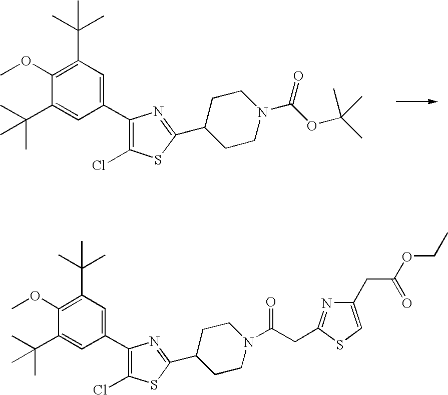

The chloride (See Example 7 Step 1, 37 mg, 0.07 mmol) was dissolved in TFA (1 mL), stirred at RT for 10 minutes and the volatiles removed in vac. The residue was dissolved in EtOAc, washed with NaOH (1N aqueous, 40 mL) and extracted with EtOAc (3 times). The combined organic fraction was washed with brine, dried over Na₂SO, filtered and volatiles removed in vac. The material was combined with 1-HOBT (15 mg, 0.10 mmol) and the acid (See Step 3, 18 mg, 0.08 mmol) and the combined materials dissolved in DMF (1 mL). Diisopropyl ethyl amine (45 mg, 0.36 mmol) and EDAC (20 mg, 0.10 mmol) were added and the solution allowed to stir at 45° C. for 1 hour. The reaction mixture was diluted with 2:1 CH₃CN:water (6 mL) and purified by RP-18 HPLC (CH₃CN:H₂O 15 minute gradient 10 to 100%:0.1% TFA) to give the titled compound.

### Step 5:

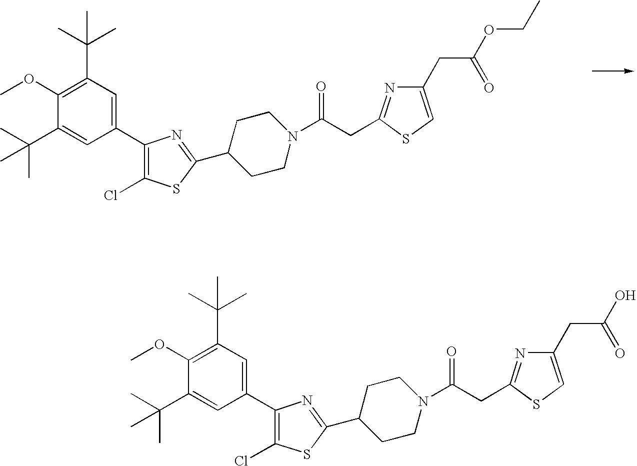

Aqueous NaOH (1N, 0.15 mL) was added to a solution of the ester (See Step 4, 22 mg, 0.04 mmol) in ethanol (1 mL). The reaction was stirred at 45° C. for 40 min, diluted in 2:1 CH₃CN:water (6 mL) and purified by RP-18 HPLC (CH₃CN:H₂O 15 minute gradient 10 to 100%:0.1% TFA) to give the titled compound.

NMR (Acetone-d6): δ 7.92 (s, 2H), 7.86 (s, 1H), 4.69 (ABq, Δδ=33.7, J=21.7 Hz, 2H), 4.65 (d, J=14.2 Hz, 1H), 4.23 (d, J=13.7 Hz, 1H), 4.09 (s, 2H), 3.77 (s, 3H), 3.44 (m, 2H), 3.03 (t, J=12.1 Hz, 1H), 2.25 (m, 2H), 1.94 (m, 1H), 1.78 (m, 1H), 1.48 (s, 18H).

MS: m/z 604.51 (M+H).

### Example 43

### Preparation of [2-(2-{4-[5-chloro-4-(3,5-di-tert-butyl-4-methoxyphenyl)-1,3-thiazol-2-yl]piperidin-1-yl}-2-oxoethyl)-5-methyl-1,3-thiazol-4-yl]acetic acid

### Step 1

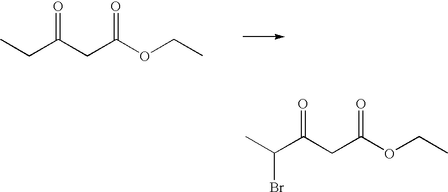

Bromine (1.82 M in chloroform, 20 mL) was added dropwise for 2 hours to a stirred solution of ethyl propionylacetate (5.25 g, 36.4 mmol) in chloroform (40 mL) with the temperature maintained at 0° C. The reaction mixture was stirred for one hour and then allowed to warm to room temperature overnight with constant stirring. The solution was bubbled with air in for 1 hour and the remaining volatiles removed in vac to give the titled compound.

### Step 2

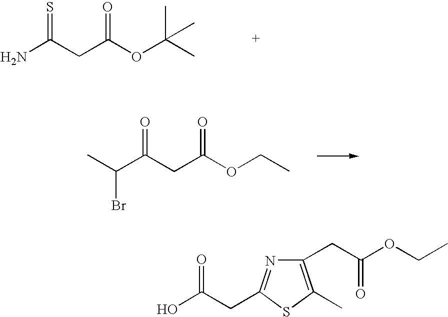

The bromo-compound (See Step 1, 255 mg, 1.14 mmol) was added dropwise to the thioamide (See Example 42 Step 2, 200 mg, 1.14 mmol) in the solution of THF:n-butanol (1:1, 8 mL) at room temperature. The reaction mixture was heated at reflux for 5 hours. Then it was cooled to room temperature and stirred overnight. The solvent was removed in vac. The residue was poured into water (200 mL), followed by aqueous/EtOAc work-up and silica gel chromatography (EtOAc:hexanes (2:3)). The chromatographed material (23 mg, 0.08 mmol) was dissolved in TFA (1 mL) and stirred for 10 minutes. The solvent were removed in vac to give the titled compound.

### Step 3

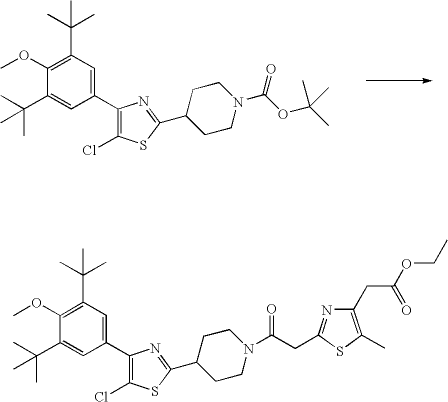

The chloride (See Example 7 Step 1, 39 mg, 0.08 mmol) was dissolved in TFA (1 mL), stirred at RT for 10 minutes, and the volatiles removed in vac. The residue was dissolved in EtOAc, washed with NaOH (1N aqueous, 40 mL) and extracted (3 times) with EtOAc. The combined organic fraction was washed with brine, dried over Na₂SO, filtered and the volatiles removed in vac. The material was combined with 1-HOBT (16 mg, 0.12 mmol) and the acid (See Step 2, 18 mg, 0.08 mmol) and the combined material dissolved in DMF (1 mL). Diisopropyl ethyl amine (76 mg, 0.59 mmol) and EDAC (23 mg, 0.12 mmol) were added and the solution stirred at 45° C. for 1 hour. The reaction mixture was diluted with 2:1 CH₃CN:water (6 mL) and purified by RP-18 HPLC (CH₃CN:H₂O 15 minute gradient 10 to 100%:0.1% TFA) to give the titled compound.

### Step 4.

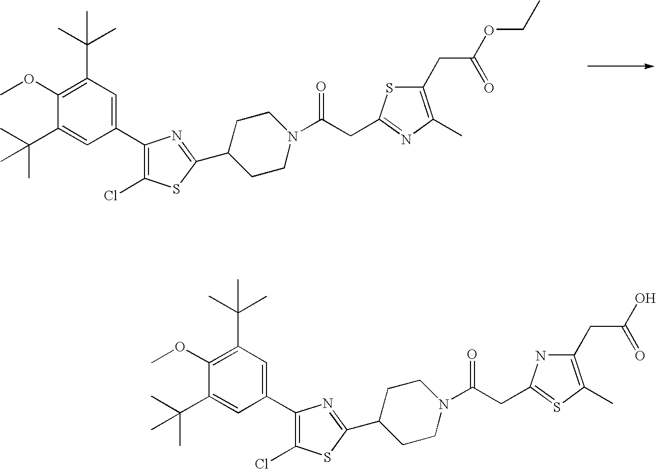

Aqueous NaOH (1N, 0.10 mL) was added to a solution of the ethyl ester (See Step 3, 31 mg, 0.05 mmol) in ethanol (1 mL). The reaction was heated at 45° C. for 40 min, diluted with 2:1 CH₃CN:water (6 mL) and purified by RP-18 HPLC (CH₃CN:H₂O 15 minute gradient 10 to 100%:0.1% TFA) to give the titled compound.

NMR (Acetone-d): δ 7.88 (s, 21), 4.79 (ABq, Δδ=32.7, J=21.7 Hz, 2H), 4.62 (d, J=14.2 Hz, 1H), 4.15 (d, J=13.7 Hz, 1H), 4.04 (s, 2H), 3.72 (s, 3H), 3.40 (m, 2H), 2.99 (t, J=12.1 Hz, 1H), 2.56 (s, 3H), 2.25 (m, 2H), 1.90 (m, 1H), 1.73 (m, 1H), 1.45 (s, 18H).

MS: m/z=618.53 (M+H).

### Example 44

### Preparation of [1-(2-{4-[5-chloro-4-(3,5-di-tert-butyl-4-methoxyphenyl)-1,3-thiazol-2-yl]piperidin-1-yl}-2-oxoethyl)-4,5-dimethyl-1H-pyrazol-3-yl]acetic acid

### Step 1

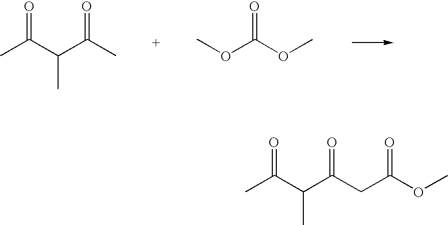

LiMDS (1.0M in THF, 26.6 mL, 26.6 mmol) was dissolved in THF (20 mL) and cooled to −78° C. 3-Methylpentane-2,4-dione (1.0 g, 8.76 mmol) was added dropwise and the cooling bath removed and the solution allowed to warm to room temperature with stirring for 4 hours. The solution was cooled to −78° C. and dimethyl carbonate added. The mixture was allowed to warm to room temperature overnight. The solvent was removed in vac and the residue added to a 10% HCl solution (aqueous), followed by aqueous/EtOAc work-up and silica gel chromatography (EtOAc:hexanes (2:3)) to give the titled compound.

### Step 2

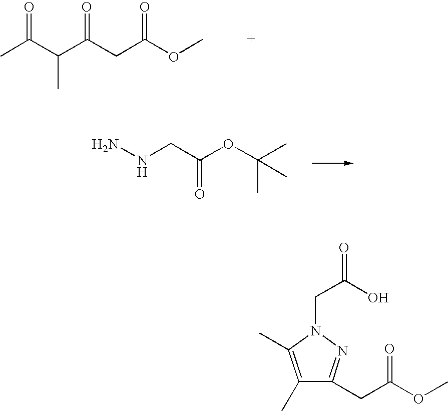

The di-ketone (See Step 1, 206.4 mg, 1.2 mmol), was dissolved in t-butanol and the hydrazine malonic acid salt (262.27 mg, 1.0 mmol) and 4-methyl-morpholine (304 mg, 3.0 mmol) were added. The mixture was heated at reflux for 3 hours. The solvent was removed in vac and the residue chromatographed on silica gel (EtOAc:hexanes (1:2)). The chromatographed material was dissolved in TFA (2 mL), stirred for 10 minutes., and the volatiles removed in vac to afford the titled compound.

### Step 3

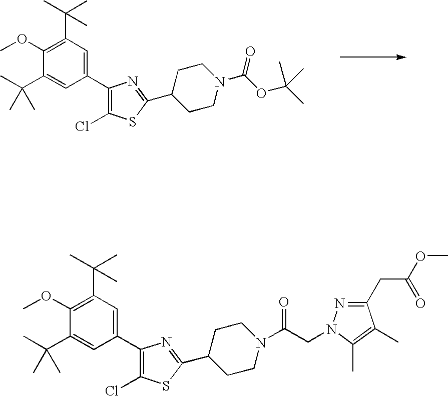

The chloride (See Example 7 Step 1, 40 mg, 0.09 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (18 mg, 0.13 mmol) and the acid (See Step 2, 18 mg, 0.08 mmol), and the combined materials dissolved in DMF (1 mL). Diisopropyl ethyl amine (57 mg, 0.44 mmol) and EDAC (25 mg, 0.13 mmol) were added and the solution allowed to stir at 45° C. for 1 hour. The reaction mixture was diluted in 2:1 CH₃CN:water (8 mL) and purified by RP-18 HPLC (CH₃CN:B₂O 15 minute gradient 10 to 100%:0.1% TFA) to give the titled compound.

### Step 4

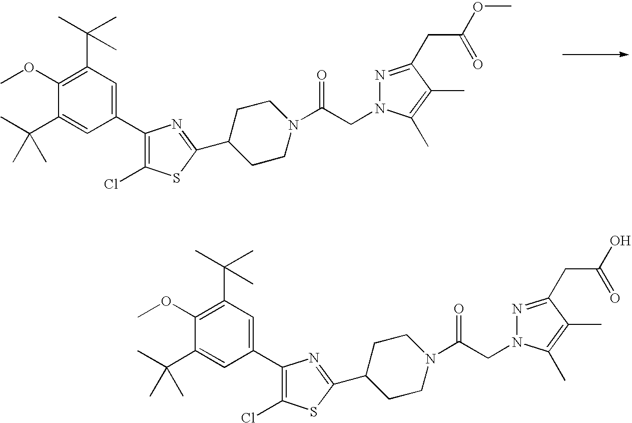

The methyl ester (See Step 7, 22 mg, 0.04 mmol) was dissolved in methanol (1 mL), aqueous NaOH (1N, 0.15 mL) added and the mixture stirred at 45° C. for 40 min. The solution was diluted with 2:1 CH₃CN:water (4 mL) and purified by RP-18 HPLC (CH₃CN:H₂O 15 minute gradient 10 to 100%:0.1% TFA) to give the titled compound.

NMR (CD₃OD) δ 7.82 (s, 2H), 5.14 (ABq, Δδ=33.3, J=16.9 Hz, 2H), 4.55 (d, J=13.5 Hz, 1H), 4.09 (d, J=14.0 Hz, 1H), 3.74 (s, 3H), 3.63 (s, 2H), 3.34 (m, 2H), 2.96 (t, J=12.6 Hz, 1H), 2.23 (m, 2H), 2.21 (s, 3H), 2.01 (s, 3H), 1.90 (m, 1H), 1.76 (m, 1H), 1.48 (s, 18H)

MS: m/z=615.60 (M+H).

### Example 45

### Preparation of [2-(2-{4-[5-bromo-4-(3,5-di-tert-butyl-4-methoxyphenyl)-1,3-thiazol-2-yl]piperidin-1-yl}-2-oxoethyl)-5-methyl-1,3-thiazol-4-yl]acetic acid

### Step 1

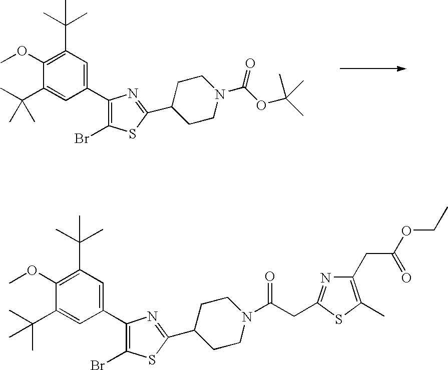

The bromide (See Example 6 Step 3, 45 mg, 0.08 mmol) was dissolved in TFA (1 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (16 mg, 0.12 mmol) and the acid (See Example 43 Step 2, 18 mg, 0.08 mmol) and the combined materials dissolved in DMF (1 mL). Diisopropyl ethyl amine (68 mg, 0.45 mmol) and EDAC (23 mg, 0.12 mmol) were added and the solution allowed to stir at 45° C. for 1 hour. The reaction mixture was diluted in 2:1 CH₃CN:water (6 mL) and purified by RP-18 HPLC (CH₃CN:H₂O 15 minute gradient 10 to 100%:0.1% TFA) to give the titled compound.

### Step 2

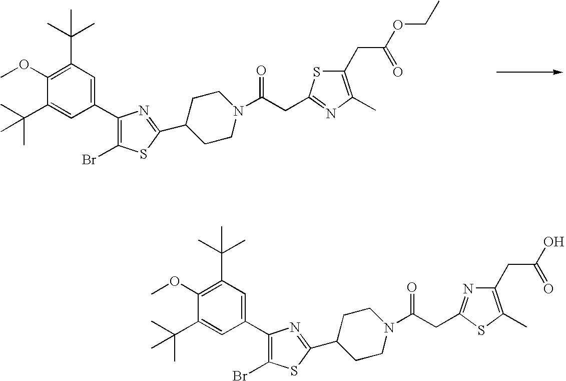

The ethyl ester (See Step 1, 35 mg, 0.05 mmol) was dissolved in ethanol (1 mL) and NaOH (1N, 0.10 mL) added. The reaction was stirred at 45° C. for 40 min., diluted with 2:1 CH₃CN:water (6 mL) and purified by RP-18 HPLC (CH₁₃CN:H₂O 15 minute gradient 10 to 100%:0.1% TFA) to give the titled compound.

NMR (Acetone-D) δ 7.91 (s, 2H), 4.79 (ABq, Δδ=35.7, J=18.7 Hz, 2H), 4.64 (d, J=13.5 Hz, 1H), 4.18 (d, J=14.0 Hz, 1H), 4.07 (s, 2H), 3.76 (s, 3H), 3.45 (m, 2H), 3.08 (t, J=11.9 Hz, 1H), 2.60 (s, 3H), 2.26 (m, 2H), 1.93 (m, 1H), 1.78 (m, 1H), 1.48 (s, 18H).

MS: m/z=664.52 (M+H).

### Example 46

### Preparation of 7-(2-{4-[5-chloro-4-(3,5-di-tert-butyl-4-methoxyphenyl)-1,3-thiazol-2-yl]piperidin-1-yl}-2-oxoethyl)-7H-purin-6-ol

### Step 1

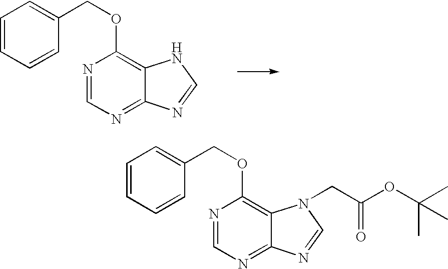

The benzyl protected compound (1.0 g, 4.42 mmol) was dissolved in DMF (4.4 mL). Cs₂CO₃ (2.9 g, 8.84 mmol) and t-butyl bromo acetate (0.86 g, 4.42 mmol) were added and the solution stirred at RT overnight. The residue was poured into 4:1 water:saturated NaHCO₃ (200 mL), followed by aqueous/EtOAc work-up and silica gel chromatography (acetone:hexanes (1:1)) to afford the titled compound.

### Step 2

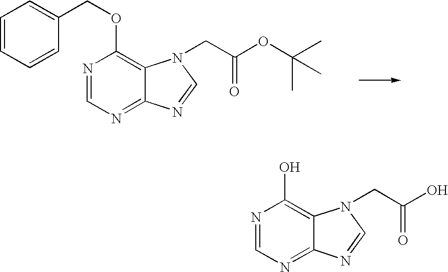

The benzyl protected compound (See Step 1, 200 mg, 0.59 mmol) was dissolved in methanol (50 mL), palladium on carbon (10 wt. %, 50 mg) added, and the slurry stirred at RT under a hydrogen atmosphere for 2 hours. The mixture was filtered and the volatiles removed in vac. The material was then dissolved in TFA (2 mL), stirred for 10 minutes, and the volatiles removed in vac to afford the titled compound.

### Step 3

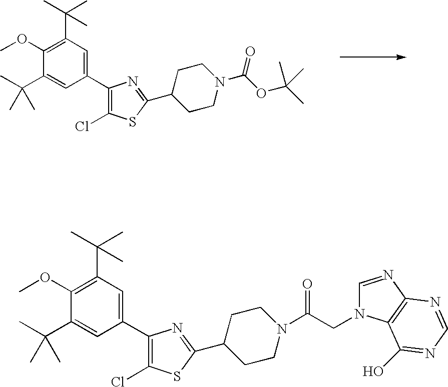

The chloride (See Example 7 Step 1, 39 mg, 0.09 mmol) was dissolved in TFA (1 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and reduced in vac. The material was combined with 1-HOBT (19 mg, 0.14 mmol) and the acid (See Step 2, 18 mg, 0.09 mmol) and the combined materials dissolved in DMF (0.5 mL). Diisopropyl ethyl amine (60 mg, 0.47 mmol) and EDAC (27 mg, 0.14 mmol) were added and the solution allowed to stir at 45° C. for 1 hour. The reaction mixture was diluted with 2:1 CH₃CN:water (6 mL) and purified by RP-18 HPLC (CH₃CN:H₂O 15 minute gradient 10 to 100%:0.1% TFA) to give the titled compound.

NMR (CD₃OD) δ 8.49 (s, 1H), 8.12 (s, 1H), 7.83 (s, 2H), 5.55 (ABq, Δδ=39.8, J=16.7 Hz, 2H), 4.54 (d, J=13.3 Hz, 1H), 4.11 (d, J=14.0 Hz, 1H), 3.75 (s, 3H), 3.42 (m, 2H), 3.02 (t, J=14.4 Hz, 1H), 2.30 (d, J=12.4 Hz, 1H), 2.21 (d, J=12.0 Hz, 1H), 2.04 (m, 1H), 1.82 (m, 1H), 1.49 (s, 18H).

MS: m/z=597.60 (M+H).

### Example 47

### Preparation of 1-(2-[4-[5-chloro-4-(3,5-di-tert-butyl-4-methoxyphenyl)-1,3-thiazol-2-yl]piperidin-1-yl]-2-oxoethyl)-1,3-dihydro-2H-indol-2-one

### Step 1

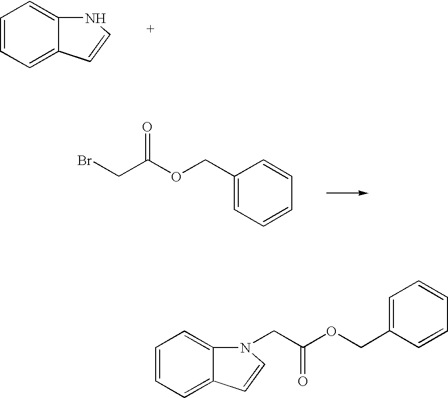

The indole (5.86 g, 50 mmol) was dissolved in DMF (25 mL) and Cs₂CO₃ (48.8 g, 150 mmol) was added. The slurry was stirred at RT for 10 minutes, benzyl 2-bromoacetate (17.2 g, 75 mmol) added dropwise, and the solution stirred overnight. The mixture was poured into water, followed by aqueous/EtOAc work-up and silica gel chromatography (EtOAc:hexanes (1:4)) to give the titled compound.

### Step 2

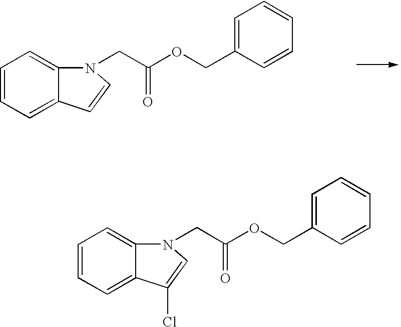

The indole (See Step 1, 2.65 g, 10 mmol) was dissolved in CH₂Cl₂ (25 mL), the solution cooled to 0° C., and NCS (1.47 g, 11 mmol) added portionwise. Upon completion of the addition the cooling bath removed and the solution stirred at RT overnight. The volatiles were removed in vac, the residue diluted with water, followed by aqueous/EtOAc work-up and silica gel chromatography (EtOAc:hexanes (1:5)) to afford the titled compound.

### Step 3

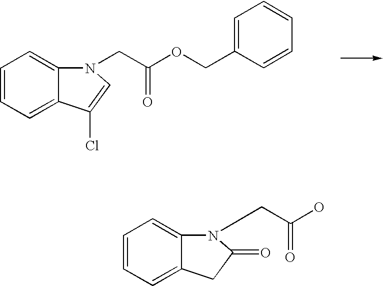

The chloride (See Step 2, 598 mg, 2.0 mmol) was dissolved in 2-methoxy-ethanol (10 mL). Phosphoric acid (85% water solution, 10 mL) was added and the solution heated at 100° C. overnight. The volatiles were removed in vac, the residue poured into water, followed by aqueous/EtOAc work-up and silica gel chromatography (acetone:hexanes:acetic acid (3:7:0.2)) to give the titled compound.

### Step 4

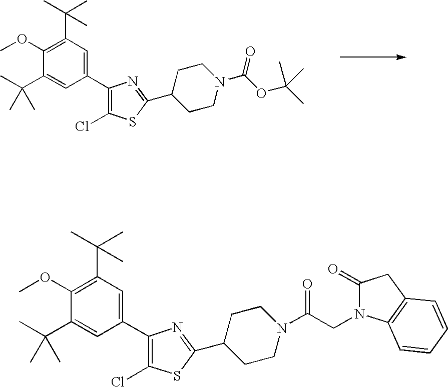

The chloride (See Example 7 Step 1, 62 mg, 0.12 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and reduced in vac. The material was combined with 1-HOBT (25 mg, 0.18 mmol) and the acid (See Step 3, 23 mg, 0.12 mmol) and the combined materials dissolved in DMF (0.5 mL). Diisopropyl ethyl amine (78 mg, 0.60 mmol) and EDAC (35 mg, 0.18 mmol) were added and the solution stirred at 45° C. for 1 hour. The reaction mixture was diluted with 2:1 CH₃CN:water (8 mL) and purified by RP-18 HPLC (CH₃CN:H₂O 15 minute gradient 10 to 100%:0.1% TFA) to give the titled compound.

NMR (CDCl₃) δ 7.67 (s, 2H), 7.34 (m, 2H), 7.16 (t, J=7.3 Hz, 1H), 6.87 (d, J=8.0 Hz, 1H), 4.73 (d, J=13.7 Hz, 1H), 4.71 (ABq, Δδ=94.1, J=16.4 Hz, 2H), 4.14 (d, J=14.0 Hz, 1H), 3.77 (s, 3H), 3.76 (s, 2H), 3.63 (m, 11H), 3.44 (t, J=14.4 Hz, 1H), 2.95 (t, J=14.4 Hz, 1H), 2.39 (d, J=13.0 Hz, 1H), 2.27 (d, J=13.0 Hz, 1H), 1.85 (m, 1H), 1.50 (s, 18H).

MS: m/z=594.62 (M+H).

### Example 48

### Preparation of [1-(2-{4-[5-chloro-4-(3,5-di-tert-butylphenyl)-3-thiazol-2-yl]piperidin-1-yl}-2-oxoethyl)-5-methyl-1H-pyrazol-3-yl]acetic acid

### Step 1

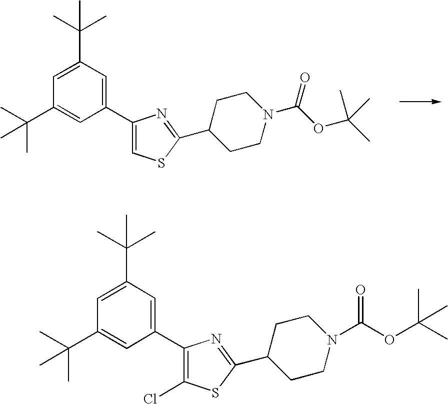

The thiazole (See Example 20 Step 3, 267 mg, 0.58 mmol) was dissolved in DMF (5.8 mL), NCS (7.4 mg, 0.056 mmol) was added and the solution stirred at 45° C. for 1 hour. The solution was allowed to cool to RT, poured into aqueous NaOH (1N, 40 mL), followed by aqueous/EtOAc work-up and silica gel chromatography (diethyl ether:hexanes (1:4)) to afford the titled compound.

### Step 2

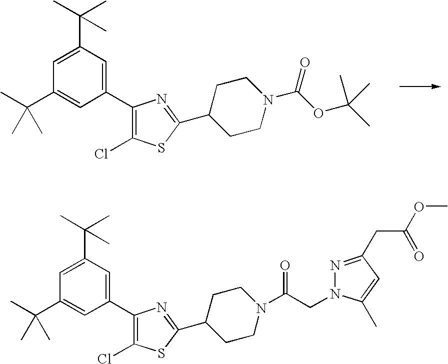

The chloride (See Step 1, 89 mg, 0.18 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and reduced in vac. The material was combined with 1-HOBT (36 mg, 0.27 mmol) and the acid (See Example 34 Step 4, 38 mg, 0.18 mmol) and the combined materials dissolved in DMF (1 mL). Diisopropyl ethyl amine (114 mg, 0.89 mmol) and EDAC (51 mg, 0.27 mmol) were added and the solution, stirred at 45° C. for 1 hour. The reaction mixture was diluted in 2:1 CH₃CN:water (8 mL) and purified by RP-18 HPLC (CH₃CN:H₂O 15 minute gradient 10 to 100%:0.1% TFA) to give the titled compound.

### Step 3

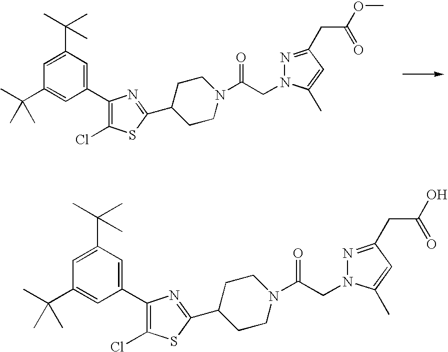

The methyl ester (See Step 3, 68 mg, 0.12 mmol) was dissolved in methanol (1 mL) and aqueous NaOH (1N, 0.25 mL) added. The solution was stirred at 45° C. for 40 min., diluted in 2:1

CH₃CN:water (6 mL) and purified by RP-18 HPLC (CH₃CN:H₂O 15 minute gradient 10 to 100%:0.1% TFA) to give the titled compound.

NMR (CDCl₃) δ 7.67 (s, 2H), 7.45 (s, 1H), 6.18 (s, 1H), 5.15 (ABq, Δδ=34.6, J=16.7 Hz, 2H), 4.55 (d, J=13.2 Hz, 1H), 3.95 (d, J=13.5 Hz, 1H), 3.72 (s, 2H), 3.32 (t, J=11.2 Hz, 2H), 2.92 (t, J=11.9 Hz, 1H), 2.28 (s, 3H), 2.26 (d, J=8.7 Hz, 1H), 2.19 (d, J=7.2 Hz, 1H), 1.82 (m, 2H), 1.36 (s, 18H).

MS: m/z=571.53 (M+fi).

### Example 49

### Preparation of [1-(2-{4-[5-chloro-4-(3,5-di-tert-butyl-4-hydroxyphenyl)-1,3-thiazol-2-yl]piperidin-1-yl}-2-oxoethyl)-5-methyl-1H-pyrazol-3-yl]acetic acid

### Step 1

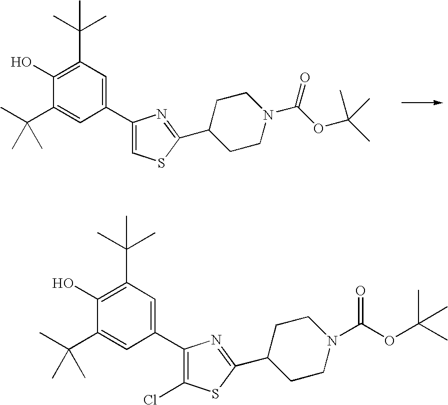

The thiazole (See Example 6 Step 1, 0.20 g, 0.42 mmol) was dissolved in DMF (3.2 mL). NCS (0.062 g, 0.465 mmol) was added and the solution stirred at 45° C. for 1.0 hour. The reaction mixture was cooled to room temperature, poured into water (400 mL), followed by aqueous/EtOAc work-up and silica gel chromatography (EtOAc:hexanes (1:9)) to afford the titled compound.

### Step 2

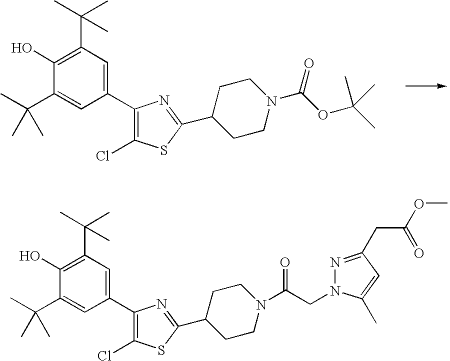

The chloride (See Step 1, 35 mg, 0.07 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and reduced in vac. The material was combined with 1-HOBT (14 mg, 0.10 mmol) and the acid (See Example 34 Step 4, 15 mg, 0.07 mmol) and the combined materials dissolved in DMF (1 mL). Diisopropyl ethyl amine (67 mg, 0.52 mmol) and EDAC (20 mg, 0.10 mmol) were added and the solution stirred at 45° C. for 1 hour. The reaction mixture was diluted with 2:1 CH₃CN:water (8 mL) and purified by RP-18 HPLC (CH₃CN:H₂O 15 minute gradient 10 to 100%:0.1% TFA) to give the titled compound.

### Step 3

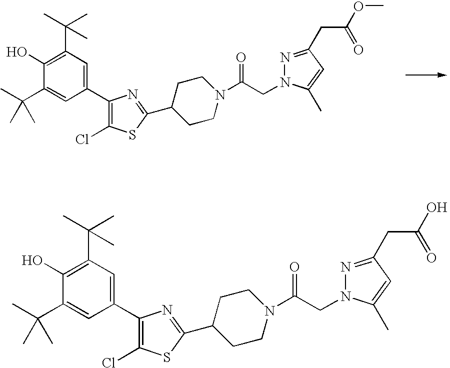

The methyl ester (See Step 2, 26 mg, 0.04 mmol) was dissolved in methanol (1 mL) and aqueous NaOH (1N, 0.09 mL,) added. The solution was stirred at 45° C. for 40 min., diluted with 2:1 CH₃CN:water (4 mL) and purified by RP-18 HPLC (CH₃CN:H₂O 15 minute gradient 10 to 100%:0.1% TFA) to give the titled compound.

NMR (CDCl₃) δ 9.19 (br, 1H), 7.67 (s, 2H), 6.14 (s, 1H), 5.38 (br, 1H), 5.11 (ABq, Δδ=23.0, J=16.7 Hz, 2H), 4.53 (d, J=13.5 Hz, 1H), 3.95 (d, J=14.2 Hz, 1H), 3.71 (s, 2H), 3.30 (m, 2H), 2.90 (t, J=11.7 Hz, 1H), 2.28 (s, 3H), 2.26 (d, 1H, J=8.7 Hz), 2.19 (d, 1H, J=7.2 Hz), 1.82 (m, 2H), 1.47 (s, 18H)

MS: m/z=587.53 (M+H).

### Example 50

### Preparation of (1-{2-[4-(4-{3-tert-butyl-5-[(trifluoromethyl)thio]phenyl}-5-chloro-1,3-thiazol-2-yl)piperidin-1-yl]-2-oxoethyl}-5-methyl-1H-pyrazol-3-yl)acetic acid

### Step 1

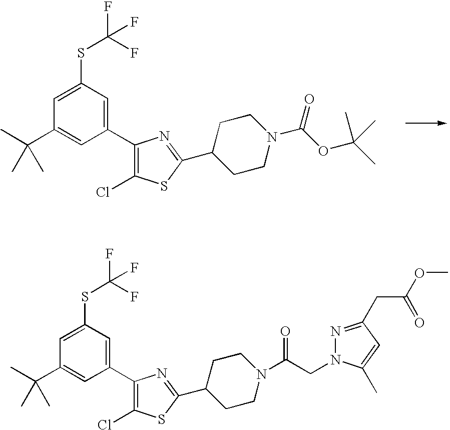

The chloride (See Example 1 Step 11, 33 mg, 0.06 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (13 mg, 0.09 mmol) and the acid (See Example 34 Step 4, 14 mg, 0.06 mmol) and the combined materials dissolved in DMF (1 mL). Diisopropyl ethyl amine (40 mg, 0.31 mmol) and EDAC (18 mg, 0.09 mmol) were added and the solution stirred at 45° C. for 1 hour. The reaction mixture was diluted with 2:1 CH₃CN:water (8 mL) and purified by RP-18 HPLC (CH₃CN:H₂O 15 minute gradient 10 to 100%:0.1% TFA) to give the titled compound.

### Step 2

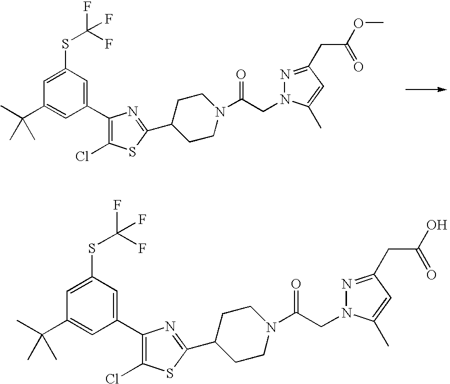

The methyl ester (See Step 1, 17 mg, 0.03 mmol) was dissolved in methanol (0.5 μL) and aqueous NaOH (1N, 0.11 mL) added. The reaction was stirred at 45° C. for 40 min., diluted with 2:1 CH₃CN:water (4 mL) and purified by RP-18 HPLC (CH₃CN:H₂O 15 minute gradient 10 to 100%:0.1% TFA) to give the titled compound.

NMR (CD₃OD) δ 8.19 (s, 1H), 8.07 (s, 1H), 7.76 (s, 1H), 6.18 (s, 1H), 5.14 (ABq, Δδ=35.6, J=17.2 Hz, 2H), 4.56 (d, J=13.2 Hz, 1H), 4.11 (d, J=13.5 Hz, 1H), 3.68 (s, 2H), 3.38 (m, 2H), 2.97 (t, J=13.5 Hz, 1H), 2.28 (s, 3H), 2.22 (m, 2H), 1.94 (m, 1H), 1.78 (m, 1H), 1.41 (s, 9H).

MS: m/z=615.31 (M+H).

### Example 51

### Preparation of 1-(2-{4-[5-chloro-4-(3,5-di-tert-butyl-4-methoxyphenyl)-1,3-thiazol-2-yl]piperidin-1-yl}-2-oxoethyl)-1,3-dihydro-2H-benzimidazol-2-one

### Step 1

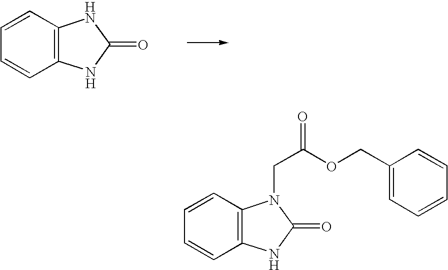

2-Hydroxybenzimidazole (4.03 g, 30 mmol) was dissolved in DMF (30 mL) and sodium hydride (60% mineral oil dispersion, 1.2 g, 30 mmol) was slowly added at RT. The mixture was stirred for 1 hour, benzyl 2-bromoacetate (6.87 mg, 30 mmol) added dropwise, and the mixture stirred overnight. The volatiles were removed in vac and the residue poured into water, followed by aqueous/EtOAc work-up and silica gel chromatography (EtOAc:hexanes (4:1)) to give a mixture of mono-, and di-alkylated compounds. A second chromatography by RP-18 HPLC (CH₃CN:H₂O 15 minute gradient 10 to 100%:0.1% TFA) afforded the titled compound.

### Step 2

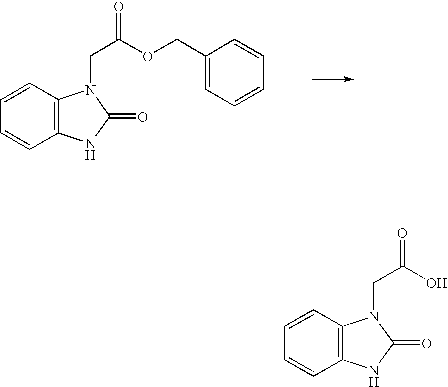

The ester (See Step 2, 50 mg, 0.18 mmol) was dissolved in methanol (10 mL) and palladium on carbon (10 wt. %, 15 mg) was added. The slurry was stirred at RT under an atmosphere of hydrogen for 1 hour. The mixture was filtered and the volatiles removed in vac to give the titled compound.

### Step 3

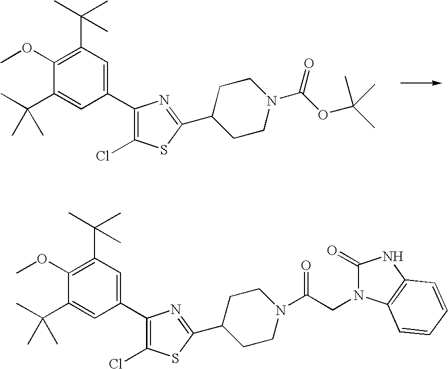

The chloride (See Example 7 Step 1, 52 mg, 0.10 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (21 mg, 0.15 mmol) and the acid (See Step 2, 19 mg, 0.10 mmol) and the combined materials dissolved in DMF (1 mL). Diisopropyl ethyl amine (65 mg, 0.50 mmol) and EDAC (29 mg, 0.15 mmol) were added and the solution stirred at 45° C. for 1 hour. The mixture was diluted with 2:1 CH₃CN:water (3 mL) and purified by RP-18 HPLC (CH₃CN:H₂O 15 minute gradient 10 to 100%:0.1% TFA) to give the titled compound.

NMR (CD₃OD) δ 7.83 (s, 2H), 7.08 (m, 4H), 4.86 (ABq, Δδ32 34.0, J=14.3 Hz, 2H), 4.53 (d, J=13.3 Hz, 1H), 4.18 (d, J=14.0 Hz, 1H), 3.73 (s, 3H), 3.38 (m, 2H), 2.92 (t, J=14.2 Hz, 1H), 2.26 (d, J=8.7 Hz, 1H), 2.16 (d, J=7.2 Hz, 1H), 1.92 (m, 1H), 1.75 (m, 1H), 1.47 (s, 18H).

MS: m/z=595.55 (M+H).

### Example 52

### Preparation of (2-{2-[4-(4-{3-tert-butyl-5-[(trifluoromethyl)thio]phenyl}-5-chloro-1,3-thiazol-2-yl)piperidin-1-yl]-2-oxoethyl}-1,3-thiazol-4-yl)acetic acid

### Step 1

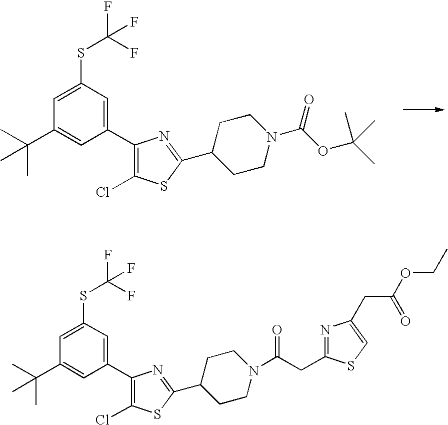

The chloride (See Example 1 Step 11, 37 mg, 0.07 mmol) was dissolved in TFA (1 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and reduced in vac. The material was combined with 1-HOBT (15 mg, 0.10 mmol) and the acid (See Example 42 Step 3, 18 mg, 0.08 mmol) and the combined materials dissolved in DMF (1 mL). Diisopropyl ethyl amine (45 mg, 0.36 mmol) and EDAC (20 mg, 0.10 mmol) were added and the solution stirred at 45° C. for 1 hour. The reaction mixture was diluted with 2:1 CH₃CN:water (6 mL) and purified by RP-18 HPLC (CH₃CN:H₂O 15 minute gradient 10 to 100%:0.1% TFA) to give the titled compound.

### Step 2:

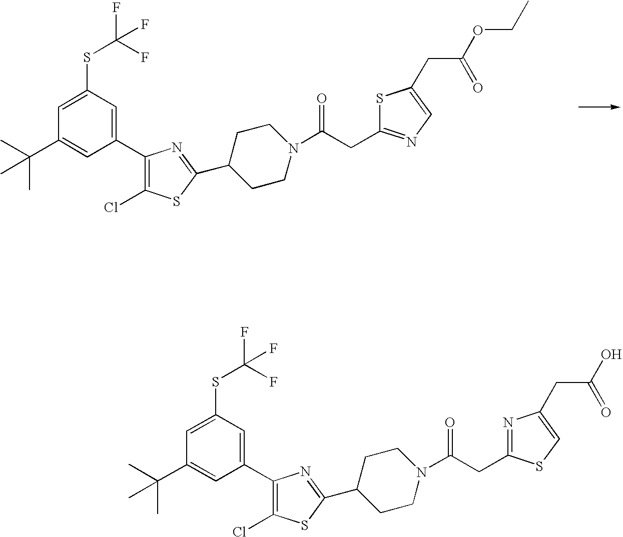

The ester (See Step 1, 29 mg, 0.04 mmol) was dissolved in ethanol (1 mL) and aqueous NaOH (1N, 0.10 mL) added. The reaction was stirred at 45° C. for 40 min., diluted with 2:1 CH₃CN:water (6 mL) and purified by RP-18 HPLC (CH₃CN:H₂O 15 minute gradient 10 to 100%:0.1% TFA) to give the titled compound.

NMR (Acetone-D) δ 8.27 (s, 1H), 8.12 (s, 1H), 7.92 (s, 1H), 7.81 (s, 1H), 4.78 (ABq, Δδ=30.1, J=18.5 Hz, 2H), 4.68 (d, J=13.1 Hz, 1H), 4.22 (d, J=13.7 Hz, 1H), 4.21 (s, 2H), 3.48 (m, 2H), 3.02 (t, J=12.6 Hz, 1H), 2.28 (m, 2H), 1.97 (m, 1H), 1.79 (m, 1H), 1.42 (s, 9H).

MS: m/z=618.38 (M+H).

### Example 53

### Preparation of (2-{2-[4-(4-{3-tert-butyl-5-[(trifluoromethyl)thio]phenyl}-5-chloro-1,3-thiazol-2-yl)piperidin-1-yl]-2-oxoethyl}-5-methyl-3-thiazol-4-yl)acetic acid

### Step 1

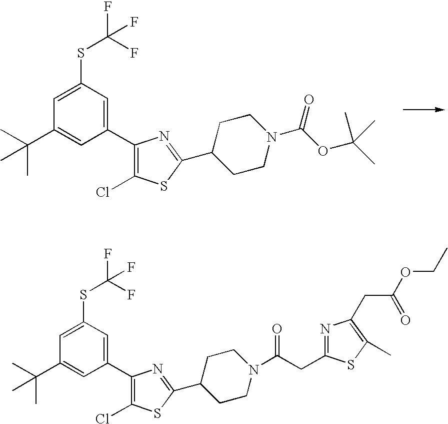

The chloride (See Example 1 Step 11, 37 mg, 0.07 mmol) was dissolved in TFA (1 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (15 mg, 0.11 mmol) and the acid (See Example 43 Step 2, 18 mg, 0.08 mmol) and the combined materials dissolved in DMF (1 mL). Diisopropyl ethyl amine (46 mg, 0.35 mmol) and EDAC (20 mg, 0.11 mmol) were added and the solution stirred at 45° C. for 1 hour. The reaction mixture was diluted with 2:1 CH₃CN:water (6 mL) and purified by RP-18 HPLC (CH₃CN:H₂O 15 minute gradient 10 to 100%:0.1% TFA) to give the titled compound.

### Step 2

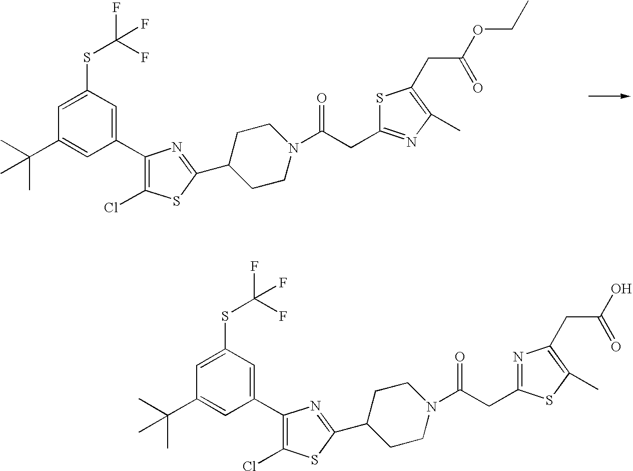

The ester (See Step 1, 33 mg, 0.05 mmol) was dissolved in ethanol (1 mL) and aqueous NaOH (1N, 0.10 mL) added. The reaction was stirred at 45° C. for 40 min., diluted with 2:1 CH₃CN:water (6 mL) and purified by RP-18 HPLC (CH₃CN:H₂O 15 minute gradient 10 to 100%:0.1% TFA) to give the titled compound.

NMR (Acetone-D) δ 8.25 (s, 1H), 8.18 (s, 1H), 7.80 (s, 1H), 4.70 (m, 11H), 4.65 (d, J=13.5 Hz, 1H), 4.22 (d, J=14.0 Hz, 1H), 4.03 (s, 2H), 3.47 (m, 2H), 3.03 (t, J=11.9 Hz, 1H), 2.58 (s, 31), 2.27 (m, 2H), 1.93 (m, 1H), 1.78 (m, 1H), 1.48 (s, 9H).

MS: m/z=632.28 (M+H).

### Example 54

### Preparation of [2-(2-{4-[5-chloro-4-(3,5-di-tert-butyl-4-methoxyphenyl)-1,3-thiazol-2-yl]piperidin-1-yl}-2-oxoethyl)-5-methyl-1,3-thiazol-4-yl](difluoro)acetic acid

### Step 1

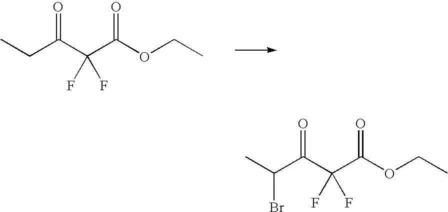

A solution of bromine (1.0 M in AcOH, 44 mL) was added dropwise to a stirred solution of methyl 2,2 difluoro-3-oxopentanoate (6.85 g, 40 mmol) in AcOH (20 mL) at 0° C. After complete addition the solution was allowed to warm to 45° C., and stirred for 6 hours. The reaction was poured into The mixture was poured into a solution of concentrated HCl (4 mL):ice water (40 mL), followed by aqueous/EtOAc work-up to give the titled compound.

### Step 2

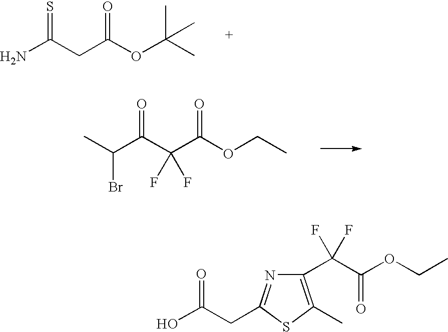

The bromide (See Step 1, 240 mg, 1.0 mmol) was added dropwise at room temperature to a solution of the thioamide (See Example 42 Step 2, 175 mg, 1.0 mmol) dissolved in THF:n-butanol (1:1, 8 mL). The reaction mixture was stirred at reflux for 5 hours, allowed to cool to room temperature and stirring continued overnight. The volatiles were removed in vac, the residue was diluted with water (200 mL), followed by aqueous/EtOAc work-up and silica gel chromatography (EtOAc:hexanes (2:3)). The chromatographed material (32 mg, 0.10 mmol) was dissolved in TFA (1 mL), stirred at RT for 10 minutes, and the volatiles removed in vac to give the titled compound.

### Step 3

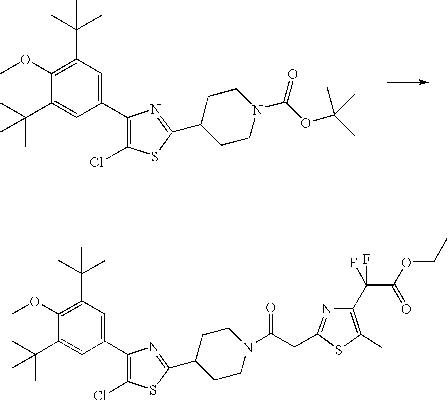

The chloride (See Example 7 Step 1, 16 mg, 0.03 mmol) was dissolved in TFA (0.5 mL), stirred at RT for 10 minutes and the volatiles removed in vac. The residue was dissolved in EtOAc, washed with aqueous NaOH, (1N, 20 mL) and extracted (3 times) with EtOAc. The combined organic fraction was washed with brine, dried over Na₂SO, filtered and reduced in vac. The material was combined with 1-HOBT (7 mg, 0.05 mmol) and the acid (See Step 2, 11 mg, 0.04 mmol) and the combined materials dissolved in DMF (0.5 mL). Diisopropyl ethyl amine (21 mg, 0.17 mmol) and EDAC (10 mg, 0.05 mmol) were added and the solution stirred at 45° C. for 1 hour. The solution was diluted with 2:1 CH₃CN:water (6 mL) and purified by RP-18 HPLC (CH₃CN:H₂O 15 minute gradient 10 to 100%:0.1% TFA) to give the titled compound.

### Step 4

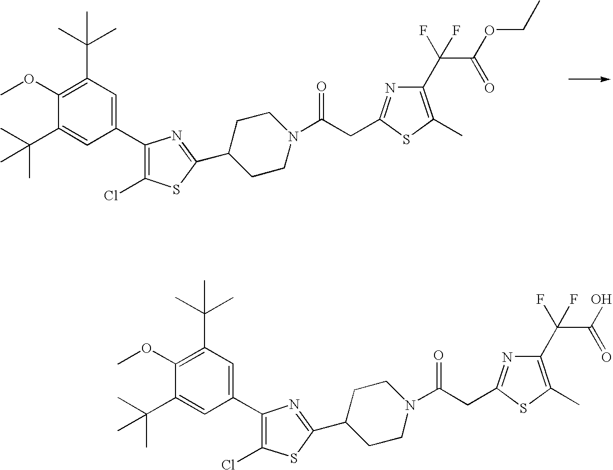

The ester (See Step 9, 17 mg, 0.03 mmol) was dissolved in ethanol (0.5 mL) and aqueous NaOH (1N, 0.06 mL) added. The solution was stirred at 45° C. for 40 min., diluted with 2:1 CH₃CN:water (4 mL) and purified by RP-18 HPLC (CH₃CN:H₂O 15 minute gradient 10 to 100%:0.1% TFA) to give the titled compound.

NMR (Acetone-D) δ 7.87 (s, 1H), 4.63 (d, J=13.5 Hz, 1H), 4.24 (d, J=14.0 Hz, 1H), 4.18 (ABq, Δδ=47.0, J=17.6 Hz, 2H), 3.78 (s, 3H), 3.41 (m, 2H), 2.91 (t, J=12.3 Hz, 1H), 2.62 (s, 3H), 2.21 (m, 2H), 1.90 (m, 1H), 1.76 (m, 1H), 1.48 (s, 18H).

MS: m/z=654.39 (M+H).

### Example 55

### Preparation of {2-[2-(4-{4-[3-bromo-5-(trifluoromethoxy-4-(trifluoromethyl)phenyl]-5-chloro-1,3-thiazol-2-yl}piperidin-1-yl)-2-oxoethyl]-5-methyl-1,3-thiazol-4-yl}acetic acid

### Step 1

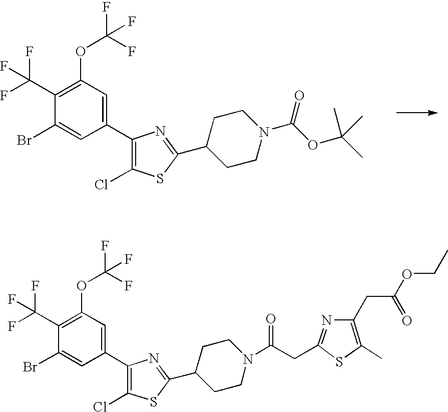

The chloride (See Example 15 Step 4, 43 mg, 0.07 mmol) was dissolved in TFA (1 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (15 mg, 0.11 mmol) and the acid (See Example 43 Step 2, 18 mg, 0.08 mmol) and the resulting mixture dissolved in DMF (1 mL). Diisopropyl ethyl amine (46 mg, 0.35 mmol) and EDAC (20 mg, 0.11 mmol) were added and the solution stirred at 45° C., for 1 hour. The solution was diluted with 2:1 CH₃CN:water (6 mL) and purified by RP-18 HPLC (CH₃CN:H₂O 15 minute gradient 10 to 100%:0.1% TFA) to give the titled compound.

### Step 2

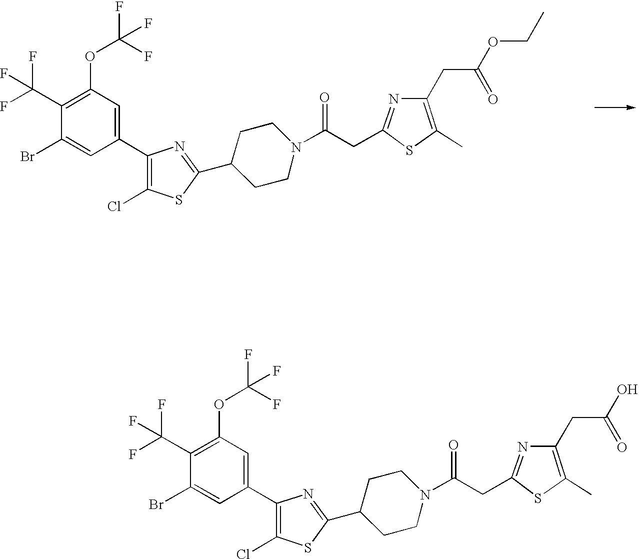

The ester (See Step 1, 26 mg, 0.04 mmol) was dissolved in ethanol (1 mL) and aqueous NaOH (1N, 0.15 mL) added. The solution was stirred at 45° C. for 40 min., diluted with 2:1 CH₃CN:water (6 mL) and purified by RP-18 HPLC (CH₃CN:H₂O 15 minute gradient 10 to 100%:0.1% TFA) to give the titled compound.

NMR (Acetone-D) δ 8.58 (s, 1H), 8.21 (s, 1H), 4.62 (d, J=13.1 Hz, 1H), 4.29 (ABq, Δδ=35.5, J=17.2 Hz, 2H), 4.27 (d, J=14.0 Hz, 1H), 3.80 (s, 2H), 3.42 (m, 2H), 2.93 (t, J=17.2 Hz, 1H), 2.42 (s, 3H), 2.21 (m, 2H), 1.88 (m, 1H), 1.76 (m, 1H).

MS: m/z=708.26 (M+H).

### Example 56

### Preparation of {1-[2-(4-{4-[3-bromo-5-(trifluoromethoxy)-4-(trifluoromethyl)phenyl]-5-chloro-1,3-thiazol-2-yl}piperidin-1-yl)-2-oxoethyl]-5-methyl-1H-pyrazol-3-yl}acetic acid

### Step 1

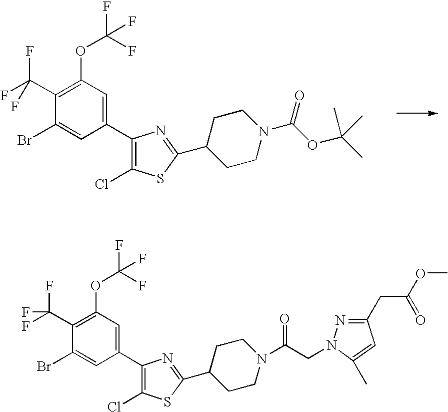

The chloride (See Example 15 Step 4, 39 mg, 0.06 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (13 mg, 0.10 mmol) and the acid (See Example 34 Step 4, 17 mg, 0.08 mmol) and the resulting mixture dissolved in DMF (1 mL). Diisopropyl ethyl amine (42 mg, 0.32 mmol) and EDAC (19 mg, 0.09 mmol) were added and the solution stirred at 45° C. for 1 hour. The reaction mixture was diluted with 2:1 CH₃CN:water (8 mL) and purified by RP-18 HPLC (CH₃CN:H₂O 15 minute gradient 10 to 100%:0.1% TFA) to give the titled compound.

### Step 2

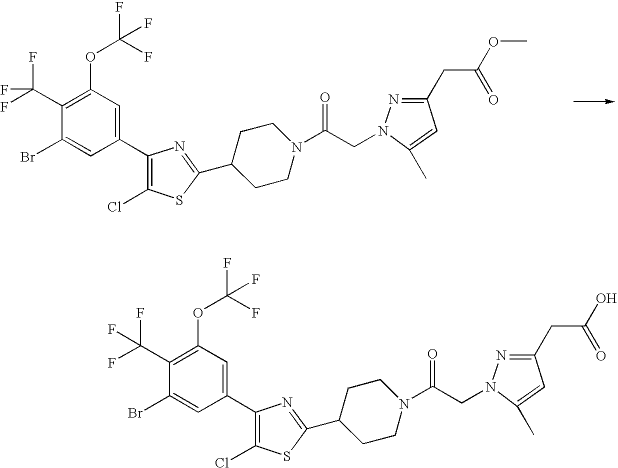

The ester (See Step 1, 24 mg, 0.03 mmol) was dissolved in methanol (0.5 mL) and aqueous NaOH (1N, 0.14 mL) added. The solution was stirred at 45° C. for 40 min., diluted with 2:1 CH₃CN:water (4 mL) and purified by RP-18 HPLC (CH₃CN:H₂O 15 minute gradient 10 to 100%:0.1% TFA) to give the titled compound.

NMR (DMSO) δ 8.42 (s, 1H), 8.16 (s, 1H), 5.90 (s, 1H), 5.05 (ABq, Δδ=42.9, J=16.9 Hz, 2H), 4.39 (d, J=12.6 Hz, 1H), 4.03 (d, J=13.3 Hz, 1H), 3.41 (s, 2H), 3.39 (m, 1H), 3.25 (t, J=12.2 Hz, 1H), 2.82 (t, J=11.0 Hz, 1H), 2.18 (s, 3H), 2.12 (m, 2H), 1.78 (m, 1H), 1.58 (m, 1H).

MS: m/z=691.24 (M+H).

### Example 57

### Preparation of 1-{2-[4-(4-{3-tert-butyl-5-[(trifluoromethyl)thio]phenyl}-5-chloro-1,3-thiazol-2-yl)piperidin-1-yl]-2-oxoethyl}-1,3-dihydro-2H-benzimidazol-2-one

### Step 1

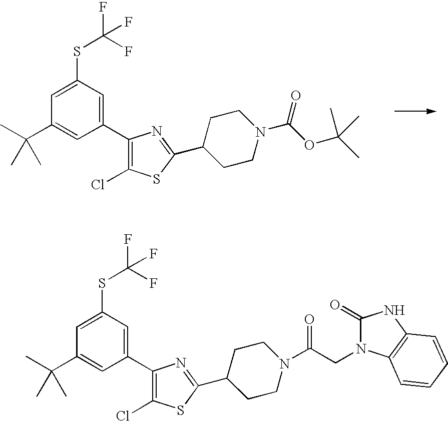

The chloride (See Example 1 Step 11, 31 mg, 0.06 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles removed in vac. The material was combined with 1-HOBT (12 mg, 0.09 mmol) and the acid (See Example 51 Step 2, 13 mg, 0.07 mmol) and the resulting mixture dissolved in DMF (1 mL). Diisopropyl ethyl amine (37 mg, 0.29 mmol) and EDAC (17 mg, 0.09 mmol) were added and the solution stirred at 45° C. for 1 hour. The reaction mixture was diluted with 2:1 CH₃CN:water (8 mL) and purified by RP-18 HPLC (CH₃CN:H₂O 15 minute gradient 10 to 100%:0.1% TFA) to give the titled compound.

NMR (CD₃OD) δ 8.19 (s, 1H), 8.06 (s, 1H), 7.75 (s, 1H), 7.08 (m, 4H), 4.86 (ABq, Δδ=46.5, J=17.1 Hz, 2H), 4.54 (d, J=13.1 Hz, 1H), 4.18 (d, J=13.5 Hz, 1H), 3.38 (m, 2H), 2.92 (t, J=11.9 Hz, 1H), 2.27 (d, J=11.9 Hz, 1H), 2.18 (d, J=11.7 Hz, 1H), 1.92 (m, 1H), 1.75 (m, 1H), 1.40 (s, 9H).

MS: m/z=609.23 (M+H).

### Example 58

### Preparation of 1-{2-[4-(5-bromo-4-{3-tert-butyl-5-[(trifluoromethyl)thio]phenyl}-1,3-thiazol-2-yl)piperidin-1-yl]-2-oxoethyl}-1,3-dihydro-2H-benzimidazol-2-one

### Step 1

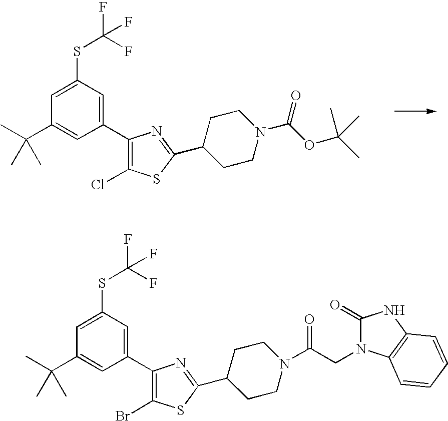

The bromide (See Example 2 Step 1, 35 mg, 0.06 mmol) was dissolved in TFA (2 mL), stirred at RT for 10 minutes, and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and reduced in vac. The material was combined with 1-HOBT (12 mg, 0.09 mmol) and the acid (See Example 51 Step 2, 14 mg, 0.07 mmol) and the resulting mixture dissolved in DMF (1 mL). Diisopropyl ethyl amine (39 mg, 0.31 mmol) and EDAC (18 mg, 0.09 mmol) were added and the solution stirred at 45° C. for 1 hour. The solution was diluted with 2:1 CH₃CN:water (8 mL) and purified by RP-18 HPLC (CH₃CN:H₂O 15 minute gradient 10 to 100%:0.1% TFA) to give the titled compound.

NMR (CD₃OD) δ 8.17 (s, 1H), 8.04 (s, 1H), 7.75 (s, 1H), 7.07 (m, 4H), 4.85 (ABq, Δδ=43.7, J=16.9 Hz, 2H), 4.53 (d, J=13.5 Hz, 1H), 4.18 (d, J=13.5 Hz, 1H), 3.38 (m, 2H), 2.95 (t, J=12.1 Hz, 1H), 2.26 (d, J=8.7 Hz, 1H), 2.17 (d, J=7.2 Hz, 1H), 1.93 (m, 1H), 1.76 (m, 1H), 1.41 (s, 9H).

MS: m/z=655.19 (M+H).

### Example 59

### Preparation of (1-{2-[4-(5-bromo-4-{3-tert-butyl-5-[(trifluoromethyl)thio]phenyl}-1,3-thiazol-2-yl)piperidin-1-yl]-2-oxoethyl}-5-methyl-1H-pyrazol-3-yl)acetic acid

### Step 1

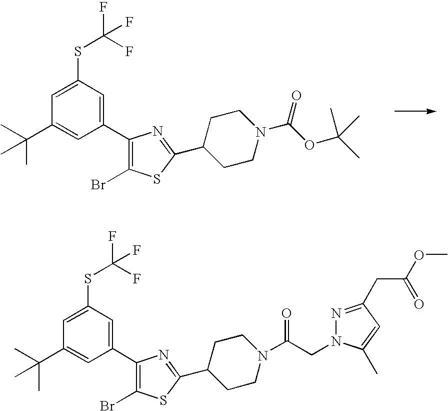

The bromide (See Example 2 Step 1, 41 mg, 0.07 mmol) was dissolved in TFA (1 mL), stirred at RT for 10 minutes and the volatiles removed in vac. The residue was dissolved in EtOAc, washed with aqueous NaOH (1N, 40 mL) and extracted (3 times) with EtOAc. The combined organic fraction was washed with brine, dried over Na₂SO, filtered and the volatiles remove in vac. The material was combined with 1-HOBT (15 mg, 0.10 mmol) and the acid (See Example 34 Step 4, 18 mg, 0.09 mmol) and the resulting mixture dissolved in DMF (1 mL). Diisopropyl ethyl amine (45 mg, 0.36 mmol) and EDAC (20 mg, 0.10 mmol) were added and the solution stirred at 45° C. for 1 hour. The solution was diluted with 2:1 CH₃CN:water (6 mL) and purified by RP-18 HPLC (CH₃CN:H₂O 15 minute gradient 10 to 100%:0.1% TFA) to give the titled compound.

### Step 2

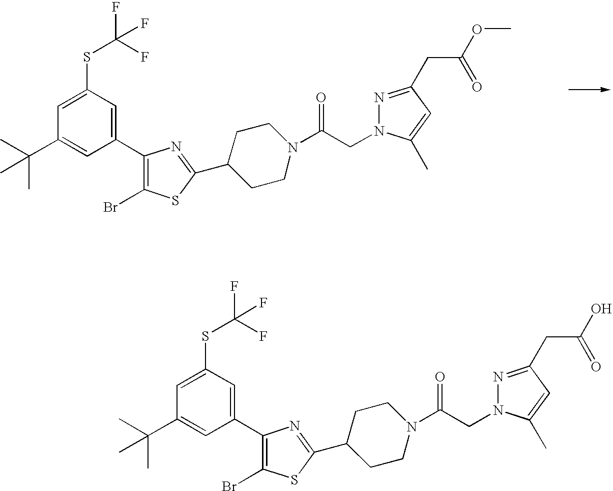

The ester (See Step 1, 27.6 mg, 0.04 mmol) was dissolved in MeOH (0.5 mL) and aqueous NaOH (1N, 0.17 mL) added. The solution was stirred at 45° C. for 40 min., diluted with 2:1 CH₃CN:water (4 mL) and purified by RP-18 HPLC (CH₃CN:H₂O 15 minute gradient 10 to 100%:0.1% TFA) to give the titled compound.

NMR (CD₃OD) δ 8.20 (s, 1H), 8.06 (s, 1H), 7.78 (s, 1H), 6.19 (s, 1H), 5.12 (ABq, Δδ=23.3, J=17.1 Hz, 2H), 4.56 (d, J=13.2 Hz, 1H), 4.11 (d, J=13.5 Hz, 1H), 3.61 (s, 2H), 3.39 (m, 2H), 2.96 (t, J=13.1 Hz, 1H), 2.27 (s, 3H), 2.22 (m, 2H), 1.92 (q, J=7.6 Hz, 1H), 1.77 (q, J=7.3 Hz, 1H), 1.42 (s, 9H).

MS: m/z=661.19 (M+H).

### Example 60

### Preparation of [1-(2-{4-[5-chloro-4-(3,5-di-tert-butyl-4-methoxyphenyl)-1,3-thiazol-2-yl]piperidin-1-yl}-2-oxoethyl)-4-iodo-5-methyl-1H-pyrazol-3-yl]acetic acid

### Step 1

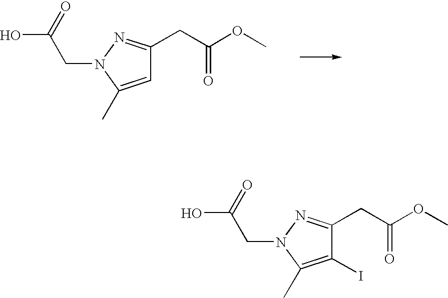

To a solution of the pyrazolylacetate prepared in Example 34 Step 4 (61 mg, 0.5 mmole) in DMF (3 mL) was added NIS (113 mg, 0.5 mmole) at RT. The mixture was stirred at RT for 12 hrs. Water (30 mL) was added, the mixture was extracted with diethyl ether (3×6 mL), the organic layer was washed with water, brine, dried over Na₂SO₄ to give the titled product, 60 mgs.

### Step 2.

### Preparation of [1-(2-{4-[5-chloro-4-(3,5-di-tert-butyl-4-methoxyphenyl)-1,3-thiazol-2-yl]piperidin-1-yl}-2-oxoethyl)-4-iodo-5-methyl-1H-pyrazol-3-yl]acetic acid

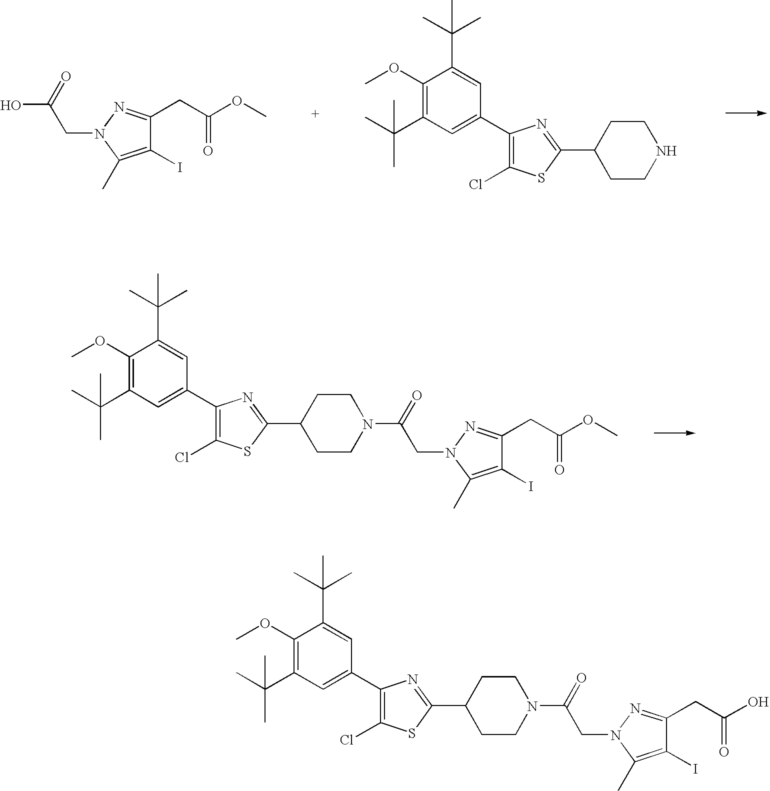

The amine prepared in Example 7 step 2 (25.2 mg, 0.06) was combined with 1-HOBT (9.4 mg, 0.07 mmol) and the acid prepared in Example 60 Step 1 (20.2 mg, 0.06 mmol) and dissolved in DMF (0-5 mL). Diisopropyl ethyl amine (30 mg, 0.23 mmol) and EDAC (13 mg, 0.07 mmol) were added and the solution allowed to stir overnight. The reaction mixture was diluted with water (6 mL), extracted with diethyl ether, the organic layer was washed with water (2×1 mL), concentrated in vacuo. The residue was redissolved in a mixture of MeOH (0.5 mL) and NaOH (1N aq., 0.07 mL), stir 6 hrs at room temperature, MeOH was removed in vacuo. The mixture was diluted in 2:1 CH₃CN:water (6 ml) and purified by RP-18 HPLC (CH₃CN:H₂O 15 minute gradient 10 to 100%:0.1% TFA) to give the titled compound.

NMR (CD₃OD) δ 7.80 (s, 2H), 5.22 (d, 1H, J=17 Hz), 5.14 (d, 1H, J=17 Hz), 4.53 (d, 1H, J=12.2 Hz), 4.09 (d, 1H, J=12.1 Hz), 3.72 (s, 3H), 3.59 (s, 2H), 3.38 (t, 2H, J=6.1 Hz), 2.95 (t, 1H, J=10.8 Hz), 2.27 (s, 3H), 2.21 (m, 2H), 1.84 (m, 2H), 1.77 (s, 6H), 0.99 (m, 1H), 1.47 (s, 18H)

MS: m/z=728.2 (M+H).

### Example 61

### Preparation of 3-(2-{4-[5-chloro-4-(2,6-di-tert-butylpyrimidin-4-yl)-1,3-thiazol-2-yl]piperidin-1-yl}-2-oxoethyl)-3H-imidazo[4,5-b]pyridine

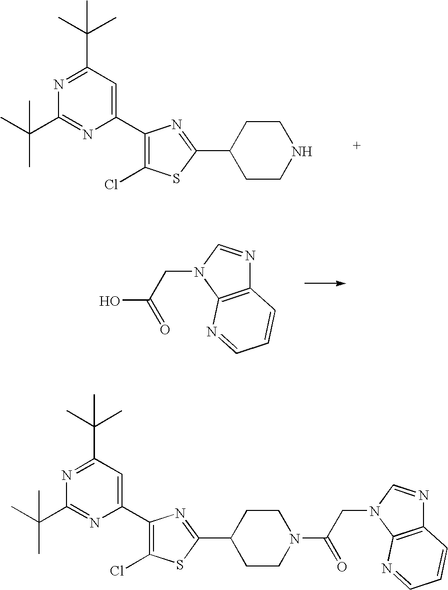

The amine prepared in Example 25 Step 7 (24.0 mg, 0.08 mmole) was combined with 1-HOBT (9.4 mg, 0.09 mmol) and the acid prepared in Example 1 Step 13 (14.2 mg, 0.08 mmol) and dissolved in DMF (0.67 mL). Diisopropyl ethyl amine (38.1 mg, 0.32 mmol) and EDAC (18 mg, 0.09 mmol) were added and the solution allowed to stir overnight. The reaction mixture was diluted in 2:1 CH₃CN:water (8 ml) and purified by RP-18 HPLC (CH₃CN:H₂O 15 minute gradient 10 to 100%:0.1% TFA) to give the titled compound.

NMR (CDCl₃) δ9.20 (br, 1H), 8.62 (d, 1H, J=4.5 Hz), 8.40 (br, 1H), 7.75 (s, 1H), 7.54 (t, 1H, J=6.1 Hz), 5.45 (d, 1H, J=18.1 Hz), 5.40 (d, 1H, J=18.1 Hz), 4.62 (d, 1H, J=12.5 Hz), 4.10 (d, 1H, J=12.7 Hz), 3.47 (t, 11, 12.1 Hz), 3.34 (t, 1H, 12.1 Hz), 3.02 (t, 1H, 12.0 Hz), 2.39 (d, 1H, J=12.2 Hz), 2.25 (d, 1H, J=12.2 Hz), 2.02 (d, 1H, J=12.2 Hz), 1.87 (d, 1H, J=12.2 Hz), 1.47 (s, 9H), 1.41 (s, 9H)

MS: m/z=552.2 (M+H).

### Example 62

### Preparation of 1-(2-{4-[5-chloro-4-(2,6-di-tert-butylpyrimidin-4-yl)-1,3-thiazol-2-yl]piperidin-1-yl}-2-oxoethyl)-1,3-dihydro-2H-benzimidazol-2-one

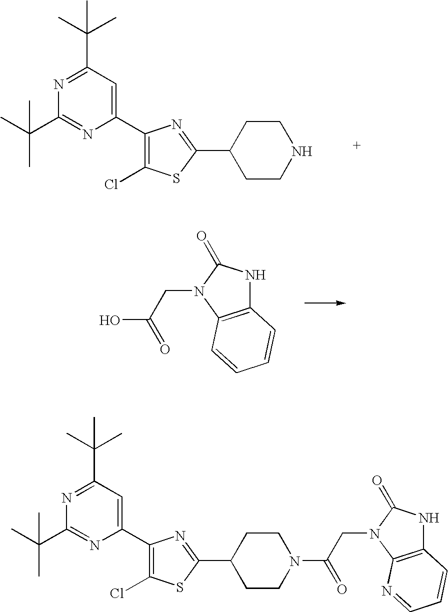

The amine prepared in Example 25 Step 7 (23.1 mg, 0.06) was combined with 1-HOBT (9.4 mg, 0.07 mmol) and the acid prepared in Example 51 Step 2 (10.4 mg, 0.06 mmol) and dissolved in DMF (0.5 mL). Diisopropyl ethyl amine (30 mg, 0.23 mmol) and EDAC (13 mg, 0.07 mmol) were added and the solution allowed to stir overnight. The reaction mixture was diluted in 2:1 CH₃CN:water (6 ml) and purified by RP-18 HPLC (CH₃CN:H₂O 15 minute gradient 10 to 100%:0.1% TFA) to give the titled compound.

NMR (CDCl₃) δ 9.19 (br, 1H), 7.73 (s, 1H), 7.17 (br, 4H), 1H, J=18.1 Hz), 4.76 (s, 2H), 4.62 (d, 1H, J=12.5 Hz), 4.12 (d, 1H, J=12.7 Hz), 3.35 (t, 1H, 12.1 Hz), 3.25 (t, 1H, 12.1 Hz), 2.91 (t, 1H, 12.0 Hz), 2.27 (d, 1H, J=12.2 Hz), 2.18 (d, 1H, J=12.2 Hz), 1.80 (m, 2H)

MS: m/z=567.2 (M+H).

### Example 63

### Preparation of [1-(2-{4-[5-chloro-4-(2,6-di-tert-butylpyrimidin-4-yl)-1,3-thiazol-2-yl]piperidin-1-yl}-2-oxoethyl)-5-methyl-1H-pyrazol-3-yl]acetic acid

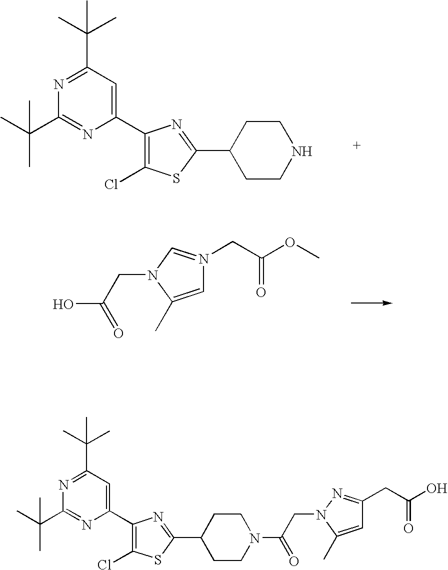

The piperidine prepared in Example 25 Step 7 (24.2 mg, 0.06) was combined with 1-HOBT (9.8 mg, 0.07 mmol) and the acid prepared in Example 34 Step 4 (11.0 mg, 0.06 mmol) and dissolved in DMF (0.5 mL). Diisopropyl ethyl amine (29.5 mg, 0.23 mmol) and EDAC (14.0 mg, 0.07 mmol) were added and the solution allowed to stir overnight. The reaction mixture was diluted with water (6 mL), extracted with diethyl ether, the organic layer was washed with water (2×1 mL), concentrated in vacuo. The residue was redissolved in a mixture of MeOH (0.5 mL) and NaOH (1N aq., 0.07 mL), stir 6 hrs at room temperature, MeOH was removed under vacuo. The mixture was diluted in 2:1 CH₃CN:water (6.2 ml) and purified by RP-18 HPLC (CH₃CN:H₂O 15 minute gradient 10 to 100%:0.1% TFA) to give the titled compound.

NMR (CD₃OD) δ 7.74 (s, 1H), 6.11 (s, 1H), 5.09 (d, 1H, J=17 Hz), 5.05 (d, 1H, J=17 Hz), 4.55 (d, 1H, J=12.2 Hz), 3.96 (d, 1H, J=12.1 Hz), 3.74 (s, 2H), 3.59 (s, 2H), 3.30 (m, 2H), 2.93 (t, 1H, J=10.8 Hz), 2.28 (s, 3H), 2.21 (m, 2H), 1.84 (m, 2H), 0.99 (m, 1H), 1.47 (s, 9H), 1.40 (s, 9H)

MS: m/z=573.3 (M+H).

### Example 64

### Preparation of 1-(2-{4-[5-bromo-4-(2,6-di-tert-butylpyrimidin-4-yl)-1,3-thiazol-2-yl]piperidin-1-yl}-2-oxoethyl)-1,3-dihydro-2H-benzimidazol-2-one

### Step 1.

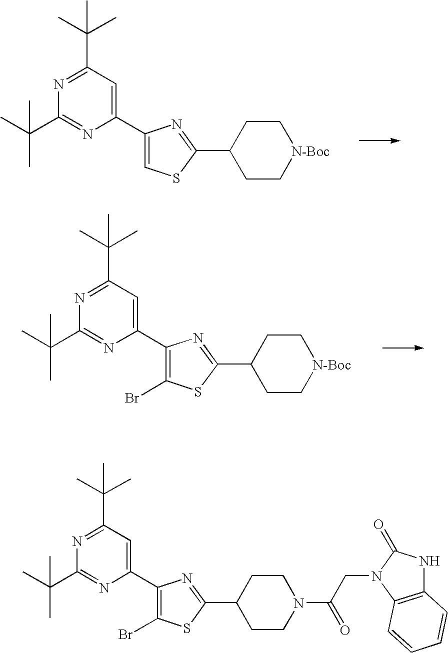

The BOC thiazole prepared in Example 25 Step 5 (1.5 g, 3.29 mmol) was dissolved in DMF (20 mL). NBS (0.65 g, 3.62 mmol) was added to the well stirred solution in one portion and the solution was allowed to stir for 1.5 hours at 90° C. The reaction mixture was cooled to room temperature, and it was poured into water (400 mL) followed by aqueous/EtOAc work-up and silica gel chromatography (diethyl ether:hexanes (1:4)) to afford the bromide.

### Step 2.

The bromide prepared in Example 64 Step 1 (31 mg, 0.06 mmol) was dissolved in TFA (2 ml) and stirred for 10 minutes. The volatiles were removed i. vac. The residue was dissolved in EtOAc, washed with 4:1 water/saturated NaHCO₃ (40 ml) and extracted (3 times) with EtOAc. The combined organic fraction was washed with brine, dried over Na₂SO₄, filtered and reduced i. vac. Step 3.
The unpurified amine was combined with 1-HOBT (9.4 mg, 0.07 mmol) and the acid prepared in Example 51 Step 2 (11.5 mg, 0.06 mmol) and dissolved in DMF (0.5 ml). Diisopropyl ethyl amine (30 mg, 0.23 mmol) and EDAC (13 mg, 0.07 mmol) were added and the solution allowed to stir overnight. The reaction mixture was diluted in 2:1 CH₃CN:water (6 ml) and purified by RP-18 HPLC (CH₃CN:H₂O 15 minute gradient 10 to 100%:0.1% TFA) to give the titled compound.

NMR (DMSO-d) δ 7.78 (s, 1H), 6.97 (m, 4H), 4.78 (d, 1H, J=18.1 Hz), 4.69 (d, 1H, J=18.1 Hz), 4.36 (d, 1H, J=12.5 Hz), 4.10 (d, 1H, J=12.7 Hz), 2.81 (t, 1H, 12.0 Hz), 2.18 (d, 1H, J=12.2 Hz), 2.10 (d, 1H, J=12.2 Hz), 1.80 (m, 1H), 1.58 (m, 1H), 1.41 (s, 9H), 1.34 (s, 9H)

MS: m/z=613.2 (M+H).

### Example 65

### Preparation of 3-[S-methyl-1-(2-oxo-2-{4-[4-(5,5,8,8-tetramethyl-5,6,7,8-tetrahydronaphthalen-2-yl)-1,3-thiazol-2-yl]piperidin-1-yl}ethyl)-1H-pyrazol-3-yl]propanoic acid

### Step 1

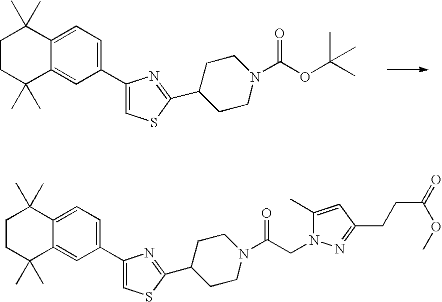

Using the method of Example 39, Step 4 and starting with the products of Example 31, Step 1 and Example 39, Step 3 the titled compound was obtained.

### Step 2

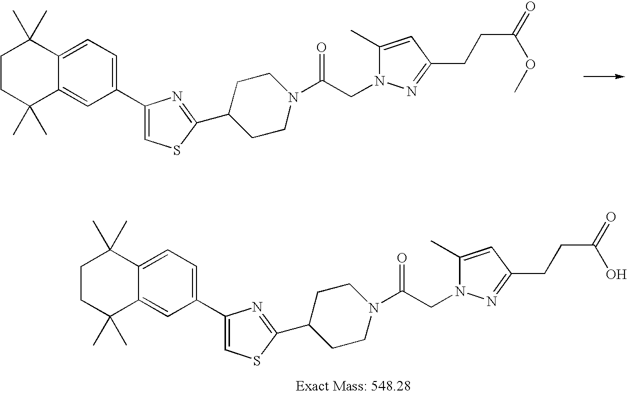

Using the method of Example 39, Step 5 and starting with the product of Example 65, Step 1 the titled compound was obtained.

¹H NMR (500 MHz, CDCl₃): δ 7.78 (d, J=1.8 Hz, 1H); 7.54 (dd, J=1.8, 8.2 Hz, 1H); 7.35 (d, J=8.2 Hz, 1H); 7.32 (s, 1H); 6.11 (s, 1H); 5.20 (ABq, Δδ=61.5, J=16.4 Hz, 2H); 4.53 (d, J=12.8 Hz, 1H); 3.93 (d, J=12.6 Hz, 1H); 3.47 (t, J=11.0 Hz, 1H); 3.31 (t, J=12.3 Hz, 1H); 2.93 (m, 2H); 2.73 (br s, 1H); 2.28 (s, 4H); 1.91-1.77 (m, 2H); 1.70 (s, 4H); 1.33 (s, 6H); 1.29 (s, 6H).

MS: m/z=549.3 (M+H).

### Example 66

### Preparation of [5-methyl-1-(2-oxo-2-{4-[4-(5,5,8,8-tetramethyl-5,6,7,8-tetrahydronaphthalen-2-yl)-1,3-thiazol-2-yl]piperidin-1-yl}ethyl)-1H-pyrazol-3-yl]acetic acid

### Step 1

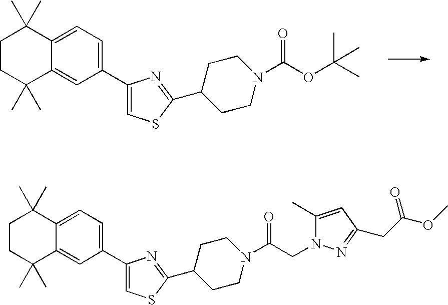

Using the method of Example 34, Step 5 and starting with the products of Example 31, Step 1 and Example 34, Step 4 the titled compound was obtained.

### Step 2

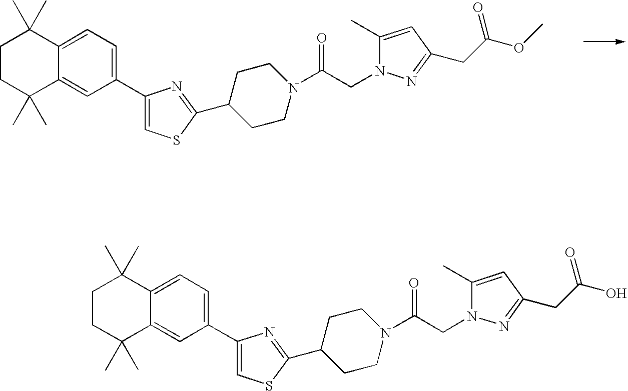

Using the method of Example 34, Step 6 and starting with the product of Example 66, Step 1 the titled compound was obtained.

¹H NMR (500 MHz, CD3OD): δ 7.85 (d, J=1.9 Hz, 1H); 7.62 (s, 1H); 7.59 (dd, J=1.9, 8.2 Hz, 1H); 7.36 (d, J=82 Hz, 1H); 6.21 (s, 1H); 5.17 (ABq, Δδ=34.3, J=17.1 Hz, 2H); 4.56 (d, J=13.3 Hz, 1H); 4.09 (d, J=13.4 Hz, 1H); 3.64 (s, 2H); 3.47-3.35 (m, 2H); 2.95 (t, J=11.5 Hz, 1H); 2.26 (m, 5H); 1.97-1.89 (m, 1H); 1.73 (m, 5H); 1.33 (s, 6H); 1.29 (s, 6H).

MS: m/z=535.3 (M+H).

### Example 67

### Preparation of 3-[2-(4-{4-[3,5-bis(trifluoromethyl)phenyl]-1,3-thiazol-2-yl}piperidin-1-yl)-2-oxoethyl]-3H-imidazo[4,5-b]pyridine

### Step 1

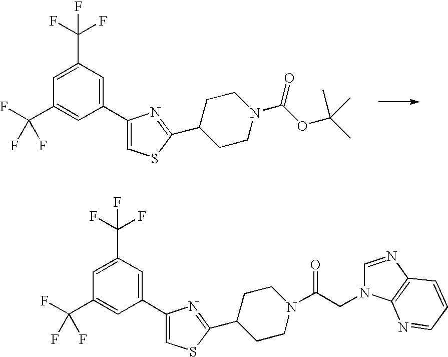

Using the method of Example 9, Step 2 and starting with the product of Example 8, Step 1 the title compound was obtained.

¹H NMR (500 MHz, CDCl₃): δ 9.59 (s, 1H); 8.67 (d, J=4.6 Hz, 1H); 8.38 (d, J=8.3 Hz, 1H); 8.33 (s, 2H); 7.84 (s, 1H); 7.61-7.58 (m, 2H); 5.51 (ABq, Δδ=20.7, J=16.5 Hz, 2H); 4.58 (d, J=14.0 Hz, 1H); 4.09 (d, J=13.4 Hz, 1H); 3.51 (t, J=11.8 Hz, 1H); 3.43 (tt, J=3.9, 11.0 Hz, 1H); 3.06-3.02 (m, 1H); 2.43 (d, J=12.6 Hz, 1H); 2.27 (d, J=13.0 Hz, 1H); 2.09 (q, J=10.1 Hz, 1H); 1.91 (q, J=9.6 Hz, 1H).

MS: m/z=540.10 (M+H).

### Example 68

### Preparation of 3-(2-{4-[4-(3,5-di-tert-butyl-4-methoxyphenyl)-1,3-thiazol-2-yl]piperidin-1-yl}-2-oxoethyl)-3H-imidazo[4,5-b]pyridine

### Step 1

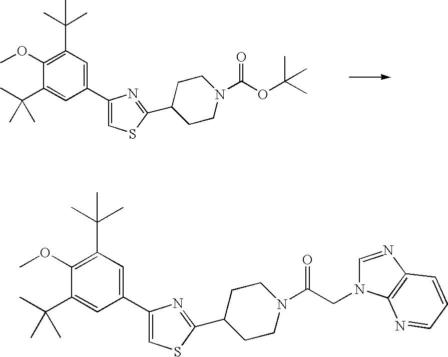

Using the method of Example 9, Step 2 and starting with the product of Example 6, Step 2 the titled compound was obtained.

¹H NMR δ (ppm)(CD3OD): 9.40 (s, 1H), 8.67 (d, J=4.7 Hz, 1H), 8.32 (d, J=8.2 Hz, 1H), 7.78 (s, 2H), 7.65 (dd, J=4.7, 8.2 Hz, 1H), 7.57 (s, 1H), 5.64 (ABq, Δδ=29.3, J=16.8 Hz, 2H), 4.55 (d, J=13.5 Hz, 1H), 4.21 (d, J=14.0 Hz, 1H), 3.69 (s, 3H), 3.52-3.44 (m, 2H), 3.00 (t, J=13.8 Hz, 1H), 2.35 (d, J=12.6 Hz, 1H), 2.21 (d, J=11.8 Hz, 1H), 2.05 (dq, J=3.7, 12.8 Hz, 1H), 1.81 (dq, J=4.0, 12.6 Hz, 1H), 1.47 (s, 18H).

MS: m/z=546.24 (M+H).

### Example 69

### Preparation of 3-[2-(4-{4-[3-bromo-4-methoxy-5-(trifluoromethoxy phenyl]-1,3-thiazol-2-yl}piperidin-1-yl)-2-oxoethyl]-3H-imidazo[4,5-b]pyridine

### Step 1

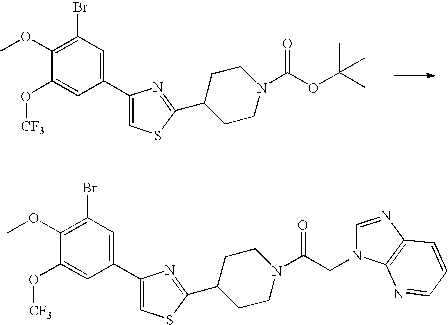

Using the method of Example 9, Step 2 and starting with the product of Example 27, Step 5 the titled compound was obtained.

¹H NMR (500 MHz, CD3OD): δ 9.15 (s, 1H); 8.61 (dd, J=1.0, 4.8 Hz, 1H); 8.27 (d, J=8.1 Hz, 1H); 8.18 (d, J=2.0 Hz, 1H); 7.89 (br s, 1H); 7.85 (s, 1H); 7.58 (dd, J=4.8, 8.2 Hz, 1H); 5.59 (ABq, Δδ=21.1, J=16.9 Hz, 2H); 4.53 (d, J=13.5 Hz, 1H); 4.21 (d, J=13.6 Hz, 1H); 3.91 (s, 3H); 3.54-3.42 (m, 2H); 3.01 (t, 1H); 2.34 (d, J=12.0 Hz, 1H); 2.21 (d, J=12.1 Hz, 1H); 2.09 (dq, J=3.9, 13.1 Hz, 1H); 1.83 (dq, J=3.9, 12.9 Hz, 1H).

MS: m/z=596.13 (M+H).

### Example 70

### Preparation of [2-(2-{4-[4-(3,5-di-tert-butyl-4-methoxyphenyl)-1,3-thiazol-2-yl]piperidin-1-yl}-2-oxoethyl)-5-methyl-1,3-thiazol-4-yl]acetic acid

### Step 1

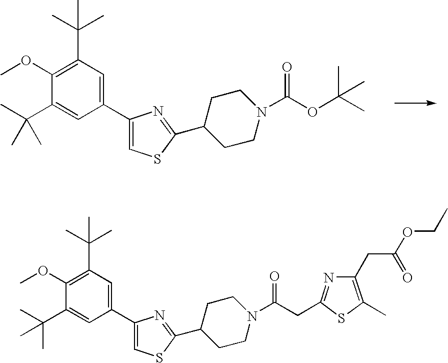

Using the method of Example 55, Step 1 and starting with the products of Example 6, Step 2 and Example 43, Step 2 the titled compound was obtained.

### Step 2

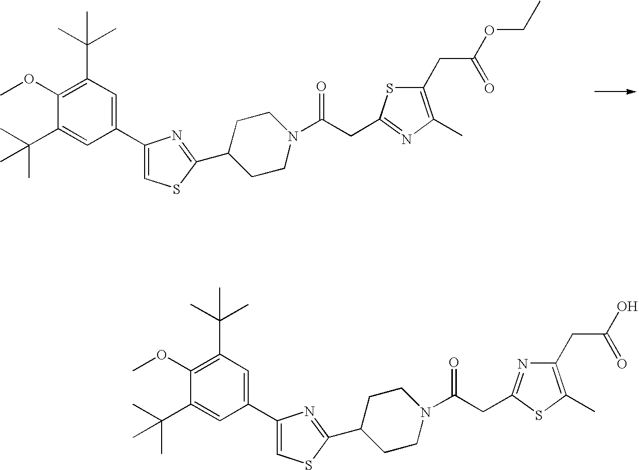

Using the method of Example 55, Step 2 and starting with the product of Example 70, Step 1 the titled compound was obtained.

NMR (Acetone-d): δ 7.92 (s, 2H), 7.38 (s, 1H), 4.79 (ABq, Δδ=32.7, J=21.7 Hz, 2H), 4.62 (d, J=13 Hz, 1H), 4.15 (d, J=13.9 Hz, 1H), 3.79 (s, 2H), 3.70 (s, 3H), 3.44 (m, 2H), 2.94 (t, J=12.1 Hz, 1H), 2.46 (s, 3H), 2.20 (m, 2H), 1.86 (dq, J=3.5, 12.4 Hz, 1H), 1.75 (dq, J=3.4, 12.1 Hz, 1H), 1.45 (s, 18H).

MS: m/z=584.55 (M+H).

### Example 71

### Preparation of 3-(2-{4-[4-(2,6-di-tert-butylpyrimidin-4-yl)-1,3-thiazol-2-yl]piperidin-1-yl}-2-oxoethyl)-3H-imidazo[4,5-b]pyridine

### Step 1

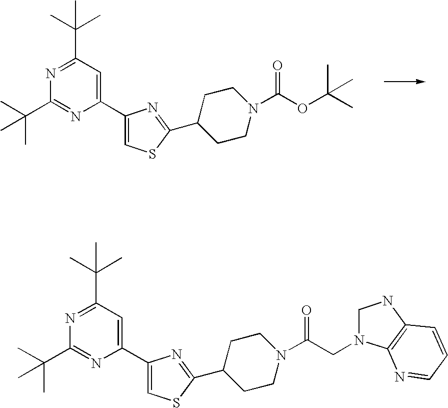

Using the method of Example 9, Step 2 and starting with the product of Example 25, Step 5 the titled compound was obtained.

¹HNMR (500 MHz, CD3OD): δ 9.32 (s, 1H); 8.66 (dd, J=1.1, 4.7 Hz, 1H); 8.30 (m, 2H); 7.87 (s, 1H); 7.64 (dd, J=4.8, 8.2 Hz, 1H); 5.64 (ABq, Δδ=23.5, J=16.7 Hz, 2H); 4.56 (d, J=13.4 Hz, 1H); 4.23 (d, J=14.1 Hz, 1H); 3.55-3.47 (m, 2H); 3.03 (t, J=13.0 Hz, 1H); 2.37 (d, J=13.2 Hz, 1H); 2.24 (d, J=12.0 Hz, 1H); 2.11 (dq, J=3.9, 12.8 Hz, 1H); 1.86 (dq, J=4.1, 12.5 Hz, 1H); 1.43 (s, 9 H); 1.39 (s, 9H).

MS: m/z=518.26 (M+H).

### Example 72

### Preparation of 3-[2-(4-{4-[2-tert-butyl-6-(trifluoromethyl)pyrimidin-4-yl]-1,3-thiazol-2-yl}piperidin-1-yl)-2-oxoethyl]-3H-imidazo[4,5-b]pyridine

### Step 1

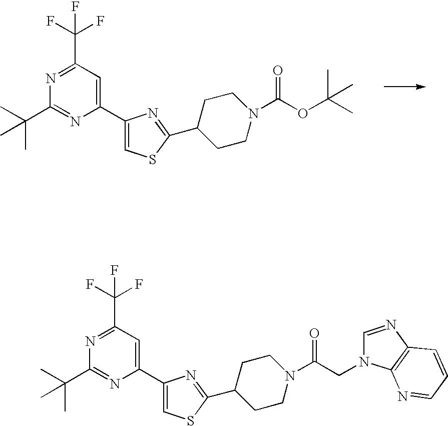

Using the method of Example 9, Step 2 and starting with the product of Example 26, Step 5 the titled compound was obtained.

¹H NMR (500 MHz, CD3OD): δ9.17 (br s, 1H); 8.61 (d, J=4.7 Hz, 1H); 8.51 (s, 1H); 8.28 (d, J=8.1 Hz, 1H); 8.16 (s, 1H); 7.59 (dd, J=4.8, 8.1 Hz, 1H); 5.60 (ABq, Δδ=21.5, J=16.8 Hz, 2H); 4.54 (d, J=13.5 Hz, 1H); 4.23 (d, J=13.6 Hz, 1H); 3.54-3.48 (m, 2H); 3.04 (t, J=13.8 Hz, 1H); 2.37 (d, J=11.8 Hz, 1H); 2.25 (d, J=11.6 Hz, 1H); 2.13 (dq, J=3.7, 12.8 Hz, 1H); 1.87 (dq, J=3.9, 12.9 Hz, 1H); 1.47 (s, 9H).

MS: m/z=530.21 (M+H).

### Example 73

### Preparation of 3-[2-(4-{4-[2-tert-butyl-6-(1-methylcyclopropyl)pyrimidin-4-yl]-1,3-thiazol-2-yl}piperidin-1-yl)-2-oxoethyl]-3H-imidazo[4,5-b]pyridine

### Step 1

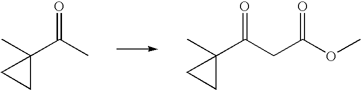

The ketone (3.5 g, 35.6 mmol) was dissolved in THF (90 mL) and the solution cooled to −78° C. LHMDS (1.0 M in THF, 78.5 mL) was added at −78° C., the cooling bath removed and the mixture stirred at RT for 30 minutes. Dimethyl carbonate (3.3 g, 36.4 mmol) was added and the mixture stirred at RT overnight. The volatiles were removed in vacuo, the residue partitioned between water (200 ml) and EtOAc (100 ml), the layers acidified with acetic acid, followed by aqueous/EtOAc work-up and silica gel chromatography (EtOAc:hexanes (1:9)) to give the titled compound.

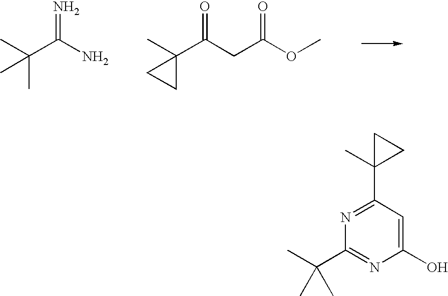

Using the method of Example, 30, Step 2 and starting with the product of Example 73, Step 1 the titled compound was obtained.

### Step 3

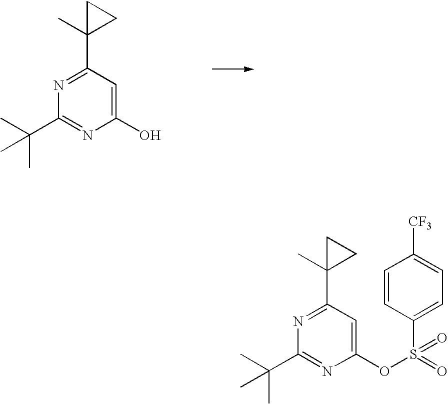

Using the method of Example 30, Step 3 and starting with the product of Example 73, Step 2 the titled compound was obtained.

### Step 4

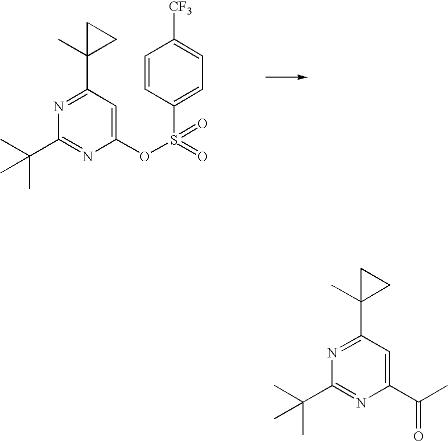

Using the method of Example 30, Step 4 and starting with the product of Example 73, Step 3 the titled compound was obtained.

### Step 5

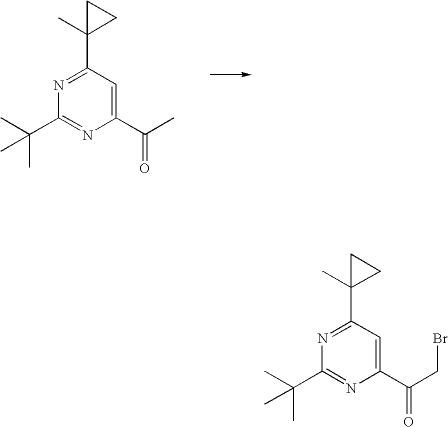

Using the method of Example 30, Step 5 and starting with the product of Example 73, Step 4 the titled compound was obtained.

### Step 6

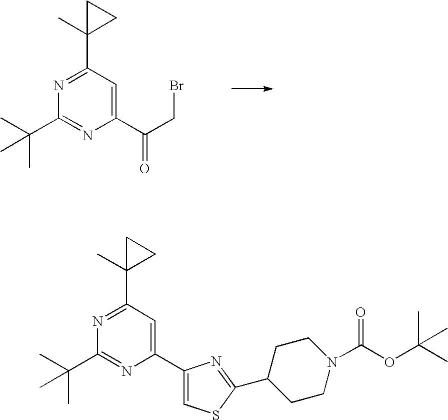

Using the method of Example 30, Step 6 and starting with the product of Example 73, Step 5 the titled compound was obtained.

### Step 7

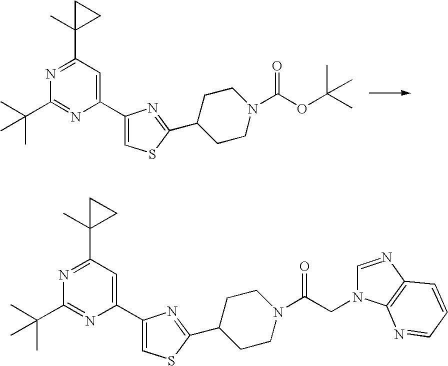

Using the method of Example 9, Step 2 and starting with the product of Example 73, Step 6 the titled compound was obtained.

¹H NMR (500 MHz, CD3OD): δ 9.40 (s, 1H); 8.67 (d, J=4.7 Hz, 1H); 8.32 (m, 2H); 7.84 (s, 1H); 7.65 (dd, J=4.7, 8.1 Hz, 1H); 5.65 (ABq, Δδ=24.1, J=16.9 Hz, 2H); 4.56 (d, J=13.4 Hz, 1H); 4.23 (d, J=13.7 Hz, 1H); 3.55-3.47 (m, 2H); 3.03 (t, J=13.4 Hz, 1H); 2.37 (d, J=12.2 Hz, 1H); 2.25 (d, J=11.8 Hz, 1H); 2.11 (dq, J=3.9, 12.8 Hz, 1H); 1.87 (dq, J=4.1, 12.6 Hz, 1H); 1.56 (s, 3H); 1.40 (m, 11H); 0.91 (q, J=3.3 Hz, 2H).

MS: m/z=516.26 (M+H).

### Example 74

### Preparation of [1-(2-{4-[4-(2,6-di-tert-butylpyrimidin-4-yl)-1,3-thiazol-2-yl]piperidin-1-yl}-2-oxoethyl)-5-methyl-1H-pyrazol-3-yl]acetic acid

### Step 1

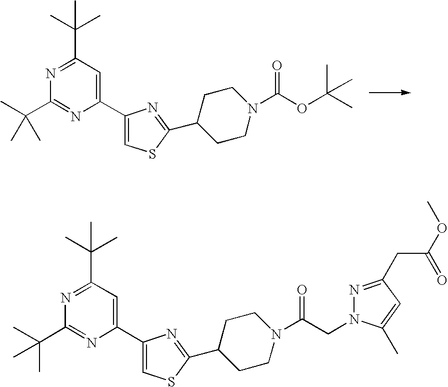

Using the method of Example 34, Step 5 and starting with the products of Example 25, Step 5 and Example 34, Step 4 the titled compound was obtained.

### Step 2

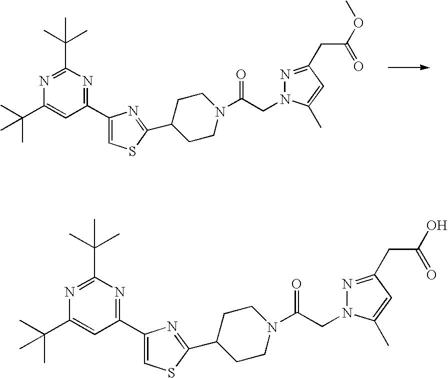

Using the method of Example 34, Step 6 and starting with the product of Example 74, Step 1 the titled compound was obtained.

¹H NMR (500 MHz, CD3OD): δ 8.31 (s, 1H); 7.87 (s, 1H); 6.19 (s, 1H); 5.15 (ABq, Δδ=36.4, J=17.2 Hz, 2H); 4.56 (d, J=13.4 Hz, 1H); 4.09 (d, J=13.8 Hz, 1H); 3.62 (s, 2H); 3.47-3.35 (m, 2H); 2.95 (t, J=11.5 Hz, 1H); 2.28 (s, 3H), 2.26 (d, J=8.5 Hz, 1H); 2.22 (d, J=13 Hz, 1H); 1.95 (dq, J=4.2, 12.4 Hz, 1H); 1.84 (dq, J=4.1, 12.4 Hz, 1H); 1.43 (s, 9H); 1.38 (s, 9H).

MS: m/z=540.34 (M+H).

### Example 75

### Preparation of 3-[2-(4-{4-[6-tert-butyl-2-(1-methylcyclopropyl)pyrimidin-4-yl]-1,3-thiazol-2-yl}piperidin-1:yl)-2-oxoethyl]-3H-imidazo[4,5-b]pyridine

### Step 1

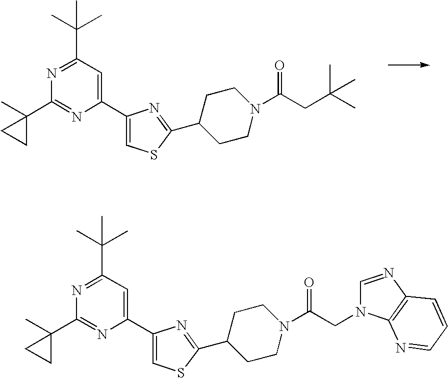

Using the method of Example 9, Step 2 and starting with the product of Example 30, Step 6 the titled compound was obtained.

1H NMR (500 MHz, CD3OD): δ 9.25 (s, 1H); 8.63 (dd, J=1.2, 4.8 Hz, 1H); 8.29 (m, 2H); 7.81 (s, 1H); 7.61 (dd, J=4.8, 8.2 Hz, 1H); 5.61 (ABq, Δδ=23.8, J=16.9 Hz, 2H); 4.56 (d, J=13.4 Hz, 1H); 4.22 (d, J=14.0 Hz, 1H); 3.53-3.45 (m, 2H); 3.03 (t, J=13.3 Hz, 1H); 2.36 (d, J=11.8 Hz, 1H); 2.23 (d, J=11.8 Hz, 1H); 2.12 (dq, J=3.9, 13.0 Hz, 1H); 1.87 (dq, J=4.1, 12.8 Hz, 1H); 1.60 (s, 3H); 1.40 (q, J=3.1 Hz, 2H); 1.36 (s, 9H); 0.86 (q, J=3.2 Hz, 2H).

MS: m/z=516.20 (M+H).

### Assays for Determining Biological Activity

### Human CXCR3 Receptor.

The compounds claimed here are assayed for affinity and functional potency at the CXCR3 receptor using the assays described below.

Since the expression of CXCR3 on naive T cells is low, PBMC's were cultured in the presence of a mixture of superantigens to provide primary cells with sufficient CXCR3 expression to use routinely in binding and functional assays. Briefly, mononuclear cells were enriched from buffy coats obtained from a local blood bank by centrifugation over Ficoll-Hypaque. Residual red blood cells were lysed in hypotonic buffer, (ACK), cells were washed with PBS and resuspended in media (RPMI containing 10% FBS, 2 mM glutamine, MEM non essential amino acids and sodium pyruvate) containing 500 Units/ml of IL-2 and 0.5 ng/ml SE cocktail (containing equal amounts of SEA, SEB, SEC1, SED and SEE all from Toxin Technology). After several days in culture, cells were switched to fresh media containing 500 units/ml of IL-2 and cultures were maintained at 2-4 million cells 1 ml for up to 21 days.

### Binding Assay.

Inhibition of binding of CXCL10 or CXCL11 to human CXCR3 was measured in whole cells, using superantigen activated T cells (SE-T) at day 7-14 post stimulation. Binding of ¹²⁵I-IP-10 (2200 Ci/mmol, typically 20 pM) in the presence of unlabeled ligands was initiated by adding intact T cells (200,000 cells/assay) in a total assay volume of 250 μl containing 50 mM HEPES, pH 7.2, 5 mM MgCl2, 1 mM CaCl2 and 0.5% BSA. Binding of ¹²⁵I-I-TAC (2200 Ci/mmol, 20 pM) was performed as described for IP-10 except for the addition of 0.15M NaCl to the binding buffer. After incubation at room temperature for 2 hours with shaking, the reaction was terminated by filtering through a 0.1% polyethylenimine (Sigma) soaked GF/C filter plate (Packard) using a Packard Filtermate cell harvester and the plate washed with approximately 750 μl of 50 mM HEPES (Sigma), pH 7.2, 500 mM NaCl chilled to 4° C. The plates were dried; scintillant added and counted on a Packard TopCount. Non-specific binding was measured in the presence of 1 μM ligand (IP-10 or I-TAC). Binding results were analyzed using Microsoft Excel and GraphPad Prism software.

The Examples disclosed herein were tested in the above receptor binding assay and demonstrated an IC₅₀ ranging from 1 to 800 nM against ¹²⁵I-IP-10. The range of IC₅₀ values is similar against ¹²⁵I-I-TAC.

### Functional Assays.

The functional potency of the claimed compounds was assessed by measuring inhibition of the chemotaxis of leukocytes in response to CXCR3 ligands. A modified Boyden chamber chemotaxis system (ChemoTx™, NeuroProbe, Gaithersburg, Md.), consisting of a 96-well microplate and a filter (6.0-mm diameter, 5-μ pore size), coated on the bottom with fibronectin (50 μl of a 10 μg/ml solution, then air-dried), was used for chemotaxis measurements. Briefly, aliquots of human T cells (day 14 to day 17 post activation) were washed and resuspended at 1×107 cells/ml in warm (37° C.) Hanks' balanced saline solution (HBSS)/bovine serum albumin [(BSA); HBSS without phenol red, calcium, or magnesium (Mediatec)+0.01% BSA] and loaded with Calcein-AM (Molecular Probes) at a concentration of 2 μM for 30 min at 37° C. The cells were washed again in HBSS/BSA and resuspended in RPMI/BSA [RPMI without phenol red (Mediatec)+0.5% BSA+1% dimethyl sulfoxide] to a concentration of 6×106 cells/ml. To initiate the chemotaxis, chemokines were diluted in warm (37° C.) RPMI/BSA and added in 30 μl to the bottom of the microplate before affixing the filter to the unit. Aliquots (50 μl) of the Calcein-loaded T cells were then added to the top of the filter over each individual well. The microplates were subsequently incubated for 1 h at 37° C. Remaining cells were suctioned off the top of the filter. The filter was rinsed with PBS and wiped with a rubber squeegee. The plate with filter intact was read in a Cytofluor™ II fluorometer (PerSeptive Biosystems, Foster City, Calif.). For assay of antagonists, compounds were diluted in DMSO and added to both cells and ligand in a final DMSO concentration of 0.5%.

The Examples disclosed herein were tested in the above assay against both TP-10 and I-TAC. The Examples demonstrated an IC₅₀ ranging from 1 to 800 nM against IP-10 and typically a somewhat higher IC₅₀ ranging from 3 to 1800 nM against I-TAC.

1. A compound of Formula I 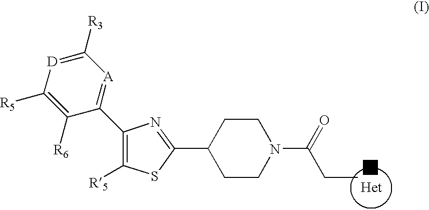
or a pharmaceutically acceptable salt thereof, wherein:
A is CH or N;
D is CR₄ or N;
R₃ is selected from the group consisting of: C₁₋₄alkyl, C₃₋₆cycloalkyl, —CF₃, —OCF₃ and —S(O)ₙCF₃, wherein n is 0 or 2;
R₄ is selected from the group consisting of: —H, —OH, —OCH₃, —OCH₂CF₃ and —CF₃;
or R₃ and R₄ may be joined together with the carbon atoms to which they are attached to form a five- or six-membered monocyclic ring, said rings tetra-substituted with methyl groups as follows: 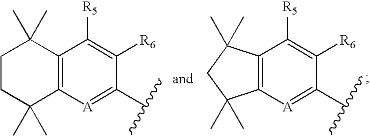
R₅ is selected from the group consisting of: —H, C₁₋₄alkyl, C₃₋₆cycloalkyl, —CF₃, —Br, —CF₂CH₃, —OCF₃ and —SCF₃;
R₆ is selected from the group consisting of —H and —OCH₃,
or R₅ and R₆ may be joined together with the carbon atoms to which they are attached to form a monocyclic 5-membered ring, said ring di-substituted with methyl as follows: 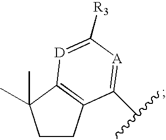
R′₅ is selected from the group consisting of: —H, —Cl, —Br and —CH₃; and 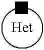
is selected from the group consisting of: 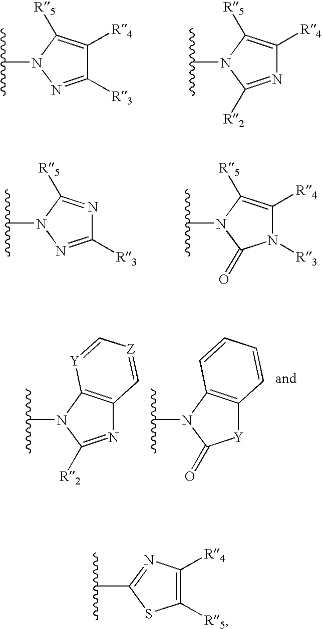
wherein Y and Z are independently C or N, and
R′₂, R′₃, R′₄ and R′₅ are selected from the group consisting of: —H, carboxy, —CF₃, halo and
C₁₋₃alkyl optionally substituted with carboxy.

2. The compound according to claim 1 wherein A and D are both N.

3. The compound according to claim 1 wherein A is CH and D is CR₄.

4. The compound according to claim 3 wherein
R₅ and R₆ are both —H; and
R₃ and R₄ are joined together with the carbon atoms to which they are attached to form a six-membered monocyclic ring, said ring tetra-substituted with methyl groups as follows:

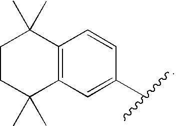

5. The compound according to claim 3 wherein:
R₃ is selected from the group consisting of: C₁₋₄alkyl, C₃₋₆cycloalkyl, —CF₃, —OCF₃ and —S(O)ₙCF₃, wherein n is 0 or 2;
R₄ is selected from the group consisting of: —H, —OH, —OCH₃, —OCH₂CF₃ and —CF₃;
R₅ is selected from the group consisting of: C₁₋₄alkyl, C₃₋₆cycloalkyl, —CF₃, —Br, —CF₂CH₃, —OCF₃ and —SCF₃; and
R₆ is —H.

6. The compound according to claim 5 wherein
R₃ is selected from the group consisting of: tert-butyl, —CF₃, —OCF₃ and —S(O)ₙCF₃, wherein n is 0 or 2; and
R₅ is selected from the group consisting of: tert-butyl, cyclopropyl, 1-methylcyclopropyl, —CF₃, —Br, —CF₂CH₃, —OCF₃ and —SCF₃.

7. The compound according to claim 1 wherein R₆ is —H.

8. The compound according to claim 1 wherein:
A is CH;
D is CR₄;
R₃ is selected from the group consisting of: tert-butyl, —CF₃, —OCF₃ and —S(O)ₙCF₃, wherein n is 0 or 2;
R₄ is selected from the group consisting of: —H, —OH, —OCH₃, —OCH₂CF₃ and —CF₃;
or R₃ and R₄ may be joined together with the carbon atoms to which they are attached to form a six-membered monocyclic ring, said ring tetra-substituted with methyl groups as follows: 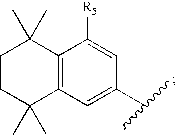
R₅ is —H when R₃ and R₄ are joined together to form a six-membered monocyclic ring, otherwise R₅ is selected from the group consisting of: tert-butyl, cyclopropyl, 1-methylcyclopropyl, —CF₃, —Br, —CF₂CH₃, —OCF₃ and —SCF₃; and
R₆ is —H.

9. The compound according to claim 8 wherein: R₃ and R₄ are joined together with the carbon atoms to which they are attached to form a six-membered monocyclic ring, said ring tetra-substituted with methyl groups as follows: 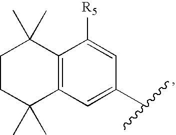
wherein R₅ is —H.

10. The compound according to claim 8 wherein:
(1) R₃ is tert-butyl, R₄ is —H and R₅ is tert-butyl;
(2) R₃ is tert-butyl, R₄ is —OCH₃ and R₅ is tert-butyl;
(3) R₃ is —SCF₃, R₄ is —H and R₅ is tert-butyl;
(4) R₃ is —SO₂CF₃, R₄ is —H and R₅ is tert-butyl; or
(5) R₃ is —OCF₃, R₄ is —CF₃ and R₅ is —Br.

11. The compound according to claim 8 wherein R′₅ is —H.

12. The compound according to claim 8 wherein R′₅ is —CH₃.

13. The compound according to claim 8 wherein R′₅ is —Cl or —Br.

14. The compound according to claim 8 wherein 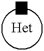
is selected from the group consisting of:

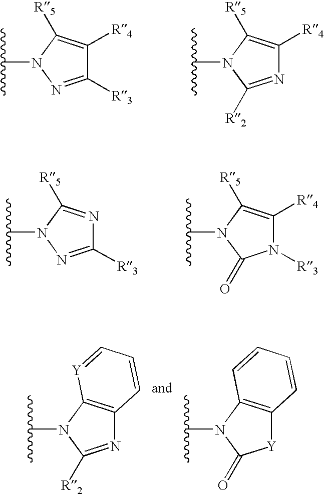

15. The compound according to claim 14 wherein:

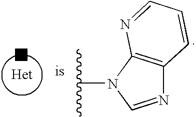

16. A compound according to claim 1 selected from the following group:
(1) 3-{2-[4-(4-{3-tert-butyl-5-[(trifluoromethyl)thio]phenyl}-5-chloro-1,3-thiazol-2-yl)piperidin-1-yl]-2-oxoethyl}-3H-imidazo[4,5-b]pyridine;
(2) 3-{2-[4-(5-bromo-4-{3-tert-butyl-5-[(trifluoromethyl)thio]phenyl}-1,3-thiazol-2-yl)piperidin-1-yl]-2-oxoethyl}-3H-imidazo[4,5-b]pyridine;
(3) 3-{2-[4-(4-{3-tert-butyl-5-[(trifluoromethyl)sulfonyl]phenyl}-5-chloro-1,3-thiazol-2-yl)piperidin-1-yl]-2-oxoethyl}-3H-imidazo[4,5-b]pyridine;
(4) 3-{2-[4-(5-bromo-4-{3-tert-butyl-5-[(trifluoromethyl)sulfonyl]phenyl}-1,3-thiazol-2-yl)piperidin-1-yl]-2-oxoethyl}-3H-imidazo[4,5-b]pyridine;
(5) 3-(2-{4-[5-bromo-4-(3,5-di-tert-butyl-4-methoxyphenyl)-1,3-thiazol-2-yl]piperidin-1-yl}-2-oxoethyl)-3H-imidazo[4,5-b]pyridine;
(6) 3-(2-{4-[5-chloro-4-(3,5-di-tert-butyl-4-methoxyphenyl)-1,3-thiazol-2-yl]piperidin-1-yl}-2-oxoethyl)-3H-imidazo[4,5-b]pyridine;
(7) 3-[2-(4-{4-[3,5-bis(trifluoromethyl)phenyl]-5-bromo-1,3-thiazol-2-yl}piperidin-1-yl)-2-oxoethyl]-3H-imidazo[4,5-b]pyridine;
(8) 3-[2-(4-{4-[3,5-bis(trifluoromethyl)phenyl]-5-chloro-1,3-thiazol-2-yl}piperidin-1-yl)-2-oxoethyl]-3H-imidazo[4,5-b]pyridine;
(9) 3-[2-(4-{4-[3,5-bis(trifluoromethyl)phenyl]-5-fluoro-1,3-thiazol-2-yl}piperidin-1-yl)-2-oxoethyl]-3H-imidazo[4,5-b]pyridine;
(10) 3-(2-{4-[4-(3-bromo-5-tert-butylphenyl)-5-chloro-1,3-thiazol-2-yl]piperidin-1-yl}-2-oxoethyl)-3H-imidazo[4,5-b]pyridine;
(11) 3-(2-{4-[4-(3-tert-butyl-5-cyclopropylphenyl)-5-chloro-1,3-thiazol-2-yl]piperidin-1-yl}-2-oxoethyl)-3H-imidazo[4,5-b]pyridine;
(12) 3-(2-{4-[5-bromo-4-(3-tert-butyl-5-cyclopropylphenyl)-1,3-thiazol-2-yl]piperidin-1-yl}-2-oxoethyl)-3H-imidazo[4,5-b]pyridine;
(13) 4-{5-bromo-2-[1-(3H-imidazo[4,5-b]pyridin-3-ylacetyl)piperidin-4-yl]-1,3-thiazol-4-yl}-2,6-di-tert-butylphenol;
(14) 3-[2-(4-{4-[3-bromo-5-(trifluoromethoxy)-4-(trifluoromethyl)phenyl]-5-chloro-1,3-thiazol-2-yl}piperidin-1-yl)-2-oxoethyl]-3H-imidazo[4,5-b]pyridine;
(15) 3-[2-(4-{5-chloro-4-[3-cyclopropyl-5-(trifluoromethoxy)-4-(trifluoromethyl)phenyl]-1,3-thiazol-2-yl}piperidin-1-yl)-2-oxoethyl]-3H-imidazo[4,5-b]pyridine;
(16) 3-[2-(4-{5-chloro-4-[3-cyclopropyl-5-(trifluoromethoxy)phenyl]-1,3-thiazol-2-yl}piperidin-1-yl)-2-oxoethyl]-3H-imidazo[4,5-b]pyridine;
(17) 3-[2-(4-{5-chloro-4-[3-(1,1-difluoroethyl)-5-(trifluoromethoxy)phenyl]-1,3-thiazol-2-yl}piperidin-1-yl)-2-oxoethyl]-3H-imidazo[4,5-b]pyridine;
(18) 3-[2-(4-{4-[3-tert-butyl-5-(trifluoromethoxy)phenyl]-5-chloro-1,3-thiazol-2-yl}piperidin-1-yl)-2-oxoethyl]-3H-imidazo[4,5-b]pyridine;
(19) 3-(2-{4-[5-bromo-4-(3,5-di-tert-butylphenyl)-1,3-thiazol-2-yl]piperidin-1-yl}-2-oxoethyl)-3H-imidazo[4,5-b]pyridine;
(20) 3-{2-[4-(4-{3-bromo-5-[(trifluoromethyl)sulfonyl]phenyl}-5-chloro-1,3-thiazol-yl)piperidin-1-yl]-2-oxoethyl}-3H-imidazo[4,5-b]pyridine;
(21) 3-[2-(4-{4-[3-bromo-4-methoxy-5-(trifluoromethyl)phenyl]-5-chloro-1,3-thiazol-2-yl}piperidin-1-yl)-2-oxoethyl]-3H-imidazo[4,5-b]pyridine;
(22) 3-(2-{4-[4-(6-tert-butyl-1,1-dimethyl-2,3-dihydro-1H-inden-4-yl)-5-chloro-1,3-thiazol-2-yl]piperidin-1-yl}-2-oxoethyl)-3H-imidazo[4,5-b]pyridine;
(23) 3-[2-(4-{4-[3-bromo-4-methoxy-5-(1-methylcyclopropyl)phenyl]-5-chloro-1,3-thiazol-2-yl}piperidin-1-yl)-2-oxoethyl]-3H-imidazo[4,5-b]pyridine;
(24) 3-(2-{4-[5-chloro-4-(2,6-di-tert-butylpyrimidin-4-yl)-1,3-thiazol-2-yl]piperidin-1-yl}-2-oxoethyl)-3H-imidazo[4,5-b]pyridine;
(25) 3-[2-(4-{4-[2-tert-butyl-6-(trifluoromethyl)pyrimidin-4-yl]-5-chloro-1,3-thiazol-2-yl}piperidin-1-yl)-2-oxoethyl]-3H-imidazo[4,5-b]pyridine;
(26) 3-[2-(4-{4-[3-bromo-4-methoxy-5-(trifluoromethoxy)phenyl]-5-chloro-1,3-thiazol-2-yl}piperidin-1-yl)-2-oxoethyl]-3H-imidazo[4,5-b]pyridine;
(27) 3-[2-(4-{5-chloro-4-[3-cyclopropyl-4-methoxy-5-(trifluoromethoxy)phenyl]-1,3-thiazol-2-yl}piperidin-1-yl)-2-oxoethyl]-3H-imidazo[4,5-b]pyridine;
(28) 3-{2-[4-(4-{3-bromo-4-methoxy-5-[(trifluoromethyl)thio]phenyl}-5-chloro-1,3-thiazol-2-yl)piperidin-1-yl]-2-oxoethyl}-3H-imidazo[4,5-b]pyridine;
(29) 3-[2-(4-{5-bromo-4-[6-tert-butyl-2-(1-methylcyclopropyl)pyrimidin-4-yl]-1,3-thiazol-2-yl}piperidin-1-yl)-2-oxoethyl]-3H-imidazo[4,5-b]pyridine;
(30) 3-(2-{4-[5-chloro-4-(5,5,8,8-tetramethyl-5,6,7,8-tetrahydronaphthalen-2-yl)-1,3-thiazol-2-yl]piperidin-1-yl}-2-oxoethyl)-3H-imidazo[4,5-b]pyridine;
(31) 3-(2-{4-[5-bromo-4-(5,5,8,8-tetramethyl-5,6,7,8-tetrahydronaphthalen-2-yl)-1,3-thiazol-2-yl]piperidin-1-yl}-2-oxoethyl)-3H-imidazo[4,5-b]pyridine;
(32) [1-(2-{4-[5-chloro-4-(3,5-di-tert-butyl-4-methoxyphenyl)-1,3-thiazol-2-yl]piperidin-1-yl}-2-oxoethyl)-5-methyl-1H-1,2,4-triazol-3-yl]acetic acid;
(33) [1-(2-{4-[5-chloro-4-(3,5-di-tert-butyl-4-methoxyphenyl)-1,3-thiazol-2-yl]piperidin-1-yl}-2-oxoethyl)-5-methyl-1H-pyrazol-3-yl]acetic acid;
(34) {1-[2-(4-{4-[3,5-bis(trifluoromethyl)phenyl]-5-chloro-1,3-thiazol-2-yl}piperidin-1-yl)-2-oxoethyl]-5-methyl-1H-pyrazol-3-yl}acetic acid;
(35) [1-(2-{4-[5-bromo-4-(3,5-di-tert-butyl-4-methoxyphenyl)-1,3-thiazol-2-yl]piperidin-1-yl}-2-oxoethyl)-5-methyl-1H-pyrazol-3-yl]acetic acid;
(36) [1-(2-{4-[5-bromo-4-(3,5-di-tert-butyl-4-hydroxyphenyl)-1,3-thiazol-2-yl]piperidin-1-yl}-2-oxoethyl)-5-methyl-1H-pyrazol-3-yl]acetic acid;
(37) [4-bromo-1-(2-{4-[5-chloro-4-(3,5-di-tert-butyl-4-methoxyphenyl)-1,3-thiazol-2-yl]piperidin-1-yl}-2-oxoethyl)-5-methyl-1H-pyrazol-3-yl]acetic acid;
(38) 3-[1-(2-{4-[5-chloro-4-(3,5-di-tert-butyl-4-methoxyphenyl)-1,3-thiazol-2-yl]piperidin-1-yl}-2-oxoethyl)-5-methyl-1H-pyrazol-3-yl]propanoic acid;
(39) 3-{2-[4-(5-chloro-4-{3-(trifluoromethoxy)-5-[(trifluoromethyl)thio]phenyl}-1,3-thiazol-2-yl)piperidin-1-yl]-2-oxoethyl}-3H-imidazo[4,5-b]pyridine;
(41) [2-(2-{4-[5-chloro-4-(3,5-di-tert-butyl-4-methoxyphenyl)-1,3-thiazol-2-yl]piperidin-1-yl}-2-oxoethyl)-1,3-thiazol-4-yl]acetic acid;
(42) [2-(2-{4-[5-chloro-4-(3,5-di-tert-butyl-4-methoxyphenyl)-1,3-thiazol-2-yl]piperidin-1-yl}-2-oxoethyl)-5-methyl-1,3-thiazol-4-yl]acetic acid;
(43) [1-(2-{4-[5-chloro-4-(3,5-di-tert-butyl-4-methoxyphenyl)-1,3-thiazol-2-yl]piperidin-1-yl}-2-oxoethyl)-4,5-dimethyl-1H-pyrazol-3-yl]acetic acid;
(44) [2-(2-{4-[5-bromo-4-(3,5-di-tert-butyl-4-methoxyphenyl)-1,3-thiazol-2-yl]piperidin-1-yl}-2-oxoethyl)-5-methyl-1,3-thiazol-4-yl]acetic acid;
(45) 7-(2-{4-[5-chloro-4-(3,5-di-tert-butyl-4-methoxyphenyl)-1,3-thiazol-2-yl]piperidin-1-yl}-2-oxoethyl)-7H-purin-6-ol;
(46) 1-(2-{4-[5-chloro-4-(3,5-di-tert-butyl-4-methoxyphenyl)-1,3-thiazol-2-yl]piperidin-1-yl}-2-oxoethyl)-1,3-dihydro-2H-indol-2-one;
(47) [1-(2-{4-[5-chloro-4-(3,5-di-tert-butylphenyl)-1,3-thiazol-2-yl]piperidin-1-yl}-2-oxoethyl)-5-methyl-1H-pyrazol-3-yl]acetic acid;
(48) [1-(2-{4-[5-chloro-4-(3,5-di-tert-butyl-4-hydroxyphenyl)-1,3-thiazol-2-yl]piperidin-1-yl}-2-oxoethyl)-5-methyl-1H-pyrazol-3-yl]acetic acid;
(49) (1-{2-[4-(4-{3-tert-butyl-5-[(trifluoromethyl)thio]phenyl}-5-chloro-1,3-thiazol-2-yl)piperidin-1-yl]-2-oxoethyl}-5-methyl-1H-pyrazol-3-yl)acetic acid;
(50) 1-(2-{4-[5-chloro-4-(3,5-di-tert-butyl-4-methoxyphenyl)-1,3-thiazol-2-yl]piperidin-1-yl}-2-oxoethyl)-1,3-dihydro-2H-benzimidazol-2-one;
(51) (2-{2-[4-(4-{3-tert-butyl-5-[(trifluoromethyl)thio]phenyl}-5-chloro-1,3-thiazol-2-yl)piperidin-1-yl]-2-oxoethyl}-1,3-thiazol-4-yl)acetic acid;
(52) (2-{2-[4-(4-{3-tert-butyl-5-[(trifluoromethyl)thio]phenyl}-5-chloro-1,3-thiazol-2-yl)piperidin-1-yl]-2-oxoethyl}-4-methyl-1,3-thiazol-4-yl)acetic acid;
(53) [2-(2-{4-[5-chloro-4-(3,5-di-tert-butyl-4-methoxyphenyl)-1,3-thiazol-2-yl]piperidin-1-yl}-2-oxoethyl)-5-methyl-1,3-thiazol-4-yl](difluoro)acetic acid;
(54) {2-[2-(4-{4-[3-bromo-5-(trifluoromethoxy)-4-(trifluoromethyl)phenyl]-5-chloro-1,3-thiazol-2-yl}piperidin-1-yl)-2-oxoethyl]-5-methyl-1,3-thiazol-4-yl}acetic acid;
(55) {1-[2-(4-{4-[3-bromo-5-(trifluoromethoxy)-4-(trifluoromethyl)phenyl]-5-chloro-1,3-thiazol-2-yl}piperidin-1-yl)-2-oxoethyl]-5-methyl-1H-pyrazol-3-yl}acetic acid;
(56) 1-{2-[4-(4-{3-tert-butyl-5-[(trifluoromethyl)thio]phenyl}-5-chloro-1,3-thiazol-2-yl)piperidin-1-yl]-2-oxoethyl}-1,3-dihydro-2H-benzimidazol-2-one;
(57) 1-{2-[4-(5-bromo-4-{3-tert-butyl-5-[(trifluoromethyl)thio]phenyl}-1,3-thiazol-2-yl)piperidin-1-yl]-2-oxoethyl}-1,3-dihydro-2H-benzimidazol-2-one; and
(58) (1-{2-[4-(5-bromo-4-{3-tert-butyl-5-[(trifluoromethyl)thio]phenyl}-1,3-thiazol-2-yl)piperidin-1-yl]-2-oxoethyl}-5-methyl-1H-pyrazol-3-yl)acetic acid;
(59) [1-(2-{4-[5-chloro-4-(3,5-di-tert-butyl-4-methoxyphenyl)-1,3-thiazol-2-yl]piperidin-1-yl}-2-oxoethyl)-4-iodo-5-methyl-1H-pyrazol-3-yl]acetic acid;
(60) 3-(2-{4-[5-chloro-4-(2,6-di-tert-butylpyrimidin-4-yl)-1,3-thiazol-2-yl]piperidin-1-yl}-2-oxoethyl)-3H-imidazo[4,5-b]pyridine;
(61) 1-(2-{4-[5-chloro-4-(2,6-di-tert-butylpyrimidin-4-yl)-1,3-thiazol-2-yl]piperidin-1-yl}-2-oxoethyl)-1,3-dihydro-2H-benzimidazol-2-one;
(62) [1-(2-{4-[5-chloro-4-(2,6-di-tert-butylpyrimidin-4-yl)-1,3-thiazol-2-yl]piperidin-1-yl}-2-oxoethyl)-5-methyl-1H-pyrazol-3-yl]acetic acid;
(63) 1-(2-{4-[5-bromo-4-(2,6-di-tert-butylpyrimidin-4-yl)-1,3-thiazol-2-yl]piperidin-1-yl}-2-oxoethyl)-1,3-dihydro-2H-benzimidazol-2-one;
(64) 3-[5-methyl-1-(2-oxo-2-{4-[4-(5,5,8,8-tetramethyl-5,6,7,8-tetrahydronaphthalen-2-yl)-1,3-thiazol-2-yl]piperidin-1-yl}ethyl)-1H-pyrazol-3-yl]propanoic acid;
(65) [5-methyl-1-(2-oxo-2-{4-[4-(5,5,8,8-tetramethyl-5,6,7,8-tetrahydronaphthalen-2-yl)-1,3-thiazol-2-yl]piperidin-1-yl}ethyl)-1H-pyrazol-3-yl]acetic acid;
(66) 3-(2-{4-[4-(2,6-di-tert-butylpyrimidin-4-yl)-1,3-thiazol-2-yl]piperidin-1-yl}-2-oxoethyl)-3H-imidazo[4,5-b]pyridine;
(67) 3-[2-(4-{4-[2-tert-butyl-6-(trifluoromethyl)pyrimidin-4-yl]-1,3-thiazol-2-yl}piperidin-1-yl)-2-oxoethyl]-3H-imidazo[4,5-b]pyridine;
(68) 3-[2-(4-{4-[3,5-bis(trifluoromethyl)phenyl]-1,3-thiazol-2-yl}piperidin-1-yl)-2-oxoethyl]-3H-imidazo[4,5-b]pyridine;
(69) 3-[2-(4-{4-[3-bromo-4-methoxy-5-(trifluoromethoxy)phenyl]-1,3-thiazol-2-yl}piperidin-1-yl)-2-oxoethyl]-3H-imidazo[4,5-b]pyridine;
(70) [2-(2-{4-[4-(3,5-di-tert-butyl-4-methoxyphenyl)-1,3-thiazol-2-yl]piperidin-1-yl}-2-oxoethyl)-5-methyl-1,3-thiazol-4-yl]acetic acid;
(71) 3-[2-(4-{4-[2-tert-butyl-6-(1-methylcyclopropyl)pyrimidin-4-yl]-1,3-thiazol-2-yl}piperidin-1-yl)-2-oxoethyl]-3H-imidazo[4,5-b]pyridine;
(72) [1-(2-{4-[4-(2,6-di-tert-butylpyrimidin-4-yl)-1,3-thiazol-2-yl]piperidin-1-yl}-2-oxoethyl)-5-methyl-1H-pyrazol-3-yl]acetic acid;
(73) 3-(2-{4-[4-(3,5-di-tert-butyl-4-methoxyphenyl)-1,3-thiazol-2-yl]piperidin-1-yl}-2-oxoethyl)-3H-imidazo[4,5-b]pyridine;
(74) 3-[2-(4-{4-[6-tert-butyl-2-(1-methylcyclopropyl)pyrimidin-4-yl]-1,3-thiazol-2-yl}piperidin-1-yl)-2-oxoethyl]-3H-imidazo[4,5-b]pyridine;
(75) 3-(2-{4-[4-(3,5-di-tert-butylphenyl)-1,3-thiazol-2-yl]piperidin-1-yl}-2-oxoethyl)-3H-imidazo[4,5-b]pyridine; and
(76) 3-(2-oxo-2-{4-[4-(5,5,8,8-tetramethyl-5,6,7,8-tetrahydronaphthalen-2-yl)-1,3-thiazol-2-yl]piperidin-1-yl}ethyl)-3H-imidazo[4,5-b]pyridine.
or a pharmaceutically acceptable salt of any of the above.

17. A pharmaceutical composition comprising a compound according to claim 1 in combination with a pharmaceutically acceptable carrier.

18. A method for treating a disease or condition mediated by CXCR3 chemokine receptor comprising administering to a patient in need of such treatment a therapeutically effective amount of a compound according to claim 1.

19. The method according to claim 18 wherein the disease or condition is selected from the group consisting of: acute and chronic transplant rejection, psoriasis, rheumatoid arthritis and multiple sclerosis.
